(19) United States
(12) Patent Application Publication
(10) Pub. No.: US 2026/0265271 A1
(43) Pub. Date: Sep. 10, 2026

(54) Cereblon Ligands and Uses Thereof

(71) Applicants: REGENTS OF THE UNIVERSITY OF MICHIGAN, Ann Arbor, MI (US); Oncopia Therapeutics, Inc. d/b/a SK Life Science Labs, King Of Prussia, PA (US)

(72) Inventors: Shaomeng Wang, Superior Township, MI (US); Rohan Rej, Ann Arbor, MI (US); Zhixiang Chen, Ann Arbor, MI (US); Longchuan Bai, Ann Arbor, MI (US); Ranjan Kumar Acharyya, Ann Arbor, MI (US); Dimin Wu, Ann Arbor, MI (US); Paul Kirchhoff, Saline, MI (US); Guozhang Xu, New York, NY (US); Zhenwu Li, New York, NY (US)

(21) Appl. No.: 18/881,932

(22) PCT Filed: Jul. 11, 2023
(86) PCT No.: PCT/US2023/027344

§ 371 Date: Jan. 7, 2025

Related U.S. Application Data
(60) Provisional application No. 63/388,297, filed on Jul. 12, 2022.
(60) Provisional application No. 63/388,299, filed on Jul. 12, 2022.
(60) Provisional application No. 63/388,300, filed on Jul. 12, 2022.
(60) Provisional application No. 63/388,302, filed on Jul. 12, 2022.
(60) Provisional application No. 63/408,744, filed on Sep. 21, 2022.
(60) Provisional application No. 63/408,758, filed on Sep. 21, 2022.
(60) Provisional application No. 63/408,601, filed on Sep. 21, 2022.
(60) Provisional application No. 63/408,633, filed on Sep. 21, 2022.
(60) Provisional application No. 63/412,164, filed on Sep. 30, 2022.
(60) Provisional application No. 63/427,277, filed on Nov. 22, 2022.
(60) Provisional application No. 63/460,734, filed on Apr. 20, 2023.

(51) Int. Cl. C07D 498/14; A61K 31/4985; A61K 31/5383; A61K 47/55; C07D 487/14; C07D 498/04; C07D 519/00

(52) U.S. Cl. CPC C07D 498/14; A61K 31/4985; A61K 31/5383; A61K 47/55; C07D 487/14; C07D 498/04; C07D 519/00

(57) ABSTRACT

Described herein are compounds or conjugates of Formulae II and I and their pharmaceutically acceptable salts, solvates, or stereoisomers, as well as their uses (e.g., as cereblon-binding agents or bifunctional degraders for degrading certain proteins).

### RELATED APPLICATIONS

This application claims the benefit of and priority to U.S. Provisional Application No. 63/412,164, filed Sep. 30, 2022; U.S. Provisional Application No. 63/388,300, filed Jul. 12, 2022; U.S. Provisional Application No. 63/408,744, filed Sep. 21, 2022; U.S. Provisional Application No. 63/427,277, filed Nov. 22, 2022; U.S. Provisional Application No. 63/460,734, filed Apr. 20, 2023; U.S. Provisional Application No. 63/388,302, filed Jul. 12, 2022; U.S. Provisional Application No. 63/408,758, filed Sep. 21, 2022; U.S. Provisional Application No. 63/388,297, filed Jul. 12, 2022; U.S. Provisional Application No. 63/408,601, filed Sep. 21, 2022; U.S. Provisional Application No. 63/388,299, filed Jul. 12, 2022; and U.S. Provisional Application No. 63/408,633, filed Sep. 21, 2022; the contents of each of which are incorporated herein by reference in their entireties.

### BACKGROUND

Cereblon (CRBN), a component of the DDB1-CUL4a-Rocl ubiquitin ligase complex, is a molecular target of immunomodulatory agents such as thalidomide, lenalidomide, and pomalidomide. Inhibition of CRBN ubiquitination by these agents may allow CRBN to accumulate, leading to the increased cullin-4 RING E3 ligase-mediated degradation of target proteins.

The discovery process of CRBN type E3 ligase ligand is related to the study of thalidomide's mechanism of action. In 2010, while studying the toxicity of thalidomide, scientists discovered that cereblon is a binding protein of thalidomide (Science 2010, 327, 1345). Cerebellar protein is part of the E3 ubiquitin ligase protein complex, which acts as a substrate receptor to select ubiquitinated proteins. The study shows that thalidomide-cerebellar protein binding in vivo may be the cause of thalidomide teratogenicity. Subsequent studies found that the compound and related structures can be used as anti-inflammatory agents, anti-angiogenic agents and anti-cancer agents. Lenalidomide and pomalidomide obtained by further modification of the structure of thalidomide have greatly improved their safety and significantly reduced their teratogenic effects. Lenalidomide has been approved by the FDA in 2006 for marketing. Two groundbreaking papers published in Science in 2014 pointed out that lenalidomide works by degrading two special B cell transcription factors, Ikaros family zinc finger structural proteins 1 and 3 (IKZF1 and IKZF3). The structure of thalidomide may be combined with the E3 ubiquitin ligase protein complex of the cerebellar protein to further play a role in degrading the target protein (Science, 2014, 343, 301; Science, 2014, 343, 305).

On this basis, CRBN ligands are widely used in protein degradation, and a series of PROTAC molecules based on CRBN ligands have been developed. Due to the influence of CRBN ligand itself on the target point, it may additionally degrade zinc finger domain proteins. Therefore, the design and synthesis of new and highly selective CRBN ligands is also particularly important in the synthesis of PROTAC molecules.

### SUMMARY

In certain aspects, the present disclosure provides compounds or conjugates of Formula II:

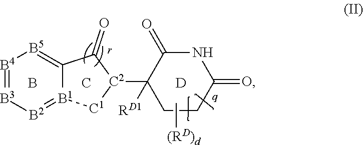

and pharmaceutically acceptable salts, solvates, or stereoisomers thereof, wherein each of the variables in Formula II is described, embodied, and exemplified herein.

In certain aspects, the present disclosure provides compounds or conjugates of Formula I:

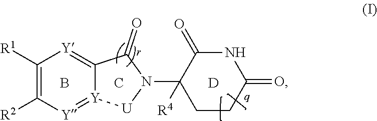

and pharmaceutically acceptable salts, solvates, or stereoisomers thereof, wherein each of the variables in Formula I is described, embodied, and exemplified herein.

In certain aspects, the present disclosure provides pharmaceutical compositions comprising a compound disclosed herein, and a pharmaceutically acceptable excipient.

In certain aspects, provided herein are methods of binding cereblon E3 ubiquitin ligase protein complex in a subject or biological sample comprising administering a compound described herein to the subject or contacting the biological sample with a compound described herein.

In certain aspects, provided herein are uses of a compound described herein in the manufacture of a medicament for binding cereblon E3 ubiquitin ligase protein complex in a subject or biological sample.

In certain aspects, provided herein are compounds described herein for use in binding cereblon E3 ubiquitin ligase protein complex in a subject or biological sample.

In certain aspects, provided herein are methods of degrading a protein in a subject or biological sample comprising administering a compound described herein to the subject or contacting the biological sample with a compound described herein.

In certain aspects, provided herein are uses of a compound described herein in the manufacture of a medicament for degrading a protein in a subject or biological sample.

In certain aspects, provided herein are compounds described herein for use in degrading a protein in a subject or biological sample.

In certain aspects, provided herein are methods of reducing a protein in a subject or biological sample comprising administering a compound described herein to the subject or contacting the biological sample with a compound described herein.

In certain aspects, provided herein are uses of a compound described herein in the manufacture of a medicament for reducing a protein in a subject or biological sample.

In certain aspects, provided herein are compounds described herein for use in reducing a protein in a subject or biological sample.

In certain aspects, provided herein are methods of treating or preventing a disease or disorder a subject in need thereof, comprising administering to the subject a compound described herein.

In certain aspects, provided herein are uses of a compound described herein in the manufacture of a medicament for treating or preventing a disease or disorder in a subject in need thereof.

In certain aspects, provided herein are compounds described herein for use in treating or preventing a disease or disorder in a subject in need thereof.

### DETAILED DESCRIPTION

The present disclosure relates to compounds that potentially show cereblon-binding activity, and compounds that bifunctional degraders comprising such compound, as well as pharmaceutical compositions thereof. The present disclosure further relates to methods of degrading a protein in a subject or biological sample comprising administering a compound described herein to the subject or contacting the biological sample with a compound described herein. The present disclosure also relates to methods of treating or preventing a disease or disorder a subject in need thereof, comprising administering to the subject a compound described herein.

### Compounds of the Application

### Cereblon Ligands

In certain aspects, the present disclosure provides compounds of Formula II:

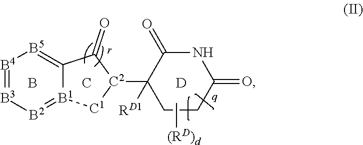

and pharmaceutically acceptable salts, solvates, or stereoisomers thereof, wherein:
B² is N or CR^{B2};
B³ is N or CR^{B3};
B⁴ is N or CR^{B4};
B⁵ is N or CR^{B5};
one of R^{B2} and R^{B3}, R^{B3} and R^{B4}, and R^{B4} and R^{B5}, together with the carbon atoms to which they are bonded, form Ring A, wherein Ring A is optionally substituted 7- to 16-membered fused carbocycle or optionally substituted 7- to 16-membered fused heterocycle;
the remaining two of R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}. —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
--- denotes an optional covalent bond between B¹ and C¹;
i) when the bond between B¹ and C¹ is present:
   r is 1;
   B¹ is C;
   C¹ is —C(R^{C1})₂— or —C(═O)—;
   each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
   two R^{C1}, together with the carbon atom to which they are attached, form C₃₋₆ carbocycle or 3- to 6-membered heterocycle, wherein the carbocycle or heterocycle is optionally substituted with one or more R^{u}; and
   C² is N;
ii) when the bond between B¹ and C¹ is absent:
   r is 0 or 1;
   B¹ is N or CR^{B1};
   R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
   C¹ is absent; or
   C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
   C² is N or O;
   wherein i) when C² is N, then C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; and ii) when C² is O, then C¹ is absent;
R^{D1} is hydrogen, deuterium, or C₁₋₆ alkyl optionally substituted with one or more R^{u};
q is an integer from 0 to 2,
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
d is an integer selected from 0 to 5,
   wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each Rb is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl, wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the compound is not a compound selected from

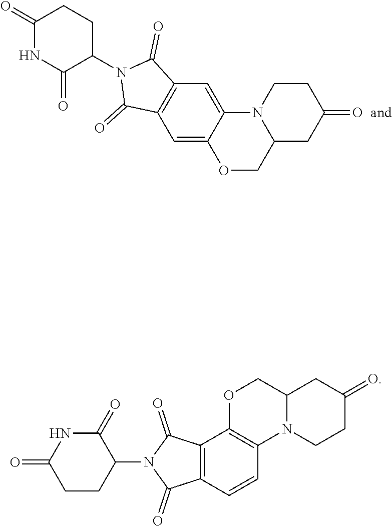 [SMILES: O=C1CCN2c3cc4c(cc3OCC2C1)C(=O)N(C1CCC(=O)NC1=O)C4=O ; O=C1CCN2c3ccc4c(c3OCC2C1)C(=O)N(C1CCC(=O)NC1=O)C4=O]

In certain embodiments, the compound is not a compound selected from

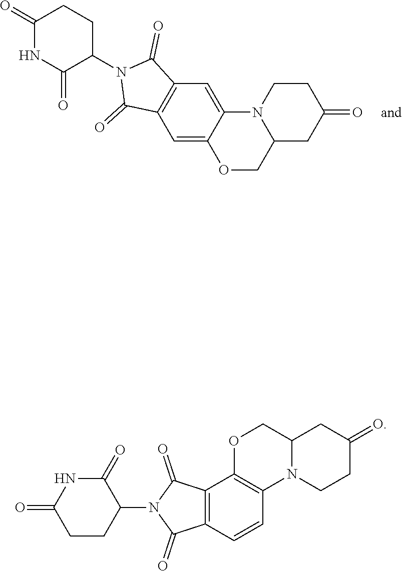 [SMILES: O=C1CCN2c3cc4c(cc3OCC2C1)C(=O)N(C1CCC(=O)NC1=O)C4=O ; O=C1CCN2c3ccc4c(c3OCC2C1)C(=O)N(C1CCC(=O)NC1=O)C4=O]

In certain embodiments, the bond between B¹ and C¹ is present.

In certain embodiments, the compound of Formula II is a compound of Formula II-1

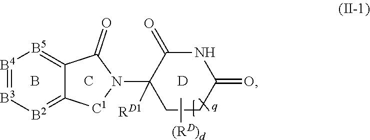

In certain embodiments, when the bond between B¹ and C¹ is present, then B¹ is C.

In certain embodiments, when the bond between B¹ and C¹ is present, then r is 0 or 1.

In certain embodiments, when the bond between B¹ and C¹ is present, then C¹ is —C(R^{C1})₂—, —C(═O)—, —(C═O)—N(R^{C1′})—*, or —N═C(R^{C1′})—*.

In certain embodiments, when the bond between B¹ and C¹ is present, then C² is N.

In certain embodiments, R^{C1′} is H or C₁₋₆ alkyl optionally substituted with one or more R^{u}, and * denotes attachment to Ring B.

In certain embodiments, each R^{C1′} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the alkyl, alkoxy, alkylamino, carbocyclyl, or heterocyclyl is optionally substituted with one or more R^{u}.

In certain embodiments, two R^{C1}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (CG), cyclohexenyl (CG), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more R^{u}.

In certain embodiments, the bond between B¹ and C¹ is absent.

In certain embodiments, the compound of Formula II is a compound of Formula II-2

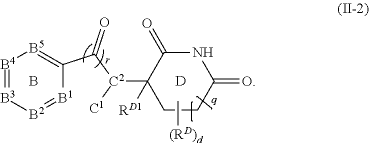

In certain embodiments, when the bond between B¹ and C¹ is present, then r is 0 or 1.

In certain embodiments, when the bond between B¹ and C¹ is absent, then B¹ is N or CR^{B1}.

In certain embodiments, R^{B1} is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{B1} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{B1} is hydrogen or halogen.

In certain embodiments, when the bond between B¹ and C¹ is absent, then C¹ is absent.

In certain embodiments, when the bond between B¹ and C¹ is absent, then C¹ is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, when the bond between B¹ and C¹ is absent, then C² is N or O. In certain embodiments, when the bond between B¹ and C¹ is absent, then C² is N. In certain embodiments, when the bond between B¹ and C¹ is absent, then C² is O.

In certain embodiments, when the bond between B¹ and C¹ is absent, and C² is N, then C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, when the bond between B¹ and C¹ is absent, and C² is O, then C¹ is absent.

In certain embodiments, B² is N or CR^{B2}. In certain embodiments, B² is N. In certain embodiments, B² is CR^{B2}.

In certain embodiments, B³ is N or CR^{B3}. In certain embodiments, B³ is N. In certain embodiments, B³ is CR^{B3}.

In certain embodiments, B⁴ is N or CR^{B4}. In certain embodiments, B² is N. In certain embodiments, B⁴ is CR^{B4}.

In certain embodiments, B⁵ is N or CR^{B5}. In certain embodiments, B² is N. In certain embodiments, B⁵ is CR^{B5}.

In certain embodiments, one of B², B³, B⁴, and B⁵ is N. In certain embodiments, two of B², B³, B⁴, and B⁵ are N.

In certain embodiments, one of R^{B2} and R^{B3}, R^{B3} and R^{B4}, and R^{B4} and R^{B5}, together with the carbon atoms to which they are bonded, form Ring A, wherein Ring A is optionally substituted 7- to 16-membered fused heterocycle.

In certain embodiments, R^{B1} and R^{B2}, together with the carbon atoms to which they are bonded, form Ring A.

In certain embodiments, R^{B2} and R^{B3}, together with the carbon atoms to which they are bonded, form Ring A.

In certain embodiments, R^{B3} and R^{B4}, together with the carbon atoms to which they are bonded, form Ring A.

In certain embodiments, the remaining two of R^{B2}, R^{B3}, R^{B4}, and R^{B5}, when applicable, are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)). C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, the remaining two of R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, the remaining two of R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, the remaining two of R^{B2}, R^{B5}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, the remaining two of R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each of the remaining two of R^{B2}, R^{B3}, R^{B4}, and R^{B5}, when applicable, is hydrogen. In certain embodiments, each of the remaining two of R^{B2}, R^{B3}, R^{B4}, and R^{B5}, when applicable, is hydrogen.

In certain embodiments, each of R^{B4} and R^{B5} is hydrogen. In certain embodiments, each of R^{B2} and R^{B5} is hydrogen.

In certain embodiments, Ring A is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, Ring A is optionally substituted with one or more R^{u}, R^{A}, R^{Ax}, R^{A1}, or R^{A2}.

In certain embodiments, R^{u} is R^{A}. In certain embodiments, R^{u} is R^{Ax}. In certain embodiments, R^{u} is R^{A1}. In certain embodiments, R^{u} is R^{A2}.

In certain embodiments, R^{A} is R^{A1}. In certain embodiments, R^{A} is R^{A2}.

In certain embodiments,
Ring A is

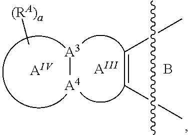

wherein:
Ring A^{III} and Ring A^{IV} are independently C₄₋₈ carbocycle or 4- to 8-membered heterocycle; wherein at least one of Ring A^{III} and Ring A^{IV} is 4- to 8-membered heterocycle;
A³ and A⁴ are independently C, CR^{Ax}, or N;
R^{Ax} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits,
   wherein each R^{A} may independently be present on either Ring A^{III} or Ring A^{IV}.

In certain embodiments, Ring A^{III} and Ring A^{IV} are independently C₄₋₈ carbocycle (e.g., cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), or cyclooctenyl (C₈)) or 4- to 8-membered heterocycle (e.g., heterocycle comprising one or two 4- to 8-membered rings and 1-3 heteroatoms selected from N, O, and S).

In certain embodiments, Ring A^{III} is 5- to 8-membered heterocycle comprising at least two nitrogen atoms.

In certain embodiments, Ring A^{III} is 5- to 8-membered heterocycle comprising two nitrogen atoms.

In certain embodiments, Ring A^{III} is 5- to 8-membered heterocycle comprising one nitrogen atom and one oxygen atom.

In certain embodiments, Ring A^{IV} is 5- to 8-membered heterocycle comprising at least two nitrogen atoms.

In certain embodiments, Ring A^{IV} is 5- to 8-membered heterocycle comprising two nitrogen atoms.

In certain embodiments, Ring A^{IV} is 5- to 8-membered heterocycle comprising one nitrogen atom and one oxygen atom.

In certain embodiments, A³ and A⁴ are independently C, CR^{Ax}, or N.

In certain embodiments, one of A³ and A⁴ is CR^{Ax}, and the other one of A³ and A⁴ is N.

In certain embodiments, each R^{Ax} is independently hydrogen, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{Ax} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{Ax} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{Ax} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{Ax} is hydrogen.

In certain embodiments,
Ring A is

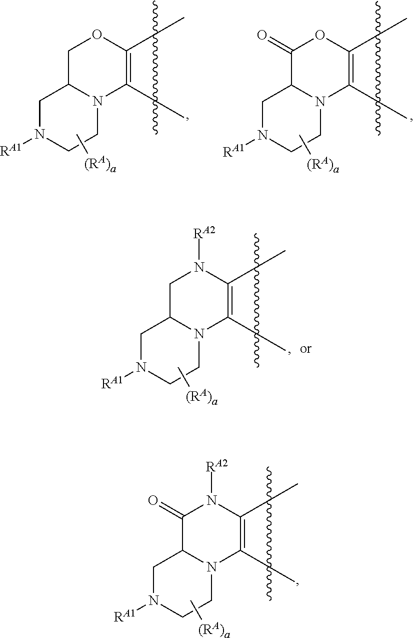

wherein:
R^{A1} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), —(C₁₋₆ alkylene)-(5- to 10-membered heteroaryl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}; or
R^{A1} is an amino-protecting group; and
R^{A2} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}; or
R^{A2} is an amino-protecting group.

In certain embodiments, the compound is a compound of Formula II-1-a-i, II-1-a-ii, II-1-a-iii, or II-1-a-iv

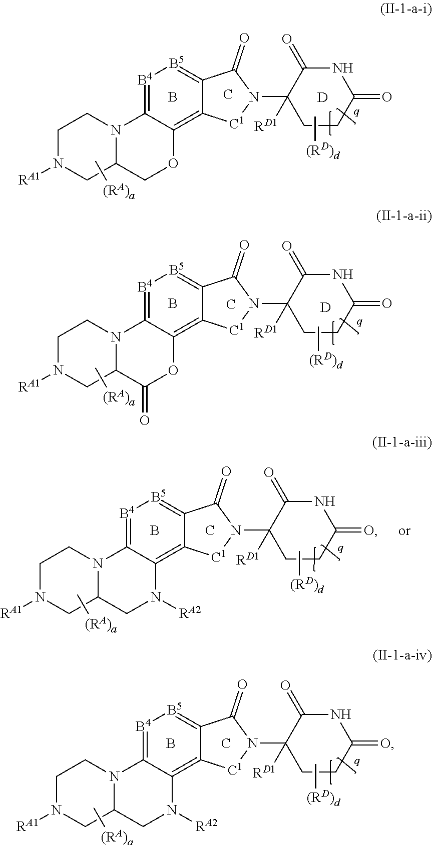

or a pharmaceutically acceptable salt, solvate, or stereoisomer thereof.

In certain embodiments, the compound is a compound of Formula II-2-a-i, II-2-a-ii, II-2-a-iii, or II-2-a-iv:

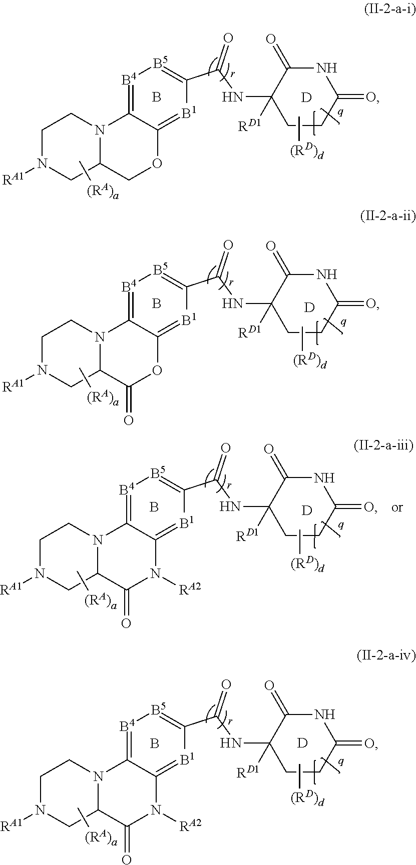

or a pharmaceutically acceptable salt, solvate, or stereoisomer thereof.

In certain embodiments, the compound is a compound of Formula II-1-a-v, II-1-a-vi, II-1-a-vii, or II-1-a-viii:

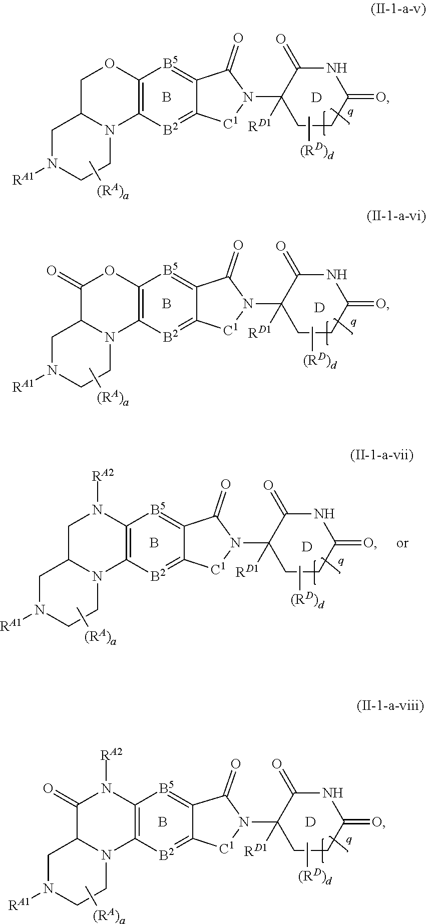

or a pharmaceutically acceptable salt, solvate, or stereoisomer thereof.

In certain embodiments, the compound is a compound of Formula II-2-a-v, II-2-a-vi, II-2-a-vii, or II-2-a-viii

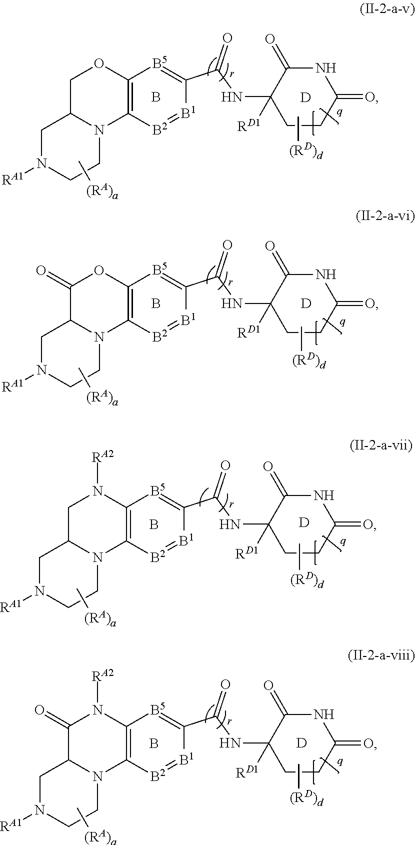

or a pharmaceutically acceptable salt, solvate, or stereoisomer thereof.

In certain embodiments, R^{A1} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), —(C₁₋₆ alkylene)-(5- to 10-membered heteroaryl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}.

In certain embodiments, R^{A1} is hydrogen. C₁₋₆ alkyl, C₃₋₆ carbocyclyl, 3- to 6-membered heterocyclyl, C₆ aryl, 5- to 6-membered heteroaryl, —(C₁₋₆ alkylene)-(C₃₋₆ carbocyclyl), —(C₁₋₆ alkylene)-(3- to 6-membered heterocyclyl), —(C₁₋₆ alkylene)-(C₆ aryl), —(C₁₋₆ alkylene)-(5- to 6-membered heteroaryl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, carbocyclyl, heterocyclyl, aryl, or heteroaryl is optionally substituted with one or more R^{u}.

In certain embodiments, R^{A1} is hydrogen. C₁₋₆ alkyl, C₃₋₆ carbocyclyl, 3- to 6-membered heterocyclyl, C₆ aryl, 5- to 6-membered heteroaryl, —(C₁₋₆ alkylene)-(C₃₋₆ carbocyclyl), —(C₁₋₆ alkylene)-(3- to 6-membered heterocyclyl), —(C₁₋₆ alkylene)-(C₆ aryl), —(C₁₋₆ alkylene)-(5- to 6-membered heteroaryl), —C(═O)R^{a}, —C(═O)OR^{b}, or —C(═O)NR^{c}R^{d}, wherein the alkyl, alkylene, carbocyclyl, heterocyclyl, aryl, or heteroaryl is optionally substituted with one or more R^{u}.

In certain embodiments, R^{A1} is hydrogen, C₁₋₆ alkyl, —(C₁₋₆ alkylene)-(C₃₋₆ carbocyclyl), —(C₁₋₆ alkylene)-(3- to 6-membered heterocyclyl), —(C₁₋₆ alkylene)-(C₆ aryl), —(C₁₋₆ alkylene)-(5- to 6-membered heteroaryl), —C(═O)R^{a}, or —C(═O)OR^{b}, wherein the alkyl, alkylene, carbocyclyl, heterocyclyl, aryl, or heteroaryl is optionally substituted with one or more R^{u}.

In certain embodiments, R^{A1} is an amino-protecting group.

In certain embodiments, R^{A2} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{A2} is hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR′R^{d}, —C(═O)R^{A}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{A2} is hydrogen, C₁₋₆ alkyl, C₂₋₆ alkenyl, C₂₋₆ alkynyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{A2} is hydrogen. C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R^{A2} is hydrogen or C₁₋₆ alkyl.

In certain embodiments, R^{A2} is an amino-protecting group.

In certain embodiments, each R^{A} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, a is 0. In certain embodiments, a is 1. In certain embodiments, a is 2. In certain embodiments, a is 3. In certain embodiments, a is 4, as valency permits. In certain embodiments, a is 5, as valency permits. In certain embodiments, a is 6, as valency permits. In certain embodiments, a is 7, as valency permits. In certain embodiments, a is 8, as valency permits.

In certain embodiments, a is 0, 1, or 2.

In certain embodiments, R^{D1} is hydrogen, deuterium, or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, q is 0. In certain embodiments, q is 1. In certain embodiments, q is 2.

In certain embodiments, each R^{D} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, d is 0. In certain embodiments, d is 1. In certain embodiments, d is 2. In certain embodiments, d is 3. In certain embodiments, d is 4. In certain embodiments, d is 5.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂₋₆ alkynyl, C₃₋₆ carbocyclyl, or 3- to 6-membered heterocyclyl.

In certain embodiments, each Rb is independently hydrogen, C_{1-_}alkyl, C₃₋₆carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6 -membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, two geminal R^{u}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain aspects, the present disclosure provides compounds of Formula I:

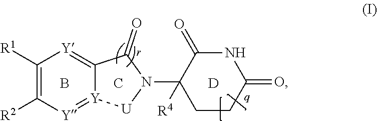

and pharmaceutically acceptable salts, solvates, or stereoisomers thereof,
wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NRCS(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R¹ and R², together with the intervening carbon atoms, form optionally substituted 7- to 16-membered fused heterocycle;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form optionally substituted 7- to 16-membered fused heterocycle;
provided that either R¹ and R² or R² and R³ form optionally substituted 7- to 16-membered fused heterocycle,
Y′ is N or CR^{Y′};
R^{Y′} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12 -membered heterocyclyl, wherein the alkyl, alkoxy, alkylamino, alkenyl, alkynyl, aryl, heteroaryl, carbocyclyl, or heterocyclyl is optionally substituted with one or more R^{u};
[symbol] denotes an optional covalent bond between Y and U;
when the bond between Y and U is absent:
r is 0 or 1;
Y is N or CR^{Y};
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
U is hydrogen or C₁₋₆ alkyl optionally substituted with one or more R^{u};
when the bond between Y and U is present:
r is 1;
Y is C;
U is —CH₂—, —C(═O)—, —(C═O)—N(R^{U})—*, —N═C(R^{U})—*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and
q is an integer from 0 to 2,
   wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the compound is not

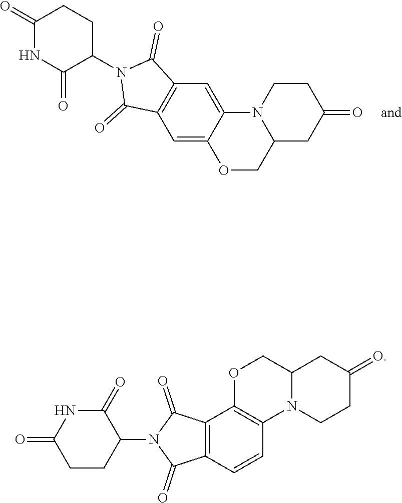 [SMILES: O=C1CCN2c3cc4c(cc3OCC2C1)C(=O)N(C1CCC(=O)NC1=O)C4=O ; O=C1CCN2c3ccc4c(c3OCC2C1)C(=O)N(C1CCC(=O)NC1=O)C4=O]

In certain embodiments, the compound is not

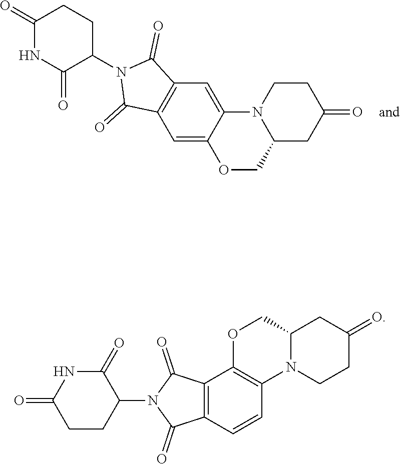 [SMILES: O=C1CCN2c3cc4c(cc3OC[C@H]2C1)C(=O)N(C1CCC(=O)NC1=O)C4=O ; O=C1CCN2c3ccc4c(c3OC[C@@H]2C1)C(=O)N(C1CCC(=O)NC1=O)C4=O]

In certain embodiments, the compound of Formula I is a compound of Formula I-1

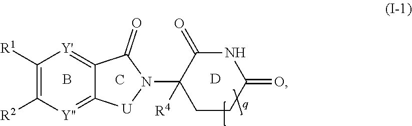

or a pharmaceutically acceptable salt, solvate, or stereoisomer thereof.

In certain embodiments, U is —CH₂— or —C(═O)—.

In certain embodiments, the compound of Formula I is a compound of Formula I-2

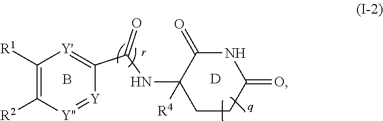

or a pharmaceutically acceptable salt, solvate, or stereoisomer thereof.

In certain embodiments, Y is N.

In certain embodiments, Y is CR^{Y}.

In certain embodiments, R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y} is hydrogen, halogen, or C₁₋₆ alkoxy.

In certain embodiments, R¹ and R², together with the intervening carbon atoms, form optionally substituted 8- to 12-membered fused bicyclic heterocycle.

In certain embodiments, the 7- to 16-membered or 8- to 12-membered fused heterocycle is optionally substituted with one or more R^{u}. In certain embodiments, the 7- to 16-membered or 8- to 12-membered fused heterocycle is optionally substituted with one or more Rⁱ. In certain embodiments, the 7- to 16-membered or 8- to 12-membered fused heterocycle is optionally substituted with one or more R⁵. In certain embodiments, the 7- to 16-membered or 8- to 12-membered fused heterocycle is optionally substituted with one or more R⁶.

In certain embodiments, R^{u} is Rⁱ. In certain embodiments, R^{u} is R⁵. In certain embodiments, R^{u} is R⁶.

In certain embodiments, Rⁱ is R⁵. In certain embodiments, R¹ is R⁶.

In certain embodiments, the 7- to 16-membered or 8- to 12-membered fused heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, — C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, R¹ and R², together with the intervening carbon atoms, form optionally substituted 8- to 12-membered fused bicyclic heterocycle of formula

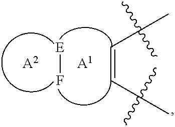

wherein E and F are independently CH or N.

In certain embodiments, Ring A¹ is optionally substituted 5- to 7-membered heterocycle.

In certain embodiments, Ring A¹ is optionally substituted 5- to 7-membered heterocycle comprising one nitrogen atom. In certain embodiments, Ring A¹ is optionally substituted 5- to 7-membered heterocycle comprising two nitrogen atoms.

In certain embodiments, Ring A¹ is optionally substituted 5-membered heterocycle. In certain embodiments, Ring A¹ is optionally substituted 6-membered heterocycle. In certain embodiments, Ring A¹ is optionally substituted 7-membered heterocycle.

In certain embodiments, Ring A¹ is optionally substituted 5-membered heterocycle comprising one nitrogen atom. In certain embodiments, Ring A¹ is optionally substituted 6-membered heterocycle comprising one nitrogen atom. In certain embodiments, Ring A¹ is optionally substituted 7-membered heterocycle comprising one nitrogen atom.

In certain embodiments, Ring A¹ is optionally substituted 5-membered heterocycle comprising two nitrogen atoms. In certain embodiments, Ring A¹ is optionally substituted 6-membered heterocycle comprising two nitrogen atoms. In certain embodiments, Ring A¹ is optionally substituted 7-membered heterocycle comprising two nitrogen atoms.

In certain embodiments, Ring A¹ is optionally substituted with one or more R^{u}. In certain embodiments, Ring A¹ is optionally substituted with one or more R¹. In certain embodiments, Ring A¹ is optionally substituted with one or more R⁵. In certain embodiments, Ring A¹ is optionally substituted with one or more R⁶.

In certain embodiments, Ring A² is optionally substituted 5- to 7-membered heterocycle.

In certain embodiments, Ring A² is optionally substituted 5- to 7-membered heterocycle comprising one nitrogen atom. In certain embodiments, Ring A² is optionally substituted 5- to 7-membered heterocycle comprising two nitrogen atoms.

In certain embodiments, Ring A² is optionally substituted 5-membered heterocycle. In certain embodiments, Ring A² is optionally substituted 6-membered heterocycle. In certain embodiments, Ring A² is optionally substituted 7-membered heterocycle.

In certain embodiments, Ring A² is optionally substituted 5-membered heterocycle comprising one nitrogen atom. In certain embodiments, Ring A² is optionally substituted 6-membered heterocycle comprising one nitrogen atom. In certain embodiments, Ring A² is optionally substituted 7-membered heterocycle comprising one nitrogen atom.

In certain embodiments, Ring A² is optionally substituted 5-membered heterocycle comprising two nitrogen atoms. In certain embodiments, Ring A² is optionally substituted 6-membered heterocycle comprising two nitrogen atoms. In certain embodiments, Ring A² is optionally substituted 7-membered heterocycle comprising two nitrogen atoms.

In certain embodiments, Ring A² is optionally substituted with one or more R^{u}. In certain embodiments, Ring A² is optionally substituted with one or more R¹. In certain embodiments, Ring A² is optionally substituted with one or more R⁵. In certain embodiments, Ring A² is optionally substituted with one or more R⁶.

In certain embodiments, R¹ and R², together with the intervening carbon atoms, form 8- to 12-membered fused bicyclic heterocycle selected from

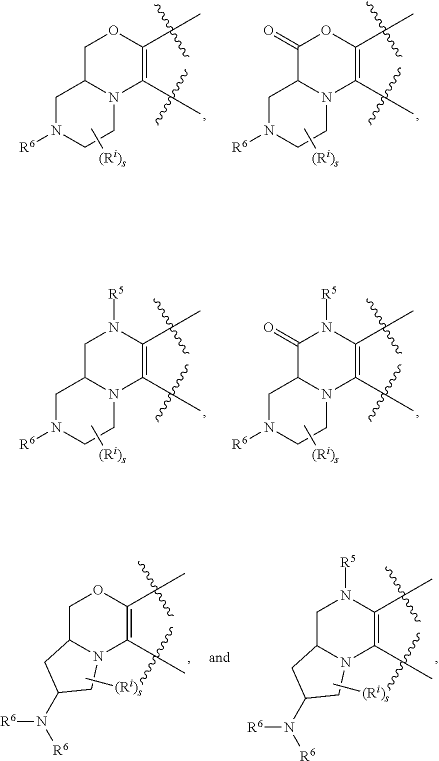

wherein:
R⁵ is hydrogen or C₁₋₆ alkyl optionally substituted with one or more R^{u}; or
R⁵ is an amino-protecting group;
R⁶ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}; or
R⁶ is an amino-protecting group;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, the compound of Formula I-1 is a compound of Formula I-1-i, I-1-ii, I-1-iii, I-1-iv, I-1-v, I-1-vi, I-1-vii, or I-1-viii:

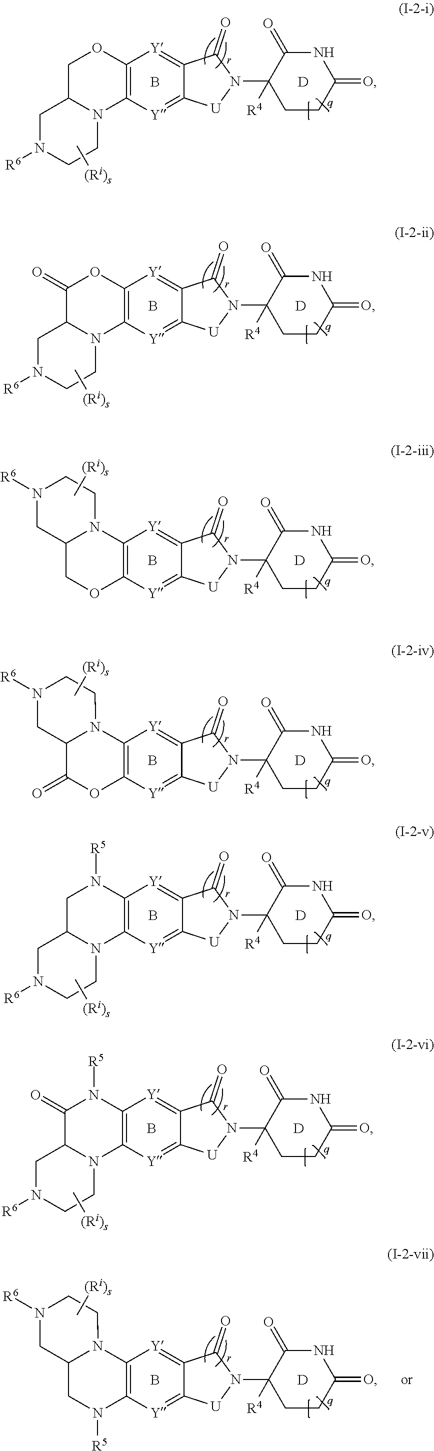

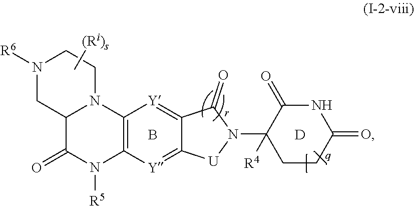

or a pharmaceutically acceptable salt, solvate, or stereoisomer thereof.

In certain embodiments, the compound of Formula I-2 is a compound of Formula I-2-i, I-2-ii, I-2-iii, I-2-iv, I-2-v, I-2-vi, I-2-vii, or I-2-viii

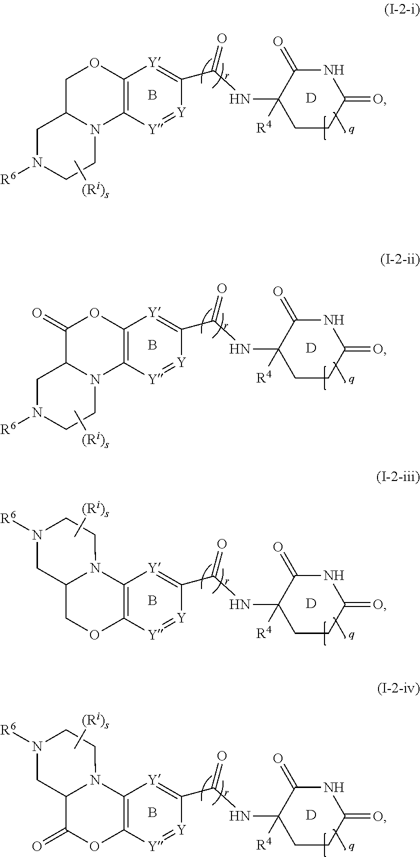

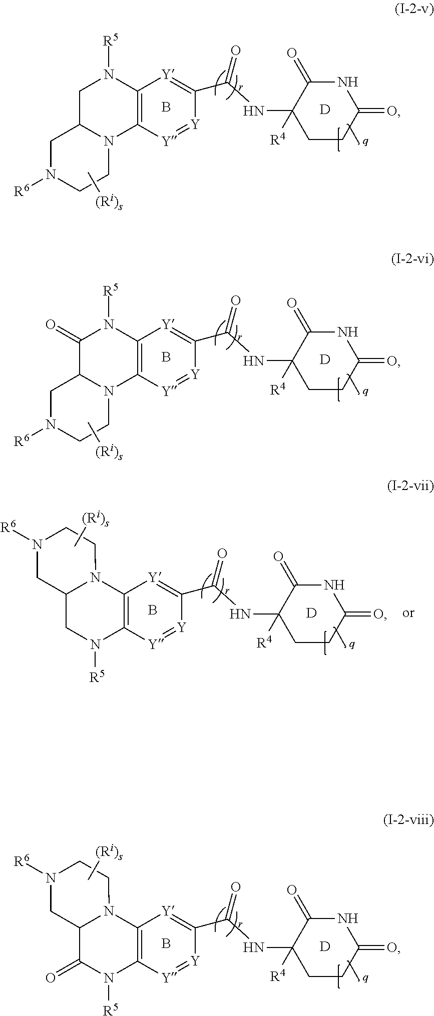

or a pharmaceutically acceptable salt, solvate, or stereoisomer thereof.

In certain embodiments, R⁵ is hydrogen or C₁₋₆ alkyl

In certain embodiments, R⁶ is hydrogen, C₁₋₆ alkyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —C(═O)R^{a}, or —C(═O)OR^{b}, wherein the alkyl, alkylene, carbocyclyl, heterocyclyl, or heteroaryl is optionally substituted with one or more R^{u}.

In certain embodiments, Y″ is N.

In certain embodiments, Y″ is CR³.

In certain embodiments, R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R³ is hydrogen.

In certain embodiments, R² and R³, together with the intervening carbon atoms, form optionally substituted 8- to 12-membered fused bicyclic heterocycle.

In certain embodiments, the 7- to 16-membered or 8- to 12-membered fused heterocycle is optionally substituted with one or more R^{u}. In certain embodiments, the 7- to 16-membered or 8- to 12-membered fused heterocycle is optionally substituted with one or more Rⁱ. In certain embodiments, the 7- to 16-membered or 8- to 12-membered fused heterocycle is optionally substituted with one or more R⁵. In certain embodiments, the 7- to 16-membered or 8- to 12-membered fused heterocycle is optionally substituted with one or more R⁶.

In certain embodiments, R^{u} is R¹. In certain embodiments, R^{u} is R⁵. In certain embodiments, R^{u} is R⁶. In certain embodiments, Rⁱ is R⁵. In certain embodiments, Rⁱ is R⁶.

In certain embodiments, the 7- to 16-membered or 8- to 12-membered fused heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, R² and R³, together with the intervening carbon atoms, form optionally substituted 8- to 12-membered fused bicyclic heterocycle of formula

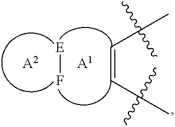

wherein E and F are independently CH or N.

In certain embodiments, Ring A¹ is optionally substituted 5- to 7-membered heterocycle.

In certain embodiments, Ring A¹ is optionally substituted 5- to 7-membered heterocycle comprising one nitrogen atom. In certain embodiments, Ring A¹ is optionally substituted 5- to 7-membered heterocycle comprising two nitrogen atoms.

In certain embodiments, Ring A¹ is optionally substituted 5-membered heterocycle. In certain embodiments, Ring A¹ is optionally substituted 6-membered heterocycle. In certain embodiments, Ring A¹ is optionally substituted 7-membered heterocycle.

In certain embodiments, Ring A¹ is optionally substituted 5-membered heterocycle comprising one nitrogen atom. In certain embodiments, Ring A¹ is optionally substituted 6 -membered heterocycle comprising one nitrogen atom. In certain embodiments, Ring A¹ is optionally substituted 7-membered heterocycle comprising one nitrogen atom.

In certain embodiments, Ring A¹ is optionally substituted 5-membered heterocycle comprising two nitrogen atoms. In certain embodiments, Ring A¹ is optionally substituted 6-membered heterocycle comprising two nitrogen atoms. In certain embodiments, Ring A¹ is optionally substituted 7-membered heterocycle comprising two nitrogen atoms.

In certain embodiments, Ring A¹ is optionally substituted with one or more R^{u}. In certain embodiments, Ring A¹ is optionally substituted with one or more R¹. In certain embodiments, Ring A¹ is optionally substituted with one or more R⁵. In certain embodiments, Ring A¹ is optionally substituted with one or more R⁶.

In certain embodiments, Ring A² is optionally substituted 5- to 7-membered heterocycle.

In certain embodiments, Ring A² is optionally substituted 5- to 7-membered heterocycle comprising one nitrogen atom. In certain embodiments, Ring A² is optionally substituted 5- to 7-membered heterocycle comprising two nitrogen atoms.

In certain embodiments, Ring A² is optionally substituted 5-membered heterocycle. In certain embodiments, Ring A² is optionally substituted 6-membered heterocycle. In certain embodiments, Ring A² is optionally substituted 7-membered heterocycle.

In certain embodiments, Ring A² is optionally substituted 5-membered heterocycle comprising one nitrogen atom. In certain embodiments, Ring A² is optionally substituted 6-membered heterocycle comprising one nitrogen atom. In certain embodiments, Ring A² is optionally substituted 7-membered heterocycle comprising one nitrogen atom.

In certain embodiments, Ring A² is optionally substituted 5-membered heterocycle comprising two nitrogen atoms. In certain embodiments, Ring A² is optionally substituted 6-membered heterocycle comprising two nitrogen atoms. In certain embodiments, Ring A² is optionally substituted 7-membered heterocycle comprising two nitrogen atoms.

In certain embodiments, Ring A² is optionally substituted with one or more R^{u}. In certain embodiments, Ring A² is optionally substituted with one or more R¹. In certain embodiments, Ring A² is optionally substituted with one or more R⁵. In certain embodiments, Ring A² is optionally substituted with one or more R⁶.

In certain embodiments, R² and R³, together with the intervening carbon atoms, form 8- to 12-membered fused bicyclic heterocycle selected from

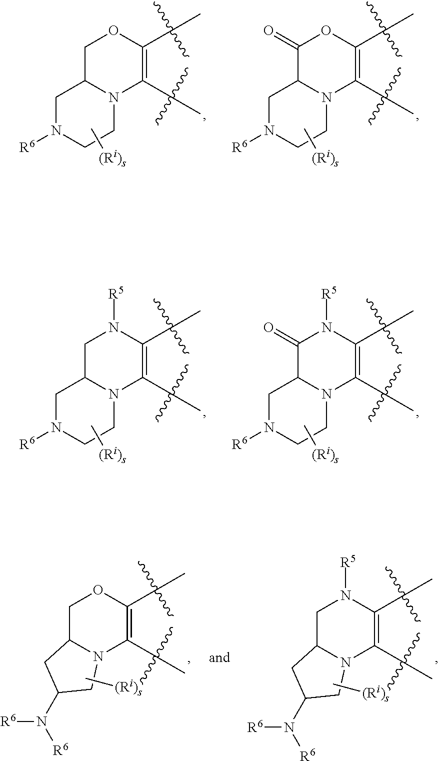

wherein:
R⁵ is hydrogen or C₁₋₆ alkyl optionally substituted with one or more R^{u}; or
R⁵ is an amino-protecting group;
R⁶ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R⁶ is an amino-protecting group;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆_o aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more Rᵤ; and
s is an integer selected from 0 to 8, as valency permits.

In certain embodiments, the compound of Formula I-1 is a compound of Formula I-1-ix, I-1-x, I-1-xi, or I-1-xii:

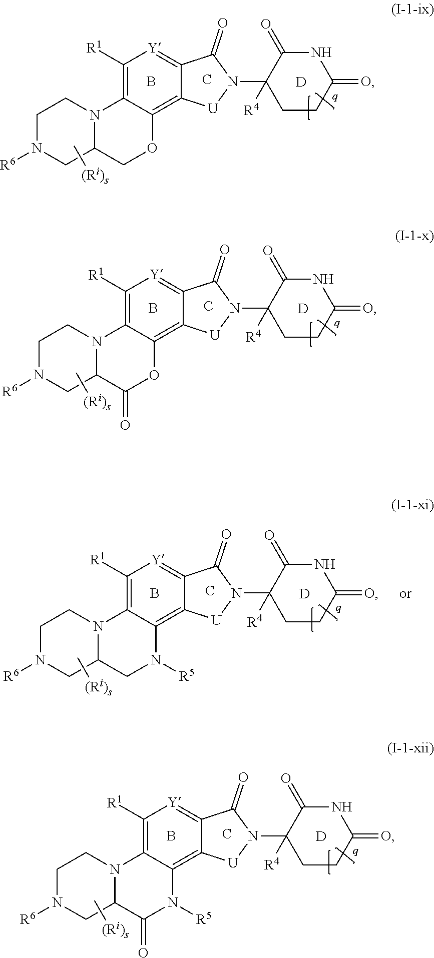

or a pharmaceutically acceptable salt, solvate, or stereoisomer thereof.

In certain embodiments, the compound of Formula I-2 is a compound of Formula I-2-ix, I-2-x, I-2-xi, or I-2-xii:

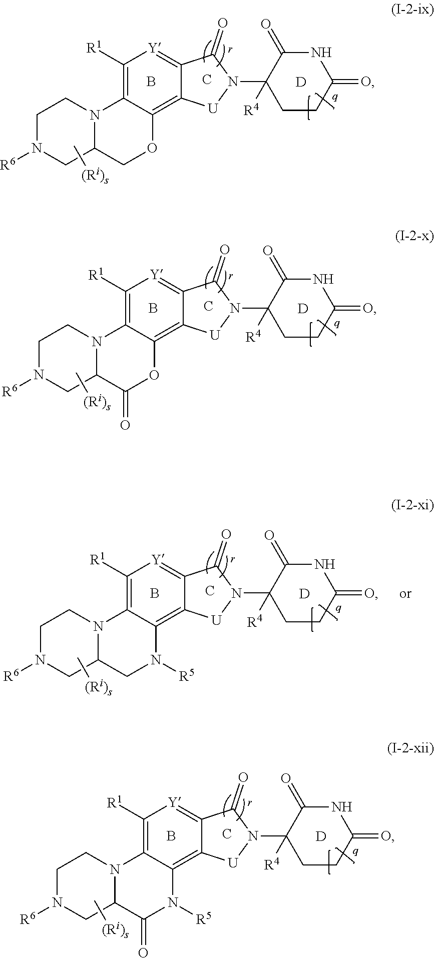

or a pharmaceutically acceptable salt, solvate, or stereoisomer thereof.

In certain embodiments, R⁵ is hydrogen or C₁₋₆ alkyl.

In certain embodiments, R⁶ is hydrogen, C₁₋₆ alkyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —C(═O)R^{a}, or —C(═O)OR^{b}, wherein the alkyl, alkylene, carbocyclyl, heterocyclyl, or heteroaryl is optionally substituted with one or more R^{u}.

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R¹ is hydrogen.

In certain embodiments, Y′ is N.

In certain embodiments, Y′ is CR^{Y′}.

In certain embodiments, R^{Y′} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y′} is hydrogen.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more Rᵤ.

In certain embodiments, s is an integer selected from 0 to 8, as valency permits. In certain embodiments, s is an integer selected from 0 to 7, as valency permits. In certain embodiments, s is an integer selected from 0 to 6, as valency permits. In certain embodiments, s is an integer selected from 0 to 5, as valency permits. In certain embodiments, s is an integer selected from 0 to 4, as valency permits. In certain embodiments, s is an integer selected from 0 to 3, as valency permits. In certain embodiments, s is an integer selected from 0 to 2, as valency permits. In certain embodiments, s is 0 or 1, as valency permits.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, R⁴ is hydrogen. In certain embodiments, R⁴ is deuterium. In certain embodiments, R⁴ is C₁₋₆haloalkyl. In certain embodiments, R⁴ is C₁₋₆ alkyl.

In certain embodiments, q is 0. In certain embodiments, q is 1. In certain embodiments, q is 2. In certain embodiments, q is 0 or 1. In certain embodiments, q is 0 or 2. In certain embodiments, q is 1 or 2.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C_-6 alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂₋₆ alkynyl, C₃₋₆ carbocyclyl, or 3- to 6-membered heterocyclyl.

In certain embodiments, each R^{b} is independently hydrogen, C₁₋₆ alkyl, C₃₋₆carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10 -membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-i-butylamino, methyl-s-butylamino, methyl-t-butylamino, methylpentylamino, methylhexylamino, ethyl-n-propylamino, ethyl-i-propylamino, ethyl-n-butylamino, ethyl-s-butylamino, ethyl-i-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{U} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, two R^{u}, together with the carbon atom(s) to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, two geminal R^{u}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, the compound disclosed herein is selected from the compounds in Tables 1-3 or a pharmaceutically acceptable salt thereof.

In certain embodiments, the compound disclosed herein is selected from the compounds in Table 1 or a pharmaceutically acceptable salt thereof.

In certain embodiments, the compound disclosed herein is selected from the compounds in Table 2 or a pharmaceutically acceptable salt thereof.

In certain embodiments, the compound disclosed herein is selected from the compounds in Table 3 or a pharmaceutically acceptable salt thereof.

In certain embodiments, the compound disclosed herein is selected from the compounds in Table 1.

In certain embodiments, the compound disclosed herein is selected from the compounds in Table 2.

In certain embodiments, the compound disclosed herein is selected from the compounds in Table 3.

TABLE 1

| Compound No. | Structure | Compound Name |
|---|---|---|
| A1 | 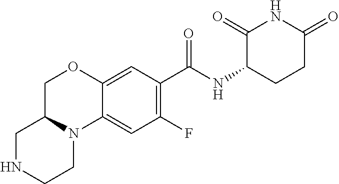 [SMILES: O=C1CC[C@H](NC(=O)c2cc3c(cc2F)N2CCNC[C@H]2CO3)C(=O)N1] | (S)-N-((S)-2,6-dioxopiperidin-3- yl)-9-fluoro-1,2,3,4,4a,5- hexahydrobenzo[b]pyrazino[1,2- d][1,4]oxazine-8-carboxamide |
| A2 | 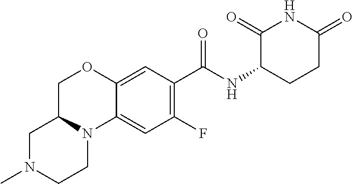 [SMILES: CN1CCN2c3cc(F)c(C(=O)N[C@H]4CCC(=O)NC4=O)cc3OC[C@@H]2C1] | (S)-N-((S)-2,6-dioxopiperidin-3- yl)-9-fluoro-3-methyl-1,2,3,4,4a,5- hexahydrobenzo[b]pyrazino[1,2- d][1,4]oxazine-8-carboxamide |
| A3 | 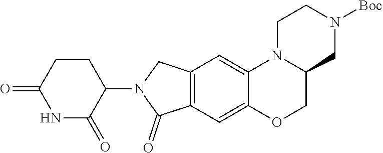 [SMILES: CC(C)(C)OC(=O)N1CCN2c3cc4c(cc3OC[C@@H]2C1)C(=O)N(C1CCC(=O)NC1=O)C4] | tert-butyl (4aS)-9-(2,6- dioxopiperidin-3-yl)-8-oxo- 1,2,4a,5,9,10-hexahydro-8H- pyrazino[1′,2′:4,5][1,4]oxazino[2,3- f]isoindole-3(4H)-carboxylate |
| A4 | 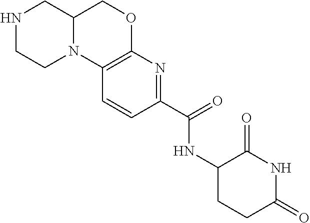 [SMILES: O=C1CCC(NC(=O)c2ccc3c(n2)OCC2CNCCN32)C(=O)N1] | N-(2,6-dioxopiperidin-3-yl)- 1,2,3,4,4a,5- hexahydropyrazino[1,2-d] pyrido[2,3-b][1,4] oxazine-8-carboxamide |
| A7 | 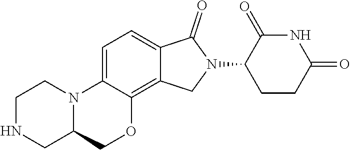 [SMILES: O=C1CC[C@H](N2Cc3c(ccc4c3OC[C@H]3CNCCN43)C2=O)C(=O)N1] | (S)-3-((R)-1-oxo-1,3,5,5a,6,7,8,9- octahydro-2H- pyrazino[1′,2′:4,5][1,4]oxazino[2,3- e]isoindol-2-yl)piperidine-2,6- dione |
| A8 | 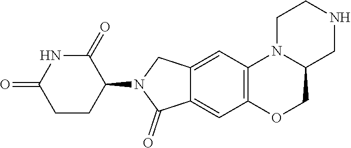 [SMILES: O=C1CC[C@H](N2Cc3cc4c(cc3C2=O)OC[C@@H]2CNCCN42)C(=O)N1] | (S)-3-((S)-8-oxo-1,2,3,4,4a,5,8,10- octahydro-9H- pyrazino[1′,2′:4,5][1,4]oxazino[2,3- f]isoindol-9-yl)piperidine-2,6- dione |
| A9 | 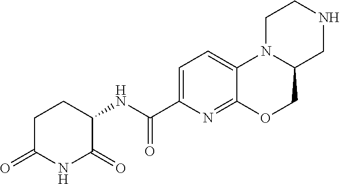 [SMILES: O=C1CC[C@H](NC(=O)c2ccc3c(n2)OC[C@@H]2CNCCN32)C(=O)N1] | (S)-N-((S)-2,6-dioxopiperidin-3- yl)-1,2,3,4,4a,5- hexahydropyrazino[1,2-d] pyrido[2,3-b][1,4]oxazine-8- carboxamide |

TABLE 2

| Compound | | |
|---|---|---|
| No. | Structure | Chemical Name |
| B0 (A7) | 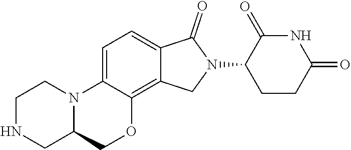 [SMILES: O=C1CC[C@H](N2Cc3c(ccc4c3OC[C@H]3CNCCN43)C2=O)C(=O)N1] | (S)-3-((R)-1-oxo-1,3,5,5a,6,7,8,9- octahydro-2H- pyrazino[1′,2′:4,5][1,4]oxazino[2,3- e]isoindol-2-yl)piperidine-2,6-dione |
| B1 | 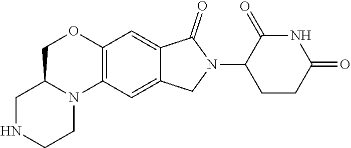 [SMILES: O=C1CCC(N2Cc3cc4c(cc3C2=O)OC[C@@H]2CNCCN42)C(=O)N1] | 3-((S)-8-oxo-1,2,3,4,4a,5,8,10- octahydro-9H- pyrazino[1′,2′:4,5][1,4]oxazino[2,3- f]isoindol-9-yl)piperidine-2,6-dione |
| B2 | 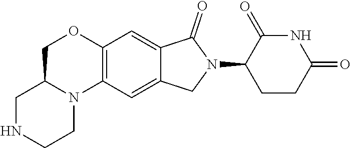 [SMILES: O=C1CC[C@@H](N2Cc3cc4c(cc3C2=O)OC[C@@H]2CNCCN42)C(=O)N1] | (R)-3-((S)-8-oxo-1,2,3,4,4a,5,8,10- octahydro-9H- pyrazino[1′,2′:4,5][1,4]oxazino[2,3- f]isoindol-9-yl)piperidine-2,6-dione |
| B3 | 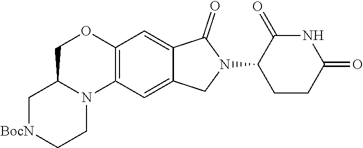 [SMILES: CC(C)(C)OC(=O)N1CCN2c3cc4c(cc3OC[C@@H]2C1)C(=O)N([C@H]1CCC(=O)NC1=O)C4] | (S)-tert-butyl (4aS)-9-(2,6- dioxopiperidin-3-yl)-8-oxo- 1,2,4a,5,9,10-hexahydro-8H- pyrazino[1′,2′:4,5][1,4]oxazino[2,3- f]isoindole-3(4H)-carboxylate |
| B4 | 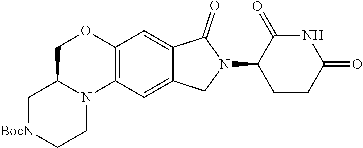 [SMILES: CC(C)(C)OC(=O)N1CCN2c3cc4c(cc3OC[C@@H]2C1)C(=O)N([C@@H]1CCC(=O)NC1=O)C4] | (R)-tert-butyl (4aS)-9-(2,6- dioxopiperidin-3-yl)-8-oxo- 1,2,4a,5,9,10-hexahydro-8H- pyrazino[1′,2′:4,5][1,4]oxazino[2,3- f]isoindole-3(4H)-carboxylate |
| B5 | 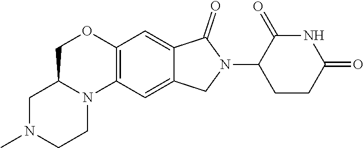 [SMILES: CN1CCN2c3cc4c(cc3OC[C@@H]2C1)C(=O)N(C1CCC(=O)NC1=O)C4] | 3-((S)-3-methyl-8-oxo- 1,2,3,4,4a,5,8,10-octahydro-9H- pyrazino[1′,2′:4,5][1,4]oxazino[2,3- f]isoindol-9-yl)piperidine-2,6-dione |
| B6 | 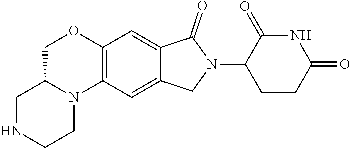 [SMILES: O=C1CCC(N2Cc3cc4c(cc3C2=O)OC[C@H]2CNCCN42)C(=O)N1] | 3-((R)-8-oxo-1,2,3,4,4a,5,8,10- octahydro-9H- pyrazino[1′,2′:4,5][1,4]oxazino[2,3- f]isoindol-9-yl)piperidine-2,6-dione |
| B7 | 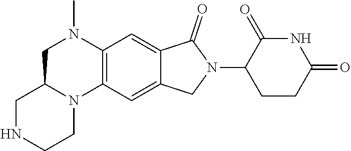 [SMILES: CN1C[C@@H]2CNCCN2c2cc3c(cc21)C(=O)N(C1CCC(=O)NC1=O)C3] | 3-((S)-6-methyl-8-oxo- 2,3,4,4a,5,6,8,10- octahydropyrazino[1,2- a]pyrrolo[3,4-g]quinoxalin-9(1H)- yl)piperidine-2,6-dione |
| B8 | 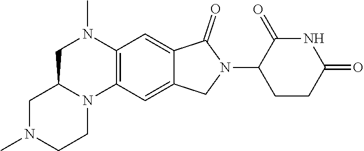 [SMILES: CN1CCN2c3cc4c(cc3N(C)C[C@@H]2C1)C(=O)N(C1CCC(=O)NC1=O)C4] | 3-((S)-3,6-dimethyl-8-oxo- 2,3,4,4a,5,6,8,10- octahydropyrazino[1,2- a]pyrrolo[3,4-g]quinoxalin-9(1H)- yl)piperidine-2,6-dione |
| B9 | 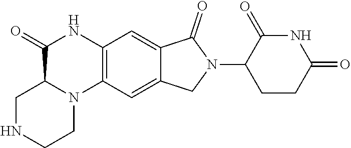 [SMILES: O=C1CCC(N2Cc3cc4c(cc3C2=O)NC(=O)[C@@H]2CNCCN42)C(=O)N1] | (4aS)-9-(2,6-dioxopiperidin-3-yl)- 2,3,4,4a,9,10- hexahydropyrazino[1,2- a]pyrrolo[3,4-g]quinoxaline- 5,8(1H,6H)-dione |
| B10 | 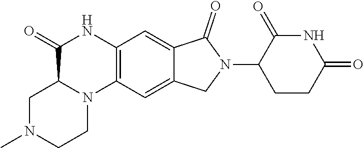 [SMILES: CN1CCN2c3cc4c(cc3NC(=O)[C@@H]2C1)C(=O)N(C1CCC(=O)NC1=O)C4] | (4aS)-9-(2,6-dioxopiperidin-3-yl)- 3-methyl-2,3,4,4a,9,10- hexahydropyrazino[1,2- a]pyrrolo[3,4-g]quinoxaline- 5,8(1H,6H)-dione |
| B11 | 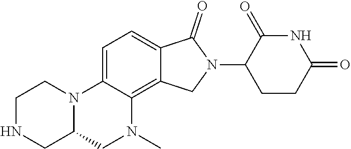 [SMILES: CN1C[C@@H]2CNCCN2c2ccc3c(c21)CN(C1CCC(=O)NC1=O)C3=O] | 3-((S)-4-methyl-1-oxo- 3,4,5,5a,6,7,8,9- octahydropyrazino[1,2- a]pyrrolo[3,4-f]quinoxalin-2(1H)- yl)piperidine-2,6-dione |
| B12 | 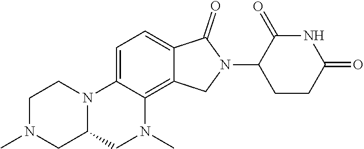 [SMILES: CN1CCN2c3ccc4c(c3N(C)C[C@@H]2C1)CN(C1CCC(=O)NC1=O)C4=O] | 3-((S)-4,7-dimethyl-1-oxo- 3,4,5,5a,6,7,8,9- octahydropyrazino[1,2- a]pyrrolo[3,4-f]quinoxalin-2(1H)- yl)piperidine-2,6-dione |
| B13 | 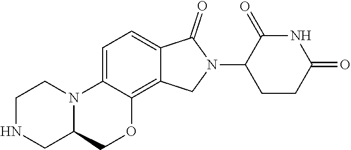 [SMILES: O=C1CCC(N2Cc3c(ccc4c3OC[C@H]3CNCCN43)C2=O)C(=O)N1] | 3-((R)-1-oxo-1,3,5,5a,6,7,8,9- octahydro-2H- pyrazino[1′,2′:4,5][1,4]oxazino[2,3- e]isoindol-2-yl)piperidine-2,6-dione |
| B14 | 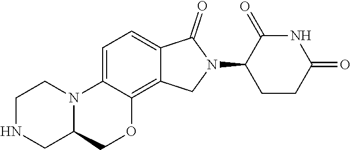 [SMILES: O=C1CC[C@@H](N2Cc3c(ccc4c3OC[C@H]3CNCCN43)C2=O)C(=O)N1] | (R)-3-((R)-1-oxo-1,3,5,5a,6,7,8,9- octahydro-2H- pyrazino[1′,2′:4,5][1,4]oxazino[2,3- e]isoindol-2-yl)piperidine-2,6-dione |
| B15 | 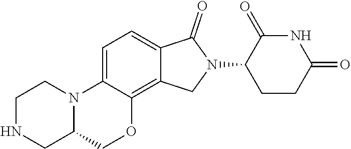 [SMILES: O=C1CC[C@H](N2Cc3c(ccc4c3OC[C@@H]3CNCCN43)C2=O)C(=O)N1] | (S)-3-((S)-1-oxo-1,3,5,5a,6,7,8,9- octahydro-2H- pyrazino[1′,2′:4,5][1,4]oxazino[2,3- e]isoindol-2-yl)piperidine-2,6-dione |
| B16 | 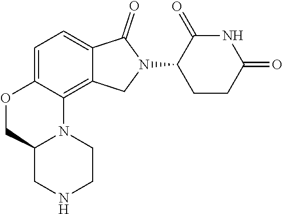 [SMILES: O=C1CC[C@H](N2Cc3c(ccc4c3N3CCNC[C@H]3CO4)C2=O)C(=O)N1] | (S)-3-((S)-3-oxo-1,3,7,7a,8,9,10,11- octahydro-2H- pyrazino[1′,2′:4,5][1,4]oxazino[3,2- e]isoindol-2-yl)piperidine-2,6-dione |
| B17 | 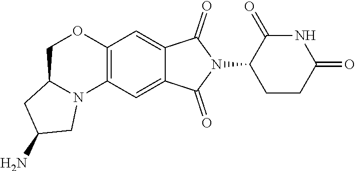 [SMILES: N[C@H]1C[C@H]2COc3cc4c(cc3N2C1)C(=O)N([C@H]1CCC(=O)NC1=O)C4=O] | (S)-3-((2S,3aS)-2-amino-7-oxo- 2,3,3a,4,7,9-hexahydro-1H,8H, pyrrolo[1′,2′:4,5][1,4]oxazino[2,3- f]isoindol-8-yl)piperidine-2,6-dione |
| B18 | 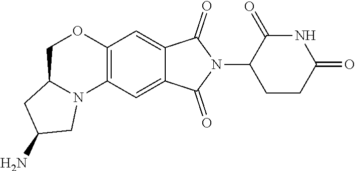 [SMILES: N[C@H]1C[C@H]2COc3cc4c(cc3N2C1)C(=O)N(C1CCC(=O)NC1=O)C4=O] | (2S,3aS)-2-amino-8-(2,6- dioxopiperidin-3-yl)-2,3,3a,4- tetrahydro-1H,7H- pyrrolo[1′,2′:4,5][1,4]oxazino[2,3- f]isoindole-7,9(8H)-dione |
| B19 | 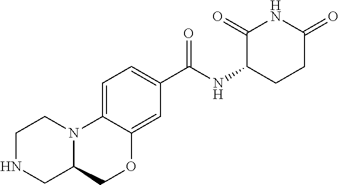 [SMILES: O=C1CC[C@H](NC(=O)c2ccc3c(c2)OC[C@H]2CNCCN32)C(=O)N1] | (R)-N-((S)-2,6-dioxopiperidin-3- yl)-1,2,3,4,4a,5- hexahydropyrazino[1,2-d]pyrido [2,3-b][1,4]oxazine-8- carboxamide |
| B20 | 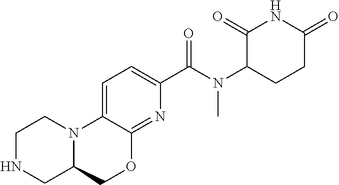 [SMILES: CN(C(=O)c1ccc2c(n1)OC[C@H]1CNCCN21)C1CCC(=O)NC1=O] | (4aR)-N-(2,6-dioxopiperidin-3-yl)- N-methyl-1,2,3,4,4a,5- hexahydropyrazino[1,2- d]pyrido[2,3-b][1,4]oxazine-8- carboxamide |
| B21 | 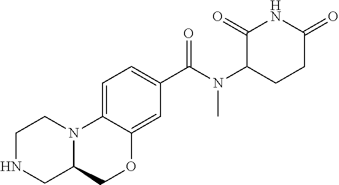 [SMILES: CN(C(=O)c1ccc2c(c1)OC[C@H]1CNCCN21)C1CCC(=O)NC1=O] | (4aR)-N-(2,6-dioxopiperidin-3-yl)- N-methyl-1,2,3,4,4a,5- hexahydrobenzo[b]pyrazino[1,2- d][1,4]oxazine-8-carboxamide |
| B22 | 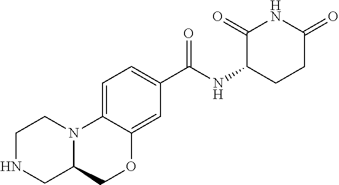 [SMILES: O=C1CC[C@H](NC(=O)c2ccc3c(c2)OC[C@H]2CNCCN32)C(=O)N1] | (R)-N-((S)-2,6-dioxopiperidin-3- yl)-1,2,3,4,4a,5- hexahydrobenzo[b]pyrazino[1,2- d][1,4]oxazine-8-carboxamide |
| B23 | 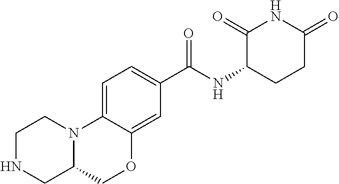 [SMILES: O=C1CC[C@H](NC(=O)c2ccc3c(c2)OC[C@@H]2CNCCN32)C(=O)N1] | (S)-N-((S)-2,6-dioxopiperidin-3- yl)-1,2,3,4,4a,5- hexahydrobenzo[b]pyrazino[1,2- d][1,4]oxazine-8-carboxamide |
| B24 | 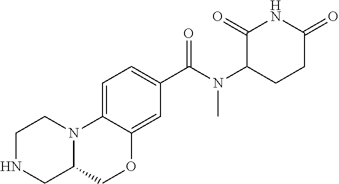 [SMILES: CN(C(=O)c1ccc2c(c1)OC[C@@H]1CNCCN21)C1CCC(=O)NC1=O] | (4aS)-N-(2,6-dioxopiperidin-3-yl)- N-methyl-1,2,3,4,4a,5- hexahydrobenzo[b]pyrazino[1,2- d][1,4]oxazine-8-carboxamide |

TABLE 3

| Compound | | |
|---|---|---|
| No. | Structures | Chemical names |
| C1 | 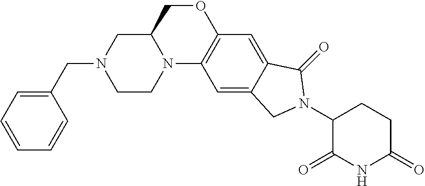 [SMILES: O=C1CCC(N2Cc3cc4c(cc3C2=O)OC[C@@H]2CN(Cc3ccccc3)CCN42)C(=O)N1] | 3-((S)-3-benzyl-8-oxo-1,2,3,4,4a,5,8,10- octahydro-9H- pyrazino[1′,2′:4,5][1,4]oxazino[2,3- f]isoindol-9-yl)piperidine-2,6-dione |
| C2 | 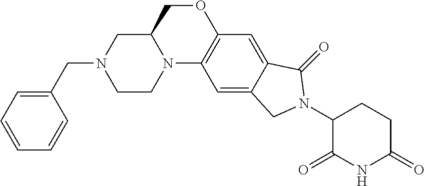 [SMILES: O=C1CCC(N2Cc3cc4c(cc3C2=O)OC[C@@H]2CN(Cc3ccccc3)CCN42)C(=O)N1] | 3-((S)-3-(cyclohexylmethyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C3 | 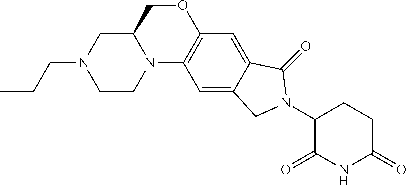 [SMILES: CCCN1CCN2c3cc4c(cc3OC[C@@H]2C1)C(=O)N(C1CCC(=O)NC1=O)C4] | 3-((S)-8-oxo-3-385.21propyl- 1,2,3,4,4a,5,8,10-octahydro-9H- pyrazino[1′,2′:4,5][1,4]oxazino[2,3- f]isoindol-9-yl)piperidine-2,6-dione |
| C4 | 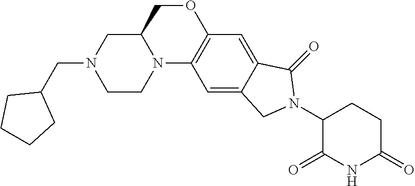 [SMILES: O=C1CCC(N2Cc3cc4c(cc3C2=O)OC[C@@H]2CN(CC3CCCC3)CCN42)C(=O)N1] | 3-((S)-3-(cyclopentylmethyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C5 | 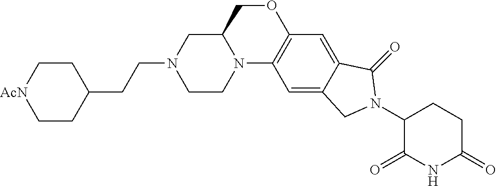 | 3-((S)-3-(2-(1-acetylpiperidin-4- yl)ethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| C6 | 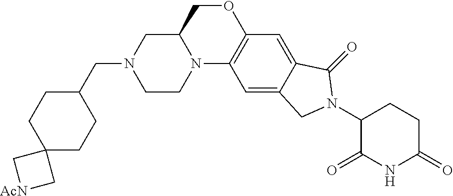 | 3-((S)-3-((2-acetyl-2- azaspiro[3.5]nonan-7-yl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C7 | 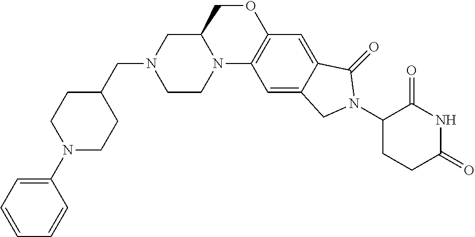 [SMILES: O=C1CCC(N2Cc3cc4c(cc3C2=O)OC[C@@H]2CN(CC3CCN(c5ccccc5)CC3)CCN42)C(=O)N1] | 3-((S)-8-oxo-3-((1-phenylpiperidin-4- yl)methyl)-1,2,3,4,4a,5,8,10-octahydro- 9H-pyrazino[1′,2′:4,5][1,4]oxazino[2,3- f]isoindol-9-yl)piperidine-2,6-dione |
| C8 | 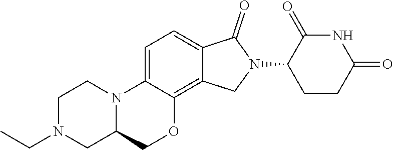 [SMILES: CCN1CCN2c3ccc4c(c3OC[C@H]2C1)CN([C@H]1CCC(=O)NC1=O)C4=O] | (S)-3-((R)-7-ethyl-1-oxo- 1,3,5,5a,6,7,8,9-octahydro-2H- pyrazino[1′,2′:4,5][1,4]oxazino[2,3- e]isoindol-2-yl)piperidine-2,6-dione |
| C9 | 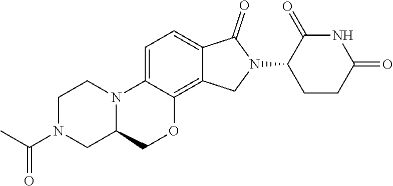 [SMILES: CC(=O)N1CCN2c3ccc4c(c3OC[C@H]2C1)CN([C@H]1CCC(=O)NC1=O)C4=O] | (S)-3-((R)-7-acetyl-1-oxo- 1,3,5,5a,6,7,8,9-octahydro-2H- pyrazino[1′,2′:4,5][1,4]oxazino[2,3- e]isoindol-2-yl)piperidine-2,6-dione |
| C10 | 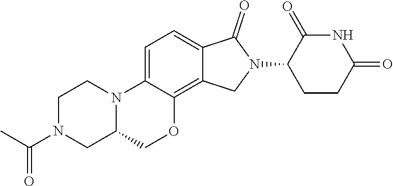 [SMILES: CC(=O)N1CCN2c3ccc4c(c3OC[C@@H]2C1)CN([C@H]1CCC(=O)NC1=O)C4=O] | (S)-3-((S)-7-acetyl-1-oxo- 1,3,5,5a,6,7,8,9-octahydro-2H- pyrazino[1′,2′:4,5][1,4]oxazino[2,3- e]isoindol-2-yl)piperidine-2,6-dione |
| C11 | 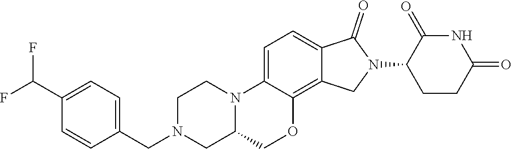 [SMILES: O=C1CC[C@H](N2Cc3c(ccc4c3OC[C@@H]3CN(Cc5ccc(C(F)F)cc5)CCN43)C2=O)C(=O)N1] | (S)-3-((S)-7-(4-(difluoromethyl)benzyl)- 1-oxo-1,3,5,5a,6,7,8,9-octahydro-2H- pyrazino[1′,2′:4,5][1,4]oxazino[2,3- e]isoindol-2-yl)piperidine-2,6-dione |
| C12 | 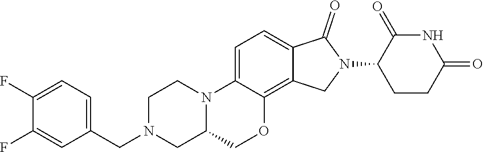 [SMILES: O=C1CC[C@H](N2Cc3c(ccc4c3OC[C@@H]3CN(Cc5ccc(F)c(F)c5)CCN43)C2=O)C(=O)N1] | (S)-3-((S)-7-(3,4-difluorobenzyl)-1-oxo- 1,3,5,5a,6,7,8,9-octahydro-2H- pyrazino[1′,2′:4,5][1,4]oxazino[2,3- e]isoindol-2-yl)piperidine-2,6-dione |
| C13 | 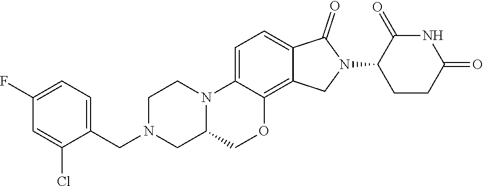 [SMILES: O=C1CC[C@H](N2Cc3c(ccc4c3OC[C@@H]3CN(Cc5ccc(F)cc5Cl)CCN43)C2=O)C(=O)N1] | (S)-3-((S)-7-(2-chloro-4-fluorobenzyl)- 1-oxo-1,3,5,5a,6,7,8,9-octahydro-2H- pyrazino[1′,2′:4,5][1,4]oxazino[2,3- e]isoindol-2-yl)piperidine-2,6-dione |
| C14 | 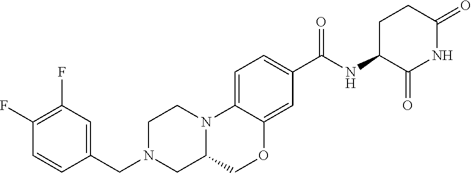 [SMILES: O=C1CC[C@H](NC(=O)c2ccc3c(c2)OC[C@@H]2CN(Cc4ccc(F)c(F)c4)CCN32)C(=O)N1] | (S)-3-(3,4-difluorobenzyl)-N-((S)-2,6- dioxopiperidin-3-yl)-1,2,3,4,4a,5- hexahydrobenzo[b]pyrazino[1,2-d] [1,4]oxazine-8-carboxamide |
| C15 | 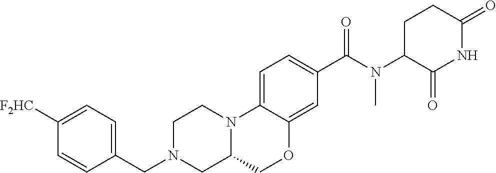 [SMILES: CN(C(=O)c1ccc2c(c1)OC[C@@H]1CN(Cc3ccc(C(F)F)cc3)CCN21)C1CCC(=O)NC1=O] | (4aS)-3-(4-(difluoromethyl)benzyl)-N- (2,6-dioxopiperidin-3-yl)-N-methyl- 1,2,3,4,4a,5- hexahydrobenzo[b]pyrazino[1,2-d] [1,4]oxazine-8-carboxamide |
| C16 | 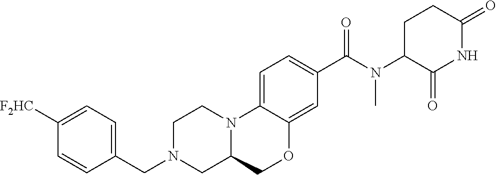 [SMILES: CN(C(=O)c1ccc2c(c1)OC[C@H]1CN(Cc3ccc(C(F)F)cc3)CCN21)C1CCC(=O)NC1=O] | (4aR)-3-(4-(difluoromethyl)benzyl)-N- (2,6-dioxopiperidin-3-yl)-N-methyl- 1,2,3,4,4a,5- hexahydrobenzo[b]pyrazino[1,2- d][1,4]oxazine-8-carboxamide |
| C17 | 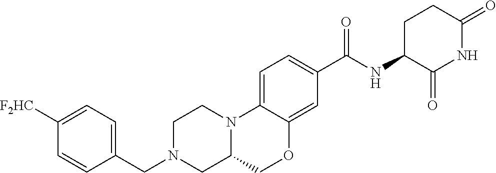 [SMILES: O=C1CC[C@H](NC(=O)c2ccc3c(c2)OC[C@@H]2CN(Cc4ccc(C(F)F)cc4)CCN32)C(=O)N1] | (S)-3-(4-(difluoromethyl)benzyl)-N-((S)- 2,6-dioxopiperidin-3-yl)-1,2,3,4,4a,5- hexahydrobenzo[b]pyrazino[1,2-d] [1,4]oxazine-8-carboxamide |
| C18 | 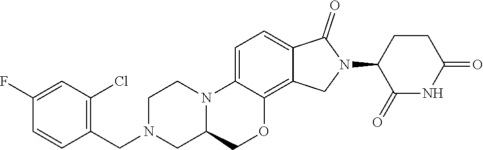 [SMILES: O=C1CC[C@H](N2Cc3c(ccc4c3OC[C@H]3CN(Cc5ccc(F)cc5Cl)CCN43)C2=O)C(=O)N1] | (S)-3-((R)-7-(2-chloro-4-fluorobenzyl)- 1-oxo-1,3,5,5a,6,7,8,9-octahydro-2H- pyrazino[1′,2′:4,5][1,4]oxazino[2,3- e]isoindol-2-yl)piperidine-2,6-dione |
| C19 | 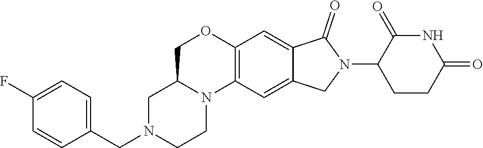 [SMILES: O=C1CCC(N2Cc3cc4c(cc3C2=O)OC[C@@H]2CN(Cc3ccc(F)cc3)CCN42)C(=O)N1] | 3-((S)-3-(4-fluorobenzyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C20 | 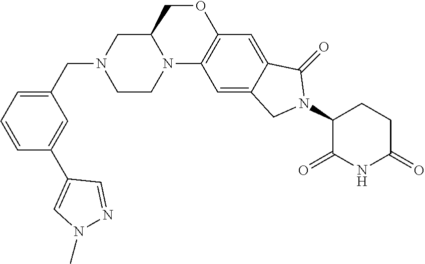 [SMILES: Cn1cc(-c2cccc(CN3CCN4c5cc6c(cc5OC[C@@H]4C3)C(=O)N([C@H]3CCC(=O)NC3=O)C6)c2)cn1] | (S)-3-((S)-3-(3-(1-methyl-1H-pyrazol-4- yl)benzyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| C21 | 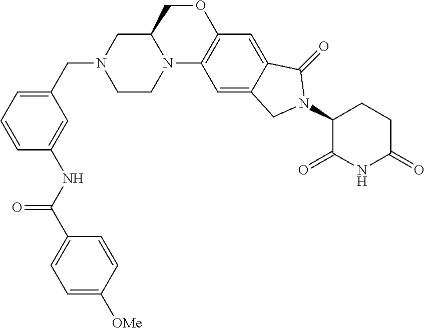 [SMILES: COc1ccc(C(=O)Nc2cccc(CN3CCN4c5cc6c(cc5OC[C@@H]4C3)C(=O)N([C@H]3CCC(=O)NC3=O)C6)c2)cc1] | N-(3-(((S)-9-((S)-2,6-dioxopiperidin-3- yl)-8-oxo-1,2,4a,5,9,10-hexahydro-8H- pyrazino[1′,2′:4,5][1,4]oxazino[2,3- f]isoindol-3(4H)-yl)methyl)phenyl)-4- methoxybenzamide |
| C22 | 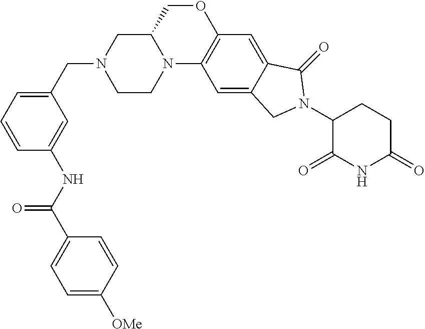 [SMILES: COc1ccc(C(=O)Nc2cccc(CN3CCN4c5cc6c(cc5OC[C@H]4C3)C(=O)N(C3CCC(=O)NC3=O)C6)c2)cc1] | N-(3-(((4aR)-9-(2,6-dioxopiperidin-3- yl)-8-oxo-1,2,4a,5,9,10-hexahydro-8H- pyrazino[1′,2′:4,5][1,4]oxazino[2,3- f]isoindol-3(4H)-yl)methyl)phenyl)-4- methoxybenzamide |
| C23 | 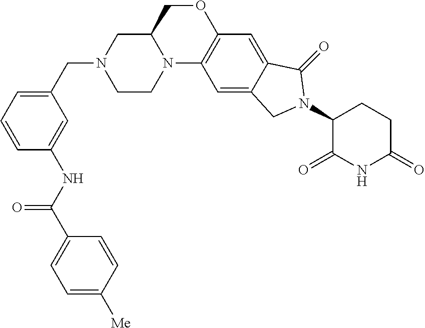 [SMILES: Cc1ccc(C(=O)Nc2cccc(CN3CCN4c5cc6c(cc5OC[C@@H]4C3)C(=O)N([C@H]3CCC(=O)NC3=O)C6)c2)cc1] | N-(3-(((S)-9-((S)-2,6-dioxopiperidin-3- yl)-8-oxo-1,2,4a,5,9,10-hexahydro-8H- pyrazino[1′,2′:4,5][1,4]oxazino[2,3- f]isoindol-3(4H)-yl)methyl)phenyl)-4- methylbenzamide |
| C24 | 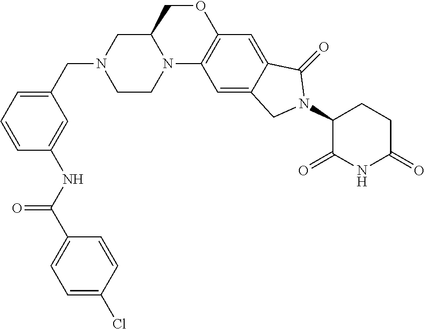 [SMILES: O=C1CC[C@H](N2Cc3cc4c(cc3C2=O)OC[C@@H]2CN(Cc3cccc(NC(=O)c5ccc(Cl)cc5)c3)CCN42)C(=O)N1] | 4-chloro-N-(3-(((S)-9-((S)-2,6- dioxopiperidin-3-yl)-8-oxo- 1,2,4a,5,9,10-hexahydro-8H- pyrazino[1′,2′:4,5][1,4]oxazino[2,3- f]isoindol-3(4H)- yl)methyl)phenyl)benzamide |
| C25 | 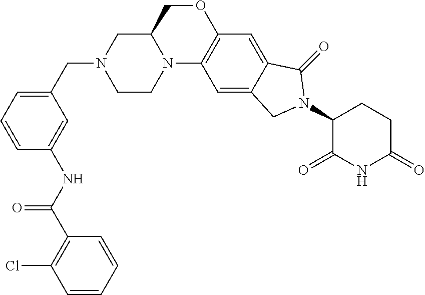 [SMILES: O=C1CC[C@H](N2Cc3cc4c(cc3C2=O)OC[C@@H]2CN(Cc3cccc(NC(=O)c5ccccc5Cl)c3)CCN42)C(=O)N1] | 2-chloro-N-(3-(((S)-9-((S)-2,6- dioxopiperidin-3-yl)-8-oxo- 1,2,4a,5,9,10-hexahydro-8H- pyrazino[1′,2′:4,5][1,4]oxazino[2,3- f]isoindol-3(4H)- yl)methyl)phenyl)benzamide |
| C26 | 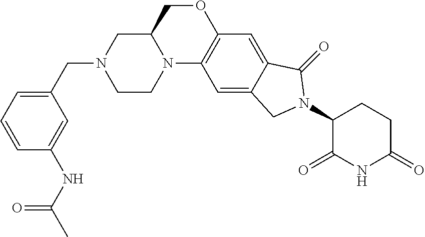 [SMILES: CC(=O)Nc1cccc(CN2CCN3c4cc5c(cc4OC[C@@H]3C2)C(=O)N([C@H]2CCC(=O)NC2=O)C5)c1] | N-(3-(((S)-9-((S)-2,6-dioxopiperidin-3- yl)-8-oxo-1,2,4a,5,9,10-hexahydro-8H- pyrazino[1′,2′:4,5][1,4]oxazino[2,3- f]isoindol-3(4H)- yl)methyl)phenyl)acetamide |
| C27 | 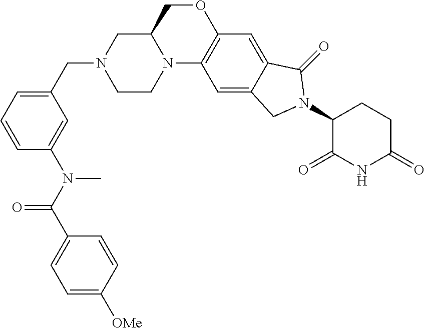 [SMILES: COc1ccc(C(=O)N(C)c2cccc(CN3CCN4c5cc6c(cc5OC[C@@H]4C3)C(=O)N([C@H]3CCC(=O)NC3=O)C6)c2)cc1] | N-(3-(((S)-9-((S)-2,6-dioxopiperidin-3- yl)-8-oxo-1,2,4a,5,9,10-hexahydro-8H- pyrazino[1′,2′:4,5][1,4]oxazino[2,3- f]isoindol-3(4H)-yl)methyl)phenyl)-4- methoxy-N-methylbenzamide |
| C28 | 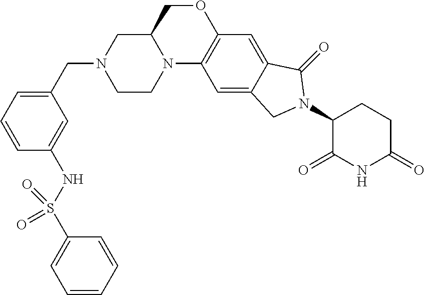 [SMILES: O=C1CC[C@H](N2Cc3cc4c(cc3C2=O)OC[C@@H]2CN(Cc3cccc(NS(=O)(=O)c5ccccc5)c3)CCN42)C(=O)N1] | N-(3-(((S)-9-((S)-2,6-dioxopiperidin-3- yl)-8-oxo-1,2,4a,5,9,10-hexahydro-8H- pyrazino[1′,2′:4,5][1,4]oxazino[2,3- f]isoindol-3(4H)- yl)methyl)phenyl)benzenesulfonamide |
| C29 | 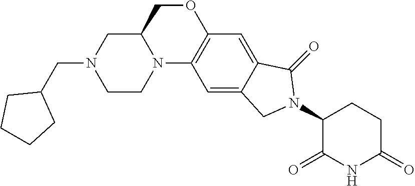 [SMILES: O=C1CC[C@H](N2Cc3cc4c(cc3C2=O)OC[C@@H]2CN(CC3CCCC3)CCN42)C(=O)N1] | (S)-3-((S)-3-(cyclopentylmethyl)-8-oxo- 1,2,3,4a,5,8,10-octahydro-9H- pyrazino[1′,2′:4,5][1,4]oxazino[2,3- f]isoindol-9-yl)piperidine-2,6-dione |
| C30 | 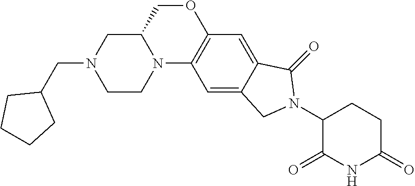 [SMILES: O=C1CCC(N2Cc3cc4c(cc3C2=O)OC[C@H]2CN(CC3CCCC3)CCN42)C(=O)N1] | 3-((R)-3-(cyclopentylmethyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C31 | 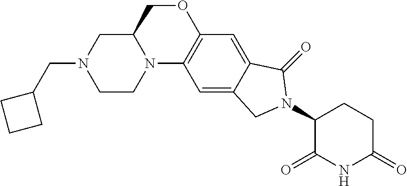 [SMILES: O=C1CC[C@H](N2Cc3cc4c(cc3C2=O)OC[C@@H]2CN(CC3CCC3)CCN42)C(=O)N1] | (S)-3-((S)-3-(cyclobutylmethyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C32 | 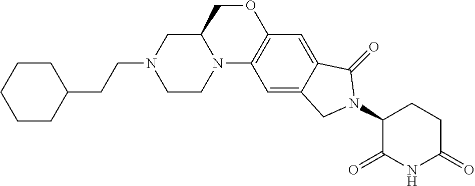 [SMILES: O=C1CC[C@H](N2Cc3cc4c(cc3C2=O)OC[C@@H]2CN(CCC3CCCCC3)CCN42)C(=O)N1] | (S)-3-((S)-3-(2-cyclohexylethyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C33 | 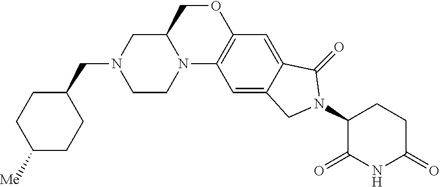 [SMILES: C[C@H]1CC[C@H](CN2CCN3c4cc5c(cc4OC[C@@H]3C2)C(=O)N([C@H]2CCC(=O)NC2=O)C5)CC1] | (S)-3-((S)-3-(((1r,4S)-4- methylcyclohexyl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C34 | 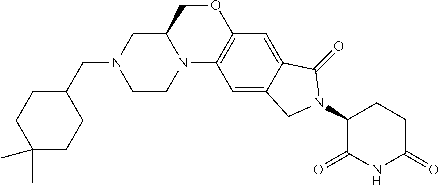 [SMILES: CC1(C)CCC(CN2CCN3c4cc5c(cc4OC[C@@H]3C2)C(=O)N([C@H]2CCC(=O)NC2=O)C5)CC1] | (S)-3-((S)-3-((4,4- dimethylcyclohexyl)methyl)-8-oxo- 1,2,3,4,4a,5,8,10-octahydro-9H- pyrazino[1′,2′:4,5][1,4]oxazino[2,3- f]isoindol-9-yl)piperidine-2,6-dione |
| C35 | 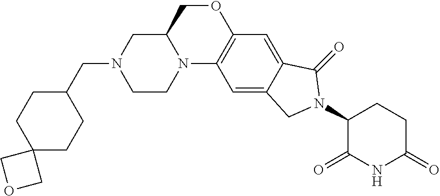 [SMILES: O=C1CC[C@H](N2Cc3cc4c(cc3C2=O)OC[C@@H]2CN(CC3CCC5(CC3)COC5)CCN42)C(=O)N1] | (S)-3-((S)-3-((2-oxaspiro[3.5]nonan-7- yl)methyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| C36 | 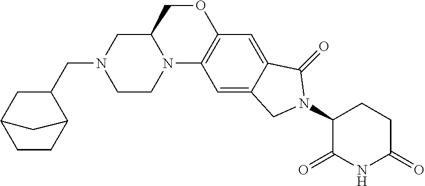 [SMILES: O=C1CC[C@H](N2Cc3cc4c(cc3C2=O)OC[C@@H]2CN(CC3CC5CCC3C5)CCN42)C(=O)N1] | (3S)-3-((4aS)-3-(bicyclo[2.2.1]heptan-2- ylmethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| C37 | 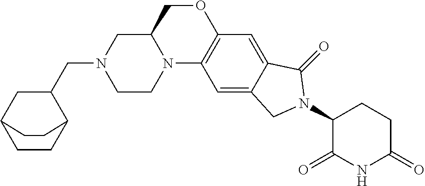 [SMILES: O=C1CC[C@H](N2Cc3cc4c(cc3C2=O)OC[C@@H]2CN(CC3CC5CCC3CC5)CCN42)C(=O)N1] | (3S)-3-((4aS)-3-(bicyclo[2.2.2]octan-2- ylmethyl)-8-oxo-1,2,3,4,4a,5,8,10- octahydro-9H- pyrazino[1′,2′:4,5][1,4]oxazino[2,3- f]isoindol-9-yl)piperidine-2,6-dione |
| C38 | 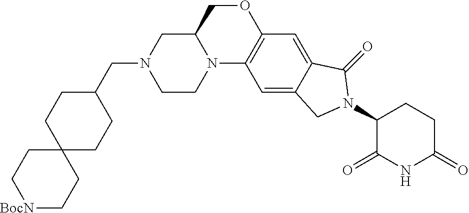 [SMILES: CC(C)(C)OC(=O)N1CCC2(CCC(CN3CCN4c5cc6c(cc5OC[C@@H]4C3)C(=O)N([C@H]3CCC(=O)NC3=O)C6)CC2)CC1] | tert-butyl 9-(((S)-9-((S)-2,6-dioxopiperidin-3- yl)-8-oxo-1,2,4a,5,9,10-hexahydro-8H- pyrazino[1′,2′:4,5][1,4]oxazino[2,3- f]isoindol-3(4H)-yl)methyl)-3- azaspiro[5.5]undecane-3-carboxylate |

### Bifunctional Degraders

In certain aspects, the present disclosure provides conjugates comprising a compound disclosed herein being connected to a ligand for a protein (e.g., via a linker).

In certain aspects, the present disclosure provides conjugates of Formula II:

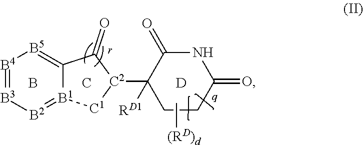

and pharmaceutically acceptable salts, solvates, or stereoisomers thereof, wherein:
B² is N or CR^{B2};
B³ is N or CR^{B3};
B⁴ is N or CR^{B4};
B⁵ is N or CR^{B5};
one of R^{B2} and R^{B3}, R^{B3} and R^{B4}, or R^{B4} and R^{B5}, together with the intervening carbon atoms, form Ring A attached to -L-T, wherein Ring A is optionally substituted 7- to 16-membered fused carbocycle or optionally substituted 7- to 16-membered fused heterocycle;
the remaining two of R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
provided that R^{B2} and R^{B3}, R^{B3} and R^{B4}, or R^{B4} and R^{B5}, together with the intervening carbon atoms, form Ring A attached to L-T, wherein Ring A is optionally substituted 7- to 16-membered fused heterocycle; and only one of R^{B2} and R^{B3}, R^{B3} and R^{B4}, and R^{B4} and R^{B5} forms Ring A attached to L-T;
[symbol] denotes an optional covalent bond between B¹ and C¹;
i) when the bond between B¹ and C¹ is present:
   r is 1;
   B¹ is C;
   C¹ is —C(R^{C1})₂— or —C(═O)—;
each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
   two R^{C1}, together with the carbon atom to which they are attached, form C₃₋₆ carbocycle or 3- to 6-membered heterocycle, wherein the carbocycle or heterocycle is optionally substituted with one or more R^{u}; and
   C² is N;
ii) when the bond between B¹ and C¹ is absent:
   r is 0 or 1;
   B¹ is N or CR^{B1};
   R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
   C¹ is absent; or
   C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
   C² is N or O;
   wherein i) when C² is N, C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; ii) when C² is O, C¹ is absent;
R^{D1} is hydrogen, deuterium, or C₁₋₆ alkyl optionally substituted with one or more R^{u};
q is an integer from 0 to 2,
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 5;
L is a linker; and
T is a ligand for a protein,
   wherein each of the variables in Formula II is described herein.

In certain embodiment, the conjugate is a conjugate selected from

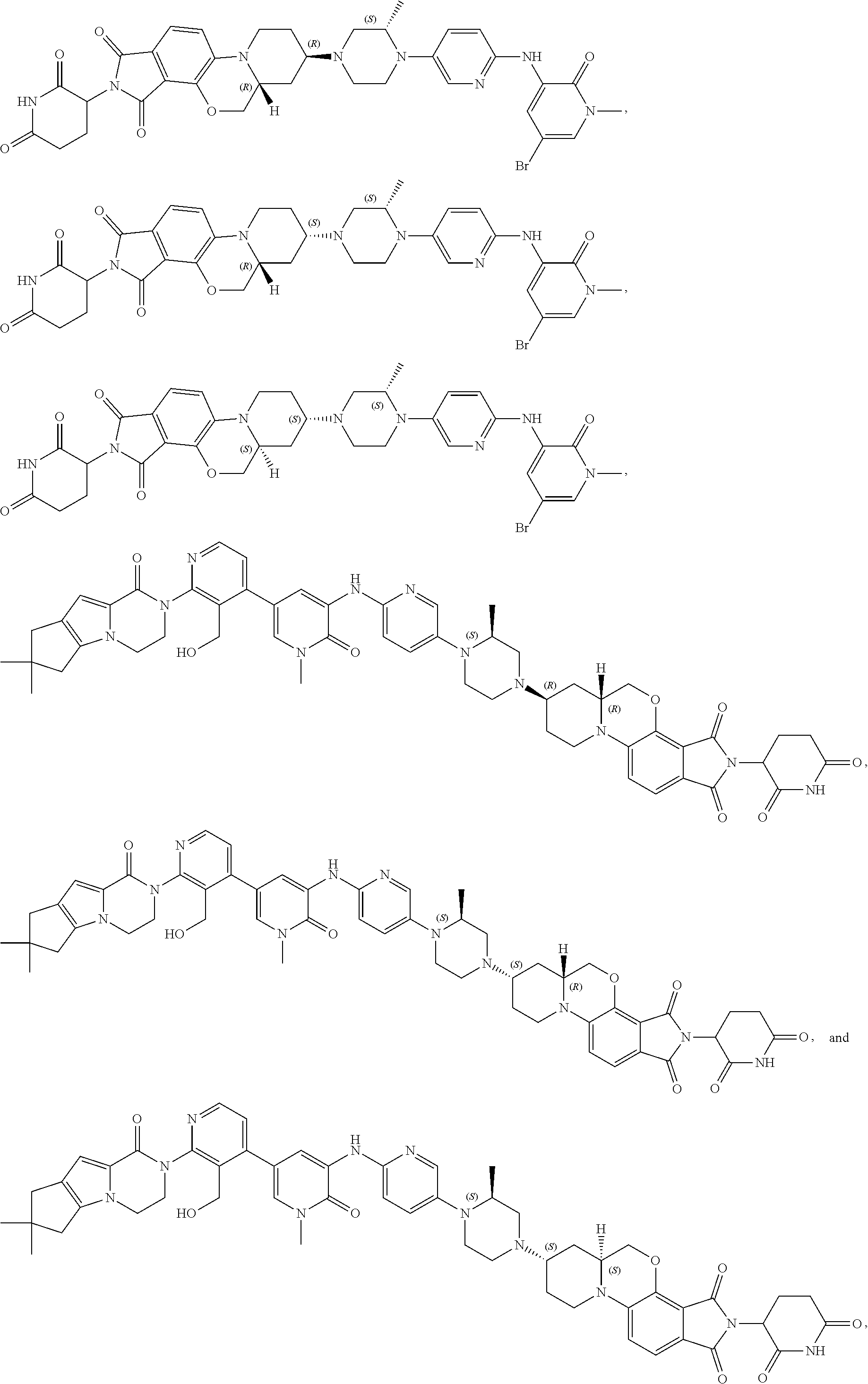

or a pharmaceutically acceptable salt, solvate, or stereoisomer thereof.

In certain embodiments, Ring A attached to -L-T is

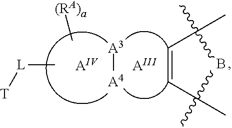

wherein each of the variables is defined herein.

In certain embodiments, Ring A attached to -L-T is

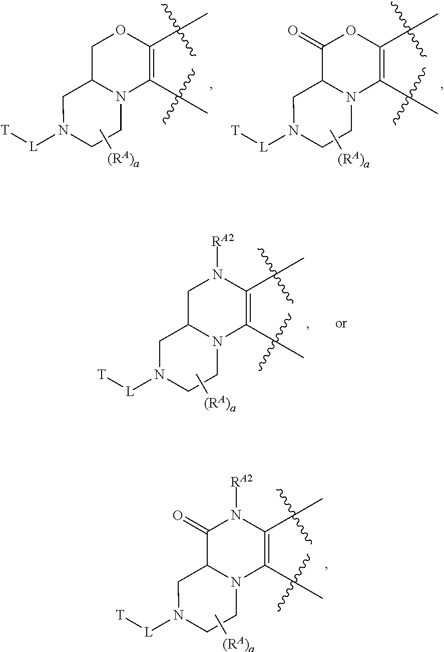

wherein each of the variables is defined herein.

In certain embodiments, the conjugate is a conjugate of Formula II-1-b-i, II-1-b-ii, II-1-b-iii, or II-1-b-iv:

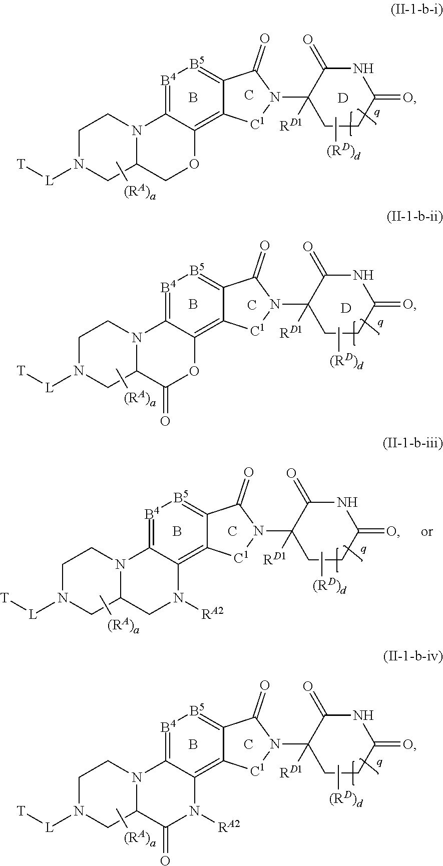

or a pharmaceutically acceptable salt, solvate, or stereoisomer thereof, wherein each of the variables is defined herein.

In certain embodiments, the conjugate is a conjugate of Formula II-2-b-i, II-2-b-ii, II-2-b-iii, or II-2-b-iv

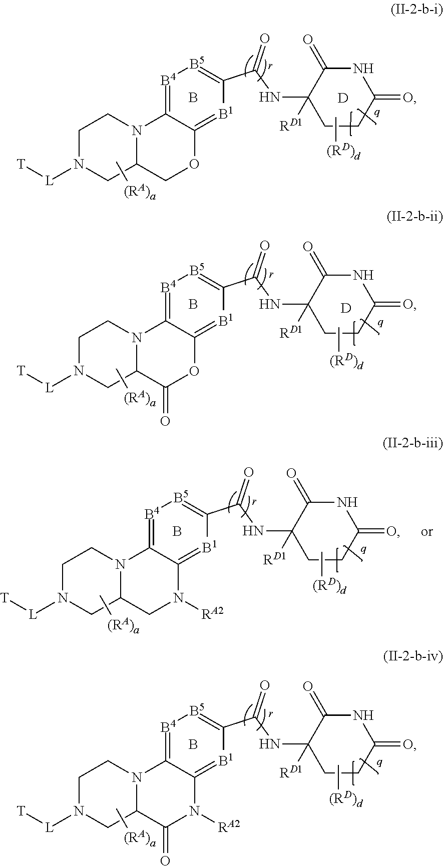

or a pharmaceutically acceptable salt, solvate, or stereoisomer thereof, wherein each of the variables is defined herein.

In certain embodiments, the conjugate is a conjugate of Formula II-1-b-v, II-1-b-vi, II-1-b-vii, or II-1-b-viii

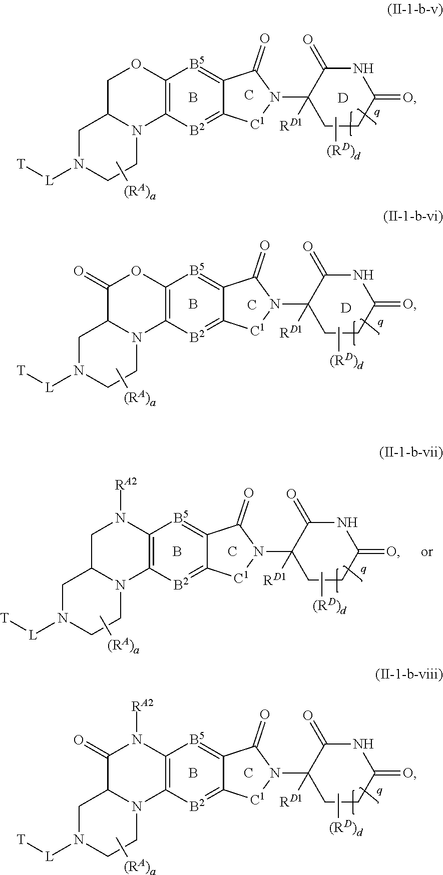

or a pharmaceutically acceptable salt, solvate, or stereoisomer thereof, wherein each of the variables is defined herein.

In certain embodiments, the conjugate is a conjugate of Formula II-2-b-v, II-2-b-vi, II-2-b-vii, or II-2-b-viii

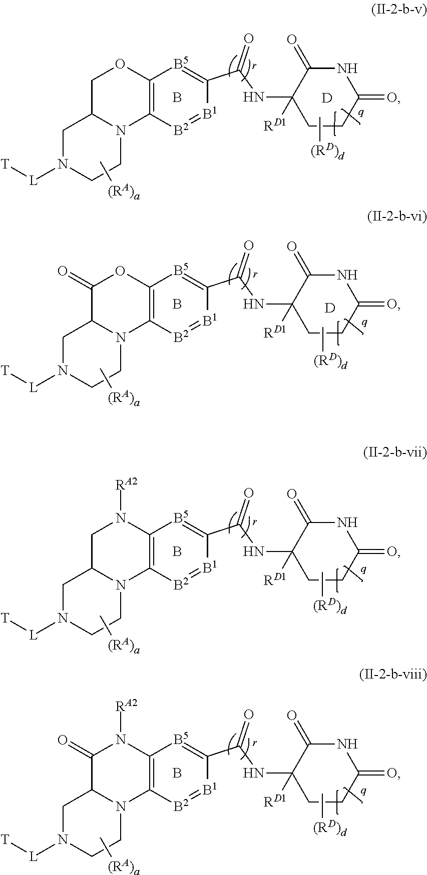

or a pharmaceutically acceptable salt, solvate, or stereoisomer thereof, wherein each of the variables is defined herein.

L, a linker, is a divalent chemical moiety that connects the ligand of a protein with the cereblon ligand disclosed herein. L configures the ligand and the cereblon ligand such that the construct functions as a bifunctional degrader which binds the cereblon ligand and selectively degrads the target protein.

In certain embodiments, L is a linker comprising C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted by one or more R^{u}.

In certain embodiments, L is of Formula II-X

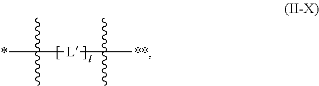

wherein:
* denotes attachment to T and ** denotes attachment to C;
each L′ is independently C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 6.

In certain embodiments, each L′ is independently C₁₋₆ alkylene (e.g., methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—)), C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)), C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), 3- to 12-membered heterocyclylene (e.g., heterocyclylene comprising one or two 3- to 8-membered rings and 1-5 heteroatoms selected from N, O, and S), C₆₋₁₀ arylene (e.g., phenylene or naphthylene), 5- to 10-membered heteroarylene (e.g., heteroarylene comprising one or two 5- or 6-membered rings and 1-5 heteroatoms selected from N, O, and S), —C(═O)—, —C(═O)N(R^{L2})—, —C(═O)O—, —N(R^{L2})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₃₋₁₂ carbocyclylene, 3- to 12-membered heterocyclylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each occurrence of R^{L′} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L′} is independently hydrogen, C₁₋₆ alkyl, C₃₋₆ carbocyclyl, 3- to 6-membered heterocyclyl, —S(═O)₂R^{A}, —S(═O)₂OR^{b}, —S(═O)₂NR^{c}R^{d}, —C(═O)R^{A}, —C(═O)OR^{b}, or —C(═O)NR^{c}R^{d}, wherein the alkyl, carbocyclyl, or heterocyclyl is optionally substituted with one or more R^{u}.

In certain embodiments, 1 is 0. In certain embodiments, t is 1. In certain embodiments, 1 is 2. In certain embodiments, 1 is 3. In certain embodiments, 1 is 4. In certain embodiments, 1 is 5. In certain embodiments, 1 is 6.

T, a ligand of a protein, is a chemical entity that competitively or non-competitively binds a protein.

In certain embodiments, the protein is B7.1 and B7, TINFR1m, TNFR2, NADPH oxidase, BclIBax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VC AM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2YT, P2Y2, P2Y4, P2Y6, P2X1-7), farnesyl transferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In certain embodiments, the protein is an androgen receptor (AR), an estrogen receptor (ER), signal transducer and activator of transcription 3 (STAT3), signal transducer and activator of transcription 5 (STAT5), CREB-binding protein/EP300(E1A) binding protein (CBP/p300), SWI/SNF Related, Matrix Associated, Actin Dependent Regulator Of Chromatin, Subfamily A, Member 2/4 (SMARCA2/4), Kirsten rat sarcoma viral oncogene homolog G12D (KRAS G12D), Src homology region 2-containing protein tyrosine phosphatase 2 (SHP2), bromodomain-containing protein 4 (BRD4) or or BRD9.

In certain embodiments, T is a small molecule.

In certain embodiments, T is an antibody.

In certain embodiments, T is a peptide. In certain embodiments, the peptide has about 5 amino acids. In certain embodiments, the peptide has about 10 amino acids. In certain embodiments, the peptide has about 15 amino acids. In certain embodiments, the peptide has about 20 amino acids. In certain embodiments, the peptide has about 25 amino acids. In certain embodiments, the peptide has about 30 amino acids. In certain embodiments, the peptide has about 35 amino acids. In certain embodiments, the peptide has about 40 amino acids. In certain embodiments, the peptide has about 45 amino acids. In certain embodiments, the peptide has about 50 amino acids.

In certain embodiments, T is a ligand for an estrogen receptor. In certain embodiments, T is ligand for an androgen receptor. In certain embodiments, T is ligand for a STAT3 protein.

In certain embodiments, T is an estrogen receptor inhibitor. In certain embodiments, T is an androgen receptor inhibitor. In certain embodiments, T is a STAT3 protein inhibitor.

In certain embodiments, the conjugate is of Formula I′:

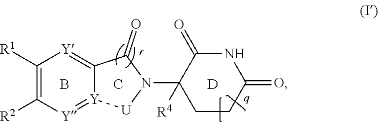

and pharmaceutically acceptable salts, solvates, or stereoisomers thereof,
wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R¹ and R², together with the intervening carbon atoms, form optionally substituted 7- to 16-membered fused heterocycle attached to -L-T;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NRCS(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more Rᵤ; or
R² and R³, together with the intervening carbon atoms, form optionally substituted 7- to 16-membered fused heterocycle attached to -L-T;
provided that either R¹ and R², or R² and R³, form optionally substituted 7- to 16-membered fused heterocycle attached to -L-T;
Y′ is N or CR^{Y′};
R^{Y′} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
--- denotes an optional covalent bond between Y and U;
when the bond between Y and U is absent:
r is 0 or 1;
Y is N or CR^{Y};
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
U is hydrogen or C₁₋₆ alkyl optionally substituted with one or more R^{u};
when the bond between Y and U is present:
r is 1;
Y is C;
U is —CH₂—, —C(═O)—, —(C═O)—N(R^{U})—*, —N═C(R^{U})—*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and
q is an integer from 0 to 2,
L is a linker, and
T is a ligand for a protein,
   wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the optionally substituted 7- to 16-membered fused heterocycle attached to -L-T is not

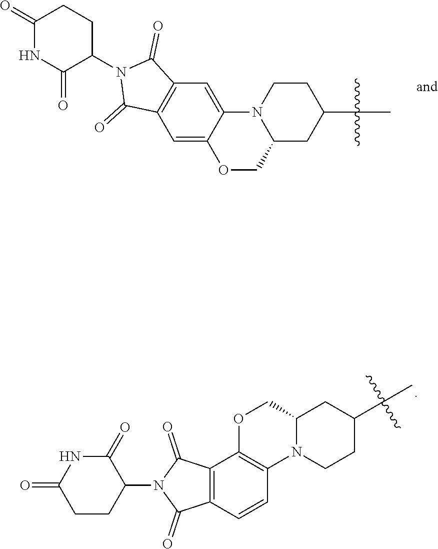 [SMILES: *C1CCN2c3cc4c(cc3OC[C@H]2C1)C(=O)N(C1CCC(=O)NC1=O)C4=O ; *C1CCN2c3ccc4c(c3OC[C@@H]2C1)C(=O)N(C1CCC(=O)NC1=O)C4=O]

In certain embodiment, the conjugate is a conjugate selected from

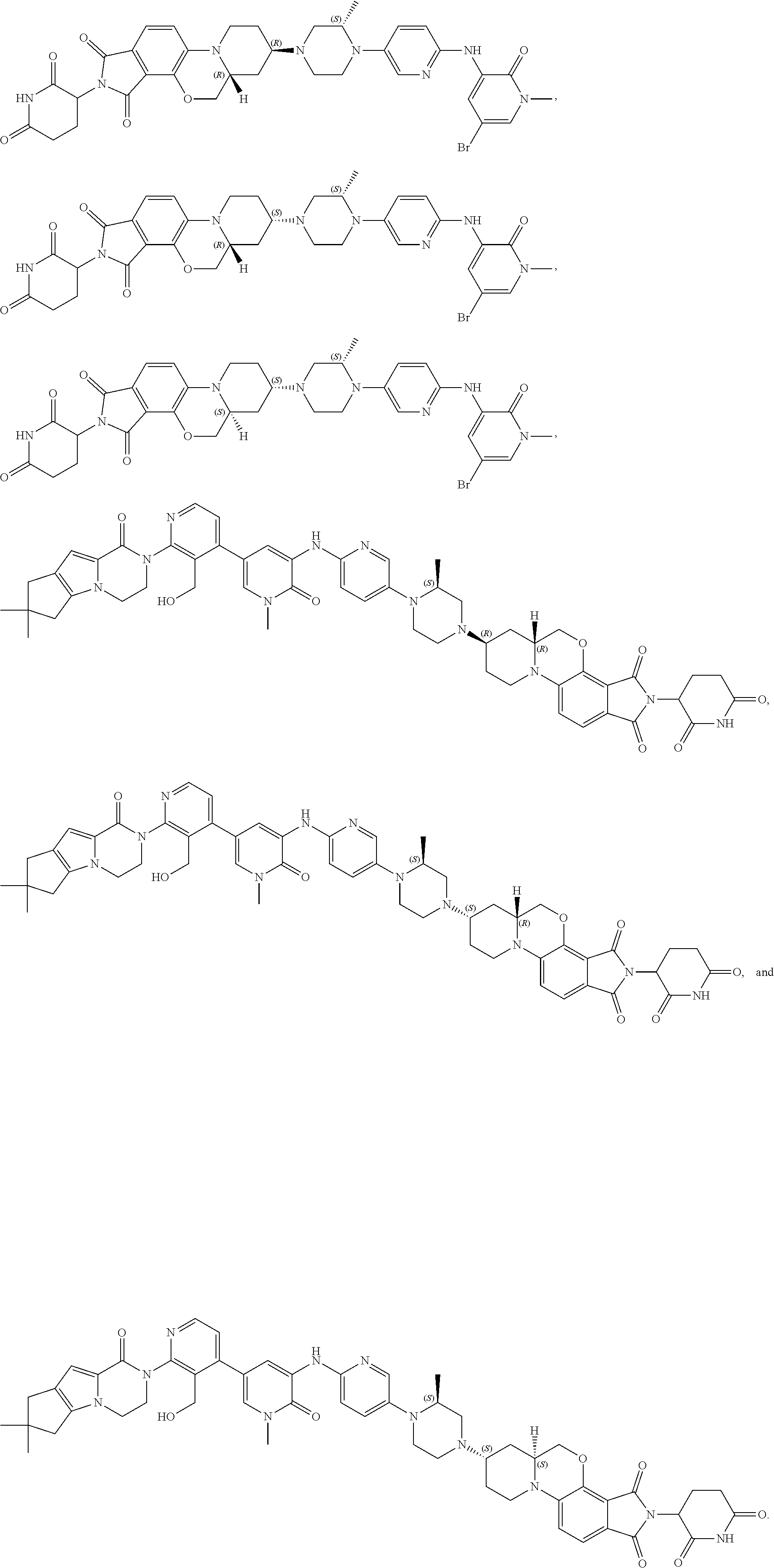

In certain embodiments, the conjugate of Formula I′ is a conjugate of Formula I′-1

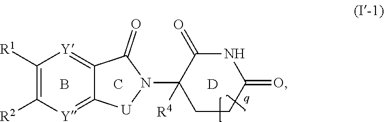

or a pharmaceutically acceptable salt, solvate, or stereoisomer thereof.

In certain embodiments, U is —CH₂— or —C(═O)—.

In certain embodiments, the conjugate of Formula I′ is a conjugate of Formula I′-2

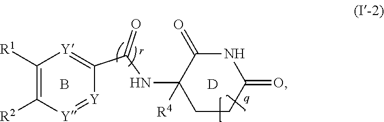

or a pharmaceutically acceptable salt, solvate, or stereoisomer thereof.

In certain embodiments, Y is N.

In certain embodiments, Y is CR^{Y}, and R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, Y is CR^{Y}.

In certain embodiments, R^{Y} is hydrogen, halogen, or C₁₋₆alkoxy.

In certain embodiments, R¹ and R², together with the intervening carbon atoms, form optionally substituted 8- to 12-membered fused bicyclic heterocycle attached to -L-T.

In certain embodiments, the 7- to 16-membered or 8- to 12-membered fused heterocycle is optionally substituted with one or more R^{u}. In certain embodiments, the 7- to 16-membered or 8- to 12-membered fused heterocycle is optionally substituted with one or more R¹. In certain embodiments, the 7- to 16-membered or 8- to 12-membered fused heterocycle is optionally substituted with one or more R⁵. In certain embodiments, the 7- to 16-membered or 8- to 12-membered fused heterocycle is optionally substituted with one or more R⁶.

In certain embodiments, R^{u} is Rⁱ. In certain embodiments, R^{u} is R⁵. In certain embodiments, R^{u} is R⁶. In certain embodiments, Rⁱ is R⁵. In certain embodiments, Rⁱ is R⁶.

In certain embodiments, the 7- to 16-membered or 8- to 12-membered fused heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, R¹ and R², together with the intervening carbon atoms, form optionally substituted 8- to 12-membered fused bicyclic heterocycle attached to -L-T

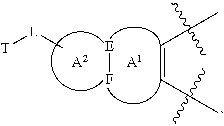

wherein E and F are independently CH or N.

In certain embodiments, Ring A¹ is optionally substituted 5- to 7-membered heterocycle.

In certain embodiments, Ring A¹ is optionally substituted 5- to 7-membered heterocycle comprising one nitrogen atom. In certain embodiments, Ring A¹ is optionally substituted 5- to 7-membered heterocycle comprising two nitrogen atoms.

In certain embodiments, Ring A¹ is optionally substituted 5-membered heterocycle. In certain embodiments, Ring A¹ is optionally substituted 6-membered heterocycle. In certain embodiments, Ring A¹ is optionally substituted 7-membered heterocycle.

In certain embodiments, Ring A¹ is optionally substituted 5-membered heterocycle comprising one nitrogen atom. In certain embodiments, Ring A¹ is optionally substituted 6-membered heterocycle comprising one nitrogen atom. In certain embodiments, Ring A¹ is optionally substituted 7-membered heterocycle comprising one nitrogen atom.

In certain embodiments, Ring A¹ is optionally substituted 5-membered heterocycle comprising two nitrogen atoms. In certain embodiments, Ring A¹ is optionally substituted 6-membered heterocycle comprising two nitrogen atoms. In certain embodiments, Ring A¹ is optionally substituted 7-membered heterocycle comprising two nitrogen atoms.

In certain embodiments, Ring A¹ is optionally substituted with one or more R^{u}. In certain embodiments, Ring A¹ is optionally substituted with one or more Rⁱ. In certain embodiments, Ring A¹ is optionally substituted with one or more R⁵. In certain embodiments, Ring A¹ is optionally substituted with one or more R⁶.

In certain embodiments, Ring A² is optionally substituted 5- to 7-membered heterocycle.

In certain embodiments, Ring A² is optionally substituted 5- to 7-membered heterocycle comprising one nitrogen atom. In certain embodiments, Ring A² is optionally substituted 5- to 7-membered heterocycle comprising two nitrogen atoms.

In certain embodiments, Ring A² is optionally substituted 5-membered heterocycle. In certain embodiments, Ring A² is optionally substituted 6-membered heterocycle. In certain embodiments, Ring A² is optionally substituted 7-membered heterocycle.

In certain embodiments, Ring A² is optionally substituted 5-membered heterocycle comprising one nitrogen atom. In certain embodiments, Ring A² is optionally substituted 6-membered heterocycle comprising one nitrogen atom. In certain embodiments, Ring A² is optionally substituted 7-membered heterocycle comprising one nitrogen atom.

In certain embodiments, Ring A² is optionally substituted 5-membered heterocycle comprising two nitrogen atoms. In certain embodiments, Ring A² is optionally substituted 6-membered heterocycle comprising two nitrogen atoms. In certain embodiments, Ring A² is optionally substituted 7-membered heterocycle comprising two nitrogen atoms.

In certain embodiments, Ring A² is optionally substituted with one or more R^{u}. In certain embodiments, Ring A² is optionally substituted with one or more R¹. In certain embodiments, Ring A² is optionally substituted with one or more R⁵. In certain embodiments, Ring A² is optionally substituted with one or more R⁶.

In certain embodiments, R¹ and R², together with the intervening carbon atoms, form 8- to 12-membered fused bicyclic heterocycle selected from

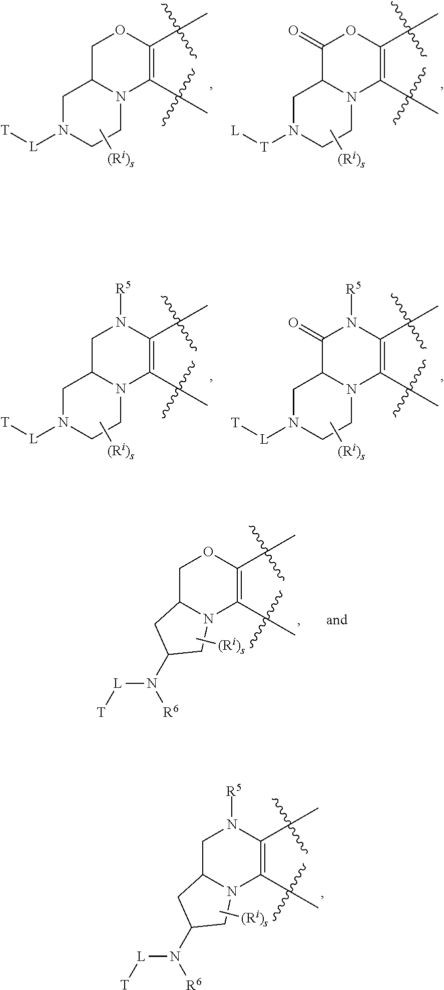

wherein:
R⁵ is hydrogen or C₁₋₆ alkyl optionally substituted with one or more R^{u}; or
R⁵ is an amino-protecting group;
R⁶ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —S(═O)₂R^{a}, —S(═O)₂OR^{b}, —S(═O)₂NR′R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R⁶ is an amino-protecting group;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, the conjugate of Formula I′-1 is a conjugate of Formula I′-1-i, I′-1-ii, I′-1-iii, I′-iv, I′-1-v, I′-1-vi, I′-1-vii, or I′-1-viii:

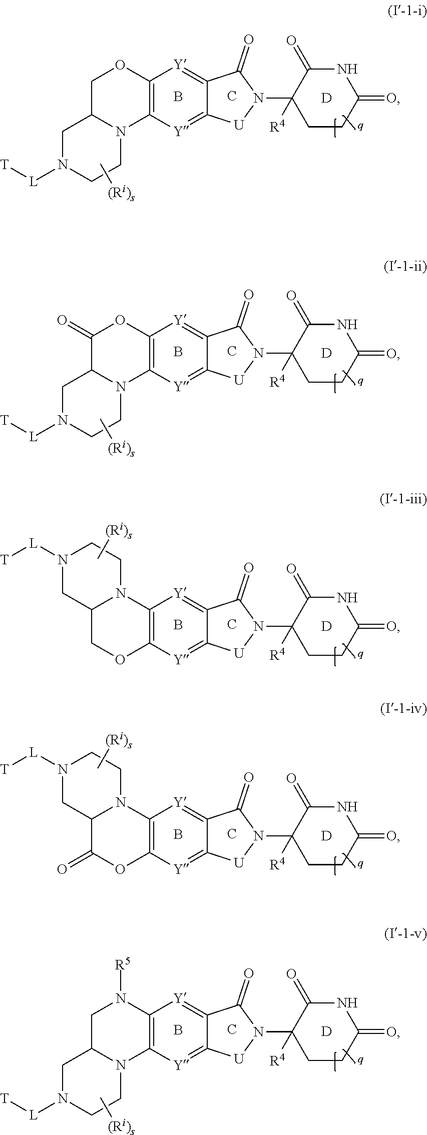

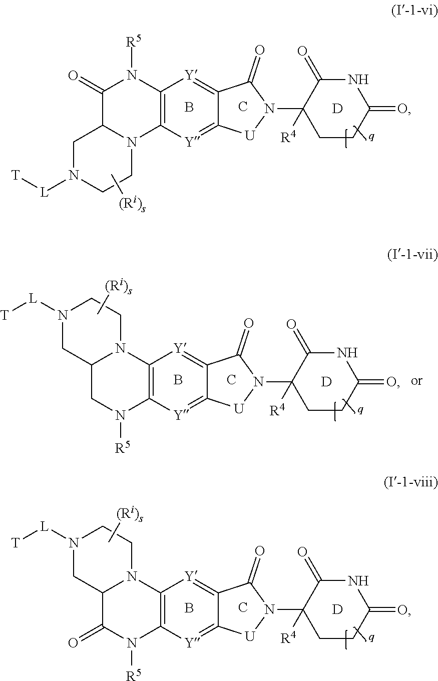

or a pharmaceutically acceptable salt, solvate, or stereoisomer thereof.

In certain embodiments, the conjugate of Formula I′-2 is a conjugate of Formula I′-2-i, I′-2-ii, I′-2-iii, I′-2-iv, I′-2-v, I′-2-vi, I′-2-vii, or I′-2-viii

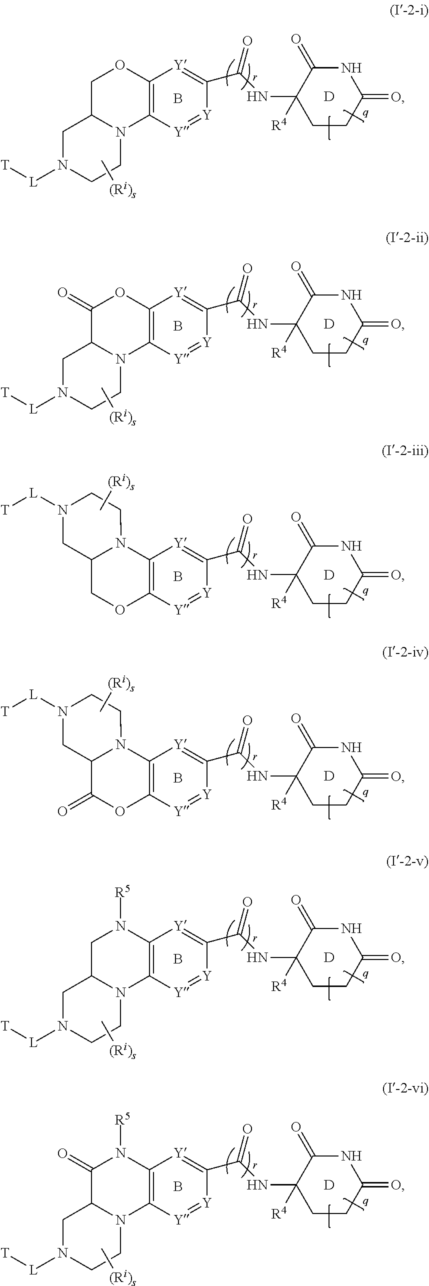

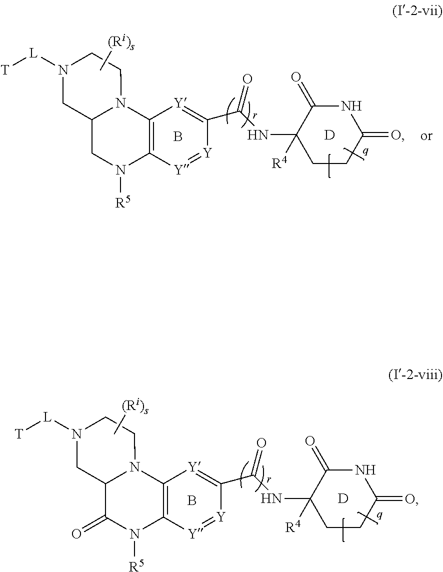

or a pharmaceutically acceptable salt, solvate, or stereoisomer thereof.

In certain embodiments, R⁵ is hydrogen or C₁₋₆ alkyl.

In certain embodiments, Y″ is N.

In certain embodiments, Y″ is CR³.

In certain embodiments, R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R³ is hydrogen.

In certain embodiments, R² and R³, together with the intervening carbon atoms, form optionally substituted 8- to 12-membered fused bicyclic heterocycle attached to -L-T.

In certain embodiments, the 7- to 16-membered or 8- to 12-membered fused heterocycle is optionally substituted with one or more R^{u}. In certain embodiments, the 7- to 16-membered or 8- to 12-membered fused heterocycle is optionally substituted with one or more Rⁱ. In certain embodiments, the 7- to 16-membered or 8- to 12-membered fused heterocycle is optionally substituted with one or more R⁵. In certain embodiments, the 7- to 16-membered or 8- to 12-membered fused heterocycle is optionally substituted with one or more R⁶.

In certain embodiments, R^{u} is Rⁱ. In certain embodiments, R^{u} is R⁵. In certain embodiments, R^{u} is R⁶. In certain embodiments, Rⁱ is R⁵. In certain embodiments, Rⁱ is R⁶.

In certain embodiments, the 7- to 16-membered or 8- to 12-membered fused heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR ^{S(═O)}₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, R² and R³, together with the intervening carbon atoms, form optionally substituted 8- to 12-membered fused bicyclic heterocycle attached to -L-T

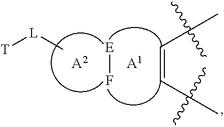

wherein E and F are independently CH or N.

In certain embodiments, Ring A¹ is optionally substituted 5- to 7-membered heterocycle.

In certain embodiments, Ring A¹ is optionally substituted 5- to 7-membered heterocycle comprising one nitrogen atom. In certain embodiments, Ring A¹ is optionally substituted 5- to 7-membered heterocycle comprising two nitrogen atoms.

In certain embodiments, Ring A¹ is optionally substituted 5-membered heterocycle. In certain embodiments, Ring A¹ is optionally substituted 6-membered heterocycle. In certain embodiments, Ring A¹ is optionally substituted 7-membered heterocycle.

In certain embodiments, Ring A¹ is optionally substituted 5-membered heterocycle comprising one nitrogen atom. In certain embodiments, Ring A¹ is optionally substituted 6-membered heterocycle comprising one nitrogen atom. In certain embodiments, Ring A¹ is optionally substituted 7-membered heterocycle comprising one nitrogen atom.

In certain embodiments, Ring A¹ is optionally substituted 5-membered heterocycle comprising two nitrogen atoms. In certain embodiments, Ring A¹ is optionally substituted 6-membered heterocycle comprising two nitrogen atoms. In certain embodiments, Ring A¹ is optionally substituted 7-membered heterocycle comprising two nitrogen atoms.

In certain embodiments, Ring A¹ is optionally substituted with one or more R^{u}. In certain embodiments, Ring A¹ is optionally substituted with one or more R¹. In certain embodiments, Ring A¹ is optionally substituted with one or more R⁵. In certain embodiments, Ring A¹ is optionally substituted with one or more R⁶.

In certain embodiments, Ring A² is optionally substituted 5- to 7-membered heterocycle.

In certain embodiments, Ring A² is optionally substituted 5- to 7-membered heterocycle comprising one nitrogen atom. In certain embodiments, Ring A² is optionally substituted 5- to 7-membered heterocycle comprising two nitrogen atoms.

In certain embodiments, Ring A² is optionally substituted 5-membered heterocycle. In certain embodiments, Ring A² is optionally substituted 6-membered heterocycle. In certain embodiments, Ring A² is optionally substituted 7-membered heterocycle.

In certain embodiments, Ring A² is optionally substituted 5-membered heterocycle comprising one nitrogen atom. In certain embodiments, Ring A² is optionally substituted 6-membered heterocycle comprising one nitrogen atom. In certain embodiments, Ring A² is optionally substituted 7-membered heterocycle comprising one nitrogen atom.

In certain embodiments, Ring A² is optionally substituted 5-membered heterocycle comprising two nitrogen atoms. In certain embodiments, Ring A² is optionally substituted 6-membered heterocycle comprising two nitrogen atoms. In certain embodiments, Ring A² is optionally substituted 7-membered heterocycle comprising two nitrogen atoms.

In certain embodiments, Ring A² is optionally substituted with one or more R^{u}. In certain embodiments, Ring A² is optionally substituted with one or more Rⁱ. In certain embodiments, Ring A² is optionally substituted with one or more R⁵. In certain embodiments, Ring A² is optionally substituted with one or more R⁶.

In certain embodiments, R² and R³, together with the intervening carbon atoms, form 8- to 12-membered fused bicyclic heterocycle selected from

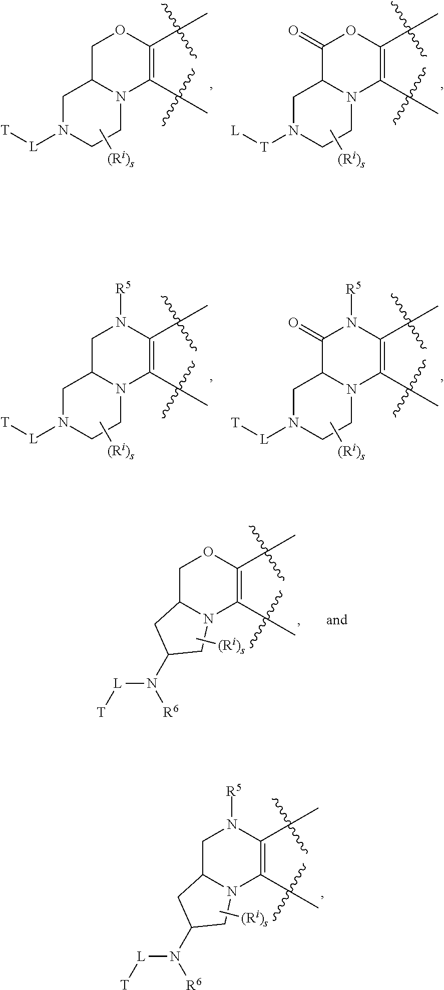

wherein:
R⁵ is hydrogen or C₁₋₆ alkyl optionally substituted with one or more R^{u}; or
R⁵ is an amino-protecting group;
R⁶ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R⁶ is an amino-protecting group;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 8, as valency permits.

In certain embodiments, the conjugate of Formula I′-1 is a conjugate of Formula I′-1-ix, I′-1-x, I′-1-xi, or I′-1-xii:

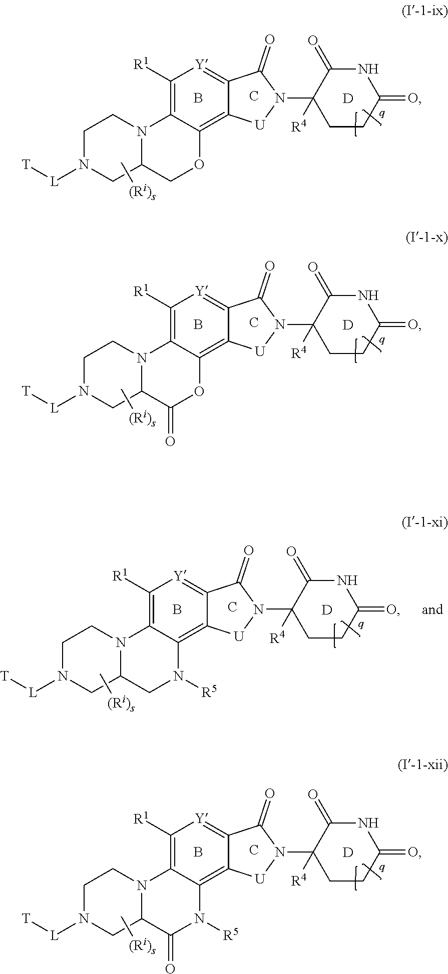

or a pharmaceutically acceptable salt, solvate, or stereoisomer thereof.

In certain embodiments, the conjugate of Formula I′-2 is a conjugate of Formula I′-2-ix, I′-2-x, I′-2-xi, or I′-2-xii:

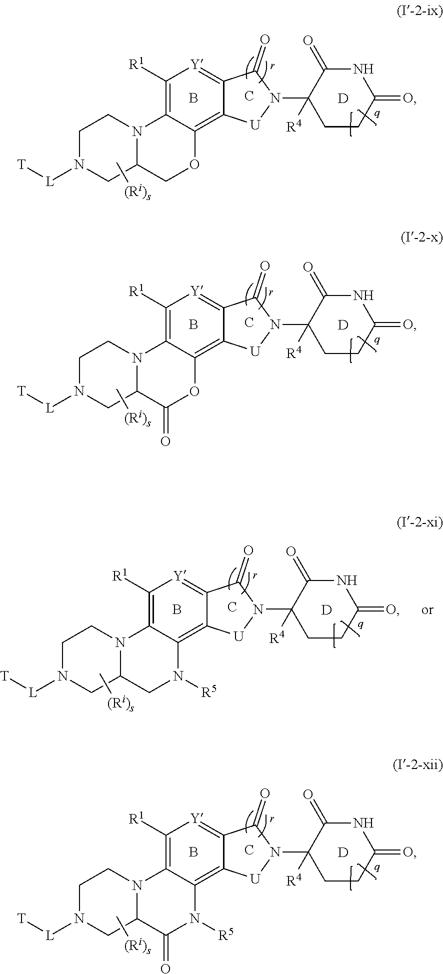

or a pharmaceutically acceptable salt, solvate, or stereoisomer thereof.

In certain embodiments, R⁵ is hydrogen or C₁₋₆ alkyl.

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R⁵ is hydrogen.

In certain embodiments, Y′ is N.

In certain embodiments, Y′ is CR^{Y′}.

In certain embodiments, R^{Y′} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y′} is hydrogen.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, s is an integer selected from 0 to 8, as valency permits. In certain embodiments, s is an integer selected from 0 to 7, as valency permits. In certain embodiments, s is an integer selected from 0 to 6, as valency permits. In certain embodiments, s is an integer selected from 0 to 5, as valency permits. In certain embodiments, s is an integer selected from 0 to 4, as valency permits. In certain embodiments, s is an integer selected from 0 to 3, as valency permits. In certain embodiments, s is an integer selected from 0 to 2, as valency permits. In certain embodiments, s is 0 or 1, as valency permits.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, R⁴ is hydrogen. In certain embodiments, R⁴ is deuterium. In certain embodiments, R⁴ is C₁₋₆ haloalkyl. In certain embodiments, R⁴ is C₁₋₆ alkyl.

In certain embodiments, q is 0. In certain embodiments, q is 1. In certain embodiments, q is 2. In certain embodiments, q is 0 or 1. In certain embodiments, q is 0 or 2. In certain embodiments, q is 1 or 2.

L, a linker, is a divalent chemical moiety that connects the ligand of a protein with the cereblon ligand disclosed herein. L configures the ligand and the cereblon ligand such that the construct functions as a bifunctional degrader which binds the cereblon ligand and selectively degrads the target protein.

In certain embodiments, L is a linker comprising 6- to 10-membered heteroarylene, C₆₋₁₀ arylene, C₃₋₁₂ membered carbocyclylene, or 3- to 12-membered heterocyclylene, wherein the arylene, heteroarylene, carbocyclylene, or heterocyclylene is optionally substituted by one or more R^{u}, and is directly attached to T.

In certain embodiments, L is of formula

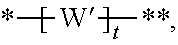

wherein:
* denotes attachment to T;
each —W′— is independently C₁₋₃ alkylene, C₂ alkenylene, C₂ alkynylene, C₃₋₁₂ carbocycylene, 3- to 12-membered heterocyclylene, C₆₋₁₀ arylene, 5- to 10-membered heteroarylene, —C(═O)—, —N(R^{L})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L} is independently hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkenyl, alkynyl, carbocyclyl, heterocyclyl, aryl, or heteroaryl is optionally substituted with one or more R^{u}; and
t is an integer selected from 1 to 15.

In certain embodiments, L is of formula

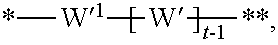

wherein:
W′¹ is 6- to 10-membered heteroarylene, C₆₋₁₀ arylene, C₃₋₁₂ membered carbocyclylene, or 3- to 12-membered heterocyclylene, wherein the arylene, heteroarylene, carbocyclylene, or heterocyclylene is optionally substituted by one or more R^{u}; and
each —W′— is independently C₁₋₃ alkylene, —C(═O)—, —N(R^{L})—, —O—, C₃₋₁₂ carbocycylene, or 3- to 12-membered heterocyclylene, wherein the alkylene, carbocyclylene, or heterocyclylene is optionally substituted with one or more R^{u}.

In certain embodiments, L is of Formula:

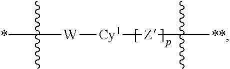

wherein:
W is absent; or
W is C₁₋₃ alkylene, —O—, —NR^{W}—, or —(C═O)—, wherein the alkylene is optionally substituted by one or more R^{u};
Cy¹ is absent; or
Cy¹ is 6-membered heteroarylene, C₆ arylene, C₃₋₁₂ membered carbocyclylene, or 3- to 12-membered heterocyclylene, wherein the arylene, heteroarylene, carbocyclylene, or heterocyclylene is optionally substituted by one or more R^{u};
Z′ is absent; or
each Z′ is independently C₁₋₃ alkylene, —O—, —NR^{W}—, —(C═O)—, C₃₋₁₂ membered carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8.

T, a ligand of a protein, is a chemical entity that competitively or non-competitively binds a protein.

In certain embodiments, the protein is B7.1 and B7, TINFR1m, TNFR2, NADPH oxidase, BclIBax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VC AM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2Y1, P2Y2, P2Y4, P2Y6, P2X1-7), farnesyl transferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In certain embodiments, the protein is an androgen receptor (AR), an estrogen receptor (ER), signal transducer and activator of transcription 3 (STAT3), signal transducer and activator of transcription 5 (STAT5), CREB-binding protein/EP300(EIA) binding protein (CBP/p300), SWI/SNF Related, Matrix Associated, Actin Dependent Regulator Of Chromatin, Subfamily A, Member 2/4 (SMARCA2/4), Kirsten rat sarcoma viral oncogene homolog G12D (KRAS G12D), Src homology region 2-containing protein tyrosine phosphatase 2 (SHP2), or bromodomain-containing protein 4 (BRD4).

In certain embodiments, T is a small molecule.

In certain embodiments, T is an antibody.

In certain embodiments, T is a peptide. In certain embodiments, the peptide has about 5 amino acids. In certain embodiments, the peptide has about 10 amino acids. In certain embodiments, the peptide has about 15 amino acids. In certain embodiments, the peptide has about 20 amino acids. In certain embodiments, the peptide has about 25 amino acids. In certain embodiments, the peptide has about 30 amino acids. In certain embodiments, the peptide has about 35 amino acids. In certain embodiments, the peptide has about 40 amino acids. In certain embodiments, the peptide has about 45 amino acids. In certain embodiments, the peptide has about 50 amino acids.

In certain embodiments, T is a ligand for an estrogen receptor. In certain embodiments, T is ligand for an androgen receptor. In certain embodiments, T is ligand for a STAT3 protein.

In certain embodiments, T is an estrogen receptor inhibitor. In certain embodiments, T is an androgen receptor inhibitor. In certain embodiments, T is a STAT3 protein inhibitor.

The compounds of the present disclosure may possess advantageous characteristics, as compared to known compounds, such as known cereblon-binding agents or known degraders comprising cereblon-binding agents. For example, the compounds of the present disclosure may display more potent cereblon-binding activity or more potent degradation activity against certain proteins, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Forms of Compounds Disclosed Herein

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the invention.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present invention.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers/Stereoisomers

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the invention.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the invention.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. All tautomeric forms of the compounds disclosed herein are contemplated and are within the scope of the invention. The exact ratio of the tautomers depends on several factors, including temperature, solvent, and pH.

### Pharmaceutical Compositions

In certain embodiments, the compound described herein is administered as a pure chemical. In certain embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In certain embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Scheme

The compounds of the present disclosure can generally be prepared by first preparing pools of intermediates, including a pool of cereblon ligands, a pool of linkers, and a pool of inhibitors, as detailed in the Example section, then followed by subsequent reactions to connect a linker to an inhibitor and a cereblon ligand via metal-catalyzed coupling reactions and reductive amination. Large pool of compounds can be prepared by selecting different combinations of cereblon ligands, linkers, and inhibitors from each pool. General synthetic routes for preparing inhibitor-linker conjugate via metal-catalyzed coupling reactions, which is further coupled to cereblon ligand via reductive amination, are summarize below.

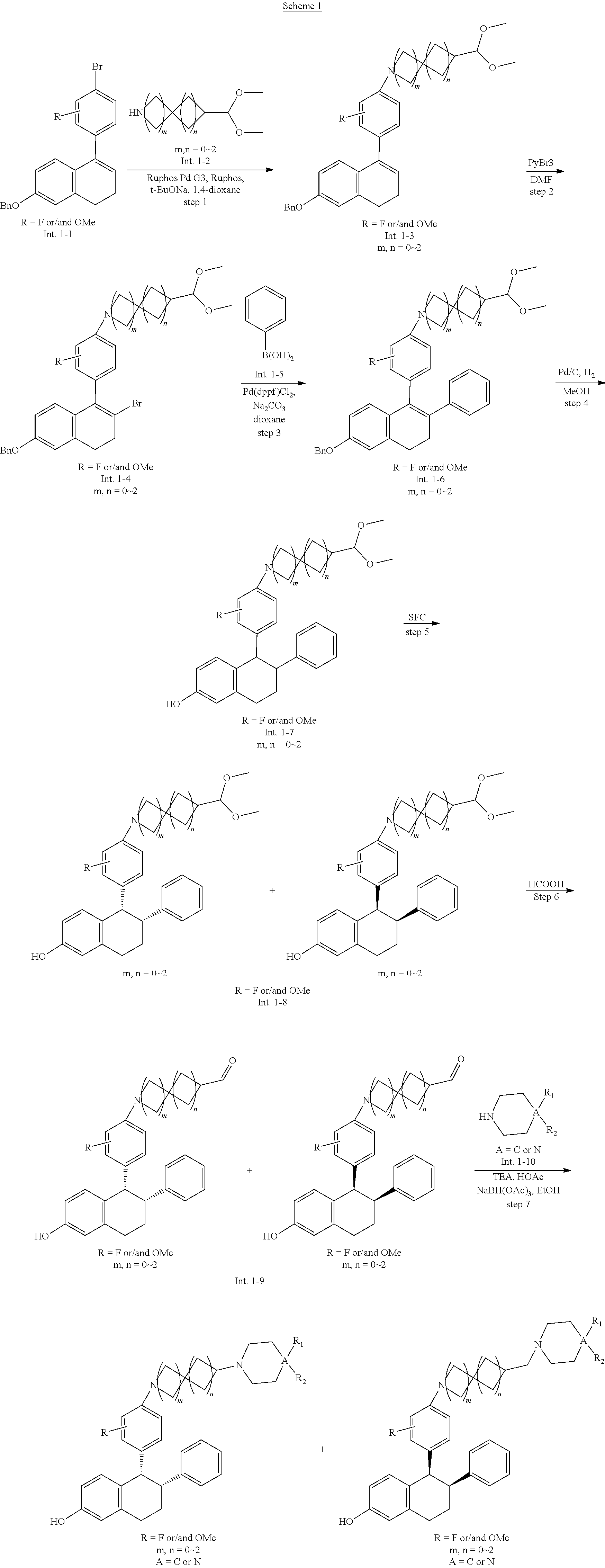

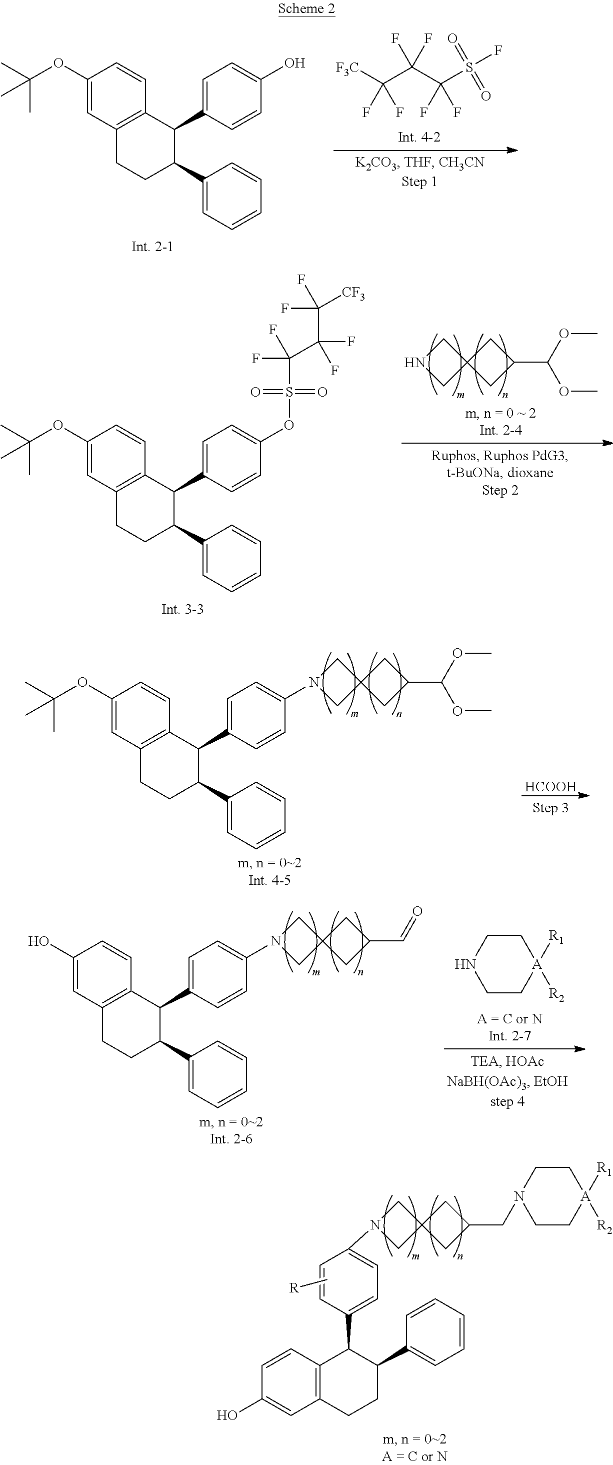

### Synthetic Procedures for Making Selective IKZF2 or IKZF1/3 Degraders

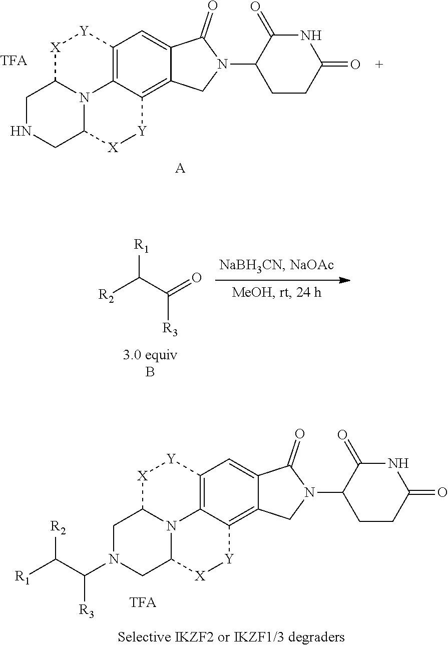

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY). Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present application can be assessed with methods and assays known in the art.

The CRBN-DDB1 binding potency of the present disclosure was determined using HTRF assay technology (Perkin Elmer). Compounds are serially diluted and are transferred multi-well plate. The reaction was conducted with addition of His tagged (e.g., CRBN+DDB-DLS7+CXU4) followed by addition of 60 nM fluorescent probe (e.g., Cy5-labeled Thalidomide), and MAb Anti-6HIS Tb cryptate Gold in the assay buffer. After one hour incubation at room temperature, the HTRF signals were read on Envision reader (Perkin Elemer).

ERa degradative activity of compounds can be assessed in MCF-7 and T47D Cells. MCF-7 and T47D cell are seeded and are subsequently treated with the compounds at certain concentrations (e.g., 0.02 to 300 nM). DMSO can be used as vehicle control. Cells are fixed and are blocked with Intercept (PBS) Blocking Buffer (e.g., Li—COR, Odyssey Blocking Buffer), and are stained with ER (e.g., 1:500, Cell signaling) primary antibody for overnight at cold room. Secondary Antibody (e.g., IRDye 800CW Goat anti-Rabbit IgG) and CellTag 700 Stain are added in Intercept (PBS) Blocking Buffer. Finally, cell plate is placed in incubator to dry. Image and signal were captured on Odyssey@DLx Imaging System.

In vitro assay can be accompolished by MCF-7 and T47D Cell Titer Glo (CTG) assay. MCF-7 and T47D cell (From HDB) are cultured in multi-well white plate with phenol red-free RPMI1640+10% CS-FBS+1% P/S medium (e.g., at 1,000 cells/well). On day 0: Cells were treated with compound at certain concentrations (e.g., 0.5 to 10000 nM) (DMSO and Staurosporine as control). On day 0 and day 6: add Cell Titer Glo reagent and read on EnVision after 30 min incubation for data generation.

In-cell western blot analysis. Cells are seeded in multi-well plates (e.g., at 40,000 or 10,000 cells/well). Diluted compounds at certain concentration are added (final 0.5% DMSO) and cells are incubated for certain period of time (e.g., 16 hours). Formaldehyde (e.g., PBS:FA=9:1) is added and followed by washing with PBS. The cells are blocked with Licor blocking buffer (Li-Cor). The relative ER percentage in treated cells were obtained by comparing the values of treated wells to those in untreated and DMSO-treated wells as 100%.

Western Blot Analysis. The cells that are treated with the compounds are lysed in Radioimmunoprecipitation Assay Protein Lysis and Extraction Buffer (e.g., 25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail. Equal amounts of total protein are electrophoresed through 10% SDS-polyacrylamide gels after determination of protein concentration by BCA assay. The separated protein bands were transferred onto PVDF membranes and blotted against different antibodies. The blots are scanned, and the band intensities were quantified (e.g., by using GelQuant.NET software provided by biochemlabsolutions.com). The relative mean intensity of target proteins is expressed after normalization to the intensity of glyceraldehyde-3-phosphate dehydrogenase bands.

Cell Growth Assay. The cells were seeded at certain concentration (e.g., at 1500/well) in multi-well plates overnight. Cells are subsequently treated with the compounds. A certain period of time (e.g., 4 days) after the compound treatment, 10% WST-8 reagent was added to the culture medium and incubate under certain condition (e.g., in a CO₂ incubator at 37° C. for 2.5 hours). The absorbance is measured on each sample using a microplate reader at certain wavelength (e.g., 450 nm). The relative absorbance is calculated against the vehicle control from three individually repeats.

In vivo pharmacodynamic and efficacy studies. To develop breast cancer cell line xenografts, mice is given 17β-Estradiol in drinking water for certain period of time. Certain number (e.g., five million) of cells in 50% Matrigel are injected subcutaneously into SCID mice to induce tumor formation. When tumors reach certain size (e.g., 100-400 mm³), mice are treated with vehicle control (e.g., 5% DMSO, 10% solutol, 85% Water) or the compound, and sacrificed at indicated time points. Tumor tissue is harvested for analysis. Tumor sizes and animal weights were measured 2-3 times per week. Tumor volume (mm³)=(length×width2)/2. Tumor growth inhibition is calculated using TGI (%)=(Vc−Vt)/(Vc−Vo)×100, where Vc, Vt are the median of control and treated groups at the end of the study and Vo at the start.

### Methods of Use

“CRBN E3 ubiquitin ligase protein complex” is art recognized and refers to an association of proteins in which CRBN, a 442-amino acid protein, forms a Cullin-4-RING E3 ubiquitin ligase (CRL4) complex and interacts with the adaptor protein damaged DNA-binding protein 1 (DDB1), Cullin-4A (CUL4A), and regulator of cullins 1 (ROC1). Within the CRL4 complex, CRBN acts as a substrate-specificity receptor.

In certain aspects, provided herein are methods of binding cereblon E3 ubiquitin ligase protein complex in a subject or biological sample comprising administering a compound described herein to the subject or contacting the biological sample with a compound described herein.

In certain aspects, provided herein are uses of a compound described herein in the manufacture of a medicament for binding cereblon E3 ubiquitin ligase protein complex in a subject or biological sample.

In certain aspects, provided herein are compounds described herein for use in binding cereblon E3 ubiquitin ligase protein complex in a subject or biological sample.

In certain aspects, provided herein are methods of degrading a protein in a subject or biological sample comprising administering a conjugate described herein to the subject or contacting the biological sample with a conjugate described herein.

In certain aspects, provided herein are uses of a conjugate described herein in the manufacture of a medicament for degrading a protein in a subject or biological sample.

In certain aspects, provided herein are conjugates described herein for use in degrading a protein in a subject or biological sample.

In certain embodiments, the protein is an androgen receptor (AR), an estrogen receptor (ER), signal transducer and activator of transcription 3 (STAT3), STAT5, CREB-binding protein/EP300(ElA) binding protein (CBP/p300), SWI/SNF Related, Matrix Associated, Actin Dependent Regulator Of Chromatin, Subfamily A, Member 2/4 (SMARCA2/4), Kirsten rat sarcoma viral oncogene homolog G12D (KRAS G12D), Src homology region 2-containing protein tyrosine phosphatase 2 (SHP2), bromodomain-containing protein 4 (BRD4), or BRD9.

In certain aspects, provided herein are methods of treating or preventing a disease or disorder a subject in need thereof, comprising administering to the subject a conjugate described herein.

In certain aspects, provided herein are uses of a conjugate described herein in the manufacture of a medicament for treating or preventing a disease or disorder in a subject in need thereof.

In certain aspects, provided herein are conjugates described herein for use in treating or preventing a disease or disorder in a subject in need thereof.

In certain embodiments, the protein is an androgen receptor (AR)-mediated disease or disorder, an estrogen receptor (ER)-mediated disease or disorder, signal transducer and activator of transcription 3-mediated disease or disorder (STAT3-mediated disease or disorder), STAT5-mediated disease or disorder, CREB-binding protein/EP300(E1A) binding protein-mediated disease or disorder (CBP/p300)-mediated disease or disorder, SWI/SNF Related, Matrix Associated, Actin Dependent Regulator Of Chromatin, Subfamily A, Member 2/4-mediated disease or disorder (SMARCA2/4-mediated disease or disorder), Kirsten rat sarcoma viral oncogene homolog G12D-mediated disease or disorder (KRAS G12D-mediated disease or disorder), Src homology region 2-containing protein tyrosine phosphatase 2-mediated disease or disorder (SHP2-mediated disease or disorder), bromodomain-containing protein 4-mediated disease or disorder (BRD4-mediated disease or disorder), or BRD9-mediated disease or disorder.

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉ alkyl”). In certain embodiments, a heteroalkyl group is a saturated group having 1 to 8 carbon atoms and 1, 2, 3, or 4 heteroatoms (“heteroC₁₋₆ alkyl”). In certain embodiments, a heteroalkyl group is a saturated group having 1 to 7 carbon atoms and 1, 2, 3, or 4 heteroatoms (“heteroC₁₋₇ alkyl”). In certain embodiments, a heteroalkyl group is a group having 1 to 6 carbon atoms and 1, 2, or 3 heteroatoms (“heteroC₁₋₆ alkyl”). In certain embodiments, a heteroalkyl group is a saturated group having 1 to 5 carbon atoms and 1 or 2 heteroatoms (“heteroC₁₋₅ alkyl”). In certain embodiments, a heteroalkyl group is a saturated group having 1 to 4 carbon atoms and/or 2 heteroatoms (“heteroC₁₋₄ alkyl”). In certain embodiments, a heteroalkyl group is a saturated group having 1 to 3 carbon atoms and 1 heteroatom (“heteroC₁₋₃ alkyl”). In certain embodiments, a heteroalkyl group is a saturated group having 1 to 2 carbon atoms and 1 heteroatom (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In certain embodiments, a heteroalkenyl group has 2 to 9 carbon atoms at least one double bond, and 1, 2, 3, or 4 heteroatoms (“heteroC₂₋₉ alkenyl”). In certain embodiments, a heteroalkenyl group has 2 to 8 carbon atoms, at least one double bond, and 1, 2, 3, or 4 heteroatoms (“heteroC₂₋₈ alkenyl”). In certain embodiments, a heteroalkenyl group has 2 to 7 carbon atoms, at least one double bond, and 1, 2, 3, or 4 heteroatoms (“heteroC₂₋₇ alkenyl”). In certain embodiments, a heteroalkenyl group has 2 to 6 carbon atoms, at least one double bond, and 1, 2, or 3 heteroatoms (“heteroC₂₋₆ alkenyl”). In certain embodiments, a heteroalkenyl group has 2 to 5 carbon atoms, at least one double bond, and 1 or 2 heteroatoms (“heteroC₂₋₅ alkenyl”). In certain embodiments, a heteroalkenyl group has 2 to 4 carbon atoms, at least one double bond, and lor 2 heteroatoms (“heteroC₂₋₄ alkenyl”). In certain embodiments, a heteroalkenyl group has 2 to 3 carbon atoms, at least one double bond, and 1 heteroatom (“heteroC₂₋₃ alkenyl”). In certain embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In certain embodiments, a heteroalkynyl group has 2 to 9 carbon atoms, at least one triple bond, and 1, 2, 3, or 4 heteroatoms (“heteroC₂₋₉ alkynyl”). In certain embodiments, a heteroalkynyl group has 2 to 8 carbon atoms, at least one triple bond, and 1, 2, 3, or 4 heteroatoms (“heteroC₂₋₈ alkynyl”). In certain embodiments, a heteroalkynyl group has 2 to 7 carbon atoms, at least one triple bond, and 1, 2, 3, or 4 heteroatoms (“heteroC₂₋₇ alkynyl”). In certain embodiments, a heteroalkynyl group has 2 to 6 carbon atoms, at least one triple bond, and 1, 2, or 3 heteroatoms (“heteroC₂₋₆ alkynyl”). In certain embodiments, a heteroalkynyl group has 2 to 5 carbon atoms, at least one triple bond, and 1 or 2 heteroatoms (“heteroC₂₋₅ alkynyl”). In certain embodiments, a heteroalkynyl group has 2 to 4 carbon atoms, at least one triple bond, and lor 2 heteroatoms (“heteroC₂₋₄ alkynyl”). In certain embodiments, a heteroalkynyl group has 2 to 3 carbon atoms, at least one triple bond, and 1 heteroatom (“heteroC₂₋₃ alkynyl”). In certain embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl, as defined herein, and refers to an optionally substituted alkyl group substituted by an optionally substituted aryl group.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl, as defined herein, and refers to an optionally substituted alkyl group substituted by an optionally substituted heteroaryl group.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₆ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share one common bond with, one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or “bridged carbocycle” refers to ring systems wherein the carbocyclyl group, as defined above, form bridged structure with, i.e., share more than one atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the bridged rings. When substitution is indicated, unless otherwise specified, substitution can occur on any of the carbocyclyl rings in which the bridged structure is embedded.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share one common bond with, one or more heterocyclyl or carbocyclyl groups, as defined above, wherein the point of attachment is on any of the fused rings. In such instances, the number of carbons designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than one atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the bridged rings.

“Hetero” when used to describe a compound or a group present on a compound means that one or more carbon atoms in the compound or group have been replaced by a nitrogen, oxygen, sulfur, boron, phosphorus, and silicon heteroatom, as valency permits. Hetero may be applied to any of the hydrocarbyl groups described above having from 1 to 5, and particularly from 1 to 3 heteroatoms.

“Acyl” as used herein, refers to a radical —C(O)R, wherein R is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative acyl groups include, but are not limited to, formyl (—CHO), acetyl (—C(═O)CH₃), cyclohexylcarbonyl, cyclohexylmethylcarbonyl, benzoyl (—C(═O)Ph), and benzylcarbonyl (—C(═O)CH₂Ph).

“Acylamino” as used herein, refers to a radical —NRC(═O)R, wherein each instance of R is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Exemplary “acylamino” groups include, but are not limited to, formylamino, acetylamino, cyclohexylcarbonylamino, cyclohexylmethyl-carbonylamino, benzoylamino and benzylcarbonylamino.

“Acyloxy” as used herein, refers to a radical —OC(═O)R, wherein R is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl.

“Alkoxy” as used herein, refers to the group —OR, wherein R is alkyl as defined herein. C₁₋₆ alkoxy refers to the group —OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Azido” refers to the radical —N₃.

“Amino” refers to the radical —NH₂.

“Hydroxy” refers to the radical —OH.

“Thioketo” refers to the group ═S.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Nitro” refers to the radical —NO₂.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroethoxycarbonyl (Troc), and benzyl carbamate (Cbz)), esters (e.g., acetyl (Ac); benzoyl (Bz), trifluoroacetyl, and phthalimide), amines (e.g, benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e.g., Berge, et al., J. Pharm. Sci. 66 (1):1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONR₂ and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“CRBN E3 ubiquitin ligase protein complex” is art recognized and refers to an association of proteins in which CRBN, a 442-amino acid protein, forms a Cullin-4-RING E3 ubiquitin ligase (CRL4) complex and interacts with the adaptor protein damaged DNA-binding protein 1 (DDB1), Cullin-4A (CUL4A), and regulator of cullins 1 (ROC1). Within the CRL4 complex, CRBN acts as a substrate-specificity receptor.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

It is also to be understood that compounds that have the same molecular formula but differ in the nature or sequence of bonding of their atoms or the arrangement of their atoms in space are termed “isomers.” Isomers that only differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### I. Cereblon Ligands

### Synthesis and Characterization

### Compound A1. (S)—N—((S)-2,6-dioxopiperidin-3-yl)-9-fluoro-1,2,3,4,4a,5-hexahydrobenzo[b]pyrazino[1,2-d][1,4]oxazine-8-carboxamide hydrochloride

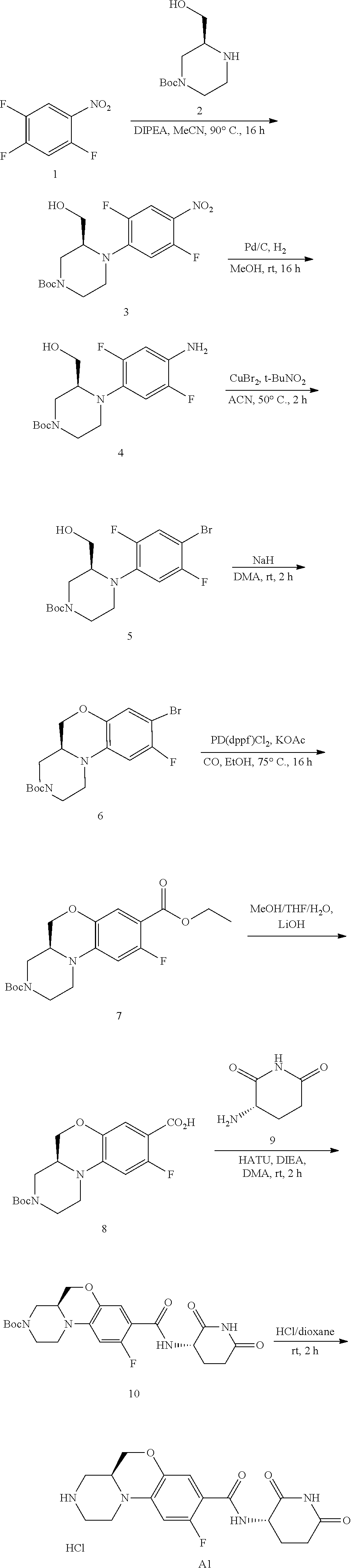

### Step 1: tert-butyl (S)-4-(2,5-difluoro-4-nitrophenyl)-3-(hydroxymethyl)piperazine-1-carboxylate

To a mixture of 1,2,4-trifluoro-5-nitrobenzene (12 g, 67.8 mmol, 1.0 eq.), tert-butyl (S)-3-(hydroxymethyl)piperazine-1-carboxylate (14.6 g, 67.8 mmol, 1 eq.) in MeCN (70 mL) was added DIEA (26.2 g, 203 mmol, 3.0 eq). The mixture was stirred at 90° C. for 16 hours and cooled to room temperature. The mixture was diluted with ethyl acetate (70 mL) and washed with water (150 mL). The organic layer was washed with brine (150 mL), dried over sodium sulfate, and filtered. The filtrate was concentrated and purified by silica gel column chromatography on silica gel eluted with 0-40% EtOAc/hexane to afford tert-butyl (S)-4-(2,5-difluoro-4-nitrophenyl)-3-(hydroxymethyl)piperazine-1-carboxylate (7 g, 27.7% yield) as yellow oil. LC-MS purity: 100% (UV at 254 nm), 374.0 [M+H]⁺.

### Step 2: tert-butyl (S)-4-(4-amino-2,5-difluorophenyl)-3-(hydroxymethyl)piperazine-1-carboxylate

To a mixture of tert-butyl (S)-4-(2,5-difluoro-4-nitrophenyl)-3-(hydroxymethyl)piperazine-1-carboxylate (6 g, 53.1 mmol, 1 eq.) in MeOH (200 mL) was added Pd/C (1 g, 10% on Carbon, wetted with c.a.55% water) stirred at rt overnight under H₂. The mixture was filtered and the filtrate was concentrated to afford tert-butyl (S)-4-(4-amino-2,5-difluorophenyl)-3-(hydroxymethyl)piperazine-1-carboxylate as brown solid (5.5 g, crude). LC-MS purity: 100% (UV at 254 nm), 343.9 [M+H]⁺.

### Step 3: tert-butyl (S)-4-(4-bromo-2,5-difluorophenyl)-3-(hydroxymethyl)piperazine-1-carboxylate

To a mixture of tert-butyl (S)-4-(4-amino-2,5-difluorophenyl)-3-(hydroxymethyl)piperazine-1-carboxylate (5.5 g, 16 mmol, 1 eq.), in MeCN (50 mL) was added CuBr₂ (7.2 g, 32 mmol, 2 eq.). The mixture was purged with nitrogen and stirred at 50° C. t-BuONO (2.5 g, 24 mmol, 1.5 eq.) was added and the mixture was stirred at 50° C. for 30 min. The mixture was diluted with ethyl acetate (50 mL) and washed with NH₄Cl solution (100 mL). The organic layer was washed with brine (150 mL), dried over sodium sulfate and filtered. The filtrate was concentrated and purified by silica gel column chromatography on silica gel eluted with 0-40% EtOAc/hexane to give tert-butyl (S)-4-(4-bromo-2,5-difluorophenyl)-3-(hydroxymethyl)piperazine-1-carboxylate as a yellow oil. (2 g, 31% yield), LC-MS purity: 100% (UV at 254 nm), 407.3 [M+H]⁺.

### Step 4: tert-butyl (S)-8-bromo-9-fluoro-1,2,4a,5-tetrahydrobenzo[b]pyrazino[1,2-d][1,4]oxazine-3(4H)-carboxylate

To a mixture of tert-butyl (S)-4-(4-bromo-2,5-difluorophenyl)-3-(hydroxymethyl)piperazine-1-carboxylate (2 g, 4.9 mmol, 1 eq.) in DMA (10 mL) was added NaH (590 mg, 14.7 mmol, 3 eq.). The mixture was stirred at room temperature for 2 hours. The mixture was diluted with ethyl acetate (30 mL) and washed with NH₄Cl solution (100 mL). The organic layer was washed with brine (100 mL), dried over sodium sulfate and filtered. The filtrate was concentrated and purified by silica gel column chromatography on silica gel eluted with 0-30% EtOAc/hexane to give tert-butyl (S)-8-bromo-9-fluoro-1,2,4a,5-tetrahydrobenzo[b]pyrazino[1,2-d][1,4]oxazine-3(4H)-carboxylate as brown oil. (1.2 g, 63% yield), LC-MS purity: 100% (UV at 254 nm), 387.3[M+H]⁺.

### Steps 5: 3-(tert-butyl) 8-ethyl (S)-9 fluoro-1,2,4a,5-tetrahydrobenzo[b]pyrazino[1,2-d][1,4]oxazine-3,8(4H)-dicarboxylate

To a mixture of tert-butyl (S)-8-bromo-9-fluoro-1,2,4a,5-tetrahydrobenzo[b]pyrazino[1,2-d][1,4]oxazine-3(4H)-carboxylate (1.2 g, 3.1 mmol, 1 eq.), KOAc (912 mg, 9.3 mmol, 3 eq.) in EtOH (20 mL) was added Pd(dppf)Cl₂ (227 mg, 0.31 mmol, 0.1 eq.). The mixture was purged with CO and stirred at 75° C. for 16 hours and cooled to room temperature. The crude was concentrated and purified by silica gel column chromatography on silica gel eluted with 0-30% EtOAc/hexane to give 3-(tert-butyl) 8-ethyl (S)-9-fluoro-1,2,4a,5-tetrahydrobenzo[b]pyrazino[1,2-d][1,4]oxazine-3,8(4H)-dicarboxylate as white solid. (0.88 g, yield 75%), LC-MS purity: 100% (UV at 254 nm), 381.2[M+H]⁺.

### Steps 6: (S)-3-(tert-butoxycarbonyl)-9-fluoro-1,2,3,4,4a,5-hexahydrobenzo[b]pyrazino[1,2-d][1,4]oxazine-8-carboxylic acid

To a mixture of 3-(tert-butyl) 8-ethyl (S)-9-fluoro-1,2,4a,5-tetrahydrobenzo[b]pyrazino[1,2-d][1,4]oxazine-3,8(4H)-dicarboxylate (0.88 g, 2.3 mmol, 1 eq.) in tetrahydrofuran (5 mL), MeOH (5 mL) and water (5 mL) was added LiOH (111 mg, 4.6 mmol, 2 eq). The mixture was stirred at room temperature for 2 hours. The mixture was adjusted to pH 5-6 with aq. hydrochloric acid (1 M) and extracted with ethyl acetate. The organic layer was washed with brine (30 mL), dried over sodium sulfate and filtered. The filtrate was concentrated to give (S)-3-(tert-butoxycarbonyl)-9-fluoro-1,2,3,4,4a,5-hexahydrobenzo[b]pyrazino[1,2-d][1,4]oxazine-8-carboxylic acid as white solid (765 mg, crude). LC-MS purity: 100% (UV at 254 nm), 352.4 [M+H]⁺.

### Steps 7: tert-butyl (S)-8-(((S)-2,6-dioxopiperidin-3-yl)carbamoyl)-9-fluoro-1,2,4a,5-tetrahydrobenzo[b]pyrazino[1,2-d][1,4]oxazine-3(4H)-carboxylate

To a mixture of (S)-3-(tert-butoxycarbonyl)-9-fluoro-1,2,3,4,4a,5-hexahydrobenzo[b]pyrazino[1,2-d][1,4]oxazine-8-carboxylic acid (765 mg, 2.2 mmol, 1 eq.) in DMA (10 mL) was added HATU (1.6 g, 4.6 mmol, 2 eq.) and DIPEA (841 mg, 6.5 mmol, 3 eq.). The reaction was stirred at room temperature for 1 hour. The mixture was stirred at room temperature for 2 hours. The mixture was diluted with ethyl acetate (30 mL) and washed with brine (100 mL). The organic layer was washed with brine (100 mL), dried over sodium sulfate, and filtered. The filtrate was concentrated and purified by silica gel column chromatography on silica gel eluted with 0-50% EtOAc/hexane to give tert-butyl (S)-8-(((S)-2,6-dioxopiperidin-3-yl)carbamoyl)-9-fluoro-1,2,4a,5-tetrahydrobenzo[b]pyrazino[1,2-d][1,4]oxazine-3(4H)-carboxylate as white solid (760 mg, 76% yield), LC-MS purity: 100% (UV at 254 nm), 463.3 [M+H]⁺.

### Steps 8: (S)—N—((S)-2,6-dioxopiperidin-3-yl)-9-fluoro-1,2,3,4,4a,5-hexahydrobenzo[b]pyrazino[1,2-d][1,4]oxazine-8-carboxamide hydrochloride

A mixture of tert-butyl (S)-8-(((S)-2,6-dioxopiperidin-3-yl)carbamoyl)-9-fluoro-1,2,4a,5-tetrahydrobenzo[b]pyrazino[1,2-d][1,4]oxazine-3(4H)-carboxylate (760 mg, 1.6 mmol, 1 eq.) in HCl/dioxane (8 mL) was stirred at room temperature for 2 hours. The mixture was concentrated to give (S)—N—((S)-2,6-dioxopiperidin-3-yl)-9-fluoro-1,2,3,4,4a,5-hexahydrobenzo[b]pyrazino[1,2-d][1,4]oxazine-8-carboxamide hydrochloride as white solid (680 mg, crude), LC-MS purity: 100% (UV at 254 nm), 363.2 [M+H]⁺.

¹H NMR (400 MHz, DMSO) δ 10.84 (s, 1H), 9.40 (m, 2H), 8.13 (t, J=8 Hz, 1H), 7.10 (d, J=7.2 Hz, 1H), 6.94 (d, J=13.8 Hz, 1H), 4.77-4.65 (m, 1H), 4.31 (dd, J=11.2, 2.8 Hz, 1H), 4.13-3.94 (m, 2H), 3.65-3.58 (m, 1H), 3.42-3.37 (m, 2H), 3.20-2.96 (m, 2H), 2.86-2.70 (m, 2H), 2.56-2.51 (m, 1H), 2.18-1.93 (m, 2H).

### Compound A2. (S)—N—((S)-2,6-dioxopiperidin-3-yl)-9-fluoro-3-methyl-1,2,3,4,4a,5-hexahydrobenzo[b]pyrazino[1,2-d][1,4]oxazine-8-carboxamide

Compound A2 was synthesized following similar procedures for A1. LC-MS purity: 95% (UV at 254 nm), 377.2 [M+H]⁺.

### Compound A3. tert-butyl (4aS)-9-(2,6-dioxopiperidin-3-yl)-8-oxo-1,2,4a,5,9,10-hexahydro-8H-pyrazino[1′,2′:4,5][1,4]oxazino[2,3-f]isoindole-3(4H)-carboxylate

Compound A3 was synthesized following the same procedures for B1 except the final step. LC-MS purity: 95% (UV at 254 nm), 457.2 [M+H]⁺.

### Compound A4. N-(2,6-dioxopiperidin-3-yl)-1,2,3,4,4a,5-hexahydropyrazino[1,2-d]pyrido[2,3-b][1,4]oxazine-8-carboxamide hydrochloride salt

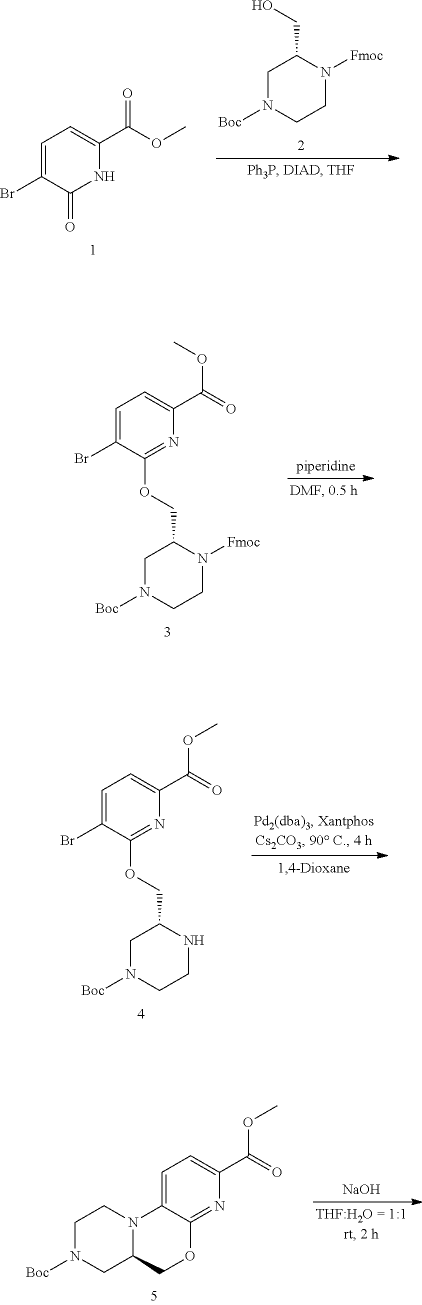

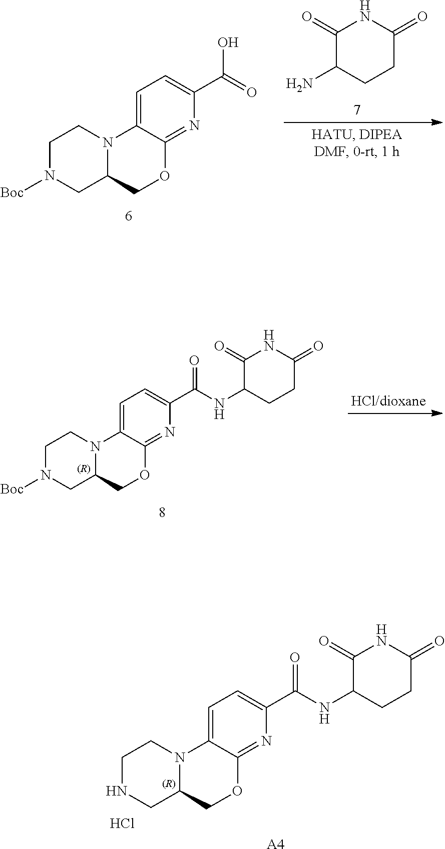

### Step 1: (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate

To a mixture of methyl 5-bromo-6-oxo-1,6-dihydropyridine-2-carboxylate (2.5 g, 10.7 mmol, 1 eq.) in THF (50 mL) was added (R)-1-((9H-fluoren-9-yl)methyl) 4-tert-butyl 2-(hydroxymethyl)piperazine-1,4-dicarboxylate (5.7 g, 12.9 mmol, 1.2 eq.) and PPh₃ (8.4 g, 32.1 mmol, 3 eq.) and the mixture was stirred at 60° C. To the mixture was added DIAD (6.5 g, 32.1 mmol, 3 eq.) dropwise and the mixture was stirred at room temperature for 12 h. The mixture was evaporated in vacuo and the residue was purified by column chromatography on silica gel eluted with 0-30% EtOAc/hexane to afford (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate (5.0 g, 70% yield) as yellow solid.

### Step 2: (R)-tert-butyl 3-(((3-bromo-6-(methoxycarbonyl)pyridin-2-yl)oxy)methyl)piperazine-1-carboxylate

To a mixture of (R)-1-((9H-fluoren-9-yl)methyl) 4-tert-butyl 2-(((3-bromo-6-(methoxycarbonyl)pyridin-2-yl)oxy)methyl)piperazine-1,4-dicarboxylate (5 g, 7.6 mmol 1 eq.) in DMF (50 mL) was added piperidine (1.1 g, 15.2 mmol, 2 eq.). The mixture was stirred at room temperature for 1 h, diluted with ethyl acetate (100 mL) and washed with water (50 mL). The organic phase was washed with brine, dried over Na₂SO₄ and filtered. The filtrate was evaporated in vacuo. The crude was purified by column chromatography on silica gel eluted with 0-5% DCM in methanol to give (R)-tert-butyl 3-(((3-bromo-6-(methoxycarbonyl)pyridin-2-yl)oxy)methyl)piperazine-1-carboxylate (2.4 g, 75% yield). LC-MS purity: 100% (UV at 254 nm), found: 430.2 [M+1]⁺.

### Step 3: (R)-3-tert-butyl 8-methyl 1,2,4a,5-tetrahydropyrazino[1,2-d]pyrido[2,3-b][1,4]oxazine-3,8(4H)-dicarboxylate

To a mixture of (R)-tert-butyl 3-(((3-bromo-6-(methoxycarbonyl)pyridin-2-yl)oxy)methyl)piperazine-1-carboxylate (2.4 g, 5.6 mmol, 1 eq.), XantPhos (486 mg, 0.84 mmol, 0.15 eq.), and Cs₂CO₃ (5.4 g, 16.8 mmol, 3 eq.) in dioxane (50 mL) was added Pd₂(dba)₃ (511 mg, 0.56 mmol, 0.1 eq.) under Ar flow and the mixture was stirred at 100° C. for 16 h. The mixture was diluted with ethyl acetate (100 mL) and washed with water (50 mL). The organic phase was washed with brine, dried over Na₂SO₄, and filtered. The filtrate was evaporated in vacuo and the residue was purified by column chromatography on silica gel eluted with using 0-50% EtOAc/hexane to give (R)-3-tert-butyl 8-methyl 1,2,4a,5-tetrahydropyrazino[1,2-d]pyrido[2,3-b][1,4]oxazine-3,8(4H)-dicarboxylate (1.3 g, 68% yield) as white solid. LC-MS purity: 100% (UV at 254 nm), 350.4 [M+H]⁺.

### Step 4: (R)-3-(tert-butoxycarbonyl)-1,2,3,4,4a,5-hexahydropyrazino[1,2-d]pyrido[2,3-b][1,4]oxazine-8-carboxylic acid

To a mixture of (R)-3-tert-butyl 8-methyl 1,2,4a,5-tetrahydropyrazino[1,2-d]pyrido[2,3-b][1,4]oxazine-3,8(4H)-dicarboxylate (1.3 g, 3.7 mmol, 1 eq.) in THF (10 mL) and water (10 mL) was added sodium hydroxide (590 mg, 14.8 mmol, 4 eq) and the mixture was stirred at room temperature for 2 h. The mixture was adjusted to pH 5-6 with aq. HCl (1 M) and extracted with ethyl acetate (20 mL). The organic layer was washed with brine, dried over sodium sulfate, and filtered. The filtrate was evaporated to afford (R)-3-(tert-butoxycarbonyl)-1,2,3,4,4a,5-hexahydropyrazino[1,2-d]pyrido[2,3-b][1,4]oxazine-8-carboxylic acid (1.3 g, crude) as white solid. LC-MS purity: 100% (UV at 254 nm), 336.3 [M+H]⁺.

### Step 5: tert-butyl (R)-8-(((S)-2,6-dioxopiperidin-3-yl)carbamoyl)-1,2,4a,5-tetrahydropyrazino[1,2-d]pyrido[2,3-b][1,4]oxazine-3(4H)-carboxylate

To a mixture of (R)-3-(tert-butoxycarbonyl)-1,2,3,4,4a,5-hexahydropyrazino[1,2-d]pyrido[2,3-b][1,4]oxazine-8-carboxylic acid (1.3 g, 3.8 mmol, 1 eq) in DMF (10 mL) was added 3-aminopiperidine-2,6-dione (0.51 g, 4.0 mmol), HATU (1.7 g, 4.6 mmol, 1.2 eq) and DIPEA (980 mg, 7.6 mmol, 2 eq) and the mixture was stirred at room temperature for 1 h. The mixture was purified directly by reverse phase column chromatography (0-90% acetonitrile/0.05% formic acid)) to afford (R)-tert-butyl 8-(((S)-2,6-dioxopiperidin-3-yl)carbamoyl)-1,2,4a,5-tetrahydropyrazino[1,2-d]pyrido[2,3-b][1,4]oxazine-3(4H)-carboxylate (1.3 g, 76% yield) as white solid. LC-MS purity: 100% (UV at 254 nm), 446.2[M+H]⁺.

### Step 6: rac-N—((S)-2,6-dioxopiperidin-3-yl)-1,2,3,4,4a,5-hexahydropyrazino[1,2-d]pyrido[2,3-b][1,4]oxazine-8-carboxamide hydrochloride

A mixture of rac-tert-butyl 8-(((S)-2,6-dioxopiperidin-3-yl)carbamoyl)-1,2,4a,5-tetrahydropyrazino[1,2-d]pyrido[2,3-b][1,4]oxazine-3(4H)-carboxylate (1.3 g, 2.9 mmol, 1 eq.) in HCV/dioxane (10 mL) was stirred at room temperature for 2 h. The reaction mixture was concentrated to afford rac-N—((S)-2,6-dioxopiperidin-3-yl)-1,2,3,4,4a,5-hexahydropyrazino[1,2-d]pyrido[2,3-b][1,4]oxazine-8-carboxamide hydrochloride (1.0 g, 91% yield) as white solid.

LC-MS purity: 100% (UV at 254 nm), ms:_346.2[M+1]⁺.

¹H NMR (400 MHz, DMSO): δ 10.84 (s, 1H), 9.63-9.33 (m, 2H), 8.56 (d, J=8.4 Hz, 1H), 7.62 (d, J=8.2 Hz, 1H), 7.44 (d, J=8.4 Hz, 1H), 4.77-4.70 (m, 1H), 4.51-4.49 (m, 2H), 4.20-4.05 (m, 2H), 3.66-3.55 (m, 1H), 3.47-3.39 (m, 2H), 3.22-2.98 (m, 2H), 2.89-2.67 (m, 2H), 2.26-2.11 (m, 1H), 2.02-1.90 (m, 1H).

### Compound A6. 3-((1,2,3,4,4a,5-hexahydrobenzo[b]pyrazino[1,2-d][1,4]oxazin-8-yl)amino)piperidine-2,6-dione hydrochloride salt

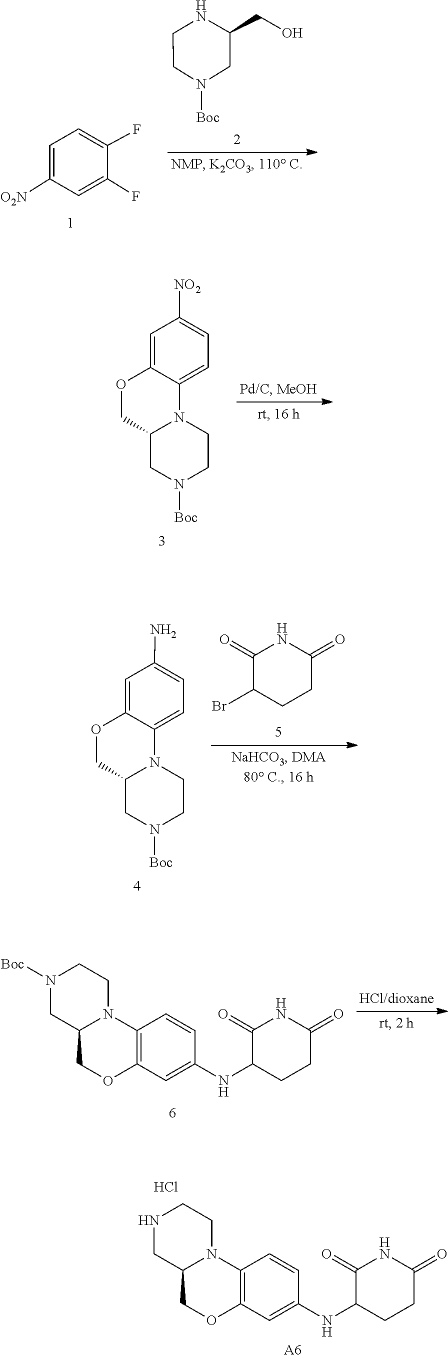

### Step 1: tert-butyl (R)-8-nitro-1,2,4a,5-tetrahydrobenzo[b]pyrazino[1,2-d][1,4]oxazine-3(4H)-carboxylate

To a solution of 1,2-difluoro-4-nitrobenzene (5.1 g, 32.4 mmol, 1.0 eq.) and tert-butyl (R)-3-(hydroxymethyl)piperazine-1-carboxylate (7.0 g, 32.4 mmol, 1.0 eq.) in DMA (50 mL) was added Cs₂CO₃ (21.0 g, 64.8 mmol, 2.0 eq.). The reaction mixture was stirred at 110° C. for 16 hours. The mixture was cooled to room temperature and poured into brine (300 mL). The mixture was extracted with EtOAc (100 mL). The organic phase was washed with brine (300 mL), dried over Na₂SO₄, and filtered. The filtrate was evaporated and the residue was purified by column chromatography on silica gel eluted with 0-10% EtOAc/hexane to afford tert-butyl (R)-8-nitro-1,2,4a,5-tetrahydrobenzo[b]pyrazino[1,2-d][1,4]oxazine-3(4H)-carboxylate (2.5 g, 23% yield) as yellow solid. LC-MS purity: 69% (UV at 254 nm), 336.4 [M+H]⁺.

### Step 2: tert-butyl (R)-8-amino-1,2,4a,5-tetrahydrobenzo[b]pyrazino[1,2-d][1,4]oxazine-3(4H)-carboxylate

To a solution of tert-butyl (R)-8-nitro-1,2,4a,5-tetrahydrobenzo[b]pyrazino[1,2-d][1,4]oxazine-3(4H)-carboxylate (2.5 g, 7.4 mmol, 1 eq.) in MeOH (30 mL) was added Pd/C (250 mg, 10% on carbon, wetted with ca. 55% water) and the reaction mixture was stirred at room temperature for 16 h. The catalyst was removed by filtration and the filtrate was concentrated to afford (R)-8-amino-1,2,4a,5-tetrahydrobenzo[b]pyrazino[1,2-d][1,4]oxazine-3(4H)-carboxylate (2.2 g, crude) as a brown solid. LC-MS purity: 86.8% (UV at 254 nm), 306.3 [M+H]⁺.

### Step 3: tert-butyl (4aR)-8-((2,6-dioxopiperidin-3-yl)amino)-1,2,4a,5-tetrahydrobenzo[b]pyrazino[1,2-d][1,4]oxazine-3(4H)-carboxylate

To a mixture of (R)-8-amino-1,2,4a,5-tetrahydrobenzo[b]pyrazino[1,2-d][1,4]oxazine-3(4H)-carboxylate (700 mg, 2.3 mmol 1.0 eq.) in DMA (8 mL) was added 3-bromopiperidine-2,6-dione (440 mg, 2.3 mmol, 1.0 eq.) and NaHCO₃ (193 mg, 2.3 mmol, 1.0 eq.). The mixture was stirred at 80° C. for 12 hours and cooled to room temperature. The mixture was concentrated and the residue was purified by column chromatography on silica gel eluted with 0-100% EtOAc/hexane to afford tert-butyl (4aR)-8-((2,6-dioxopiperidin-3-yl)amino)-1,2,4a,5-tetrahydrobenzo[b]pyrazino[1,2-d][1,4]oxazine-3(4H)-carboxylate (660 mg, 69% yield) as white solid. LC-MS purity: 89.7% (UV at 254 nm), 417.4 [M+H]⁺. ¹H NMR (400 MHz, DMSO): δ 10.73 (s, 1H), 6.67 (d, J=8.4 Hz 2H), 6.21-6.14 (m, 2H), 5.33 (d, J=7.2 Hz, 1H), 4.24-4.13 (m, 2H), 3.96-3.80 (m, 3H), 3.64-3.61 (m, 1H), 2.75-2.56 (m, 3H), 2.39-2.05 (m, 1H), 1.81-1.78 (m, 1H), 1.41 (s, 9H).

### Step 4: 3-(((R)-1,2,3,4,4a,5-hexahydrobenzo[b]pyrazino[1,2-d][1,4]oxazin-8-yl)amino)piperidine-2,6-dione hydrochloride salt

A mixture of tert-butyl 4-(4-((2,6-dioxopiperidin-3-yl)amino)-2-fluorophenyl)piperazine-1-carboxylate (50 mg, 0.1 mmol, 1 eq.) in EA/HCl (1 ml) was stirred at room temperature for 2 h. The mixture was concentrated to afford 3-(((R)-1,2,3,4,4a,5-hexahydrobenzo[b]pyrazino[1,2-d][1,4]oxazin-8-yl)amino)piperidine-2,6-dione hydrochloride salt (40 mg, crude) as a yellow solid. LC-MS purity: 100% (UV at 254 nm), 317.3 [M+H]⁺. ¹H NMR (400 MHz, DMSO): δ 10.99 (s, 1H), 10.78-10.84 (m, 2H), 6.85-6.83 (m, 1H), 6.55-6.48 (m, 2H), 4.36-4.26 (m, 2H), 3.93-3.86 (m, 2H), 3.39-3.31 (m, 3H), 3.04-2.51 (m, 5H), 1.99-1.51 (m, 2H).

### Compound A7. (S)-3-((R)-1-oxo-5,5a,6,7,8,9-hexahydro-1H-pyrazino [1′,2′:4,5][1,4]oxazino[2,3-e]isoindol-2(3H)-yl)piperidine-2,6-dione hydrochloride salt

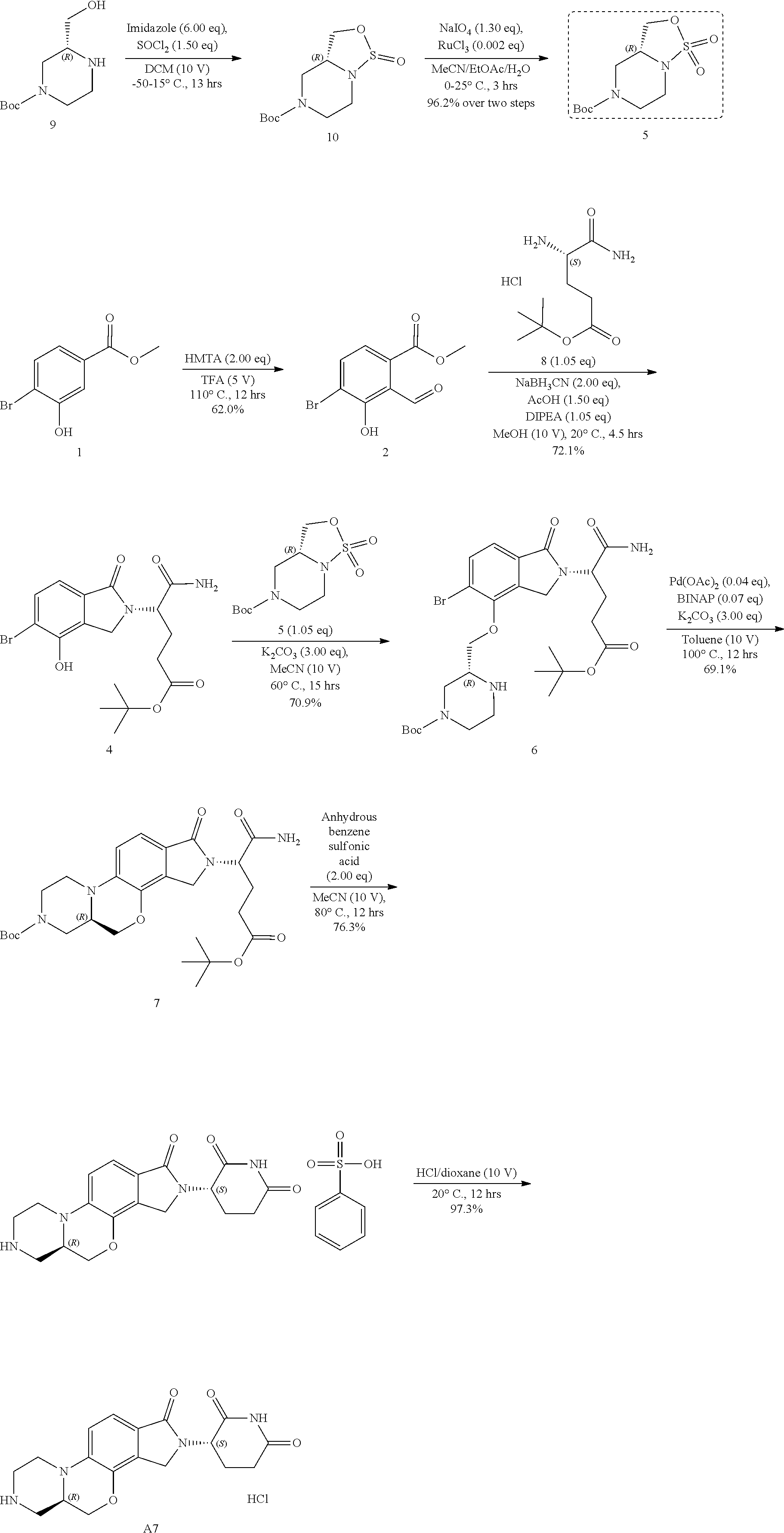

### Step 1: Synthesis of tert-butyl (3aR)-tetrahydro-[1,2,3]oxathiazolo[3,4-a]pyrazine-5(3H)-carboxylate 1-oxide

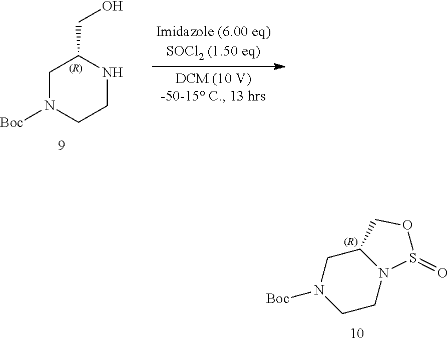

Imidazole (377 g, 5.55 mol, 6.00 eq) was dissolved in DCM (2.0 L) at 15° C. and cool to 0° C., before SOCl₂ (165 g, 100 mL, 1.39 mol, 1.50 eq) was added dropwise under N₂ atmosphere. Precipitate was observed upon the addition of SOCl₂. The suspension was stirred at 15° C. for further 1 hr, and during which period the temperature was maintained below 15° C. After cooled to −50° C., a solution of compound 9 (200 g, 924 mmol, 1.00 eq) in DCM (2.0 L) was added dropwise under N₂ atmosphere, before the reaction mixture was stirred at 15° C. for further 12 hrs. The reaction was quenched by water (8.0 L), and the resulting mixture was stirred for 15 min. The aqueous phase was extracted with DCM (2×4.0 L), and the combined organic phase was washed with brine (5.0 L), dried over anhydrous Na₂SO₄, filtered, and concentrated in vacuo. Crude compound 10 (240 g) was obtained as a yellow solid. ¹HNMR (400 MHz, CDCl₃-d) δ=4.64 (dd, J=6.2, 8.1 Hz, 1H), 4.32-4.20 (m, 2H), 4.17-3.97 (m, 1H), 3.75-3.58 (m, 1H), 3.46 (br d, J=11.6 Hz, 1H), 3.25-3.07 (m, 1H), 3.03-2.87 (m, 2H), 1.48 (s, 9H)

### Step 2: Synthesis of tert-butyl (R)-tetrahydro-[1,2,3]oxathiazolo[3,4-a]pyrazine-5(3H)-carboxylate 1,1-dioxide

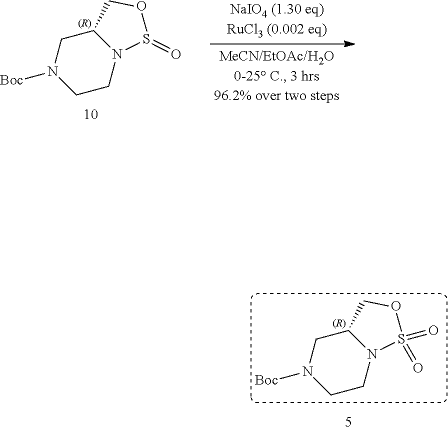

Compound 10 (240 g, 914 mmol, 1.00 eq) was dissolved in MeCN (1.0 L) and EtOAc (1.0 L) at 25° C., before being cooled to 0° C., and a solution of RuCl₃ (379 mg, 1.83 mmol, 122 μL, 0.002 eq) and NaIO₄ (293 g, 1.37 mol, 76.0 mL, 1.5 eq) in H₂O (2.5 L) was added dropwise to the mixture over 1 hour under N₂ atmosphere. The resulting mixture was stirred at 25° C. for further 3 hrs. The reaction mixture was filtered, and the filter cake was washed with EtOAc (3.0 L). The aqueous phase was extracted with EtOAc (2×2.0 L), and the combined organic phase was washed with brine (3.0 L), dried over anhydrous Na₂SO₄, filtered and concentrated in vacuo. Compound 5 (250 g) was obtained as a yellow solid, and the typical yield was 96.2% over two steps. ¹HNMR (400 MHz, CDCl₃-d) 4.63 (dd, J=6.3, 8.0 Hz, 1H), 4.32-3.96 (m, 3H), 3.64 (ddt, J=3.5, 6.1, 9.4 Hz, 1H), 3.45 (br d, J=11.6 Hz, 1H), 3.13 (br s, 1H), 2.96 (dt, J=3.3, 11.4 Hz, 2H), 1.47 (s, 9H)

### Step 3: Synthesis of methyl 4-bromo-2-formyl-3-hydroxybenzoate

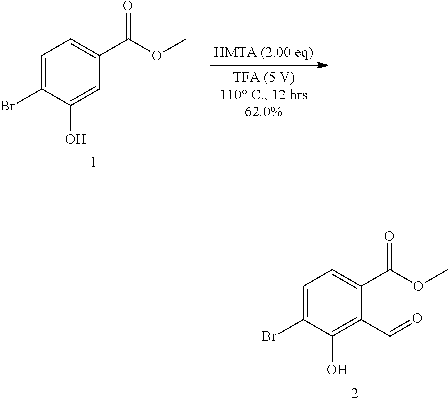

Compound 1 (300 g, 1.30 mol, 1.00 eq) was dissolved in TFA (1.5 L) at 20° C., before HMTA (364 g, 2.60 mol, 2.00 eq) was added to the mixture portion-wise at 20° C., and the reaction mixture was stirred at 110° C. for 12 hrs. The mixture was cooled to 20° C., and quenched with 2N HCl solution (5 V, 1.5 L). Yellow precipitate was observed, and the mixture was stirred for further 10 min. Additional water (5 V, 1.5 L) was added and the mixture was stirred for 1 hour, before the resulting mixture was filtered. The filtered cake was dissolved in DCM (3.0 L) and filtered over celite. The filtrate was dried over anhydrous Na₂SO₄ and concentrated in vacuo at 45° C. Compound 2 (210 g) was obtained as a yellow solid, and the typical yield was 62.4%. ¹HNMR (400 MHz, CDCl₃-d) 12.05 (br s, 1H), 10.38 (s, 1H), 7.99 (d, J=8.2 Hz, 1H), 7.29 (d, J=8.2 Hz, 1H), 3.88 (s, 3H)

Note: The actual reflux temperature was 110° C. (inside temperature). The reactant could not be fully consumed below 110° C.

### Step 4: Synthesis of tert-butyl (S)-5-amino-4-(5-bromo-4-hydroxy-1-oxoisoindolin-2-yl)-5-oxopentanoate

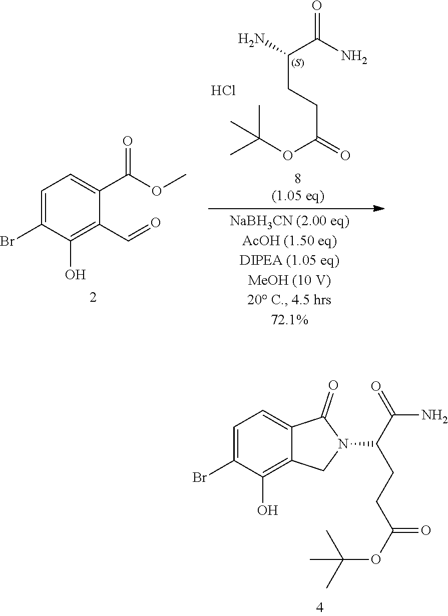

Compound 8 (193 g, 810 mmol, 1.05 eq, HCl salt) was suspended in MeOH (2.0 L), before DIPEA (104 g, 810 mmol, 141 mL, 1.05 eq) was added in one portion at 20° C. Compound 2 (200 g, 772 mmol, 1.00 eq) was added to the mixture in one portion, followed by the addition of AcOH (66.2 mL, 1.16 mol, 1.50 eq) at 20° C. and stirred for 1.5 hrs. NaBH₃CN (97.0 g, 1.54 mol, 2.00 eq) was added to the mixture portion-wise, and the resulting mixture was stirred at 20° C. for further 3 hrs. The reaction mixture was quenched by H₂O (4.0 L) at 20° C., and concentrated under reduced pressure at 40° C. The solvent residue was extracted with EtOAc (3×3.0 L), and the combined organic layer was washed with brine (2×5.0 L), dried over anhydrous Na₂SO₄, filtered and concentrated in vacuo. The crude product was purified by silica gel column chromatography (column height: 250 mm, diameter: 100 mm, 100-200 mesh silica gel, Petroleum ether/Ethyl acetate=100/1 to 1/1). To the crude product was added H₂O (4.0 L) at 15° C. and stirred for 2 hrs to remove DIPEA-HCl salt. The mixture was filtered, and the filter cake was dried under reduced pressure at 45° C. Compound 4 (230 g) was obtained as a yellow solid and the typical yield was 72.1%. ¹HNMR (400 MHz, DMSO-d₆) 10.46 (br s, 1H), 7.71-7.55 (m, 2H), 7.22 (br s, 1H), 7.11 (d, J=7.9 Hz, 1H), 4.79-4.69 (m, 1H), 4.64-4.54 (m, 1H), 4.48-4.36 (m, 1H), 2.23-2.09 (m, 3H), 2.03-1.92 (m, 1H), 1.33 (s, 9H)

### Step 5: Synthesis of tert-butyl (R)-3-(((2-((S)-1-amino-5-(tert-butoxy)-1,5-dioxopentan-2-yl)-5-bromo-1-oxoisoindolin-4-yl)oxy)methyl) piperazine-1-carboxylate

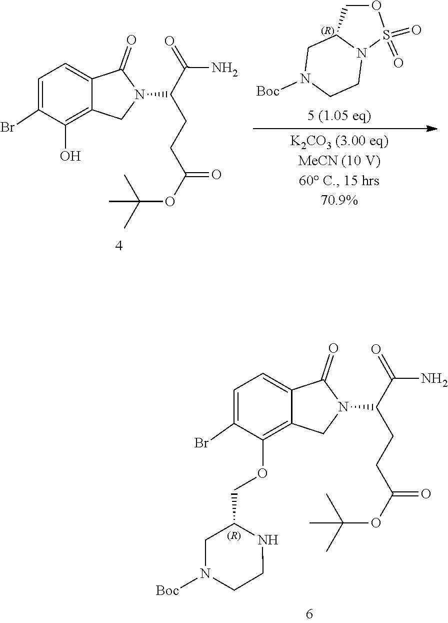

Compound 4 (200 g, 484 mmol, 1.00 eq) was dissolved in MeCN (2.0 L), before K₂CO₃ (200 g, 1.45 mol, 3.00 eq) was added portion-wise, followed by the addition of Compound 5 (141 g, 508 mmol, 1.05 eq) in one portion at 15° C. The reaction mixture was stirred at 60° C. for 12 hrs, and cooled to 15° C. The resulting mixture was filtered, and the filter cake was washed with DCM (4.0 L). The organic layer was concentrated under reduced pressure at 45° C., and the residue was diluted with DCM (3.0 L) again. TsOH·H₂O (184 g, 967 mmol, 2.00 eq) was added to the resulting solution and stirred at 15° C. for further 3 hrs, before saturated NaHCO₃ solution (3.0 L) was added and stirred for another 30 min. The aqueous phase was extracted with EtOAc (3×3.0 L), and the combined organic phase was washed with brine (3.0 L), dried over anhydrous Na₂SO₄, filtered, and concentrated in vacuo at 45° C. The crude product was purified by silica gel column chromatography (column height: 250 mm, diameter: 100 mm, 100-200 mesh silica gel, Petroleum ether/Ethyl acetate=100/1 to 1/1). Compound 6 (210 g) was obtained as a brown solid, and the typical yield was 70.9%. ¹HNMR (400 MHz, DMSO-d₆) 7.75 (d, J=7.9 Hz, 1H), 7.57 (br s, 1H), 7.36 (d, J=8.1 Hz, 1H), 7.23 (br s, 1H), 4.79-4.64 (m, 3H), 4.20-4.04 (m, 3H), 3.77 (br d, J=12.0 Hz, 1H), 3.33 (s, 1H), 2.96-2.54 (m, 6H), 2.26-2.12 (m, 3H), 1.48-1.28 (m, 18H).

### Step 6: Synthesis of tert-butyl (R)-2-((S)-1-amino-5-(tert-butoxy)-1,5-dioxopentan-2-yl)-1-oxo-2,3,5a,6,8,9-hexahydro-1H-pyrazino[1′,2′:4,5][1,4]oxazino[2,3-e]isoindole-7(5H)-carboxylate

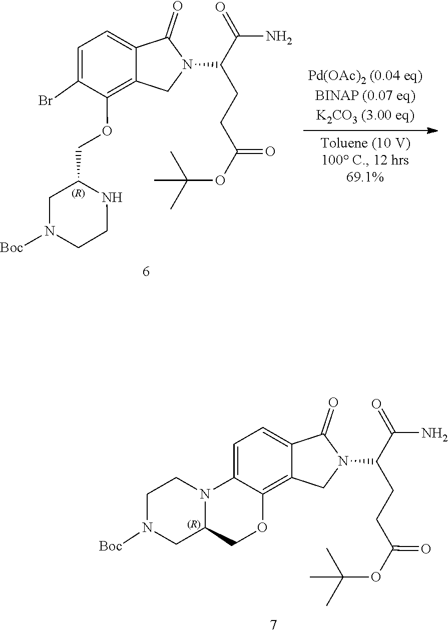

Compound 6 (100 g, 163 mmol, 1.00 eq) was dissolved in toluene (1.0 L), before K₂CO₃ (67.8 g, 490 mmol, 3.00 eq), Pd(OAc)₂ (1.47 g, 6.54 mmol, 0.04 eq) and BINAP (7.13 g, 11.4 mmol, 0.07 eq) was added at 20° C. The reaction mixture was degassed and purged with N₂ for 3 times at 20° C. and stirred at 100° C. for 12 hrs under N₂ atmosphere. The reaction was filtered, and the filter cake was washed with EtOAc (3×2 L). The resulting solution was concentrated under reduced pressure at 45° C., and the crude product was purified by silica gel column chromatography (column height: 250 mm, diameter: 100 mm, 100-200 mesh silica gel, Petroleum ether/Ethyl acetate=100/1 to 1/1). Compound 7 (60 g) was obtained as a white solid and the typical yield was 69.1%. ¹H NMR (400 MHz, DMSO-d₆) 7.50 (br s, 1H), 7.15 (br d, J=10.5 Hz, 1H), 7.04 (br d, J=8.2 Hz, 1H), 4.66 (br d, J=9.3 Hz, 1H), 4.51-4.37 (m, 2H), 4.33-4.21 (m, 1H), 4.05-3.87 (m, 4H), 3.21-3.10 (m, 1H), 2.96 (br s, 1H), 2.69 (br t, J=11.2 Hz, 2H), 2.23-1.91 (m, 5H), 1.53-1.25 (m, 18H)

### Step 7: Synthesis of (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione benzenesulfonate

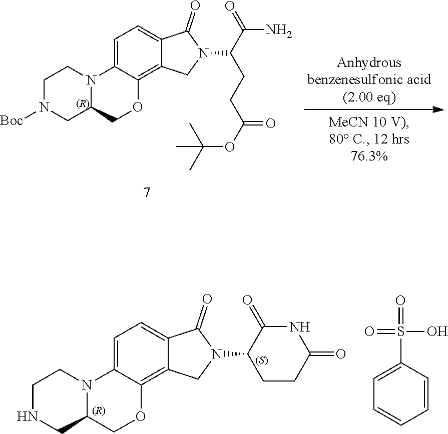

Anhydrous benzene sulfonic acid (59.6 g, 376 mmol, 2.00 eq) was dissolved in MeCN (1.0 L) at 20° C., and heated to reflux, before a solution of compound 7 (100 g, 188 mmol, 1.00 eq) in MeCN (200 mL) was added drop-wise to the mixture and a suspension was formed. The mixture was stirred under reflux temperature for further 12 hrs and cooled to 20° C. The precipitate was filtered and the filter cake was washed with MeCN (500 mL), and dried under reduced pressure at ° C. (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione benzenesulfonate (74 g) was obtained as a gray solid, and the typical yield was 76.3%.

### Step 8: Synthesis of (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride

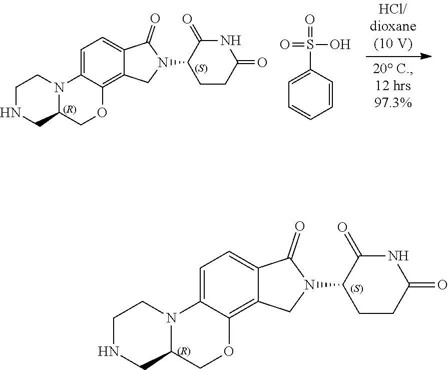

(S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione benzenesulfonate (175 g, 340 mmol, 1.00 eq) was added to the solution of HCl/dioxane (4N, 400 mL), and stirred at 20° C. for 12 hrs. The mixture was filtered, and the filter cake was washed with MeCN (1.0 L). The filter cake was added to the solution of HCl/dioxane (4N, 400 mL) again, and stirred for another 12 hrs. The mixture was filtered, and the filter cake was washed with MeCN (1.0 L). The filter cake was dried under reduced pressure at 45° C. (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride (130 g) was obtained as an off-white solid, and the typical yield was 97.3%. ¹H NMR (400 MHz, D₂O-d₂) 7.25 (d, J=8.4 Hz, 1H), 7.00 (d, J=8.4 Hz, 1H), 4.98 (br dd, J=5.2, 13.3 Hz, 1H), 4.38-4.19 (m, 3H), 4.13-4.01 (m, 2H), 3.62-3.37 (m, 3H), 3.22-3.06 (m, 2H), 3.03-2.70 (m, 3H), 2.39 (dq, J=5.4, 12.9 Hz, 1H), 2.19-2.07 (m, 1H)

### Compound A8. (S)-3-((S)-8-oxo-3,4,4a,5-tetrahydro-1H-pyrazino [1′,2′:4,5][1,4]oxazino[2,3-f]isoindol-9(2H,8H,10H)-yl)piperidine-2,6-dione hydrochloride

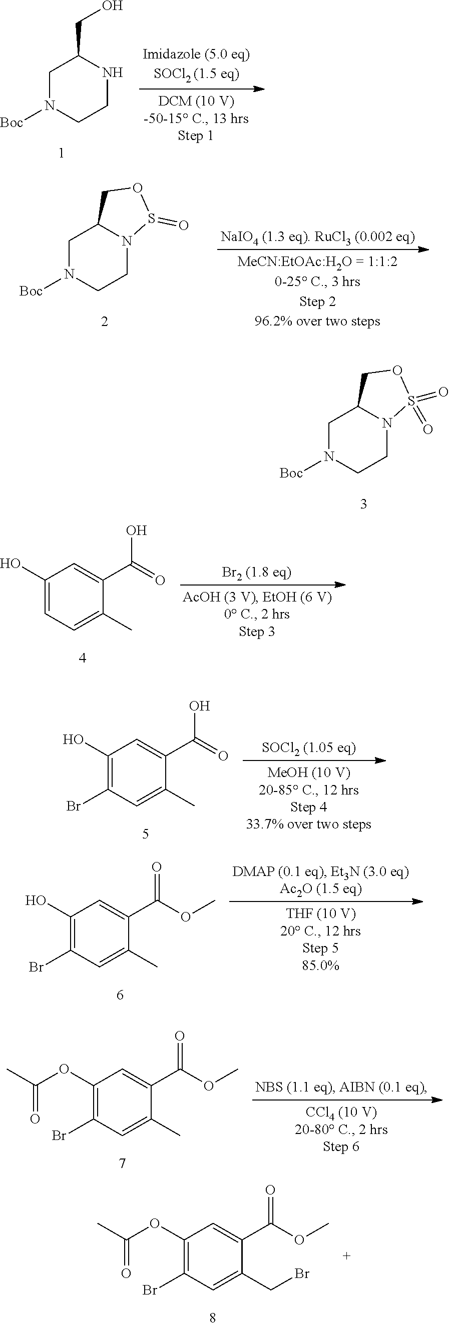

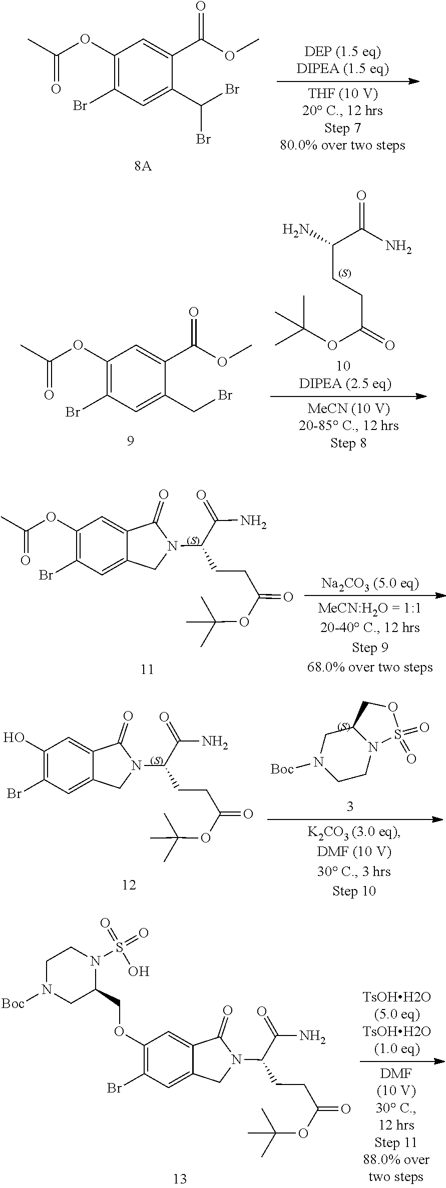

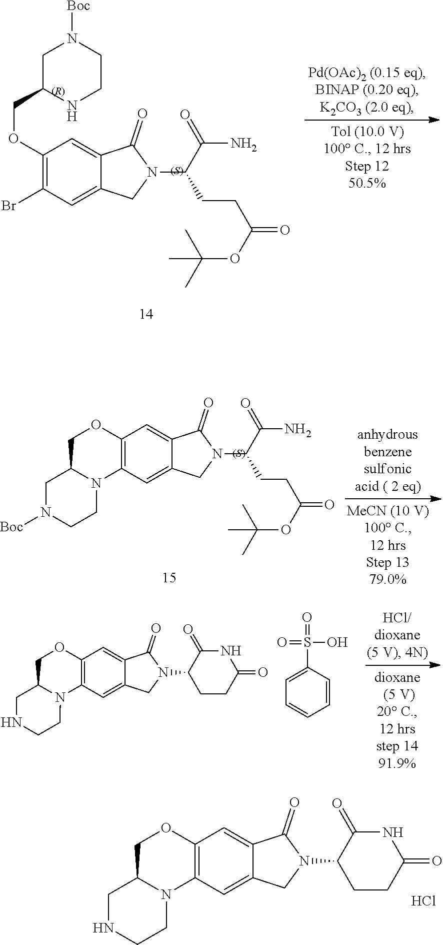

### Step 1: Synthesis of (3aS)-tert-butyl tetrahydro-[1,2,3]oxathiazolo [3,4-a]pyrazine-5(3H)-carboxylate 1-oxide

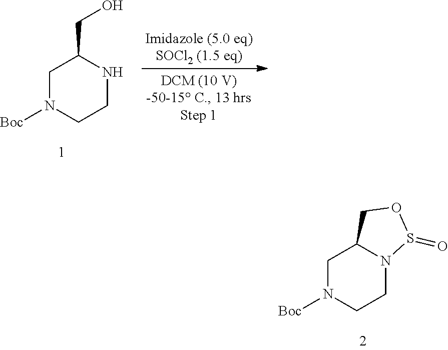

Two reactions were carried out in parallel. To the solution of imidazole (377.74 g, 5.55 mol, 6.00 eq) in DCM (2.0 L) was added SOCl₂ (100.62 mL, 1.39 mol, 1.50 eq) dropwise at 0° C. under N₂ atmosphere. Upon the addition of SOCl₂, a white precipitate was observed, and the reaction mixture was stirred at 15° C. for further 1 hr. During which period the temperature was maintained below 15° C. To the resulting mixture was added the solution of compound 1 (200.00 g, 924.74 mmol, 1.00 eq) in DCM (2.0 L) dropwise at −50° C. under N₂ atmosphere. The reaction mixture was warmed to 15° C., and stirred for further 12 hrs. Two reactions were combined for workup. The reaction was quenched by water (1.0 L), and stirred for 15 min, before the aqueous phase was extracted with DCM (2×1.0 L). The combined organic phase was washed with brine (500 mL), dried over anhydrous Na₂SO₄, filtered, and concentrated in vacuo. Crude compound 2 (480 g) was obtained as a yellow solid. ¹HNMR (400 MHz, CDCl₃-d) δ=4.74 (br t, J=7.0 Hz, 1H), 4.57-4.49 (m, 1H), 4.48-4.39 (m, 2H), 4.27-3.92 (m, 4H), 3.85 (br t, J=8.9 Hz, 1H), 3.57 (br d, J=11.2 Hz, 3H), 3.29 (br d, J=10.3 Hz, 1H), 3.22-3.10 (m, 2H), 3.06-2.74 (m, 4H), 2.66 (br s, 1H), 1.44 (s, 18H)

### Step 2: Synthesis of (S)-tert-butyl tetrahydro-[1,2,3]oxathiazolo[3,4-a]pyrazine-5(3H)-carboxylate 1,1-dioxide

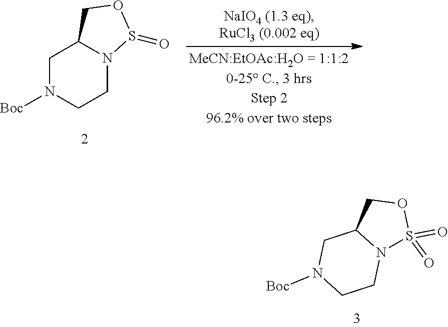

Two reactions were carried out in parallel. To a solution of compound 2 (240.00 g, 914.89 mmol, 1.00 eq) in MeCN (1.0 L) and EtOAc (1.0 L) was added a solution of RuCl₃ (379.55 mg, 1.83 mmol, 0.002 eq) and NaIO₄ (293.53 g, 1.37 mol, 76.04 mL, 1.50 eq) in H₂O (2.5 L) dropwise over 1 hr at 0° C. under N₂ atmosphere. The mixture was stirred at 25° C. for 3 hrs. Two reactions were combined for workup. The residue mixture was filtered under reduced pressure, and the filtered cake was washed with EA (2.0 L). The aqueous phase was extracted with EA (2×1.0 L), and the combined organic phase was washed with brine (1.0 L), dried over anhydrous Na₂SO₄, filtered and concentrated in vacuo. Compound 3 (490 g) was obtained as a yellow solid, and the typical yield was 96.2%. ¹HNMR (400 MHz, CDCl₃-d) δ=4.63 (dd, J=6.3, 8.0 Hz, 1H), 4.34-3.95 (m, 3H), 3.63 (dt, J=3.4, 6.2, 9.4 Hz, 1H), 3.45 (br d, J=11.5 Hz, 1H), 3.12 (br s, 1H), 2.95 (dt, J=3.1, 11.3 Hz, 2H), 1.47 (s, 9H)

### Step 3: Synthesis of 4-bromo-5-hydroxy-2-methylbenzoic acid

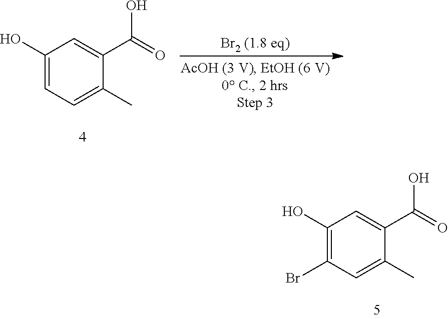

To the solution of compound 4 (100 g, 657.26 mmol, 1.00 eq) in EtOH (600 mL) and AcOH (300 mL) was added Br₂ (189.06 g, 1.18 mol, 60.99 mL, 1.80 eq) portion-wise at 0° C. under N₂ atmosphere. The resulting mixture was stirred at 0° C. for 2 hrs, before the reaction solution was poured into saturated sodium thiosulfate solution (500 mL) and stirred for 30 min. The resulting mixture was filtered and washed with EA (500 mL). The aqueous phase was extracted with EA (3×1.0 L), and the combined organic phase was washed with brine (1.0 L), dried over anhydrous Na₂SO₄, filtered and concentrated in vacuo. Crude compound 5 (150 g, crude) was obtained as a yellow oil.

### Step 4: Synthesis of methyl 4-bromo-5-hydroxy-2-methylbenzoate

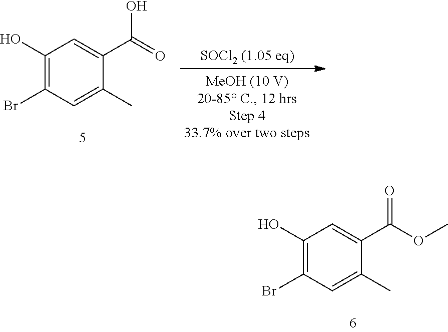

To the solution of crude compound 5 (150 g, 649.23 mmol, 1.00 eq) in MeOH (2.0 L) was added SOCl₂ (49.45 mL, 681.69 mmol, 1.05 eq) portion-wise at 20° C. under N₂ atmosphere, before being stirred at 85° C. for 12 hours. The reaction mixture was cooled to 20° C., and concentrated under reduced pressure. The solvent residue was diluted with H₂O (1.0 L), and pH was adjusted to 7.0 with saturated NaHCO₃ solution. The aqueous phase was extracted with EA (2×1.0 L), and the combined organic phase was washed with brine (500 mL), dried over anhydrous Na₂SO₄, filtered and concentrated in vacuo. Compound 6 (54.0 g) was obtained as a white solid, and the typical yield was 31.6%. ¹HNMR (400 MHz, DMSO-d6) δ=10.41 (s, 1H), 7.44 (d, J=9.7 Hz, 2H), 3.80 (s, 3H), 2.38 (s, 3H)

### Step 5: Synthesis of methyl 5-acetoxy-4-bromo-2-methylbenzoate

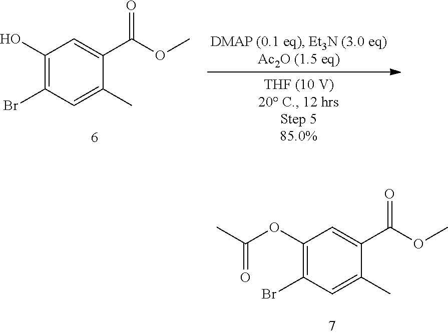 [SMILES: COC(=O)c1cc(O)c(Br)cc1C ; COC(=O)c1cc(OC(C)=O)c(Br)cc1C]

To the mixture of compound 6 (54 g, 220.35 mmol, 1.00 eq) and Et₃N (92.01 mL, 661.04 mmol, 3.00 eq) in THF (600 mL) was added DMAP (2.69 g, 22.03 mmol, 0.10 eq) and Ac₂O (33.74 g, 330.52 mmol, 30.96 mL, 1.50 eq) in one portion at 20° C. under N₂ atmosphere. The resulting mixture was stirred at 20° C. for 12 hours, before the mixture was concentrated under reduced pressure. The residue was diluted with EA (2.0 L) and washed with 0.1N HCl (500 mL), saturated NaHCO₃ solution (500 mL), and brine (500 mL), dried over anhydrous Na₂SO₄, filtered and concentrated in vacuo. Compound 7 (60.0 g) was obtained as a brown solid, and the typical yield was 94.8%. ¹HNMR (400 MHz, DMSO-d6) δ=7.74 (s, 1H), 7.69 (s, 1H), 3.82 (s, 3H), 2.50 (s, 3H), 2.32 (s, 3H)

### Step 6: Synthesis of methyl 5-acetoxy-4-bromo-2-(bromomethyl) benzoate and methyl 5-acetoxy-4-bromo-2-(dibromomethyl)benzoate

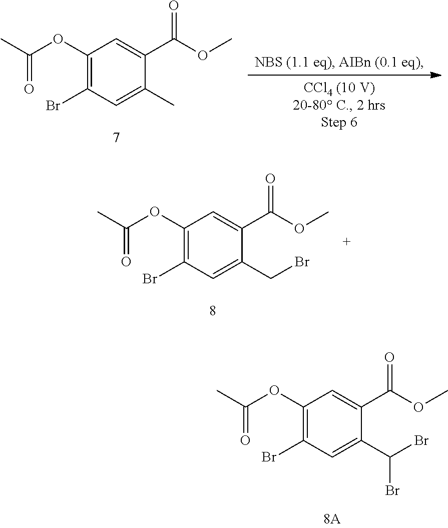 [SMILES: COC(=O)c1cc(OC(C)=O)c(Br)cc1C ; COC(=O)c1cc(OC(C)=O)c(Br)cc1CBr ; COC(=O)c1cc(OC(C)=O)c(Br)cc1C(Br)Br]

To the solution of compound 7 (60 g, 208.98 mmol, 1.00 eq) in CCl₄ (600 mL) was added NBS (63.23 g, 355.27 mmol, 1.70 eq) and AIBN (6.86 g, 41.80 mmol, 0.20 eq) in one portion at 20° C. under N₂ atmosphere, before being stirred at 80° C. for 12 hours. The reaction mixture was concentrated under reduced pressure, and the crude product was purified by silica gel column chromatography (column height: 250 mm, diameter: 100 mm, 100-200 mesh silica gel, Petroleum ether/Ethyl acetate=100/1 to 1/1). Compound 8 and Compound 8A (83 g in total) was obtained as a white solid.

### Step 7: Synthesis of methyl 5-acetoxy-4-bromo-2-(bromomethyl) benzoate

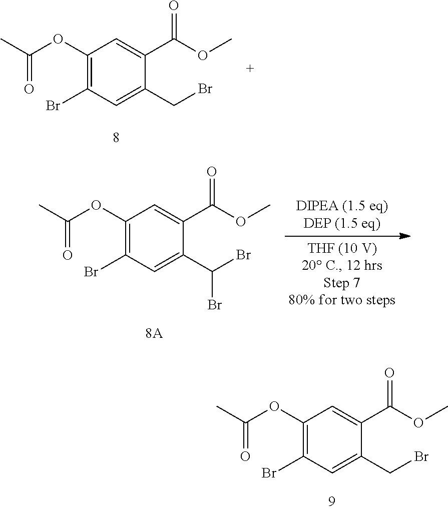 [SMILES: COC(=O)c1cc(OC(C)=O)c(Br)cc1CBr ; COC(=O)c1cc(OC(C)=O)c(Br)cc1C(Br)Br ; COC(=O)c1cc(OC(C)=O)c(Br)cc1CBr]

To the mixture of compound 8 and compound 8A (60 g, 134.86 mmol, 1.00 eq) and DIPEA (35.24 mL, 202.29 mmol, 1.50 eq) in THF (600 mL) was added DEP (27.94 g, 202.29 mmol, 26.11 mL, 1.50 eq) in one portion at 20° C. under N₂ atmosphere. The resulting mixture was stirred at 20° C. for 12 hours, before the reaction mixture was poured into water (500 mL) and stirred for 15 min. The aqueous phase was extracted with EA (2×500 mL), dried over anhydrous Na₂SO₄, filtered and concentrated in vacuo. The crude product was purified by silica gel column chromatography (column height: 250 mm, diameter: 100 mm, 100-200 mesh silica gel, Petroleum ether/Ethyl acetate=100/1 to 1/1). Crude compound 9 (54 g) was obtained as a white solid.

### Step 8: Synthesis of (S)-tert-butyl 4-(6-acetoxy-5-bromo-1-oxoisoindolin-2-yl)-5-amino-5-oxopentanoate

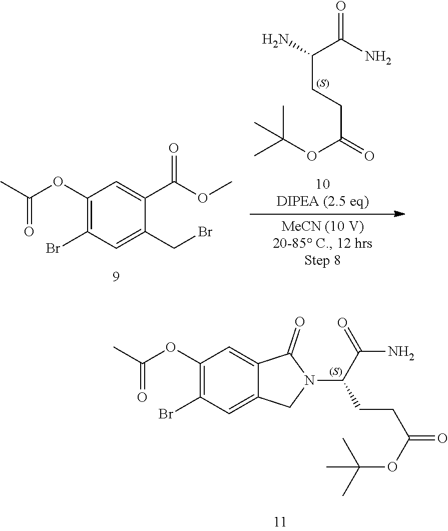

Two reactions were carried out in parallel. To the solution of compound 9 (50 g, 136.61 mmol, 1.00 eq) and compound 10 (48.87 g, 177.59 mmol, 1.30 eq, HCl salt) in MeCN (500 mL) was added DIPEA (44.14 g, 341.53 mmol, 59.49 mL, 2.50 eq) in one portion at 20° C. under N₂ atmosphere, before being stirred at 85° C. for 12 hours. The crude compound 11 (124 g) was used directly for the next step without further purification.

### Step 9: Synthesis of (S)-tert-butyl 5-amino-4-(5-bromo-6-hydroxy-1-oxoisoindolin-2-yl)-5-oxopentanoate

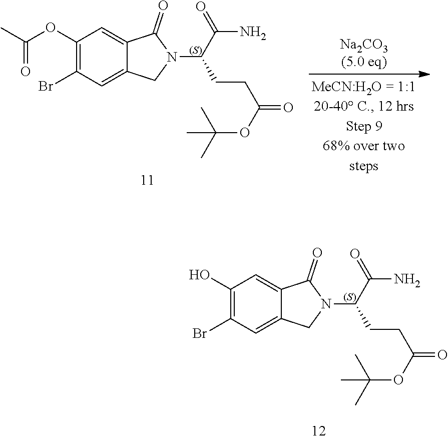

To the solution of compound 11 (124 g, 272.35 mmol, 1.00 eq) in MeCN (1.0 L) was added a solution of Na₂CO₃ (144.33 g, 1.36 mol, 5.00 eq) in H₂O (1.0 L) dropwise at 20° C. under N₂ atmosphere, before being stirred at 40° C. for 12 hours. The reaction mixture was cooled to 20° C., before being poured into water (500 mL) and stirred for 15 min, and the aqueous phase was extracted with EA (3×500 mL). The combined organic phase was washed with brine (500 mL), dried over anhydrous Na₂SO₄, filtered, and concentrated in vacuo. The crude product was triturated with MeCN (500 ml) at 20° C. for 12 hrs, and the solid was filtered and concentrated under reduced pressure. Compound 12 (77.0 g) was obtained as a white solid, and the typical yield was 68.4%.

### Step 10: Synthesis of (R)-2-(((2-((S)-1-amino-5-(tert-butoxy)-1,5-dioxopentan-2-yl)-6-bromo-3-oxoisoindolin-5-yl)oxy)methyl)-4-(tert-butoxycarbonyl)piperazine-1-sulfonic acid

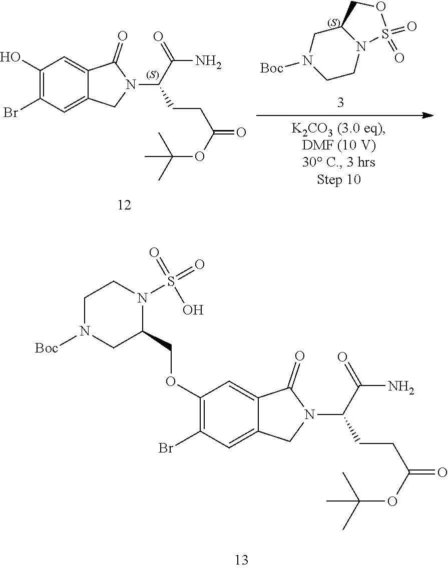

To the solution of compound 12 (100 g, 241.98 mmol, 1.00 eq) and compound 3 (67.35 g, 241.98 mmol, 1.00 eq) in DMF (1.0 L) was added K₂CO₃ (100.33 g, 725.93 mmol, 3.00 eq) portion-wise at 30° C. under N₂ atmosphere, before being stirred at 30° C. for 3 hrs. The crude compound 13 (167 g) was used directly for the next step without further purification.

### Step 11: Synthesis of (R)-tert-butyl 3-(((2-((S)-1-amino-5-(tert-butoxy)-1,5-dioxopentan-2-yl)-6-bromo-3-oxoisoindolin-5-yl)oxy)methyl) piperazine-1-carboxylate

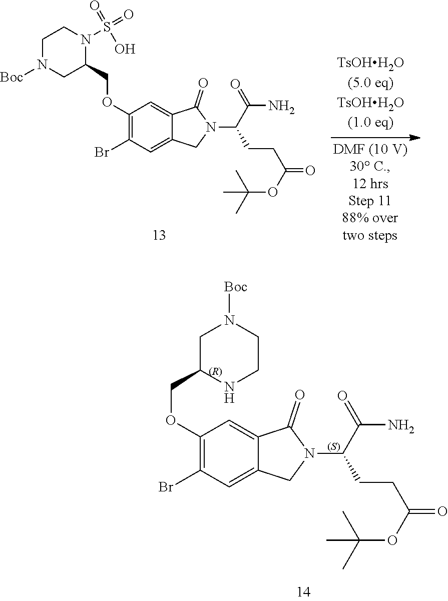

The solution of compound 13 (167 g, 241.47 mmol, 1.00 eq) in DMF (1.0 L) was adjusted pH to 7.0 with TsOH·H₂O (229.67 g, 1.21 mol, 5.00 eq) portion-wise at 30° C., before extra TsOH·H₂O (45.93 g, 241.47 mmol, 1.00 eq) was added in one portion at 30° C. The resulting mixture was stirred at 30° C. for 12 hrs, before the PH was adjusted to 8 with saturated Na₂CO₃ solution at 30° C. The aqueous phase was extracted with EA (2×2.0 L). The combined organic phase was washed with saturated Na₂CO₃ solution (3.0 L), brine (1.0 L), dried over anhydrous Na₂SO₄, filtered, and concentrated in vacuo. Compound 14 (130 g) was obtained as a white solid, and the typical yield was 88%. ¹H NMR (400 MHz, DMSO-d6) δ=7.89 (s, 1H), 7.58 (s, 1H), 7.35 (s, 1H), 7.20 (s, 1H), 4.77-4.69 (m, 1H), 4.58-4.49 (m, 1H), 4.44-4.32 (m, 1H), 4.11 (br s, 1H), 4.06-3.97 (m, 2H), 3.73 (br d, J=12.8 Hz, 1H), 2.94-2.88 (m, 3H), 2.77 (br d, J=11.6 Hz, 1H), 2.57 (br d, J=2.6 Hz, 1H), 2.46 (br s, 1H), 2.20-2.07 (m, 3H), 1.98 (br s, 1H), 1.38 (s, 9H), 1.32 (s, 9H)

### Step 12: Synthesis of (S)-tert-butyl 9-((S)-1-amino-5-(tert-butoxy)-1,5-dioxopentan-2-yl)-8-oxo-4,4a,5,8,9,10-hexahydro-1H-pyrazino[1′,2′:4,5][1,4]oxazino[2,3-f]isoindole-3(2H)-carboxylate

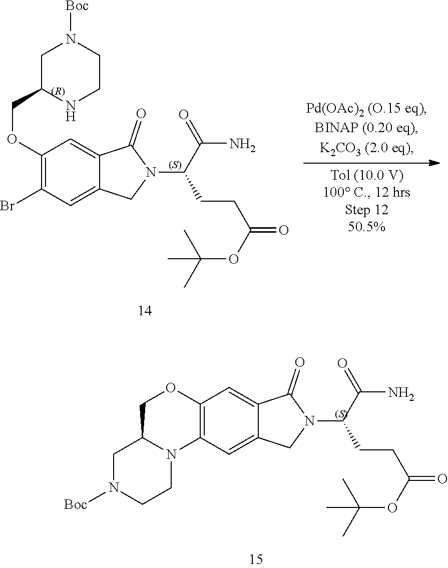

To the solution of compound 14 (8.50 g, 13.90 mmol, 1.00 eq) in toluene (100 mL) was added Pd(OAc)₂ (468.09 mg, 2.08 mmol, 0.15 eq), K₂CO₃ (3.84 g, 27.80 mmol, 2 eq) and BINAP (1.73 g, 2.78 mmol, 0.20 eq) in one portion at 20° C. under N₂ atmosphere, before being stirred at 100° C. for 12 hrs. The reaction mixture was filtered and washed with EA (2×500 ml), and the filtrate was concentrated in vacuo. The crude product was purified by silica gel column chromatography (column height: 250 mm, diameter: 100 mm, 100-200 mesh silica gel, Petroleum ether/Ethyl acetate=100/1 to 1/1). Compound 15 (5.1 g) was obtained as a white solid, and the typical yield was 50.5%. ¹HNMR (400 MHz, DMSO-d6) δ=7.50 (br s, 2H), 7.17-7.06 (m, 2H), 6.94 (s, 1H), 4.74-4.63 (m, 1H), 4.45 (br d, J=16.9 Hz, 1H), 4.35 (dd, J=2.8, 10.9 Hz, 1H), 4.26 (br d, J=17.0 Hz, 1H), 4.10-3.80 (m, 5H), 3.27-2.89 (m, 2H), 2.80-2.57 (m, 2H), 2.13 (br s, 3H), 1.43 (s, 10H), 1.34 (s, 9H).

### Step 13: Synthesis of (S)-3-((S)-8-oxo-3,4,4a,5-tetrahydro-1H-pyrazino[1′,2′:4,5][1,4]oxazino[2,3-f]isoindol-9(2H,8H,10H)-yl)piperidine-2,6-dione benzenesulfonate

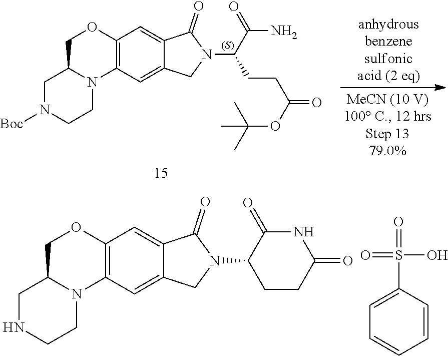

To the solution of compound 15 (2 g, 3.77 mmol, 1.00 eq) in MeCN (20 mL) was added anhydrous benzene sulfonic acid (1.19 g, 7.54 mmol, 2.00 eq) in one portion at 20° C. under N₂ atmosphere, being stirred at 100° C. for 12 hrs. The reaction mixture was filtered, and the filtered cake was washed with MeCN (10 mL). The solid was dried under reduced pressure. (S)-3-((S)-8-oxo-3,4,4a,5-tetrahydro-1H-pyrazino[1′,2′:4,5][1,4]oxazino[2,3-f]isoindol-9(2H,8H,10H)-yl)piperidine-2,6-dione benzenesulfonate (1.4 g) was obtained as a white solid, and the typical yield was 79.1%.

### Step 14: Synthesis of (S)-3-((S)-8-oxo-3,4,4a,5-tetrahydro-1H-pyrazino[1′,2′:4,5][1,4]oxazino[2,3-f]isoindol-9(2H,8H,10H)-yl)piperidine-2,6-dione hydrochloride

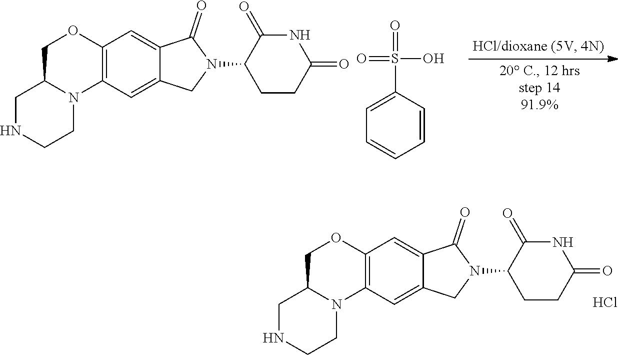

The suspension of (S)-3-((S)-8-oxo-3,4,4a,5-tetrahydro-1H-pyrazino[1′,2′:4,5][1,4]oxazino[2,3-f]isoindol-9(2H,8H,10H)-yl)piperidine-2,6-dione benzenesulfonate (1.3 g, 2.77 mmol, 1.00 eq) in HCl/dioxane (4 M, 10 mL) was stirred at 20° C. for 12 hrs, before being diluted with MeCN (50 mL). The precipitate was filtered and dried under reduced pressure. (S)-3-((S)-8-oxo-3,4,4a,5-tetrahydro-1H-pyrazino[1′,2′:4,5][1,4]oxazino[2,3-f]isoindol-9(2H,8H,10H)-yl)piperidine-2,6-dione hydrochloride (1.0 g) was obtained as a white solid, and the typical yield was 91.9%. ¹HNMR (400 MHz, D₂O-d₂) δ=7.09 (s, 1H), 7.02 (s, 1H), 5.00 (dd, J=5.2, 13.3 Hz, 1H), 4.42-4.19 (m, 3H), 4.11-3.96 (m, 2H), 3.66-3.55 (m, 1H), 3.52-3.36 (m, 2H), 3.16 (d, J=9.8 Hz, 2H), 2.95 (t, J=12.3 Hz, 1H), 2.86-2.67 (m, 2H), 2.50-2.29 (m, 1H), 2.23-2.08 (m, 1H).

### Compound A9. (S)—N—((S)-2,6-dioxopiperidin-3-yl)-1,2,3,4,4a,5-hexahydropyrazino[1,2-d]pyrido[2,3-b][1,4]oxazine-8-carboxamide

Compound A9 was synthesized following the similar procedures for B22. LC-MS purity: 95% (UV at 254 nm), 346.2 [M+H]⁺.

### Compound B0 (A7). (S)-3-((R)-1-oxo-5,5a,6,7,8,9-hexahydro-1H-pyrazino [1′,2′:4,5][1,4]oxazino[2,3-e]isoindol-2(3H)-yl)piperidine-2,6-dione hydrochloride salt

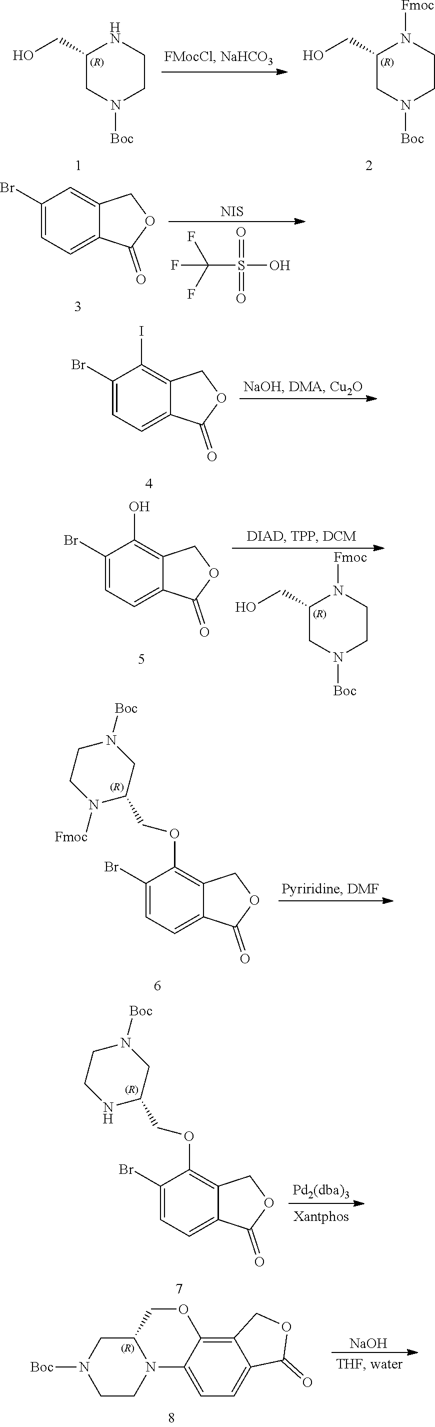

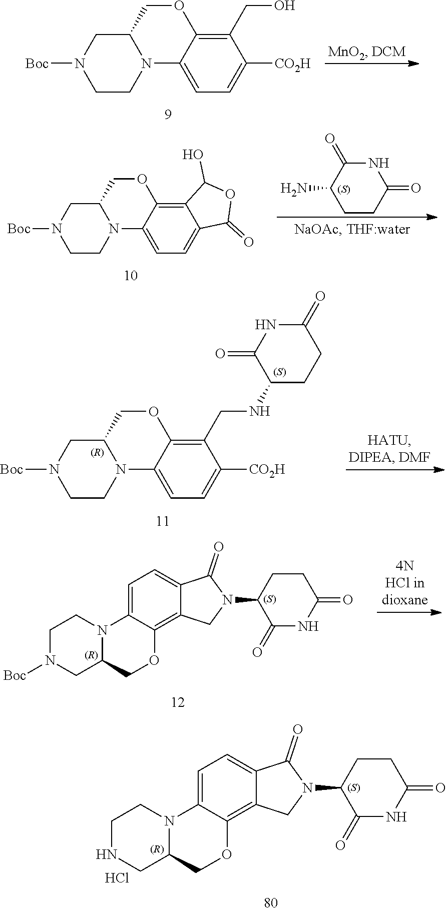

### Step 1: 1-((9H-fluoren-9-yl)methyl) 4-(tert-butyl) (R)-2-(hydroxymethyl)piperazine-1,4-dicarboxylate

(R)-1-Boc-3-(Hydroxymethyl)piperazine (1, 10 g, 46.2 mmol) was dissolved in a mixture of DCM (180 mL) and sat. NaHCO₃ (180 mL). FMocCl (46.2 mmol) was dissolved in DCM (15 mL) and added dropwise with vigorous stirring. The mixture was stirred for 1 hour. The layers were separated and the aqueous phase was extracted with DCM. The combined organic phases were washed with brine, dried over Na₂SO₄ and the solvent was removed in vacuo to give an oil. The crude product was purified by silica gel column chromatography using 0-80% EtOAc/hexane (80% yield).

### Step 2: 5-bromo-4-iodoisobenzofuran-1(3H)-one

To a solution of 5-Bromo-3H-isobenzofuran-1-one (3, 5 g, 23.4 mmol, 1 eq.) in trifluoromethanesulfonic acid (68 g, 40 mL, 19.30 eq) was added NIS (5.5 g, 24.6 mmol, 1.05 eq.) at 0° C. in portions. The mixture was allowed to warm to room temperature and stirred overnight. TLC (hexane:ethyl acetate=5:1) showed no starting material remained and two new spots (R_{f}=0.4, 0.5) formed. The reaction mixture was poured into ice-water (100 mL) and yellow solid precipitated. The mixture was filtered and the filter cake was washed with ice cold water. The filter cake was dissolved in DCM (500 mL) and washed with 1 (M) Na₂S₂O₃ followed by dried over sodium sulfate. The mixture was filtered and the filtrate was concentrated to afford a yellow solid. The crude product was purified on a 120 g silica column running a 0-10% EtOAc/hexane gradient over 70 min. ¹H NMR (400 MHz, CDCl₃): δ 7.83 (d, J=8.0 Hz, 1H), 7.77 (d, J=8.0 Hz, 1H), 5.10 (s, 2H).

### Step 3: 5-bromo-4-hydroxyisobenzofuran-1(3H)-one

To a mixture of 5-Bromo-4-iodo-3H-isobenzofuran-1-one (4, 4 g, 1 eq), sodium hydroxide (2.3 g, 5 eq) in water (40 mL, 1.5 M) and N,N-dimethylacetamide (20 ml) was added cuprous oxide (0.338 g, 0.2 eq). The reaction mixture was heated to 80° C. and held for 12 h. TLC (Hexane: ethyl acetate=1:1, Rf=0.3) showed the reaction was completed. The reaction mixture neutralized using 1 (N) hydrochloride solution and extracted with ethyl acetate (40 mL×2), washed with brine (150 mL), and then dried over sodium sulfate. The crude product was purified by silica gel column chromatography using 0-100% EtOAc/hexane. 5-Bromo-4-hydroxy-3H-isobenzofuran-1-one (5, 50% yield) was obtained as a white solid. ¹H NMR (400 MHz, DMSO) δ 10.90 (s, 1H), 7.72 (d, J=8.0 Hz, 1H), 7.23 (d, J=8.0 Hz, 1H), 5.35 (s, 2H).

### Step 4: 1-((9H-fluoren-9-yl)methyl) 4-(tert-butyl) (R)-2-(((5-bromo-1-oxo-1,3 dihydroisobenzofuran-4-yl)oxy)methyl)piperazine-1,4-dicarboxylate

To a solution of 5-Bromo-4-hydroxyisobenzofuran-1(3H)-one (5, 700 mg, 3 mmol, 1 eq.) in 12 mL of THF/DCM, 1-((9H-fluoren-9-yl)methyl) 4-(tert-butyl) (R)-2-(hydroxymethyl)piperazine-1,4-dicarboxylate (2 gm, 4.5 mmol, 1.5 eq.) and PPh₃ (1.17 gm, 4.5 mmol, 1.5 eq.) was added. The reaction mixture was cooled to 0° C. and DIAD (0.9 mL, 4.5 mmol, 1.5 eq.) was added dropwise. The resultant mixture was then stirred overnight at room temperature. The solvent was evaporated at reduced pressure; the crude product was purified by silica gel column chromatography using 0-100% EtOAc/hexane. LC/MS (ESI) m/z: 649.15

### Step 5: tert-butyl (R)-3-(((5-bromo-1-oxo-1,3-dihydroisobenzofuran-4-yl)oxy)methyl)piperazine-1-carboxylate

To a solution of 1-((9H-fluoren-9-yl)methyl) 4-(tert-butyl) (R)-2-(((5-Bromo-1-oxo-1,3-dihydroisobenzofuran-4-yl)oxy)methyl)piperazine-1,4-dicarboxylate (6, 1 gm) was added 20% (v/v) piperidine in DM F (5 mL/gm of SM). The resulting mixture was stirred at room temperature overnight. The mixture was diluted with ethyl acetate and washed with water. The combined organic phases were washed with brine, dried over Na₂SO₄ and the solvent was removed in vacuo to give an oil. The crude product was purified by silica gel column chromatography using 0-5% DCM in methanol. Yield 70%. LC/MS (ESI) m/z: 426.08 [M+1]⁺.

### Step 6: tert-butyl (R)-1-oxo-1,3,5a,6,8,9-hexahydroisobenzofuro[4,5-b]pyrazino[1,2-d][1,4]oxazine-7(5H)-carboxylate

A vial was charged with tert-butyl (R)-3-(((5-bromo-1-oxo-1,3-dihydroisobenzofuran-4-yl)oxy)methyl)piperazine-1-carboxylate (7, 170 mg, 0.38 mmol, 1 eq.), Pd₂(dba)₃ (0.1 eq.), XantPhos (0.2 eq.), Cs₂CO₃ (3 eq.) and dioxane (5 mL). The mixture was purged with nitrogen and heated to 100° C. for 6 h. TLC (ethyl acetate: petroleum ether=1:2) showed reaction was complete. The mixture was diluted with ethyl acetate and washed with water. The organic layer was washed with brine and dried over sodium sulfate. The crude product was purified by silica gel column chromatography using 0-50% EtOAc/hexane. LC/MS (ESI) m/z: 347.15 [M+1]⁺. Yield 60%

### Step 7: (R)-3-(tert-butoxycarbonyl)-7-(hydroxymethyl)-1, 2,3,4,4a,5-hexahydrobenzo[b]pyrazino[1,2-d][1,4]oxazine-8-carboxylic acid

To a solution of tert-butyl (R)-1-oxo-1,3,5a,6,8,9-hexahydroisobenzofuro[4,5-b]pyrazino[1,2-d][1,4]oxazine-7(5H)-carboxylate (8, 346 mg, 1 mmol, 1 eq) in tetrahydrofuran (4 mL) and water (4 mL) was added sodium hydroxide (200 mg, 5 eq). The mixture was stirred at 20° C. for 16 h. TLC (ethyl acetate:hexane=1:1) showed reaction was complete. The mixture was adjusted to pH=5 with aq. hydrochloric acid (1 M) and extracted with ethyl acetate (10 mL×3). The organic layer was washed with brine (10×2 mL) and dried over sodium sulfate. The crude material was not further purified and used as crude for the next steps. LC/MS (ESI) m/z: 365.16

### Step 8: tert-butyl (5aR)-3-hydroxy-1-oxo-1,3,5a,6,8,9-hexahydroisobenzofuro[4,5-b]pyrazino[1,2-d][1,4]oxazine-7(5H)-carboxylate

To a solution of (R)-3-(tert-butoxycarbonyl)-7-(hydroxymethyl)-1,2,3,4,4a,5-hexahydrobenzo[b]pyrazino[1,2-d][1,4]oxazine-8-carboxylic acid (9, 1 eq.) in dichloromethane (10 mL) was added manganese dioxide (15 eq.). The mixture was stirred at 20° C. for overnight. TLC (ethyl acetate:hexane=1:1) showed reaction was complete. The mixture was diluted with dichloromethane (10 mL) and filtered through a pad of Celite. The filtrate was concentrated in vacuum. The crude product was purified by silica gel column chromatography. LC/MS (ESI) m/z: 363.16. ¹H NMR (400 MHz, CD₃OD) δ 7.32 (d, J=8.3 Hz, 1H), 7.14 (d, J=8.4 Hz, 1H), 6.64-6.40 (m, 1H), 4.42 (dd, J=11.0, 3.0 Hz, 1H), 4.23-4.01 (m, 3H), 3.95 (d, J=12.4 Hz, 1H), 3.34-3.23 (m, 1H), 3.08 (brs, 1H), 2.87 (td, J=12.2, 3.5 Hz, 1H), 2.74 (s, 1H) 1.50 (S, 9H).

### Step 9: (R)-3-(tert-butoxycarbonyl)-7-((((S)-2,6-dioxopiperidin-3-yl)amino)methyl)-1,2,3,4,4a,5-hexahydrobenzo[b]pyrazino[1,2-d][1,4]oxazine-8-carboxylic acid

To a mixture of (S) 3-aminopiperidine-2,6-dione (10, 1.5 eq., HCl salt) in methanol (2 ml) and dichloromethane (4 ml) was added sodium acetate (4 eq.). The mixture was stirred at 20° C. for 15 min, then tert-butyl (5aR)-3-hydroxy-1-oxo-1,3,5a,6,8,9-hexahydroisobenzofuro[4,5-b]pyrazino[1,2-d][1,4]oxazine-7(5H)-carboxylate (1 eq.) was added and the mixture was stirred for 30 mins. Sodium cyanoborohydride (2 eq.) was added and the mixture was further stirred for 1 hour. LCMS showed the reaction was complete. The mixture was adjusted to pH=4-5 with an aqueous hydrochloric acid solution (1 M) and extracted with ethyl acetate (10 mL×3). The crude material was not further purified and used as crude for the next steps. LC/MS (ESI) m/z: 475.21

### Step 10: tert-butyl (R)-2-((S)-2,6-dioxopiperidin-3-yl)-1-oxo-2,3,5a,6,8,9-hexahydro-1H-pyrazino[1′,2′:4,5][1,4]oxazino[2,3-e]isoindole-7(5H)-carboxylate

To a solution of (R)-3-(tert-butoxycarbonyl)-7-((((S)-2,6-dioxopiperidin-3-yl)amino)methyl)-1,2,3,4,4a,5-hexahydrobenzo[b]pyrazino[1,2-d][1,4]oxazine-8-carboxylic acid (11, 90 mg, 0.18 mmol, 1 eq.) in dimethylformamide (5 mL) was added HATU (72 mg, 1.0 eq.) followed by addition of DIPEA (3 eq.). The solution was stirred for 15 mins, at 0° C. The residue was purified by reverse phase HPLC to get the desired compound 12. LC/MS (ESI) m/z: 457.20. ¹H NMR (400 MHz, Methanol-d4) δ 7.32 (d, J=8.3 Hz, 1H), 7.08 (d, J=8.4 Hz, 1H), 5.10 (dd, J=13.3, 5.2 Hz, 1H), 4.46-4.30 (m, 3H), 4.23-3.98 (m, 3H), 3.93 (d, J=12.4 Hz, 1H), 3.22 (ddd, J=11.2, 8.2, 3.0 Hz, 1H), 3.07 (s, 1H), 2.99-2.61 (m, 4H), 2.59-2.42 (m, 1H), 2.21-2.07 (m, 1H), 1.51 (s, 9H).

### Step 11: (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride

A mixture of tert-butyl (R)-2-((S)-2,6-dioxopiperidin-3-yl)-1-oxo-2,3,5a,6,8,9-hexahydro-1H-pyrazino[1′,2′:4,5][1,4]oxazino[2,3-e]isoindole-7(5H)-carboxylate (456 mg, 1.0 mmol, 1 eq.) in HCl/dioxane (10 mL) was stirred at room temperature for 2 h. The reaction mixture was concentrated to afford (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride (13, 400 mg, crude) as white solid.

### Compound B1: 3-((S)-8-oxo-1,2,3,4,4a,5,8,10-octahydro-9H-pyrazino[1′,2′:4,5][1,4]oxazino[2,3-f]isoindol-9-yl)piperidine-2,6-dione hydrochloride salt

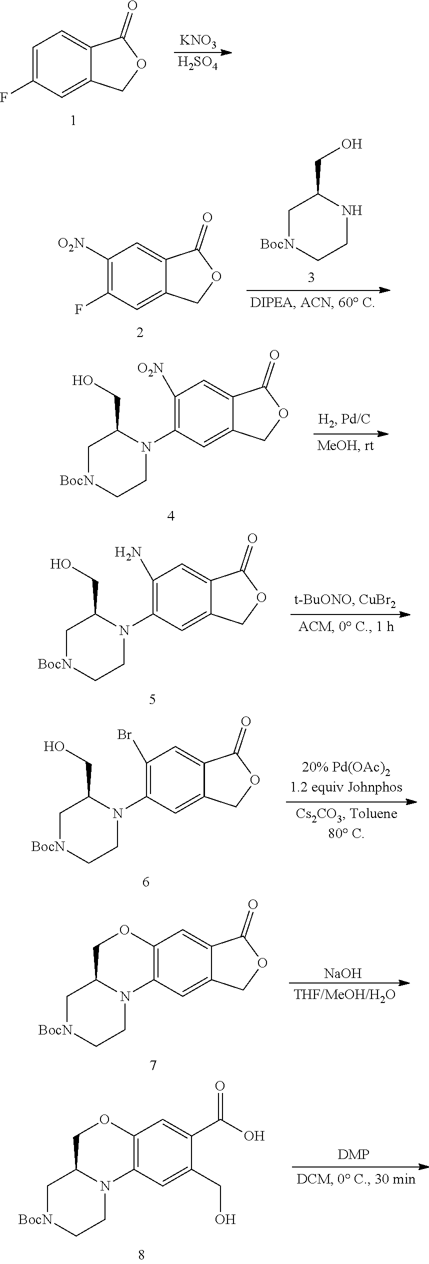

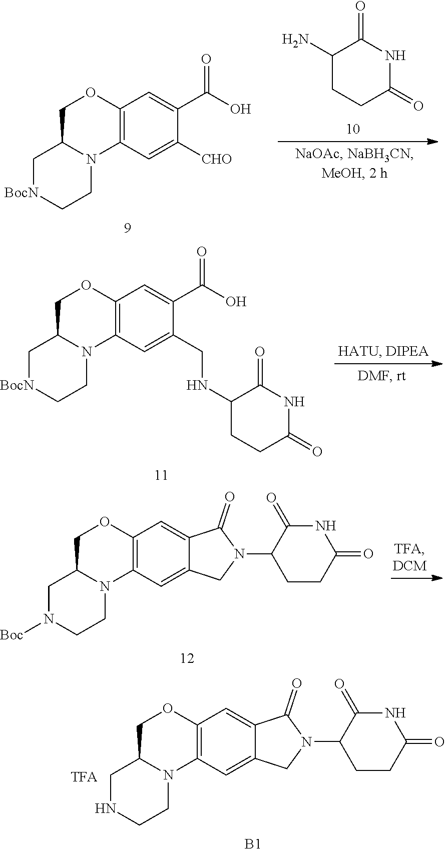 [SMILES: CC(C)(C)OC(=O)N1CCN2c3cc(C=O)c(C(=O)O)cc3OC[C@@H]2C1 ; NC1CCC(=O)NC1=O ; CC(C)(C)OC(=O)N1CCN2c3cc(CNC4CCC(=O)NC4=O)c(C(=O)O)cc3OC[C@@H]2C1 ; CC(C)(C)OC(=O)N1CCN2c3cc4c(cc3OC[C@@H]2C1)C(=O)N(C1CCC(=O)NC1=O)C4 ; O=C1CCC(N2Cc3cc4c(cc3C2=O)OC[C@@H]2CNCCN42)C(=O)N1 ; "B1"]

### Step 1: 5-fluoro-6-nitroisobenzofuran-1(3H)-one

To a solution of 5-fluoroisobenzofuran-1(3H)-one (10 g, 65.8 mmol, 1.0 eq.) in H₂SO₄ (50 mL) was added KNO₃ (9.97 g, 98.7 mmol, 1.5 eq.) in portions. The reaction mixture was stirred at room temperature for 3 h and slowly poured into ice water. The organic phase was washed with brine, dried over Na₂SO₄ and filtered. The filtrate was evaporated in vacuo and the residue was purified by column chromatography on silica gel eluted with 0-50% EtOAc/hexane to afford 5-fluoro-6-nitroisobenzofuran-1(3H)-one as white solid (10.4 g, 80% yield).

### Step 2: tert-butyl (S)-3-(hydroxymethyl)-4-(6-nitro-1-oxo-1,3-dihydroisobenzofuran-5-yl)piperazine-1-carboxylate

To a solution of 5-fluoro-6-nitroisobenzofuran-1(3H)-one (1 g, 5.0 mmol, 1 eq.) and tert-butyl (S)-3-(hydroxymethyl)piperazine-1-carboxylate (1.7 g, 7.5 mmol, 1.5 eq.) in acetonitrile (10 mL) was added DIPEA (2.2 mL, 12.5 mmol, 2.5 eq.) and the mixture was stirred at 60° C. for 6 h. The mixture was concentrated and the residue was purified by column chromatography on silica gel eluted with 0-5% MeOH/DCM to afford tert-butyl (S)-3-(hydroxymethyl)-4-(6-nitro-1-oxo-1,3-dihydroisobenzofuran-5-yl)piperazine-1-carboxylate as yellow foam (1.3 g, 66% yield).

### Step 3: tert-butyl (S)-4-(6-amino-1-oxo-1,3-dihydroisobenzofuran-5-yl)-3-(hydroxymethyl)piperazine-1-carboxylate

To a solution of tert-butyl (S)-3-(hydroxymethyl)-4-(6-nitro-1-oxo-1,3-dihydroisobenzofuran-5-yl)piperazine-1-carboxylate (1.0 g, 2.8 mmol, 1 eq.) in MeOH (15 mL) was added Pd/C (300 mg, 10% on carbon, wetted with ca. 55% water). The mixture was degassed and purged with H₂ three times and stirred at room temperature for 4 h. The catalyst was removed by filtration and the filtrate was evaporated to afford tert-butyl (S)-4-(6-amino-1-oxo-1,3-dihydroisobenzofuran-5-yl)-3-(hydroxymethyl)piperazine-1-carboxylate as light yellow foam (860 mg, 93% yield).

### Step 4: tert-butyl (S)-4-(6-bromo-1-oxo-1,3-dihydroisobenzofuran-5-yl)-3-(hydroxymethyl)piperazine-1-carboxylate

To a solution of tert-butyl tert-butyl (S)-4-(6-amino-1-oxo-1,3-dihydroisobenzofuran-5-yl)-3-(hydroxymethyl)piperazine-1-carboxylate (468 mg, 1.3 mmol, 1 eq.) in acetonitrile (25 mL) cooled in ice bath was added t-BuONO (0.2 mL, 1.7 mmol, 1.3 eq.) and the mixture was stirred for 30 min. Then a solution of CuBr₂ (300 mg, 1.3 mmol, 1 eq.) in acetonitrile (6 mL) was added to the solution dropwise and the mixture was stirred at room temperature for 3 h. Then the mixture was diluted with EA (120 mL) and water (120 mL). The organic phase was washed with brine, dried over Na₂SO₄ and filtered. The filtrate was evaporated in vacuo and the residue was purified by column chromatography on silica gel eluted with 0-5% MeOH/DCM to afford tert-butyl (S)-4-(6-bromo-1-oxo-1,3-dihydroisobenzofuran-5-yl)-3-(hydroxymethyl)piperazine-1-carboxylate as brown oil (415 mg, 75% yield).

### Step 5: tert-butyl (S)-8-oxo-1,2,4a,5,8,10-hexahydroisobenzofuro[5,6-b]pyrazino[1,2-d][1,4]oxazine-3(4H)-carboxylate

A mixture of tert-butyl (S)-4-(6-bromo-1-oxo-1,3-dihydroisobenzofuran-5-yl)-3-(hydroxymethyl)piperazine-1-carboxylate (140 mg, 0.3 mmol, 1 eq.), Pd(OAc)₂ (36.8 mg, 0.15 mmol, 0.5 eq.), JohnPhos (118 mg, 0.36 mmol, 1.2 eq.) and Cs₂CO₃ (214 mg, 0.7 mmol, 2 eq.) in toluene was degassed and purged with N₂ three times, and then the mixture was stirred at 90° C. for 3 h. The mixtures was cooled to room temperature, filtered through Celite, and the filtrate was concentrated. The residue was triturated with MeOH, and the solid was collected by filtration to afford tert-butyl (S)-8-oxo-1,2,4a,5,8,10-hexahydroisobenzofuro[5,6-b]pyrazino[1,2-d][1,4]oxazine-3(4H)-carboxylate as yellow solid (90 mg, 80% yield).

### Step 6: (S)-3-(tert-butoxycarbonyl)-9-(hydroxymethyl)-1,2,3,4,4a,5-hexahydrobenzo[b]pyrazino[1,2-d][1,4]oxazine-8-carboxylic acid

To a solution of tert-butyl (S)-8-oxo-1,2,4a,5,8,10-hexahydroisobenzofuro[5,6-b]pyrazino[1,2-d][1,4]oxazine-3(4H)-carboxylate (87 mg, 0.25 mmol, 1 eq.) in THF (3 mL) was added a solution of NaOH (60 mg, 1.3 mmol, 6 eq.) in H₂O (1 mL) and the mixture was stirred at 40° C. for 6 h. Then the mixture was concentrated and the residue was diluted with water (4 mL) and acidified to PH 3-4 with 2 N HCl. The mixture was extracted with DCM (10 mL) and the organic phase was washed with brine, dried over Na₂SO₄ and filtered. The filtrate was evaporated in vacuo to afford (S)-3-(tert-butoxycarbonyl)-9-(hydroxymethyl)-1,2,3,4,4a,5-hexahydrobenzo[b]pyrazino[1,2-d][1,4]oxazine-8-carboxylic acid as white powder (76 mg, 83% yield).

### Step 7: (S)-3-(tert-butoxycarbonyl)-9-formyl-1,2,3,4,4a,5-hexahydrobenzo[b]pyrazino[1,2-d][1,4]oxazine-8-carboxylic acid

To a solution of (S)-3-(tert-butoxycarbonyl)-9-(hydroxymethyl)-1,2,3,4,4a,5-hexahydrobenzo[b]pyrazino[1,2-d][1,4]oxazine-8-carboxylic acid (54 mg, 0.15 mmol, 1 eq.) in DCM (10 mL) cooled at 0° C. was added DMP (93.7 mg, 0.23 mmol, 1.5 eq.) in small portions and the mixture was stirred at 0° C. for 30 min. Then the mixture was diluted with DCM and washed with brine. The organic phase was dried over Na₂SO₄ and filtered. The filtrate was evaporated in vacuo to afford (S)-3-(tert-butoxycarbonyl)-9-formyl-1,2,3,4,4a,5-hexahydrobenzo[b]pyrazino[1,2-d][1,4]oxazine-8-carboxylic acid as yellow solid (50 mg, crude).

### Step 7: (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid

To a mixture of (S)-3-(tert-butoxycarbonyl)-9-formyl-1,2,3,4,4a,5-hexahydrobenzo[b]pyrazino[1,2-d][1,4]oxazine-8-carboxylic acid (70 mg, 0.2 mmol, 1 eq.), 3-aminopiperidine-2,6-dione (47.6 mg, 0.3 mmol, 1.5 eq.) and NaOAc (23.7 mg, 0.3 mmol, 1.5 eq.) dissolved in MeOH (6 mL) was added NaBH₃CN (36 mg, 0.6 mmol, 3 eq.) and the mixture was stirred at room temperature for 1 h. Then the reaction was quenched with water and the mixture was purified by reverse phase column chromatography (0-50% Acetonitrile/0.05% formic acid) to afford (4aS)-3-(tert-butoxycarbonyl)-9-(((2,6-dioxopiperidin-3-yl)amino)methyl)-1,2,3,4,4a,5 -hexahydrobenzo[b]pyrazino[1,2-d][1,4]oxazine-8-carboxylic acid as white powder (35 mg, 38% yield) after lyophilized.

### Step 8: tert-butyl (4aS)-9-(2,6-dioxopiperidin-3-yl)-8-oxo-1,2,4a,5,9,10-hexahydro-8H-pyrazino[1′,2′:4,5][1,4]oxazino[2,3-f]isoindole-3(4H)-carboxylate

To a solution of (4aS)-3-(tert-butoxycarbonyl)-9-(((2,6-dioxopiperidin-3-yl)amino)methyl)-1,2,3,4,4a,5-hexahydrobenzo[b]pyrazino[1,2-d][1,4]oxazine-8-carboxylic acid (47 mg, 0.1 mmol, 1 eq.) in DMF (2.5 mL) was added HATU (54 mg, 0.15 mmol, 1.5 eq.) followed by DIPEA (40 mg, 0.3 mmol, 3 eq.) and the mixture was stirred at room temperature for 1 h. Then the reaction was quenched with water and the mixture was purified by reverse phase column chromatography (0-50% acetonitrile/0.05% formic acid) to afford tert-butyl (4aS)-9-(2,6-dioxopiperidin-3-yl)-8-oxo-1,2,4a,5,9,10-hexahydro-8H-pyrazino[1′,2′:4,5][1,4]oxazino[2,3-f]isoindole-3(4H)-carboxylate (30 mg, 66% yield) as white powder.

### Step 9: 3-((S)-8-oxo-1,2,3,4,4a,5,8,10-octahydro-9H-pyrazino[1′,2′:4,5][1,4]oxazino[2,3-f]isoindol-9-yl)piperidine-2,6-dione hydrochloride salt trifluoroacetate salt

A mixture of tert-butyl (4aS)-9-(2,6-dioxopiperidin-3-yl)-8-oxo-1,2,4a,5,9,10-hexahydro-8H-pyrazino[1′,2′:4,5][1,4]oxazino[2,3-f]isoindole-3(4H)-carboxylate (30 mg, 1.0 eq) and HCl/dioxane (2 mL) was stirred at room temperature for 2 h. The mixture was concentrated to afford 3-((S)-8-oxo-1,2,3,4,4a,5,8,10-octahydro-9H-pyrazino[1′,2′:4,5][1,4]oxazino[2,3-f]isoindol-9-yl)piperidine-2,6-dione trifluoroacetate salt as white solid (26 mg, crude).

LC-MS: [M+H]⁺=356.90. ¹H NMR (400 MHz, Methanol-d4) δ 7.15 (s, 1H), 7.11 (d, J=5.7 Hz, 1H), 5.13-5.02 (m, 1H), 4.41-4.27 (m, 3H), 4.20 (d, J=13.6 Hz, 1H), 4.12-4.01 (m, 1H), 3.62-3.43 (m, 3H), 3.30-3.10 (m, 2H), 3.03-2.94 (m, 1H), 2.94-2.82 (m, 1H), 2.82-2.71 (m, 1H), 2.52-2.38 (m, 1H), 2.20-2.09 (m, 1H). ¹³C NMR (101 MHz, MeOD) δ 174.68, 172.52, 172.49, 171.63, 171.59, 146.42, 146.37, 139.22, 139.20, 138.12, 138.08, 123.85, 123.78, 111.88, 108.70, 108.65, 66.94, 53.72, 53.57, 50.79, 50.75, 48.90, 48.68, 44.36, 44.17, 44.10, 43.63, 43.61, 32.35, 24.08.

### Compound B6: 3-((R)-8-oxo-1,2,3,4,4a,5,8,10-octahydro-9H-pyrazino[1′,2′:4,5][1,4]oxazino[2,3-f]isoindol-9-yl)piperidine-2,6-dione trifluoroacetate salt

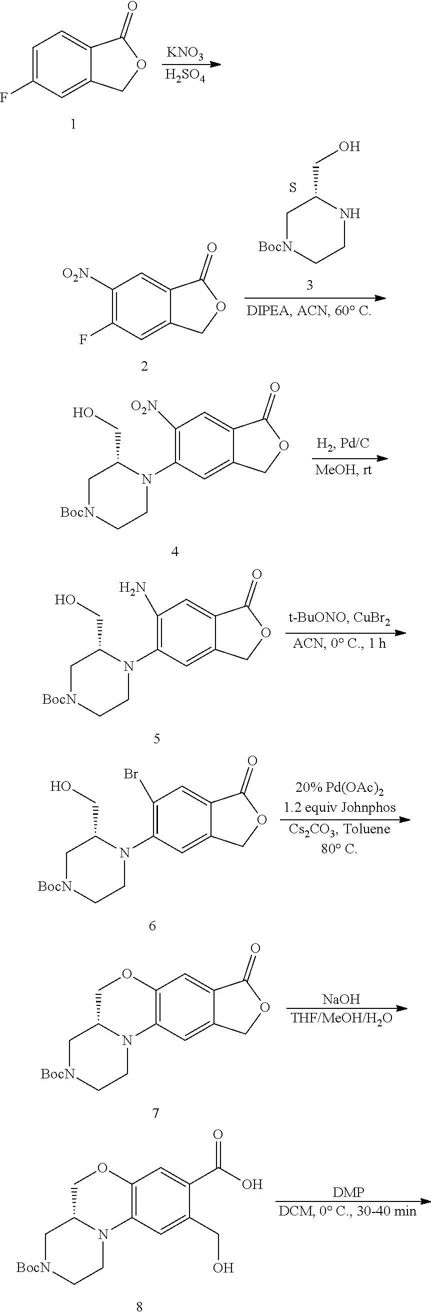

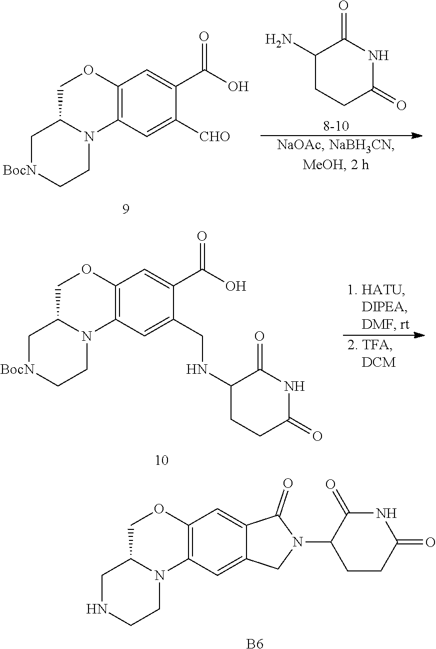

Compound B6 was made using the similar procedure for making Compound B1. LC-MS: [M+H]⁺=356.91. ¹H NMR (400 MHz, Methanol-d4) δ 7.17 (s, 1H), 7.13 (d, J=1.7 Hz, 1H), 5.14-5.03 (m, 1H), 4.44-4.28 (m, 3H), 4.27-4.17 (m, 1H), 4.12-4.03 (m, 1H), 3.61-3.42 (m, 3H), 3.30-3.21 (m, 1H), 3.21-3.11 (m, 1H), 3.04-2.95 (m, 1H), 2.95-2.83 (m, 1H), 2.81-2.72 (m, 1H), 2.52-2.38 (m, 1H), 2.19-2.10 (m, 1H). ¹³C NMR (101 MHz, MeOD) δ 174.66, 172.46, 172.44, 171.64, 171.61, 146.51, 146.47, 139.24, 139.20, 138.14, 123.98, 123.91, 111.93, 108.73, 108.70, 66.97, 53.71, 53.60, 50.88, 50.86, 44.38, 44.24, 44.20, 43.62, 32.38, 24.13.

### Compound B7: 3-((S)-6-methyl-8-oxo-2,3,4,4a,5,6,8,10-octahydropyrazino[1,2-a]pyrrolo[3,4-g]quinoxalin-9(1H)-yl)piperidine-2,6-dione trifluoroacetate salt

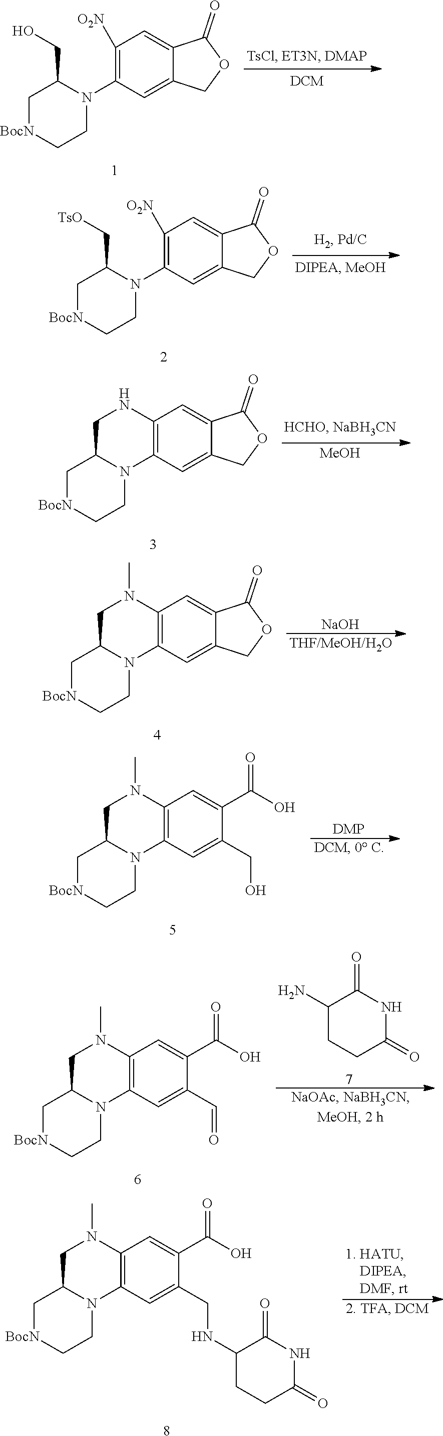

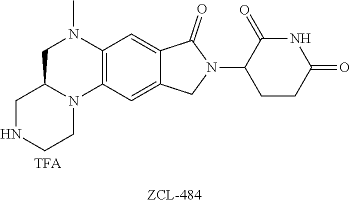 [SMILES: CN1C[C@@H]2CNCCN2c2cc3c(cc21)C(=O)N(C1CCC(=O)NC1=O)C3 ; "ZCL-484"]

### Step 1: tert-butyl (S)-4-(6-nitro-1-oxo-1,3-dihydroisobenzofuran-5-yl)-3-((tosyloxy)methyl)piperazine-1-carboxylate

To a solution of 1 (1 equiv, 1.49 g) in DCM (30 mL) was TsCl (2.0 equiv, 1.44 g), Et₃N (4.0 equiv, 2.11 mL) and DMAP (0.2 equiv, 92 mg), and the mixture was stirred at rt overnight. TLC (n-Hexane:EA=1:1) indicated the starting material 1 was completely conversion and an new spot detected. Then the reaction mixture was diluted with DCM, washed with brine, dried over Na₂SO₄, and concentrated under reduced pressure to give a residue which was purified by silica gel flash chromatography (n-Hexane:EA=100:0 to 60:40). The desired product 2 was obtained as a yellow foam (1.67 g, yield=81%).

### Step 2: tert-butyl (R)-8-oxo-1,2,4,4a,5,6,8,10-octahydro-3H-furo[3,4-g]pyrazino[1,2-a]quinoxaline-3-carboxylate

To a solution of 2 (1.0 equiv, 1.67 g) in MeOH (20 mL) was added DIPEA (2.0 equiv, 1.06 mL), followed by Pd/C (0.5 equiv, 835 mg). The reaction mixture was degassed and purged with H₂ three times and keep stirred at rt overnight. UPLC-MS showed the starting material completely conversed to desired product 3. Then the reaction mixture was filtered through Celite, and the filtrate was concentrated under reduced pressure to give a residue which was purified by silica gel flash chromatography (DCM:MeOH=100:0 to 95:5). The desired product 3 was obtained as a yellow solid (957 mg, yield=91%).

### Step 3: tert-butyl (R)-6-methyl-8-oxo-1,2,4,4a,5,6,8,10-octahydro-3H-furo[3,4-g]pyrazino[1,2-a]quinoxaline-3-carboxylate

To a solution of 3 (1.0 equiv, 410 mg) in MeOH/AcOH/DCM (10 mL/1 mL/3 mL) was added HCHO (5.0 equiv, 470 mg), and the mixture was kept stirring for 2 h. Then NaBH₃CN (5.0 equiv, 361 mg) was added. 15 min Later, UPLC-MS showed the starting material 3 all converted to desired product 4. The reaction mixture was concentrated under reduced pressure, diluted with DCM, washed with brine, dried over Na₂SO₄ and concentrated to give a yellow powder which is directly used in the next step.

### Step 4: (R)-3-(tert-butoxycarbonyl)-9-(hydroxymethyl)-6-methyl-2,3,4,4a,5,6-hexahydro-1H-pyrazino[1,2-a]quinoxaline-8-carboxylic acid

4 (1.0 equiv, 427 mg) was dissolved in THF/MeOH/H₂O (3 mL/3 mL/1 mL), and NaOH (5.0 equiv, 238 mg) was added. The reaction was kept stirring at 40° C. overnight. Then the reaction mixture was concentrated under reduced pressure to remove the solvent. The residue was diluted with 3-4 mL H₂O, followed by acidified with 2 N aq. HCl to PH 3-4. White solid was precipitated, which was collected and dried to give desired product 5 as a white powder 358 mg (yield=80% in two steps).

### Step 5: (R)-3-(tert-butoxycarbonyl)-9-formyl-6-methyl-2,3,4,4a,5,6-hexahydro-1H-pyrazino[1,2-a]quinoxaline-8-carboxylic acid

To a solution of 5 (1.0 equiv, 305 mg) in DCM (20 mL) was added DMP (1.65 equiv, 565 mg) into 3 portions at 0° C. 30 min Later, UPLC-MS indicated that 5 was completely conversion and a new main peak with desired MS formed, then the reaction was immediately diluted with DCM, washed with brine, dried over and concentrated under reduced pressure to give a crude product 6 which is directly used in the next step.

### Step 6: (4aR)-3-(tert-butoxycarbonyl)-9-(((2,6-dioxopiperidin-3-yl)amino)methyl)-6-methyl-2,3,4,4a,5,6-hexahydro-1H-pyrazino[1,2-a]quinoxaline-8-carboxylic acid

A mixture of 6 (1.0 equiv, 303 mg), 7 (1.5 equiv, 199.5 mg) and NaOAc (1.5 equiv, 99.4 mg) was dissolved in MeOH (20 mL), and kept stirring at rt for 20 min. Then NaBH₃CN (3.0 equiv, 151 mg) was added in 3 portions. 2 h Later, UPLC-MS showed the starting material 6 was completely conversion and a new main peak with desired MS formed. Next, the reaction mixture was quenched with 4 mL water and concentrated under reduced pressure to give a residue which was purified by pre-HPLC (20% to 100% acetonitrile (0.1% HCOOH, not TFA) in 80 min, 60 mL/min, 27% acetonitrile come out). The desired product 8 was obtained as a dark solid 138 mg (yield=35% in two steps) after lyophilization.

### Step 7: 3-((S)-6-methyl-8-oxo-2,3,4,4a,5,6,8,10-octahydropyrazino[1,2-a]pyrrolo[3,4-g]quinoxalin-9(1H)-yl)piperidine-2,6-dione trifluoroacetate salt

To a solution of 8 (1.0 equiv, 138 mg) in DMF (5 mL) was added HATU (1.1 equiv, 118 mg) and DIPEA (3.0 equiv, 148 uL), and the reaction was stirred at rt for 20-30 min. UPLC-MS indicated a new main peak with desired MS formed, then quenched with 3 mL water and purified by HPLC-MS (acetonitrile 35% to 100% in 65 min, 60 mL/min, 44% acetonitrile come out). Collected the solution and concentrated to give a solid which was dissolved into TFA/DCM to deprotect the Boc group. The title compound Compound B7 was obtained as a light purple solid 40 mg (yield is much higher than here because much product was lost when purified) after removed the solvent and lyophilized. LC-MS: [M+H]⁺=370.02. ¹H NMR (400 MHz, Methanol-d₄) δ 6.99-6.93 (m, 2H), 5.12-5.04 (m, 1H), 4.34-4.29 (m, 1H), 4.27-4.18 (m, 1H), 3.69-3.60 (m, 1H), 3.51-3.40 (m, 2H), 3.39-3.33 (m, 1H), 3.27-3.12 (m, 4H), 3.07-3.00 (m, 1H), 2.92 (s, 3H), 2.88-2.85 (m, 1H), 2.80-2.73 (m, 1H), 2.51-2.38 (m, 1H), 2.18-2.10 (m, 1H).

### Compound B11: (3S)-3-(4-methyl-1-oxo-3,4,5,5a,6,7,8,9-octahydropyrazino[1,2-a]pyrrolo[3,4-f]quinoxalin-2(1H)-yl)piperidine-2,6-dione trifluoroacetate salt

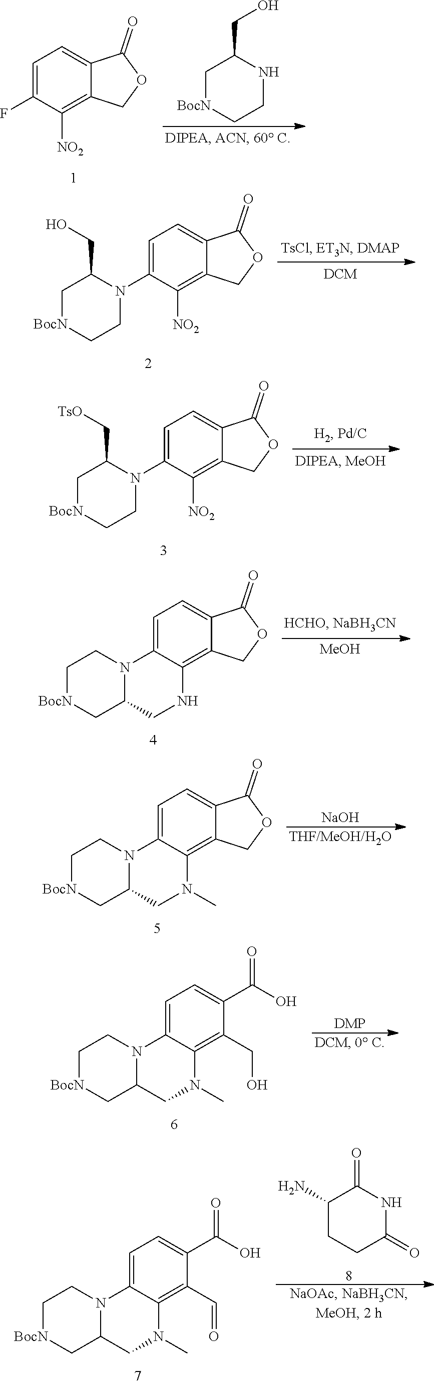

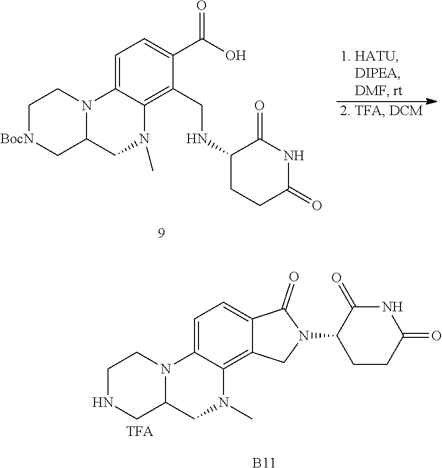

Compound B11 was made using the similar procedure for making Compound B7. LC-MS: [M+H]⁺=370.28. ¹H NMR (400 MHz, Methanol-d4) δ 7.32 (dd, J=8.4, 2.3 Hz, 1H), 7.09 (dd, J=8.6, 2.5 Hz, 1H), 5.14-5.04 (m, 1H), 4.63-4.44 (m, 2H), 4.30-4.16 (m, 1H), 3.60-3.51 (m, 1H), 3.49-3.39 (m, 2H), 3.29-3.21 (m, 2H), 3.16-3.02 (m, 2H), 2.98-2.83 (m, 5H), 2.82-2.72 (m, 1H), 2.58-2.45 (m, 1H), 2.21-2.10 (m, 1H). ¹³C NMR (101 MHz, MeOD) δ 174.70, 172.53, 172.50, 171.69, 171.63, 141.77, 141.73, 133.94, 133.82, 133.63, 124.97, 118.36, 118.32, 115.17, 53.85, 53.82, 53.67, 53.63, 47.98, 47.86, 46.28, 44.83, 44.39, 43.81, 43.75, 32.37, 24.03, 23.99.

### Compound B9: (4aS)-9-(2,6-dioxopiperidin-3-yl)-2,3,4,4a,9,10-hexahydropyrazino[1,2-a]pyrrolo[3,4-g]quinoxaline-5,8(1H,6H)-dione

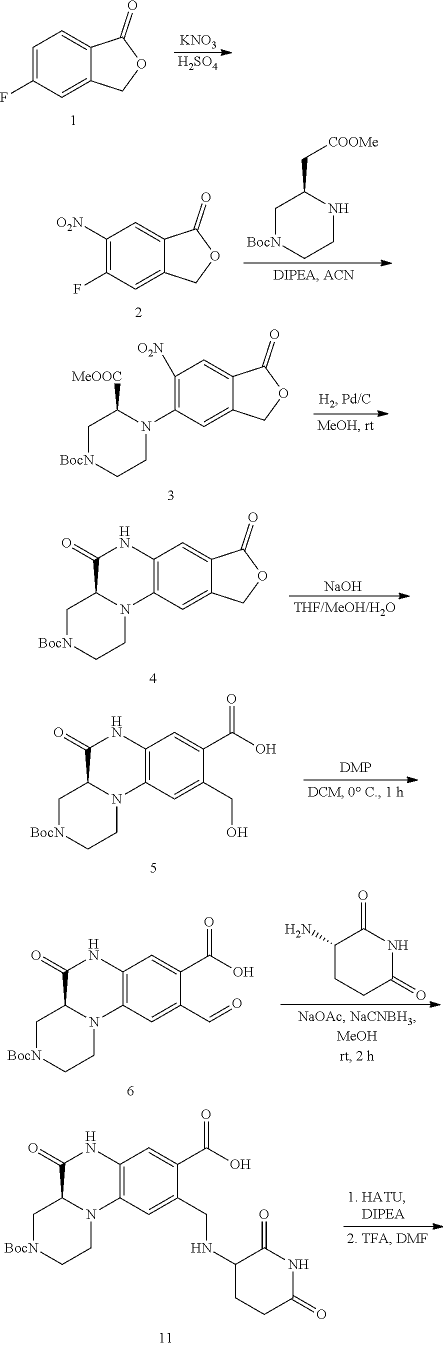

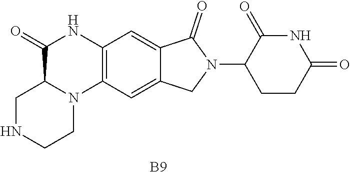

Compound B9 was made using the similar procedure for making Compound B7. LC-MS: [M+H]⁺=370.20.

### Compound B15. (S)-3-((S)-1-oxo-1,3,5,5a,6,7,8,9-octahydro-2H-pyrazino[1′,2′:4,5][1,4]oxazino[2,3-e]isoindol-2-yl)piperidine-2,6-dione

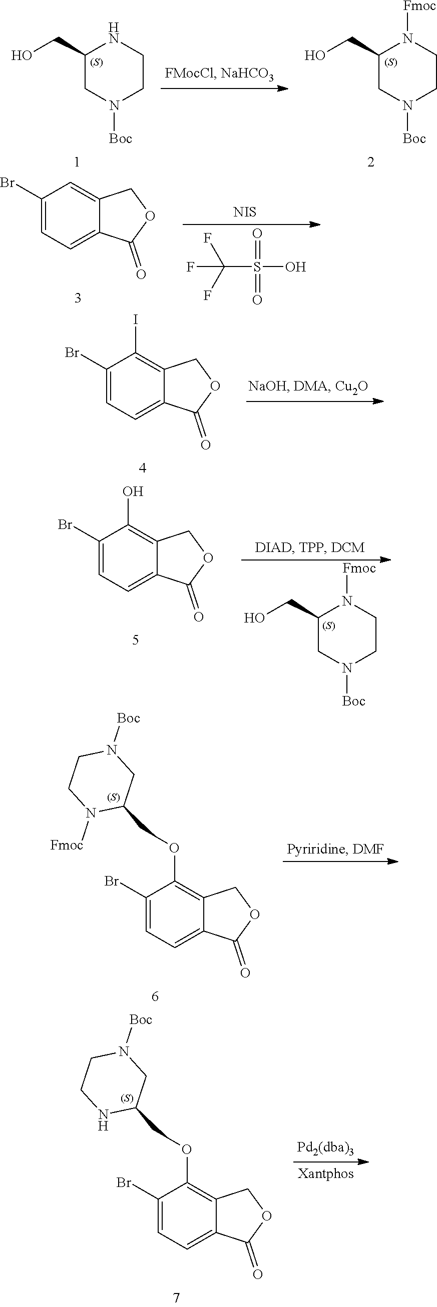

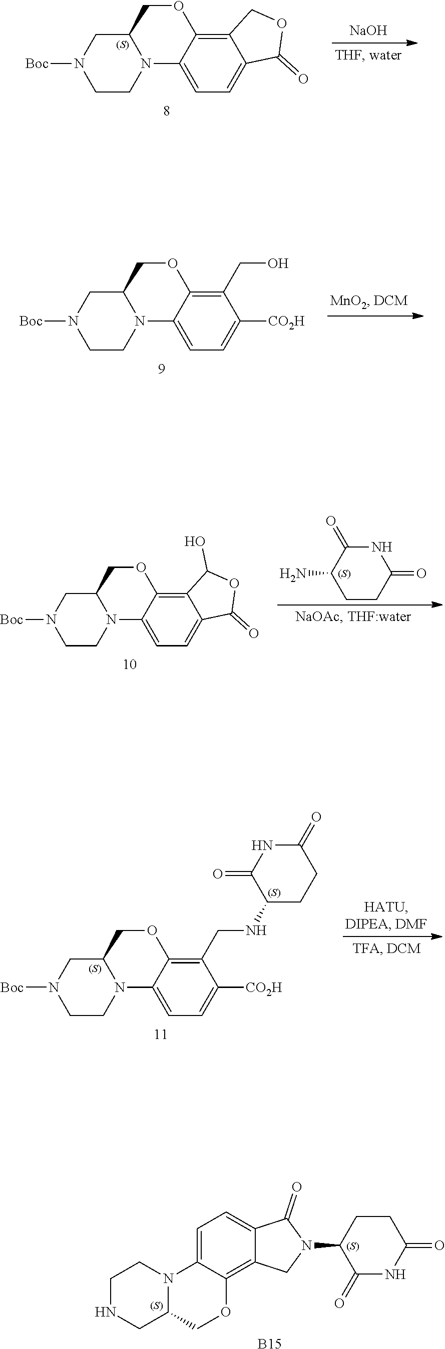

Compound B15 was made using the similar procedure for making intermediate B0.

¹H NMR (400 MHz, Methanol-d₄) δ 7.37 (d, J=8.3 Hz, 1H), 7.14 (d, J=8.5 Hz, 1H), 5.11 (dcd, J=13.3, 5.2, 2.2 Hz, 1H), 4.50-4.34 (m, 3H), 4.34-4.10 (m, 3H), 3.67-3.42 (m, 4H), 3.30-3.22 (m, 1H), 3.22-3.09 (m, 1H), 3.02 (td, J=12.2, 5.7 Hz, 1H), 2.91 (ddd, J=18.5, 13.4, 5.4 Hz, 1H), 2.79 (ddd, J=17.6, 4.7, 2.4 Hz, 1H), 2.57-2.41 (m, 1H), 2.16 (dtd, J=12.9, 5.3, 2.5 Hz, 1H).

### Compound B17. (S)-3-((2S,3aS)-2-amino-7-oxo-2,3,3a,4,7,9-hexahydro-1H,8H-pyrrolo[1′,2′:4,5][1,4]oxazino[2,3-f]isoindol-8-yl)piperidine-2,6-dione Compound B18. tert-butyl ((2S,3aS)-8-(2,6-dioxopiperidin-3-yl)-7,9-dioxo-2,3,3a,4,8,9-hexahydro-1H,7H-pyrrolo[1′,2′:4,5][1,4]oxazino[2,3-f]isoindol-2-yl)carbamate

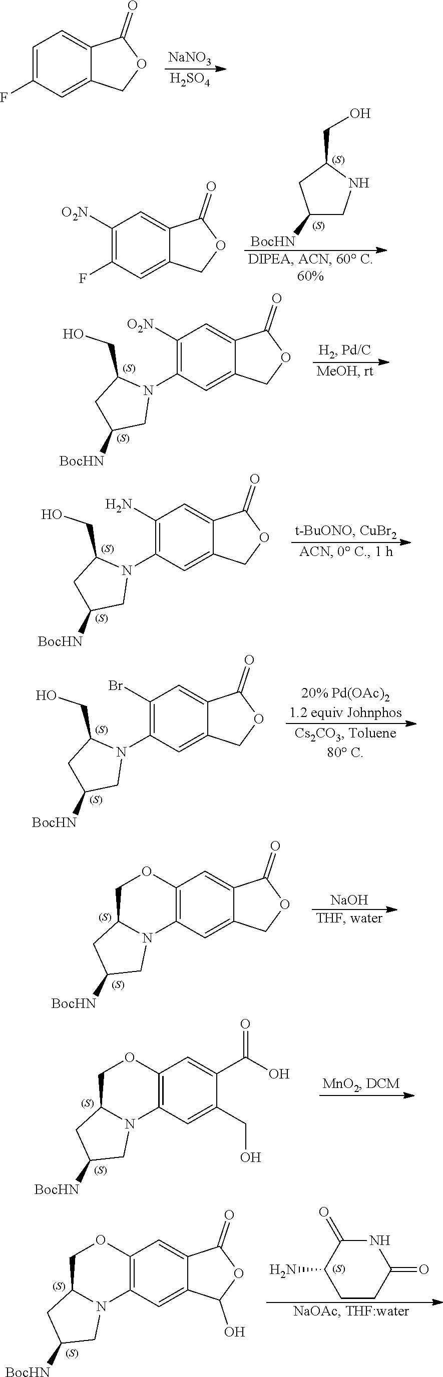

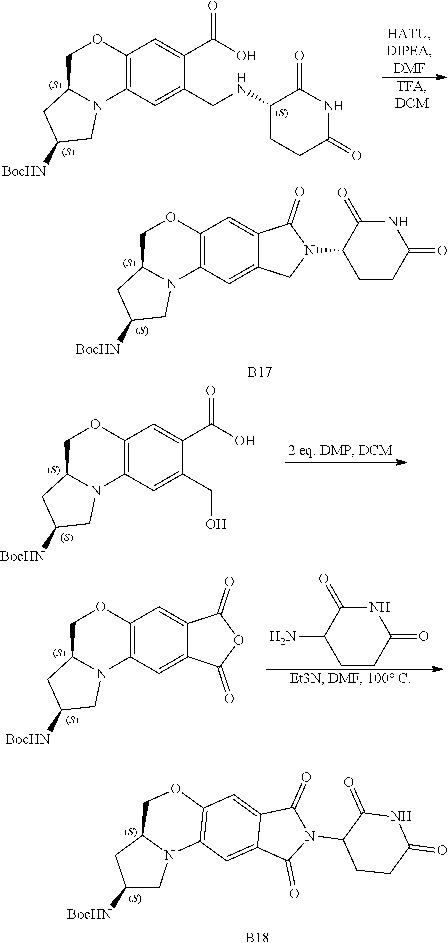

Compound B17 and Compound B18 were made using the similar procedure for making intermediate B1.

¹H NMR of compound Compound B17 (400 MHz, Methanol-d4) δ 7.16 (s, 1H), 6.72 (s, 1H), 5.09 (dt, J=13.3, 5.1 Hz, 1H), 4.58 (d, J=7.1 Hz, 2H), 4.36 (d, J=6.7 Hz, 2H), 4.15 (d, J=3.6 Hz, 1H), 3.79 (dd, J=10.4, 7.9 Hz, 1H), 3.72-3.62 (m, 2H), 3.53-3.40 (m, 1H), 2.96-2.84 (m, 1H), 2.78 (ddd, J=17.4, 4.8, 2.5 Hz, 1H), 2.61 (ddd, J=12.5, 8.6, 4.1 Hz, 1H), 2.55-2.37 (m, 1H), 2.16 (ddq, J=10.4, 5.3, 2.7 Hz, 1H), 1.79-1.59 (m, 1H).

¹H NMR Compound B18: (400 MHz, Methanol-d4) δ 7.25 (d, J=1.1 Hz, 1H), 7.02 (s, 1H), 5.06 (dd, J=12.4, 5.5 Hz, 1H), 4.70 (d, J=7.1 Hz, 1H), 4.18 (p, J=7.6 Hz, 1H), 3.91 (dd, J=10.6, 8.0 Hz, 1H), 3.74 (dd, J=8.2, 3.4 Hz, 2H), 3.60-3.46 (m, 1H), 2.96-2.80 (m, 1H), 2.80-2.61 (m, 3H), 2.18-2.04 (m, 1H), 1.70 (dt, J=12.2, 9.3 Hz, 1H).

### Compound B16. (R)-3-((S)-3-oxo-1,3,7,7a,8,9,10,11-octahydro-2H-pyrazino[1′,2′:4,5][1,4]oxazino[3,2-e]isoindol-2-yl)piperidine-2,6-dione

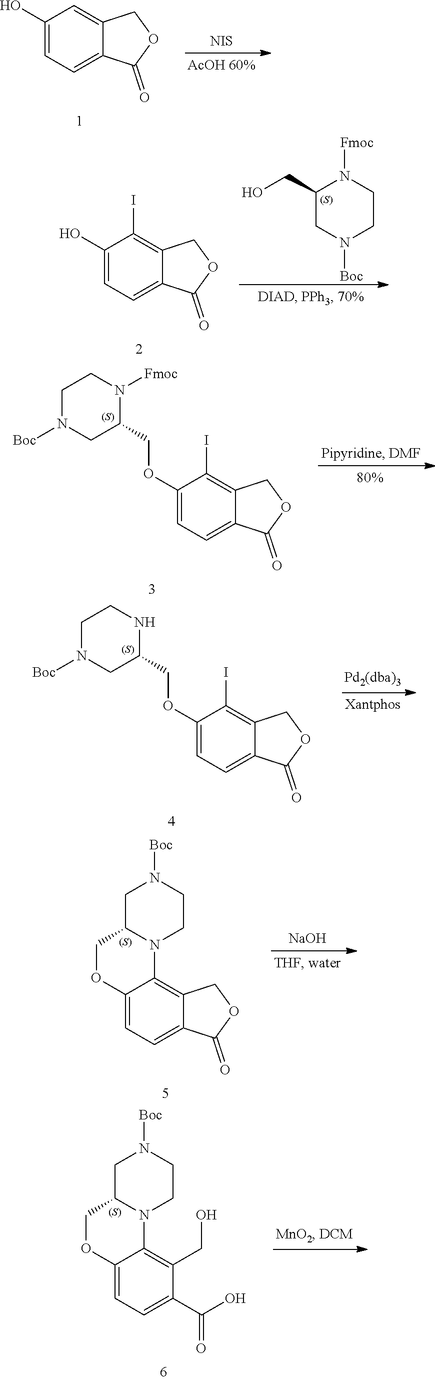

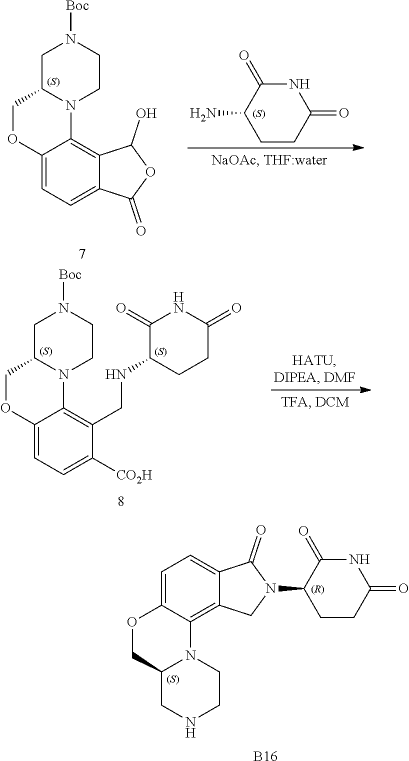

Intermediate Compound B16 was made using the similar procedure for making intermediate B0.

¹H NMR (400 MHz, Methanol-d₄) δ 7.32 (d, J=8.1 Hz, 1H), 7.02 (dd, J=8.1, 0.9 Hz, 1H), 5.15 (dd, J=13.4, 5.2 Hz, 1H), 4.70-4.47 (m, 2H), 4.34 (ddd, J=11.3, 4.2, 2.8 Hz, 1H), 4.14 (ddd, J=11.3, 9.8, 7.2 Hz, 1H), 4.04-3.91 (m, 1H), 3.65 (ddq, J=10.4, 7.1, 3.4, 2.8 Hz, 1H), 3.54-3.39 (m, 2H), 3.30-3.22 (m, 2H), 3.14 (dt, J=12.8, 10.6 Hz, 1H), 2.94 (ddd, J= 17.6, 13.5, 5.4 Hz, 1H), 2.80 (ddd, J=17.6, 4.7, 2.4 Hz, 1H), 2.60-2.42 (m, 1H), 2.19 (ddq, J=10.5, 5.4, 2.8 Hz, 1H).

### Compound B23: (S)—N—((S)-2,6-dioxopiperidin-3-yl)-1,2,3,4,4a,5-hexahydrobenzo[b]pyrazino[1,2-d][1,4]oxazine-8-carboxamide

### Compound B24: (4aS)—N-(2,6-dioxopiperidin-3-yl)-N-methyl-1,2,3,4,4a,5-hexahydrobenzo[b]pyrazino[1,2-d][1,4]oxazine-8-carboxamide

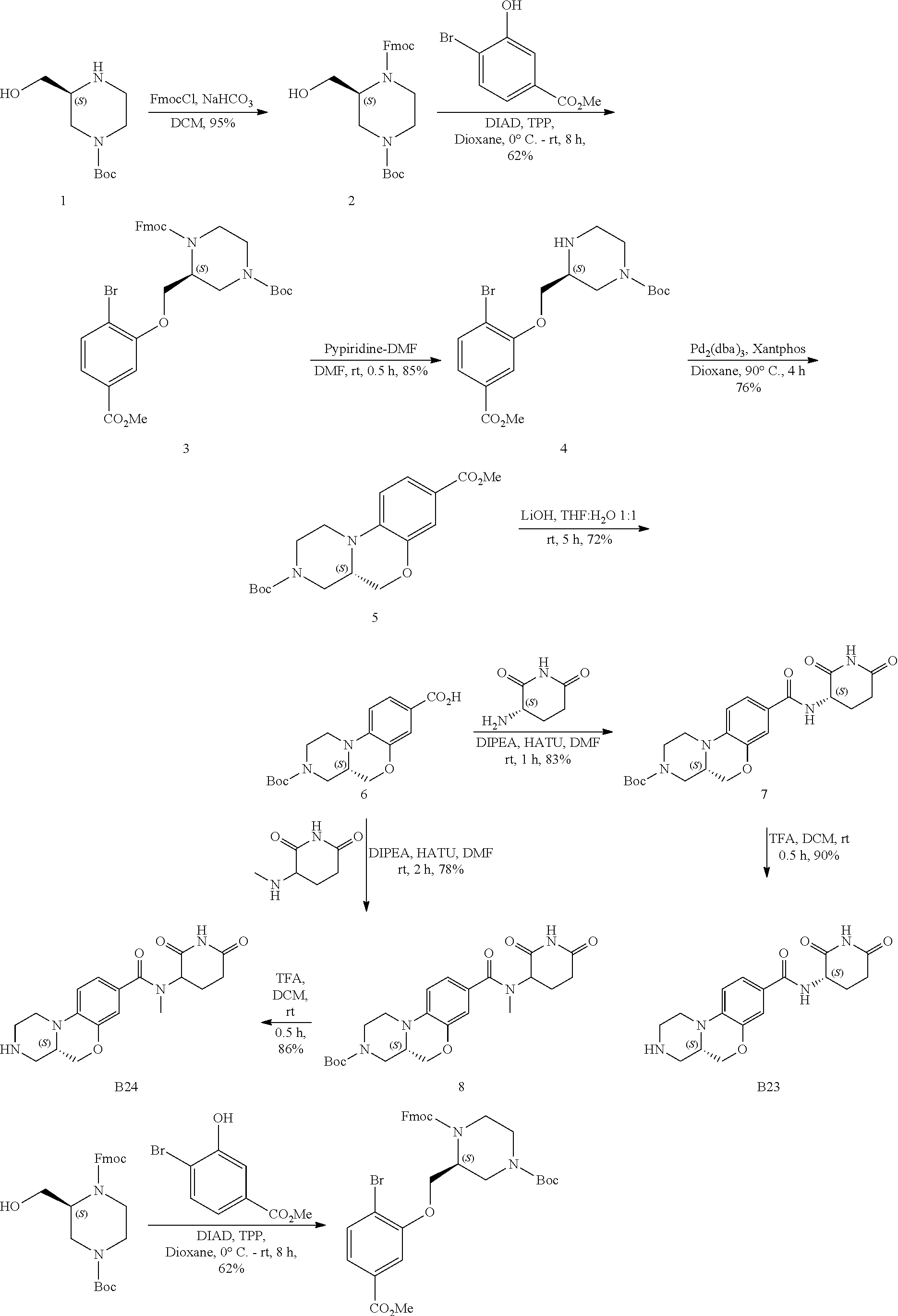

To a solution of methyl 4-bromo-3-hydroxybenzoate (1.38 g, 6 mmol, 1 eq.) in 18 ml of dioxane, 1-((9H-fluoren-9-yl)methyl) 4-(tert-butyl) (S)-2-(hydroxymethyl)piperazine-1,4-dicarboxylate (2, 3.94 g, 9 mmol, 1.5 eq.) and PPh₃ (2.35 g, 9 mmol, 1.5 eq.) was added. The reaction mixture was cooled to 0° C. and DIAD (1.77 mL, 9 mmol, 1.5 eq.) was added dropwise. The resultant mixture was then stirred overnight at room temperature. The solvent was evaporated at reduced pressure; the crude product was purified by silica gel column chromatography using 0-100% EtOAc/hexane. Yield 62%. LC/MS (ESI) m/z: 651.16 [M+1]⁺.

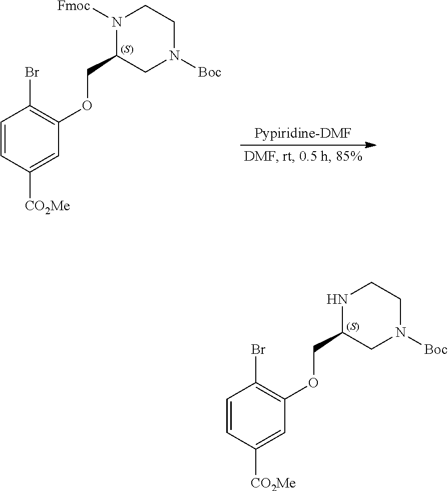

To a solution of 1-((9H-fluoren-9-yl)methyl) 4-(tert-butyl) (S)-2-((2-bromo-5-(methoxycarbonyl)phenoxy)methyl)piperazine-1,4-dicarboxylate (3, 2 g, 3.07 mmol) was added 20% (v/v) piperidine in DMF (5 ml/gm of SM). The resulting mixture was stirred at room temperature for 0.5 h. The mixture was diluted with ethyl acetate and washed with water. The combined organic phases were washed with brine, dried over Na₂SO₄ and the solvent was removed in vacuo to give an oil. The crude product was purified by silica gel column chromatography using 0-5% DCM in methanol. Yield 85%. LC/MS (ESI) m/z: 429.09 [M+1]⁺.

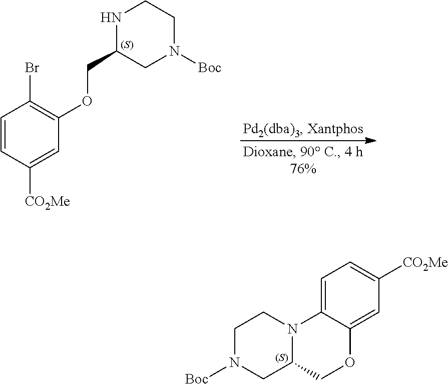

A vial was charged with tert-butyl tert-butyl (S)-3-((2-bromo-5-(methoxycarbonyl)phenoxy)methyl)piperazine-1-carboxylate (4, 600 mg, 1.39 mmol, 1 eq.), Pd₂(dba)₃ (194 mg, 0.21 mmol, 0.15 eq.), XantPhos (160 mg, 0.27 mmol, 0.2 eq.), Cs₂CO₃ (1.35 g, 4.17 mmol, 3 eq.) and dioxane (10 ml). The mixture was purged with nitrogen and heated to 90° C. for 4 h. TLC (ethyl acetate: petroleum ether=1:2) showed reaction was complete. The mixture was diluted with ethyl acetate and washed with water. The organic layer was washed with brine and dried over sodium sulfate. The crude product was purified by silica gel column chromatography using 0-50% EtOAc/hexane. Yield 76%. LC/MS (ESI) m/z: 349.17 [M+1]⁺.

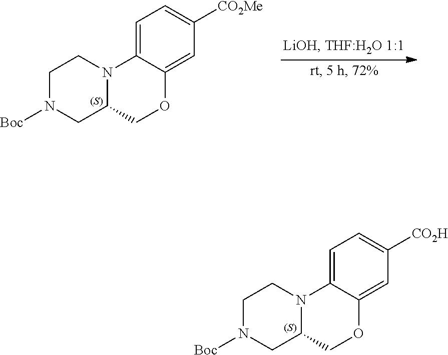

To a solution of 3-(tert-butyl) 8-methyl (S)-1,2,4a,5-tetrahydrobenzo[b]pyrazino[1,2-d][1,4]oxazine-3,8(4H)-dicarboxylate (5, 220 mg, 0.63 mmol, 1 eq) in tetrahydrofuran (2 ml) and water (2 ml) was added Lithium hydroxide (30 mg, 2 eq). The mixture was stirred at rt for 5 h. TLC (ethyl acetate:hexane=1:1) showed reaction was complete. The mixture was adjusted to pH=5 with aq. hydrochloric acid (1 M) and extracted with ethyl acetate (10 ml×3). The organic layer was washed with brine (10×2 ml) and dried over sodium sulfate. The crude material was purified by silica gel chromatography. Yield 72% LC/MS (ESI) m/z: 335.15 [M+1]⁺.

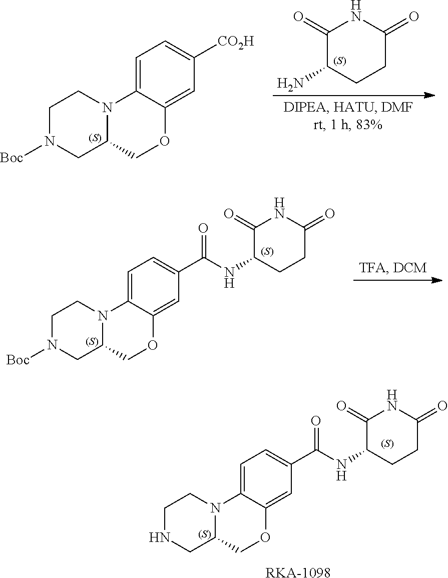

To a solution of (S)-3-(tert-butoxycarbonyl)-1,2,3,4,4a,5-hexahydrobenzo[b]pyrazino[1,2-d][1,4]oxazine-8-carboxylic acid (6, 120 mg, 0.36 mmol, 1 eq.) and (S)-3-aminopiperidine-2,6-dione (46 mg, 0.36 mmol, 1 eq) in dimethylformamide (2 ml) was added HATU (136 mg, 0.36 mmol, 1.0 eq.) followed by addition of DIPEA (0.18 mL, 1.07 mmol, 3 eq.). The solution was stirred for 15 mins, at 0° C. and allowed to warm at rt for 1 h. After completion of the reaction the residue was purified by reverse phase HPLC which on TFA deprotection yielded the final compound Compound B23. LC/MS (ESI) m/z: 345.17 [M+H]⁺

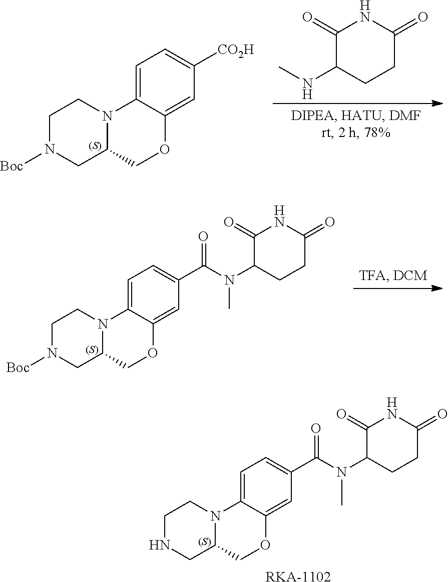

To a solution of (S)-3-(tert-butoxycarbonyl)-1,2,3,4,4a,5-hexahydrobenzo[b]pyrazino[1,2-d][1,4]oxazine-8-carboxylic acid (7, 98 mg, 0.29 mmol, 1 eq.) and 3-(methylamino)piperidine-2,6-dione (62 mg, 0.44 mmol, 1.5 eq) in dimethylformamide (2 ml) was added HATU (110 mg. 0.29 mmol, 1.0 eq.) followed by addition of DIPEA (0.15 mL, 0.87 mmol, 3 eq.). The solution was stirred for 15 mins, at 0° C. and allowed to warm at rt for 2 h. After completion of the reaction, the residue was purified by reverse phase HPLC, which on TFA deprotection yielded the final compound Compound B24.

¹H NMR of compound Compound B24 (400 MHz, Methanol-d₄) δ 7.19-6.85 (m, 3H), 4.95 (d, J=35.8 Hz, 1H), 4.36 (dd, J=11.0, 2.7 Hz, 1H), 4.26-4.00 (m, 2H), 3.59-3.40 (m, 3H), 3.40-3.34 (m, 1H), 3.31-3.19 (m, 1H), 3.10 (d, J=12.9 Hz, 1H), 3.07-2.90 (m, 3H), 2.90-2.59 (m, 2H), 2.51 (d, J=14.0 Hz, 1H), 2.18 (d, J=23.1 Hz, 1H).

### Compound B21: (4aR)—N-(2,6-dioxopiperidin-3-yl)-N-methyl-1,2,3,4,4a,5-hexahydrobenzo[b]pyrazino[1,2-d][1,4]oxazine-8-carboxamide

### Compound B22: (R)—N—((S)-2,6-dioxopiperidin-3-yl)-1,2,3,4,4a,5-hexahydrobenzo[b]pyrazino[1,2-d][1,4]oxazine-8-carboxamide

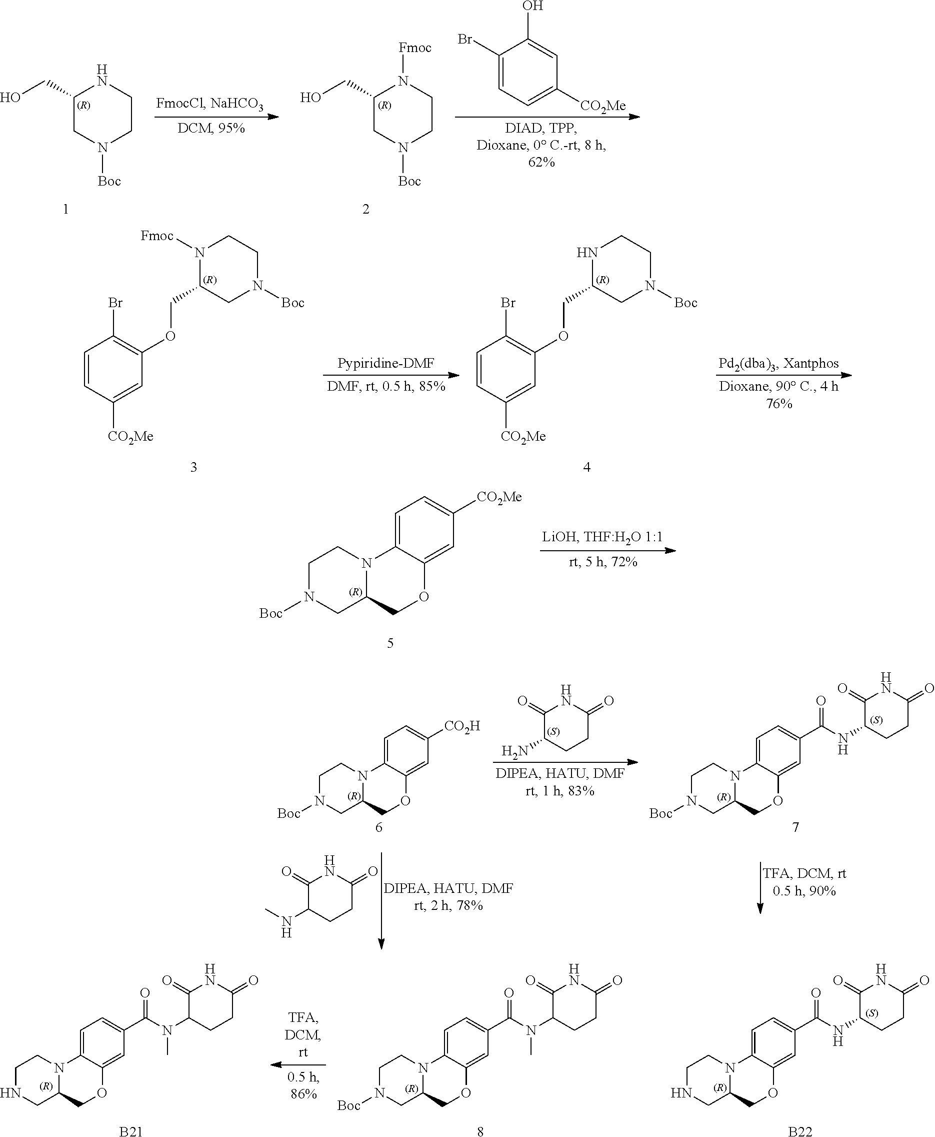

¹H NMR of compound B21 (400 MHz, Methanol-d4) δ 7.22-6.79 (m, 3H), 4.95 (d, J=35.8 Hz, 1H), 4.36 (dd, J=11.0, 2.7 Hz, 1H), 4.28-3.97 (m, 2H), 3.60-3.41 (m, 3H), 3.40-3.34 (m, 1H), 3.31-3.19 (m, 1H), 3.10 (d, J=12.9 Hz, 1H), 3.07-2.90 (m, 3H), 2.90-2.59 (m, 2H), 2.51 (d, J=14.0 Hz, 1H), 2.15 (s, 1H).

¹H NMR of compound B22 (400 MHz, Methanol-d4) δ 7.48 (dd, J=8.6, 2.1 Hz, 1H), 7.37 (d, J=2.2 Hz, 1H), 7.04 (d, J=8.7 Hz, 1H), 4.84-4.79 (m, 1H), 4.37 (dd, J=11.2, 2.8 Hz, 1H), 4.27-4.16 (m, 1H), 4.09 (dd, J=11.1, 7.3 Hz, 1H), 3.60-3.43 (m, 3H), 3.26 (td, J=12.6, 3.5 Hz, 1H), 3.12 (ddd, J=13.7, 12.7, 3.0 Hz, 1H), 2.99 (t, J=12.1 Hz, 1H), 2.90-2.77 (m, 1H), 2.77-2.66 (m, 1H), 2.27-2.13 (m, 2H).

TABLE E1

| | | LC-MS | |
|---|---|---|---|
| Compound No. | Structure | [M + H]⁺ | NMR |
| B1 | 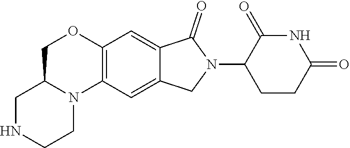 [SMILES: O=C1CCC(N2Cc3cc4c(cc3C2=O)OC[C@@H]2CNCCN42)C(=O)N1] | 356.90 | ¹H NMR (400 MHz, Methanol-d₄) δ 7.15 (s, 1H), 7.11 (d, J = 5.7 Hz, 1H), 5.13-5.02 (m, 1H), 4.41-4.27 (m, 3H), 4.20 (d, J = 13.6 Hz, 1H), 4.12-4.01 (m, 1H), 3.62-3.43 (m, 3H), 3.30-3.10 (m, 2H), 3.03-2.94 (m, 1H), 2.94-2.82 (m, 1H), 2.82-2.71 (m, 1H), 2.52- 2.38 (m, 1H), 2.20-2.09 (m, 1H). ¹³C NMR (101 MHz, MeOD) δ 174.68, 172.52, 172.49, 171.63, 171.59, 146.42, 146.37, 139.22, 139.20, 138.12, 138.08, 123.85, 123.78, 111.88, 108.70, 108.65, 66.94, 53.72, 53.57, 50.79, 50.75, |
| | | | 48.90, 48.68, 44.36, 44.17, 44.10, 43.63, 43.61, 32.35, |
| | | | 24.08. |
| B2 | 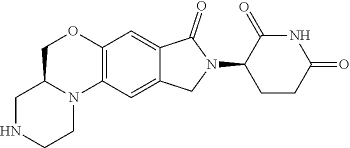 [SMILES: O=C1CC[C@@H](N2Cc3cc4c(cc3C2=O)OC[C@@H]2CNCCN42)C(=O)N1] | 356.88 | |
| B3 | 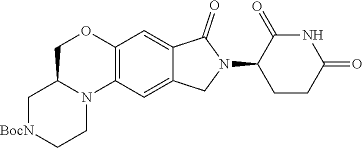 [SMILES: CC(C)(C)OC(=O)N1CCN2c3cc4c(cc3OC[C@@H]2C1)C(=O)N([C@@H]1CCC(=O)NC1=O)C4] | 457.15 | |
| B4 | 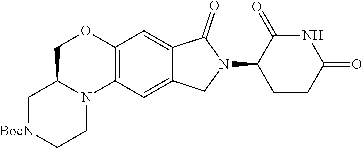 [SMILES: CC(C)(C)OC(=O)N1CCN2c3cc4c(cc3OC[C@@H]2C1)C(=O)N([C@@H]1CCC(=O)NC1=O)C4] | 457.19 | |
| B5 | 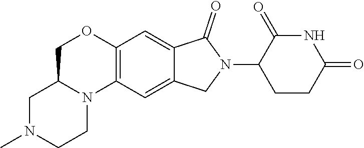 [SMILES: CN1CCN2c3cc4c(cc3OC[C@@H]2C1)C(=O)N(C1CCC(=O)NC1=O)C4] | 370.90 | |
| B6 | 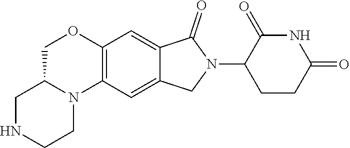 [SMILES: O=C1CCC(N2Cc3cc4c(cc3C2=O)OC[C@H]2CNCCN42)C(=O)N1] | 356.91 | ¹H NMR (400 MHz, Methanol-d₄) δ 7.17 (s, 1H), 7.13 (d, J = 1.7 Hz, 1H), 5.14-5.03 (m, 1H), 4.44-4.28 (m, 3H), 4.27-4.17 (m, 1H), 4.12-4.03 (m, 1H), 3.61- 3.42 (m, 3H), 3.30-3.21 (m, 1H), 3.21-3.11 (m, 1H), 3.04-2.95 (m, 1H), 2.95-2.83 (m, 1H), 2.81- 2.72 (m, 1H), 2.52-2.38 (m, 1H), 2.19-2.10 (m, 1H). ¹³C NMR (101 MHz, MeOD) δ 174.66, 172.46, 172.44, 171.64, 171.61, 146.51, 146.47, 139.24, 139.20, 138.14, 123.98, 123.91, 111.93, 108.73, 108.70, 66.97, 53.71, 53.60, 50.88, 50.86, 44.38, |
| | | | 44.24, 44.20, 43.62, 32.38, 24.13. |
| B7 | 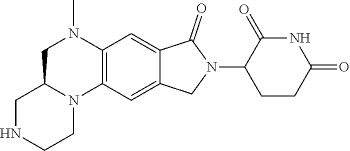 [SMILES: CN1C[C@@H]2CNCCN2c2cc3c(cc21)C(=O)N(C1CCC(=O)NC1=O)C3] | 370.02 | ¹H NMR (400 MHz, Methanol-d₄) δ 6.99-6.93 (m, 2H), 5.12-5.04 (m, 1H), 4.34-4.29 (m, 1H), 4.27- 4.18 (m, 1H), 3.69-3.60 (m, 1H), 3.51-3.40 (m, 2H), 3.39-3.33 (m, 1H), 3.27-3.12 (m, 4H), 3.07-3.00 (m, 1H), 2.92 (s, 3H), 2.88-2.85 (m, 1H), 2.80-2.73 (m, 1H), 2.51-2.38 (m, 1H), 2.18-2.10 (m, 1H). |
| B8 | 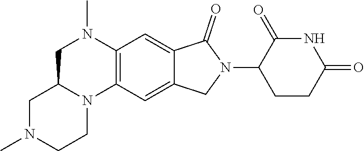 [SMILES: CN1CCN2c3cc4c(cc3N(C)C[C@@H]2C1)C(=O)N(C1CCC(=O)NC1=O)C4] | 383.98 | |
| B9 | 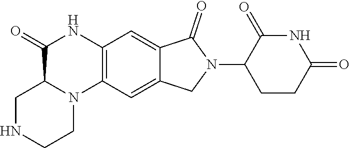 [SMILES: O=C1CCC(N2Cc3cc4c(cc3C2=O)NC(=O)[C@@H]2CNCCN42)C(=O)N1] | 370.20 | |
| B10 | 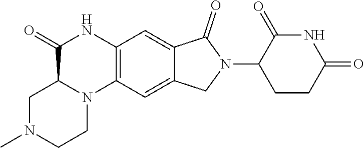 [SMILES: CN1CCN2c3cc4c(cc3NC(=O)[C@@H]2C1)C(=O)N(C1CCC(=O)NC1=O)C4] | 384.04 | |
| B11 | 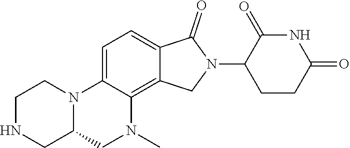 [SMILES: CN1C[C@@H]2CNCCN2c2ccc3c(c21)CN(C1CCC(=O)NC1=O)C3=O] | 370.28 | ¹H NMR (400 MHz, Methanol-d₄) δ 7.32 (dd, J = 8.4, 2.3 Hz, 1H), 7.09 (dd, J = 8.6, 2.5 Hz, 1H), 5.14-5.04 (m, 1H), 4.63-4.44 (m, 2H), 4.30-4.16 (m, 1H), 3.60- 3.51 (m, 1H), 3.49-3.39 (m, 2H), 3.29-3.21 (m, 2H), 3.16-3.02 (m, 2H), 2.98-2.83 (m, 5H), 2.82- 2.72 (m, 1H), 2.58-2.45 (m, 1H), 2.21-2.10 (m, 1H). ¹³C NMR (101 MHz, MeOD) δ 174.70, 172.53, 172.50, 171.69, 171.63, 141.77, 141.73, 133.94, 133.82, 133.63, 124.97, 118.36, 118.32, 115.71, 53.85, |
| | | | 58.82, 53.67, 53.63, 47.98, 47.86, 46.28, 44.83, 44.39, |
| | | | 43.81, 43.75, 32.37, 24.03, 23.99. |
| B12 | 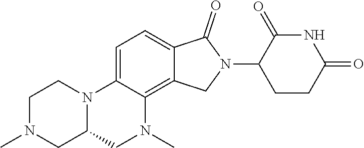 [SMILES: CN1CCN2c3ccc4c(c3N(C)C[C@@H]2C1)CN(C1CCC(=O)NC1=O)C4=O] | 384.23 | |
| B13 | 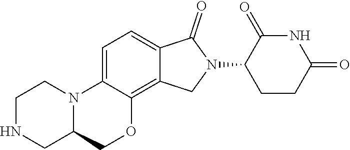 [SMILES: O=C1CC[C@H](N2Cc3c(ccc4c3OC[C@H]3CNCCN43)C2=O)C(=O)N1] | 356.92 | |
| B14 | 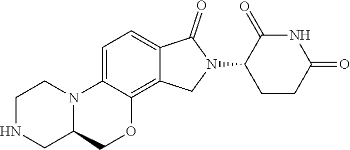 [SMILES: O=C1CC[C@H](N2Cc3c(ccc4c3OC[C@H]3CNCCN43)C2=O)C(=O)N1] | 356.92 | ¹H NMR (400 MHz, Methanol-d₄) δ 7.32 (d, J = 8.3 Hz, 1H), 7.08 (d, J = 8.4 Hz, 1H), 5.10 (dd, J = 13.3, 5.2 Hz, 1H), 4.46-4.30 (m, 3H), 4.23-3.98 (m, 3H), 3.93 (d, J = 12.4 Hz, 1H), 3.22 (ddd, J = 11.2, 8.2, 3.0 Hz, 1H), 3.07 (s, 1H), 2.99-2.61 (m, 4H), 2.59-2.42 (m, 1H), 2.21-2.07 (m, 1H), 1.51 (s, 9H). |
| B15 | 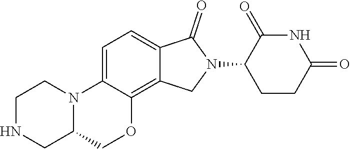 [SMILES: O=C1CC[C@H](N2Cc3c(ccc4c3OC[C@@H]3CNCCN43)C2=O)C(=O)N1] | 356.92 | ¹H NMR (400 MHz, Methanol-d₄) δ 7.37 (d, J = 8.3 Hz, 1H), 7.14 (d, J = 8.5 Hz, 1H), 5.11 (ddd, J = 13.3, 5.2, 2.2 Hz, 1H), 4.50-4.34 (m, 3H), 4.34-4.10 (m, 3H), 3.67-3.42 (m, 4H), 3.30-3.22 (m, 1H), 3.22- 3.09 (m, 1H), 3.02 (td, J = 12.2, 5.7 Hz, 1H), 2.91 (ddd, J = 18.5, 13.4, 5.4 Hz, 1H), 2.79 (ddd, J = 17.6, 4.7, 2.4 Hz, 1H), 2.57-2.41 (m, 1H), 2.16 (dtd, J = 12.9, 5.3, 2.5 Hz, 1H). |
| B16 | 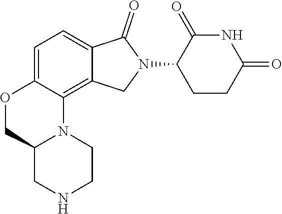 [SMILES: O=C1CC[C@H](N2Cc3c(ccc4c3N3CCNC[C@H]3CO4)C2=O)C(=O)N1] | 357.10 | ¹H NMR (400 MHz, Methanol-d₄) δ 7.32 (d, J = 8.1 Hz, 1H), 7.02 (dd, J = 8.1, 0.9 Hz, 1H), 5.15 (dd, J = 13.4, 5.2 Hz, 1H), 4.70-4.47 (m, 2H), 4.34 (ddd, J = 11.3, 4.2, 2.8 Hz, 1H), 4.14 (ddd, J = 11.3, 9.8, 7.2 Hz, 1H), 4.04-3.91 (m, 1H), 3.65 (ddq, J = 10.4, 7.1, 3.4, 2.8 Hz, 1H), 3.54-3.39 (m, 2H), 3.30-3.22 (m, 2H), 3.14 (dt, J = 12.8, 10.6 Hz, 1H), 2.94 (ddd, J = 17.6, 13.5, 5.4 Hz, 1H), 2.80 (ddd, J = 17.6, 4.7, 2.4 Hz, 1H), 2.60-2.42 (m, 1H), 2.19 (ddq, J = 10.5, 5.4, 2.8 Hz, 1H). |
| B17 | 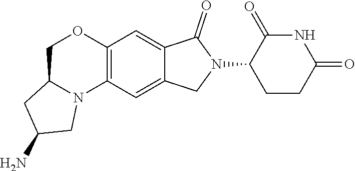 [SMILES: N[C@H]1C[C@H]2COc3cc4c(cc3N2C1)CN([C@H]1CCC(=O)NC1=O)C4=O] | 357.12 | ¹H NMR (400 MHz, Methanol-d4) d 7.16 (s, 1H), 6.72 (s, 1H), 5.09 (dt, J = 13.3, 5.1 Hz, 1H), 4.58 (d, J = 7.1 Hz, 2H), 4.36 (d, J = 6.7 Hz, 2H), 4.15 (d, J = 3.6 Hz, 1H), 3.79 (dd, J = 10.4, 7.9 Hz, 1H), 3.72-3.62 (m, 2H), 3.53-3.40 (m, 1H), 2.96-2.84 (m, 1H), 2.78 (ddd, J = 17.4, 4.8, 2.5 Hz, 1H), 2.61 (ddd, J = 12.5, 8.6, 4.1 Hz, 1H), 2.55-2.37 (m, 1H), 2.16 (ddq, J = 10.4, 5.3, 2.7 Hz, 1H), 1.79-1.59 (m, 1H). |
| B18 | 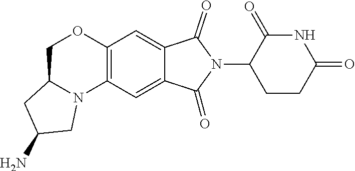 [SMILES: N[C@H]1C[C@H]2COc3cc4c(cc3N2C1)C(=O)N(C1CCC(=O)NC1=O)C4=O] | 371.08 | ¹H NMR (400 MHz, Methanol-d₄) δ 7.25 (d, J = 1.1 Hz, 1H), 7.02 (s, 1H), 5.06 (dd, J = 12.4, 5.5 Hz, 1H), 4.70 (d, J = 7.1 Hz, 1H), 4.18 (p, J = 7.6 Hz, 1H), 3.91 (dd, J = 10.6, 8.0 Hz, 1H), 3.74 (dd, J = 8.2, 3.4 Hz, 2H), 3.60-3.46 (m, 1H), 2.96-2.80 (m, 1H), 2.80- 2.61 (m, 3H), 2.18-2.04 (m, 1H), 1.70 (dt, J = 12.2, 9.3 Hz, 1H). |
| B19 | 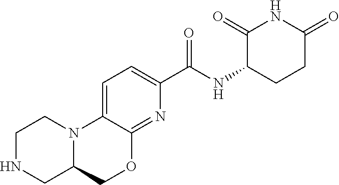 [SMILES: O=C1CC[C@H](NC(=O)c2ccc3c(n2)OC[C@H]2CNCCN32)C(=O)N1] | 346.17 | ¹H NMR (400 MHz, DMSO): δ 10.84 (s, 1H), 9.63- 9.33 (m, 2H), 8.56 (d, J = 8.4 Hz, 1H), 7.62 (d, J = 8.2 Hz, 1H), 7.44 (d, J = 8.4 Hz, 1H), 4.77-4.70 (m, 1H), 4.51- 4.49 (m, 2H), 4.20-4.05 (m, 2H), 3.66-3.55 (m, 1H), 3.47-3.39 (m, 2H), 3.22-2.98 (m, 2H), 2.89-2.67 (m, 2H), 2.26-2.11 (m, 1H), 2.02-1.90 (m, 1H) |
| B20 | 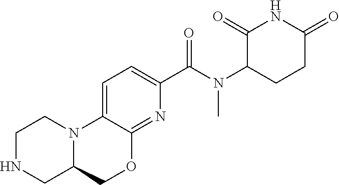 [SMILES: CN(C(=O)c1ccc2c(n1)OC[C@H]1CNCCN21)C1CCC(=O)NC1=O] | 360.18 | ¹H NMR (400 MHz, Methanol-d4 ) δ 7.36-7.30 (m, 2H), 5.48-5.21 (m, 1H), 4.52 (q, J = 10.4 Hz, 2H), 4.21-4.11 (m, 4H), 3.84 (d, J = 11.9 Hz, 1H), 3.14-3.04 (m, 3H), 2.97 (d, J = 5.1 Hz, 2H), 2.87-2.65 (m, 4H), 2.59-2.40 (m, 1H), 1.50 (s, 9H). |
| B21 | 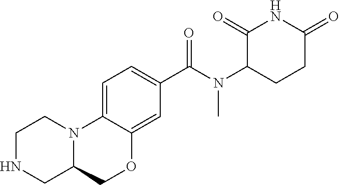 [SMILES: CN(C(=O)c1ccc2c(c1)OC[C@H]1CNCCN21)C1CCC(=O)NC1=O] | 359.12 | ¹H NMR (400 MHz, Methanol-d₄) δ 7.22-6.79 (m, 3H), 4.95 (d, J = 35.8 Hz, 1H), 4.36 (dd, J = 11.0, 2.7 Hz, 1H), 4.28-3.97 (m, 2H), 3.60-3.41 (m, 3H), 3.40- 3.34 (m, 1H), 3.31-3.19 (m, 1H), 3.10 (d, J = 12.9 Hz, 1H), 3.07-2.90 (m, 3H), 2.90-2.59 (m, 2H), 2.51 (d, J = 14.0 Hz, 1H), 2.15 (s, 1H). |
| B22 | 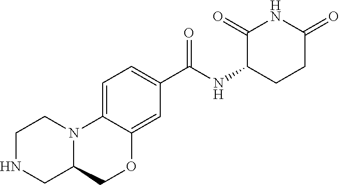 [SMILES: O=C1CC[C@H](NC(=O)c2ccc3c(c2)OC[C@H]2CNCCN32)C(=O)N1] | 345.05 | ¹H NMR (400 MHz, Methanol-d₄) δ 7.48 (dd, J = 8.6, 2.1 Hz, 1H), 7.37 (d, J = 2.2 Hz, 1H), 7.04 (d, J = 8.7 Hz, 1H), 4.84-4.79 (m, 1H), 4.37 (dd, J = 11.2, 2.8 Hz, 1H), 4.27-4.16 (m, 1H), 4.09 (dd, J = 11.1, 7.3 Hz, 1H), 3.60-3.43 (m, 3H), 3.26 (td, J = 12.6, 3.5 Hz, 1H), 3.12 (ddd, J = 13.7, 12.7, 3.0 Hz, 1H), 2.99 (t, J = 12.1 Hz, 1H), 2.90-2.77 (m, 1H), 2.77-2.66 (m, 1H), 2.27-2.13 (m, 2H). |
| B23 | 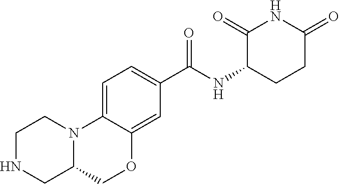 [SMILES: O=C1CC[C@H](NC(=O)c2ccc3c(c2)OC[C@@H]2CNCCN32)C(=O)N1] | 345.17 | ¹H NMR (400 MHz, Methanol-d₄) δ 7.48 (dd, J = 8.6, 2.1 Hz, 1H), 7.37 (d, J = 2.2 Hz, 1H), 7.04 (d, J = 8.7 Hz, 1H), 4.84-4.79 (m, 1H), 4.37 (dd, J = 11.2, 2.8 Hz, 1H), 4.29-4.16 (m, 1H), 4.05 (dd, J = 11.1, 7.3 Hz, 1H), 3.60-3.43 (m, 3H), 3.26 (td, J = 12.6, 3.5 Hz, 1H), 3.12 (ddd, J = 13.7, 12.7, 3.0 Hz, 1H), 2.99 (t, J = 12.1 Hz, 1H), 2.90-2.77 (m, 1H), 2.72-2.62 (m, 1H), 2.27-2.13 (m, 2H). |
| B24 | 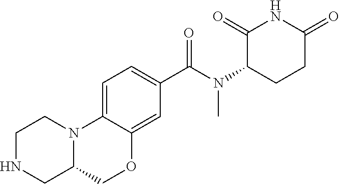 [SMILES: CN(C(=O)c1ccc2c(c1)OC[C@@H]1CNCCN21)[C@H]1CCC(=O)NC1=O] | 359.09 | ¹H NMR (400 MHz, Methanol-d₄) δ 7.19-6.85 (m, 3H), 4.95 (d, J = 35.8 Hz, 1H), 4.36 (dd, J = 11.0, 2.7 Hz, 1H), 4.26-4.00 (m, 2H), 3.59-3.40 (m, 3H), 3.40- 3.34 (m, 1H), 3.31-3.19 (m, 1H), 3.10 (d, J = 12.9 Hz, 1H), 3.07-2.90 (m, 3H), 2.90-2.59 (m, 2H), 2.51 (d, J = 14.0 Hz, 1H), 2.18 (d, J = 23.1 Hz, 1H). |

### Biological Activity of Cereblon Ligands

### For Compound A1 to A9

### In Vitro Assay: IC₅₀ Measurements for Binding to CRBN/DDB1

The binding potency was determined using HTRF assay technology (Perkin Elmer). Compounds were serially diluted in DMSO and 0.2 μL volume was transferred to white 384-well plate. The reaction was conducted in total volume of 20 μL with addition of 2 nM His tagged CRBN+DDB-DLS7+CXU4 (Wuxi, catalogue #RP210521GA) to compounds followed by addition of 60 nM Fluorescent probe Cy5-labeled Thalidomide (Tenova Pharma, catalogue #T52461), and 0.4 nM of MAb Anti-6HIS Tb cryptate Gold (Cisbio, catalogue #61HI2TLA in the assay buffer (50 mM HEPES pH 7.5, 1 mM TCEP, 0.01% Brij-35, 50 mM NaCl, and 0.1% BSA). After one hour incubation at room temperature, the HTRF signals were read on Envision reader (Perkin Elemer). Data were analyzed using XLfit using four parameters dose response curve to determine IC₅₀s and shown in Table E2.

TABLE E2
CRBN binding IC₅₀

| Compound No. | CRBN HTRF IC₅₀ (nM) |
|---|---|
| A1 | 21759 |
| A2 | 3644 |
| A3 | 330 |
| A4 | 10,000 |
| A7 | TBD |
| A8 | 430 |
| A9 | 10,000 |

### For Compound B1 to B24

### Cereblon Binding Assay

The binding to cereblon (CRBN) was determined using the Cereblon Binding Kit (Cisbio, #64BDCRBNPEG) following the manufacturer's instruction. Briefly, serially diluted compounds were incubated with GST-tagged wild-type human CRBN protein, XL665-labelled Thalidomide and Europium Cryptate labelled GST antibody at room temperature for about 3 hours. Time Resolved Fluorescence Resonance Energy Transfer (TR-FRET) measurements were acquired on a CALRIOstar plate reader with MARS data analysis software (BMG Labtech), with the following settings: 665/10 nm and 620/10 nm emission, 60 s delay and 400 s integration. The TR-FRET ratio was taken as the 665/620 nm intensity ratio. The readings were normalized to the control (0.5%) and the IC₅₀ was calculated by nonlinear regression (four parameters sigmoid fitted with variable slope) analysis using the GraphPad Prism 8 software.

### Immunoblotting

Cells were maintained in the appropriate culture medium with 10% FBS at 37° C. and an atmosphere of 5% CO₂. All the cell lines were used within three months of thawing fresh vials.

Cells were lysed 1× Cell Lysis Buffer (Cell Signaling Technology, #9803), resolved by SDS-PAGE NuPAGE gel (Thermo Fisher Scientific), and transferred to a PVDF membrane (Millipore). Membranes were blocked using Odyssey TBS Blocker Buffer (LI-COR). IRDye 680RD and 800CW Dye-labeled secondary antibodies (LI-COR) were used. The washed membranes were scanned using Odyssey CLx imager (LI-COR). The intensity of Western blot signaling was quantitated using the Odyssey software. Primary antibodies used are: Helios (D8W4X) XP® Rabbit mAb (Cell Signaling Technology, #42427) and GAPDH mouse monoclonal antibody (Santa Cruz Biotechnology, sc-47724).

### IKZF2 HiBiT Assay

Degradation of IKZF2 protein was determined by IKZF2 HiBiT assay using the Jurkat-IKZF2-HiBiT (Promega) cell line. Briefly, cells were seeded in 384-well flat bottom (Corning #07-201-4423595) at a density of 10,000 cells/well in 20 μl of culture medium. Compounds were serially diluted in culture medium, and 20 μl of the diluted compounds were added to the appropriate wells of the plate. After the addition of compounds, the cells were incubated at 37° C. in an atmosphere of 5% CO₂ for 24 hours. At the end of treatment, 40 ul of Nano-Glo HiBiT Lytic Detection Reagent (Promega) was added to each well, and then the plates were incubated at room temperature for 10-20 minutes. The luminescent signal was measured using a CALRIOstar plate reader (BMG Labtech). The readings were normalized to the DMSO-treated cells and the IC₅₀ was calculated by nonlinear regression (four parameters sigmoid fitted with variable slope, least squares fit, and no constraint) analysis using the GraphPad Prism 8 software.

TABLE E3
Binding activity for cereblon ligands

| Compound No. | CRBN TR-FRET IC₅₀ (μM) |
|---|---|
| B0 | 0.22 |
| B1 | 2.1 |
| B2 | 5.2 |
| B3 | 1.7 |
| B4 | 3.5 |
| B5 | 0.92 |
| B6 | 3.1 |
| B7 | 0.74 |
| B8 | 0.94 |
| B10 | 0.60 |
| B12 | 0.58 |
| B13 | 0.44 |
| B14 | 3.3 |
| B15 | 0.12 |
| B16 | 1.4 |
| B17 | 0.13 |
| B18 | 0.22 |
| B19 | 30.16 |
| B20 | 17.54 |
| B21 | 4.6 |
| B22 | 23 |
| B23 | 25 |
| B24 | 3.4 |
| C8 | 0.47 |
| C9 | 0.18 |
| C10 | 0.20 |
| C11 | 0.090 |
| C12 | 0.24 |
| C13 | 0.090 |
| C19 | 0.33 |

### II. Bifunctional Degraders

### Synthesis and Characterization

### 1. ER Degraders

### Tetrahydronaphtalene Series

### Compound THP-A8. (3R)-3-((4aR)-3-((2-(4-((1S,2R)-6-hydroxy-2-phenyl-1,2,3,4-tetrahydronaphthalen-1-yl)phenyl)-5-oxa-2-azaspiro[3.4]octan-7-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

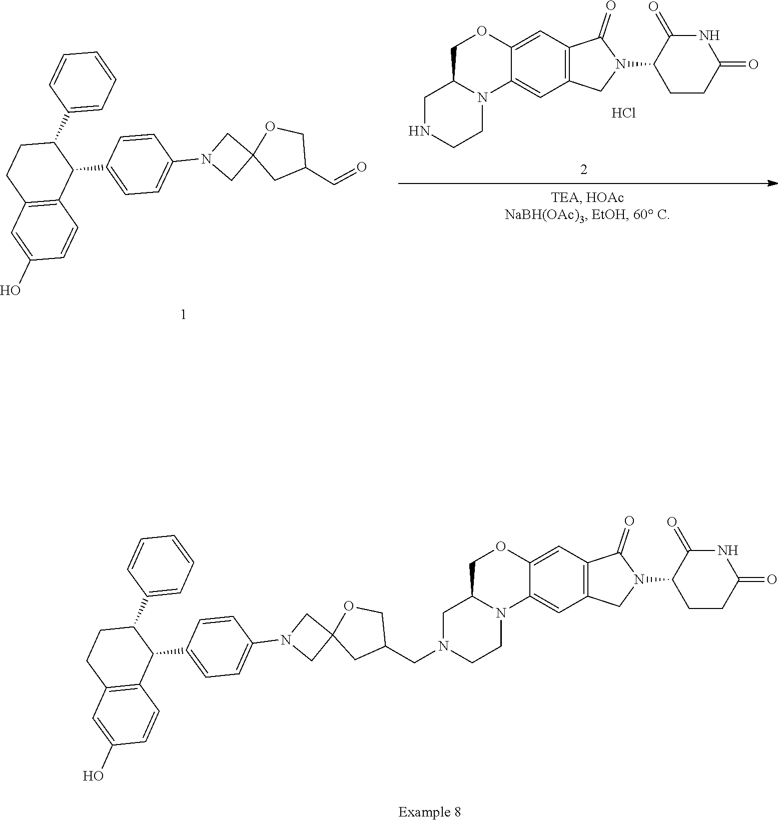 [SMILES: O=CC1COC2(C1)CN(c1ccc([C@@H]3c4ccc(O)cc4CC[C@@H]3c3ccccc3)cc1)C2 ; O=C1CC[C@H](N2Cc3cc4c(cc3C2=O)OC[C@@H]2CNCCN42)C(=O)N1 ; Cl ; O=C1CC[C@H](N2Cc3cc4c(cc3C2=O)OC[C@@H]2CN(CC3COC5(C3)CN(c3ccc([C@@H]6c7ccc(O)cc7CC[C@@H]6c6ccccc6)cc3)C5)CCN42)C(=O)N1]

To a mixture of 2-(4-((1R,2S)-6-hydroxy-2-phenyl-1,2,3,4-tetrahydronaphthalen-1-yl)phenyl)-5-oxa-2-azaspiro[3.4]octane-7-carbaldehyde (30 mg, 0.07 mmol, 1.0 eq) and rac-(R)-3-((R)-8-oxo-1,2,3,4,4a,5,8,10-octahydro-9H-pyrazino[1′,2′:4,5][1,4]oxazino[2,3-f]isoindol-9-yl)piperidine-2,6-dione hydrochloride (27 mg, 0.07 mmol, 1.0 eq) in EtOH (5 mL) was added triethylamine (27.5 mg, 0.27 mmol, 4.0 eq), followed by the addition of AcOH (163 mg, 2.72 mmol, 40 eq) and stirred at 25° C. for 0.5 hour. The mixture was added NaBH(OAc)₃ (58 mg, 0.27 mmol, 4.0 eq) and stirred at 50° C. for 2 hours. LCMS showed the reaction was completed. The reaction was cooled to 20° C. and concentrated under vacuum. The residue was purified by Prep-HPLC (ACN/0.05% FA) to afford (3R)-3-((4aR)-3-((2-(4-((1S,2R)-6-hydroxy-2-phenyl-1,2,3,4-tetrahydronaphthalen-1-yl)phenyl)-5-oxa-2-azaspiro[3.4]octan-7-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (5.77 mg, 19%) as a white solid. LC-MS purity: 100% (UV at 254 nm), 780.2 [M+H]⁺. ¹H NMR (400 MHz, DMSO) δ 10.94 (s, 1H), 9.12 (s, 1H), 7.19-7.07 (m, 4H), 7.04-6.95 (m, 2H), 6.80 (dd, J=16.7, 7.5 Hz, 2H), 6.66-6.57 (m, 2H), 6.47 (dd, J=8.3, 2.6 Hz, 1H), 6.31 (d, J=8.3 Hz, 1H), 6.18 (d, J=8.4 Hz, 1H), 6.06 (d, J=8.3 Hz, 1H), 5.03 (dd, J=13.1, 4.9 Hz, 1H), 4.31 (d, J=9.6 Hz, 1H), 4.21 (d, J=22.7 Hz, 2H), 4.15-4.10 (m, 1H), 4.00-3.87 (m, 3H), 3.87-3.71 (m, 3H), 3.70-3.60 (m, 2H), 3.59-3.49 (m, 31H), 3.03-2.76 (m, 6H), 2.71-2.58 (m, 3H), 2.44-2.29 (m, 3H), 2.09 (dd, J=14.0, 8.4 Hz, 1H), 2.01-1.82 (m, 3H), 1.69 (d, J=11.3, 8 Hz, 1H).

### Compound THP-A21. (S)-3-((S)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

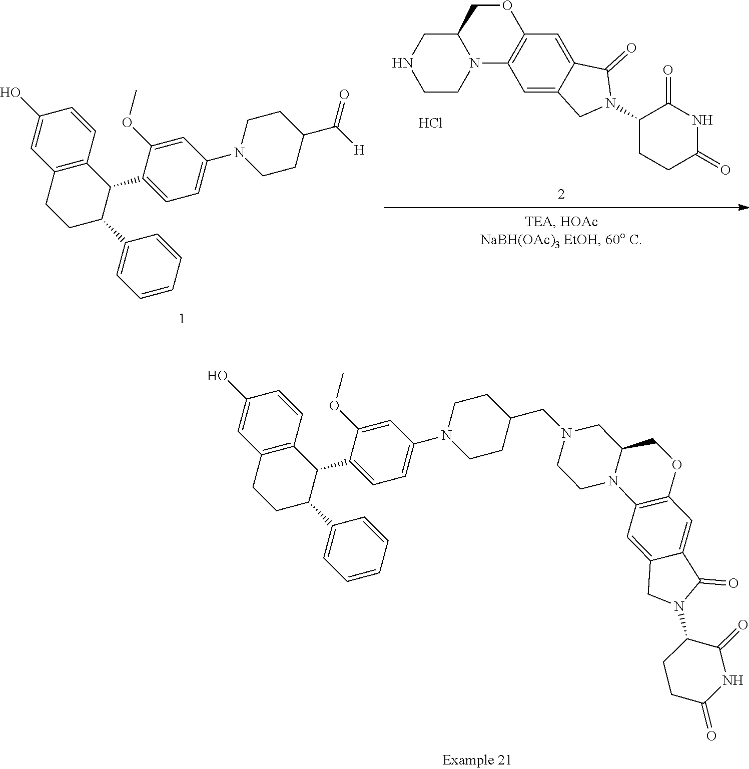 [SMILES: COc1cc(N2CCC(C=O)CC2)ccc1[C@H]1c2ccc(O)cc2CC[C@H]1c1ccccc1 ; O=C1CC[C@H](N2Cc3cc4c(cc3C2=O)OC[C@@H]2CNCCN42)C(=O)N1 ; Cl ; COc1cc(N2CCC(CN3CCN4c5cc6c(cc5OC[C@@H]4C3)C(=O)N([C@H]3CCC(=O)NC3=O)C6)CC2)ccc1[C@H]1c2ccc(O)cc2CC[C@H]1c1ccccc1]

To a mixture of 1-(4-((1R,2R)-6-hydroxy-2-phenyl-1,2,3,4-tetrahydronaphthalen-1-yl)-3-methoxyphenyl)piperidine-4-carbaldehyde (45.4 mg, 0.10 mmol, 1.0 eq) and (S)-3-((S)-8-oxo-1,2,3,4,4a,5,8,10-octahydro-9H-pyrazino[1′,2′:4,5][1,4]oxazino[2,3-f]isoindol-9-yl)piperidine-2,6-dione hydrochloride (40.4 mg, 0.10 mmol, 1.0 eq) in EtOH (5 mL) was added triethylamine (0.06 mL, 0.41 mmol, 4.0 eq), followed by the addition of AcOH (0.32 mL, 4.11 mmol, 40 eq) and stirred at 25° C. for 0.5 hour. The mixture was added NaBH(OAc)₃ (87.2 mg, 0.41 mmol, 4.0 eq) and stirred at 60° C. for 2 hours. LCMS showed the reaction was completed. The reaction was cooled to 20° C. and concentrated under vacuum. The residue was purified by prep-HPLC (ACN/0.05% FA) to afford (S)-3-((S)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (37.96 mg, 47%) as a white solid. LCMS purity: 100% (UV at 254 nm), MS: 782.2 [M+H]⁺; Retention time R_{f}=5.256 min. ¹H NMR (400M Hz, MeOD-d4) δ 7.15 (s, 1H), 7.07 (dd, J=7.4, 4.0 Hz, 4H), 6.80-6.73 (m, 2H), 6.67-6.61 (m, 2H), 6.57 (d, J=8.3 Hz, 1H), 6.51 (dd, J=8.3, 2.6 Hz, 2H), 6.28 (s, 1H), 5.51 (s, 1H), 5.10 (dd, J=13.3, 5.2 Hz, 1H), 4.81 (d, J=5.3 Hz, 1H), 4.41-4.27 (m, 3H), 4.03 (dd, J=11.0, 7.9 Hz, 2H), 3.62 (d, J=9.2 Hz, 2H), 3.46 (dd, J=17.5, 12.1 Hz, 2H), 3.26 (d, J=7.3 Hz, 2H), 3.05 (s, 4H), 2.94 (ddd, J=18.3, 15.7, 5.4 Hz, 3H), 2.80 (dd, J=11.9, 7.6 Hz, 3H), 2.68 (s, 2H), 2.53-2.41 (m, 1H), 2.38-2.25 (m, 2H), 2.16 (dd, J=11.2, 6.2 Hz, 1H), 1.95 (t, J=10.1 Hz, 3H), 1.68 (dd, J=15.3, 5.5 Hz, 1H), 1.52-1.42 (m, 2H).

### Compound THP-A34. (R)-3-((R)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

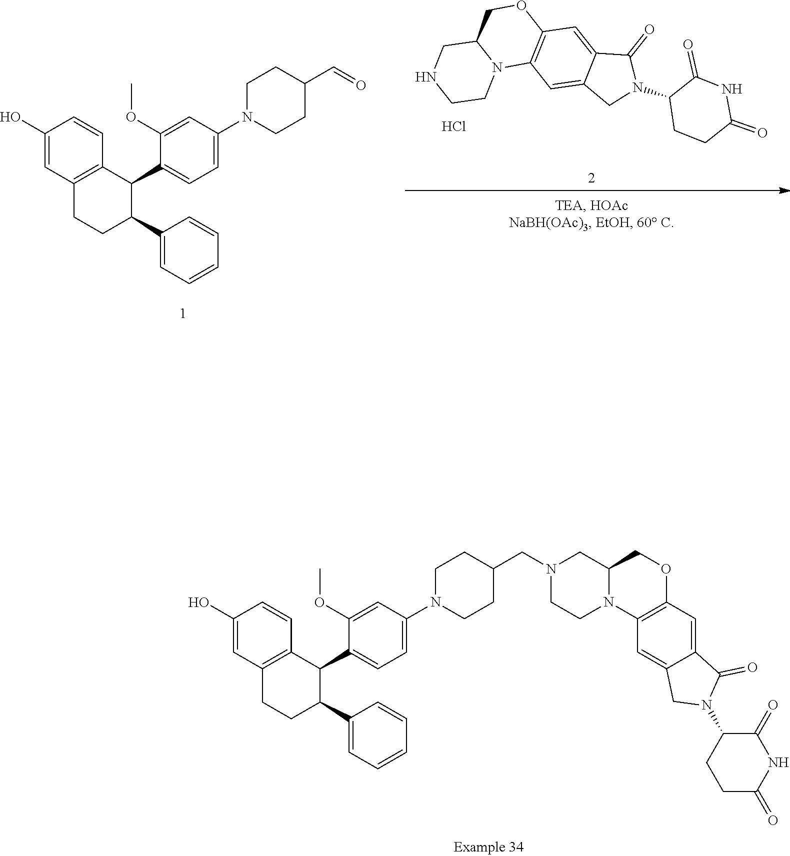 [SMILES: COc1cc(N2CCC(C=O)CC2)ccc1[C@@H]1c2ccc(O)cc2CC[C@@H]1c1ccccc1 ; O=C1CC[C@H](N2Cc3cc4c(cc3C2=O)OC[C@@H]2CNCCN42)C(=O)N1 ; Cl ; COc1cc(N2CCC(CN3CCN4c5cc6c(cc5OC[C@@H]4C3)C(=O)N([C@H]3CCC(=O)NC3=O)C6)CC2)ccc1[C@@H]1c2ccc(O)cc2CC[C@@H]1c1ccccc1]

To a mixture of 1-(4-((1R,2R)-6-hydroxy-2-phenyl-1,2,3,4-tetrahydronaphthalen-1-yl)-3-methoxyphenyl)piperidine-4-carbaldehyde with structure being tentatively assigned (40 mg, 0.09 mmol, 1.0 eq) and (R)-3-((R)-8-oxo-1,2,3,4,4a,5,8,10-octahydro-9H-pyrazino[1′,2′:4,5][1,4]oxazino[2,3-f]isoindol-9-yl)piperidine-2,6-dione hydrochloride (32 mg, 0.09 mmol, 1.0 eq) in EtOH (5 mL) was added triethylamine (37 mg. 0.36 mmol, 4.0 eq), followed by the addition of AcOH (219 mg, 3.64 mmol, 40 eq) and stirred at 25° C. for 0.5 hour. The mixture was added NaBH(OAc)₃ (77 mg, 0.36 mmol, 4.0 eq) and stirred at 50° C. for 2 hours. LCMS showed the reaction was completed. The reaction was cooled to 20° C. and concentrated under vacuum. The residue was purified by Prep-HPLC (ACN/0.05% FA) to afford (R)-3-((R)-3-((1-(4-((1R,2R)-6-hydroxy-2-phenyl-1,2,3,4-tetrahydronaphthalen-1-yl)-3-methoxyphenyl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (11.29 mg, 28%) as a white solid. LC-MS purity: 100% (UV at 254 nm), 782.2 [M+H]. ¹H NMR (400 MHz, DMSO) δ 10.94 (s, 1H), 9.06 (s, 1H), 7.22-6.92 (m, 5H), 6.73 (d, J=5.8 Hz, 2H), 6.60-6.51 (m, 2H), 6.49-6.42 (m, 1H), 6.40-6.28 (m, 2H), 6.13 (t, J=6.0 Hz, 1H), 5.03 (d, J=13.0 Hz, 1H), 4.65 (d, J=5.2 Hz, 1H), 4.28-4.16 (m, 2H), 4.03-3.85 (m, 1H), 3.61 (dd, J=21.7, 12.2 Hz, 3H), 3.15 (d, J=35.1 Hz, 4H), 3.00-2.85 (m, 8H), 2.63 (d, J=29.1 Hz, 2H), 2.33 (s, 1H), 2.23-2.10 (m, 3H), 1.96 (dd, J=10.7, 5.9 Hz, 2H), 1.85-1.73 (m, 3H), 1.58 (dd, J=11.1, 8.2 Hz, 3H), 1.24 (s, 2H).

### Compound THP-A35. (R)-3-((S)-7-((1-(2-fluoro-4-((1S,2S)-6-hydroxy-2-phenyl-1,2,3,4-tetrahydronaphthalen-1-yl)-5-methoxyphenyl)piperidin-4-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

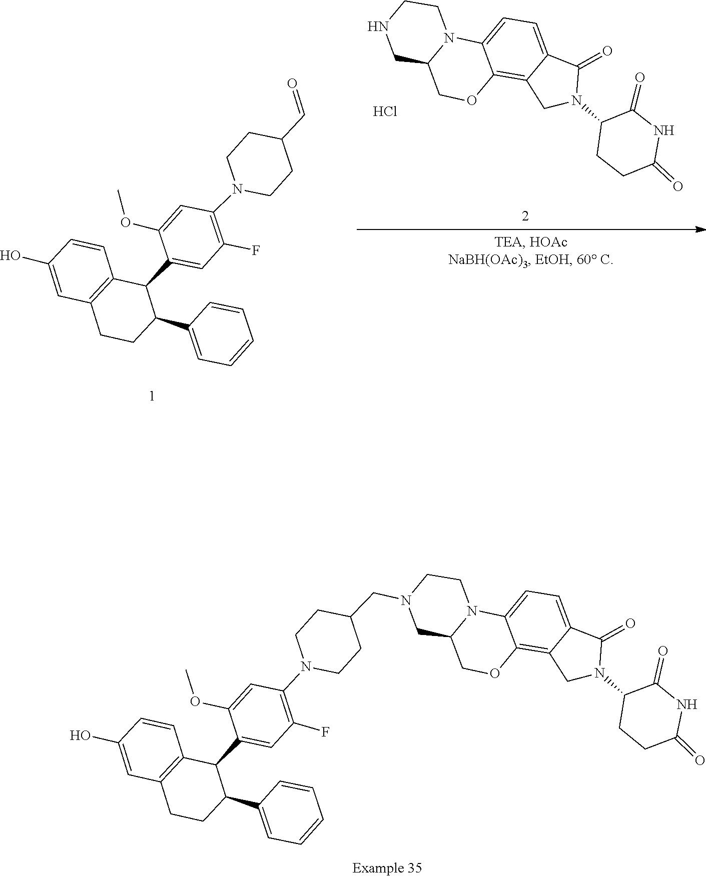 [SMILES: COc1cc(N2CCC(C=O)CC2)c(F)cc1[C@@H]1c2ccc(O)cc2CC[C@@H]1c1ccccc1 ; Cl ; O=C1CC[C@H](N2Cc3c(ccc4c3OC[C@H]3CNCCN43)C2=O)C(=O)N1 ; COc1cc(N2CCC(CN3CCN4c5ccc6c(c5OC[C@H]4C3)CN([C@H]3CCC(=O)NC3=O)C6=O)CC2)c(F)cc1[C@@H]1c2ccc(O)cc2CC[C@@H]1c1ccccc1]

To a mixture of 1-(2-fluoro-4-((1S,2S)-6-hydroxy-2-phenyl-1,2,3,4-tetrahydronaphthalen-1-yl)-5-methoxyphenyl)piperidine-4-carbaldehyde (19 mg, 0.05 mmol, 1.0 eq) and rac-(R)-3-((S)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride (15 mg, 0.05 mmol, 1.0 eq) in EtOH (5 mL) was added triethylamine (26 mg, 0.2 mmol, 4.0 eq), followed by the addition of AcOH (150 mg, 2 mmol, 40 eq) and stirred at 25° C. for 0.5 hour. The mixture was added NaBH(OAc)₃ (59 mg, 0.2 mmol, 4.0 eq) and stirred at 50° C. for 2 hours. LCMS showed the reaction was completed. The reaction was cooled to 20° C. and concentrated under vacuum. The residue was purified by prep-HPLC (ACN/0.05% FA) to afford (S)-3-((R)-7-((1-(2-fluoro-4-((1S,2S)-6-hydroxy-2-phenyl-1,2,3,4-tetrahydronaphthalen-1-yl)-5-methoxyphenyl)piperidin-4-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (5.79 mg, 30%) as a white solid. LC-MS purity: 100% (UV at 254 nm), 800.1 [M+H]⁺. ¹H NMR (400 MHz, MeOD) δ 7.32 (d, J=8.3 Hz, 1H), 7.09 (d, J=7.1 Hz, 3H), 7.03 (d, J=8.5 Hz, 1H), 6.81-6.76 (m, 2H), 6.65 (dd, J=8.2, 5.4 Hz, 2H), 6.54 (dd, J=8.4, 2.4 Hz, 1H), 6.29 (d, J=13.6 Hz, 1H), 6.24 (d, J=7.5 Hz, 1H), 5.10 (dd, J=13.3, 5.0 Hz, 1H), 4.80 (d, J=5.0 Hz, 1H), 4.41-4.27 (m, 3H), 4.10-4.01 (m, 1H), 3.86 (d, J=12.1 Hz, 1H), 3.19-3.05 (m, 2H), 3.01 (d, J=22.5 Hz, 6H), 2.89 (dd, J=13.1, 5.4 Hz, 2H), 2.82-2.75 (m, 1H), 2.72-2.58 (m, 2H), 2.50 (dd, J=13.2, 4.7 Hz, 1H), 2.35 (d, J=7.0 Hz, 2H), 2.28 (dd, J=22.3, 10.0 Hz, 2H), 2.20-2.11 (m, 1H), 1.88 (dd, J=7.6, 3.8 Hz, 3H), 1.73 (dt, J=14.3, 7.0 Hz, 2H), 1.38 (ddd, J=25.5, 18.7, 13.9 Hz, 5H).

### Compound THP-A52. (3S)-3-((5aR)-7-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-1 -oxo-1,3,5,5a,6,7,8,9-octahydro-2H-pyrazino[1′,2′:4,5][1,4]oxazino[2,3-e]isoindol-2-yl)piperidine-2,6-dione

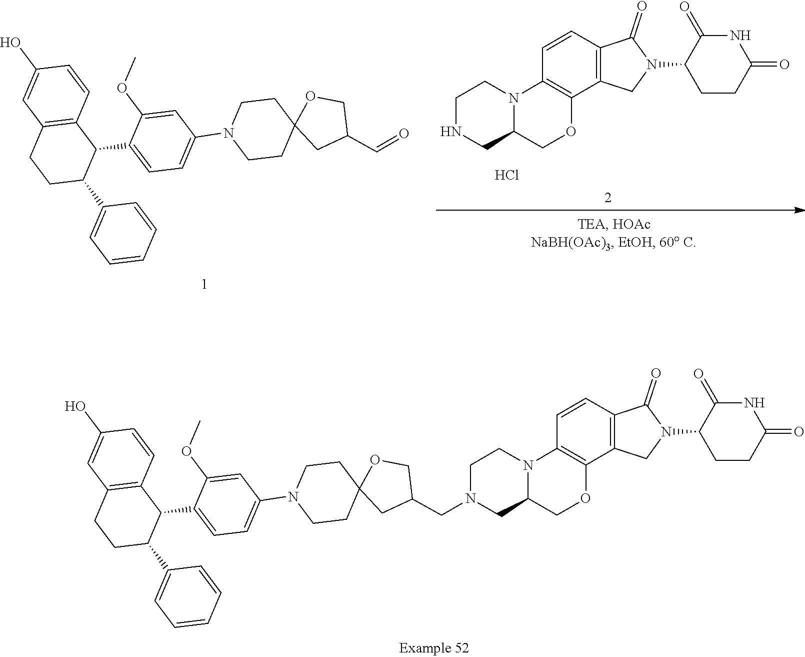 [SMILES: COc1cc(N2CCC3(CC2)CC(C=O)CO3)ccc1[C@H]1c2ccc(O)cc2CC[C@H]1c1ccccc1 ; O=C1CC[C@H](N2Cc3c(ccc4c3OC[C@H]3CNCCN43)C2=O)C(=O)N1 ; Cl ; COc1cc(N2CCC3(CC2)CC(CN2CCN4c5ccc6c(c5OC[C@H]4C2)CN([C@H]2CCC(=O)NC2=O)C6=O)CO3)ccc1[C@H]1c2ccc(O)cc2CC[C@H]1c1ccccc1]

To a mixture of 8-(4-((1R,2R)-6-hydroxy-2-phenyl-1,2,3,4-tetrahydronaphthalen-1-yl)-3-methoxyphenyl)-1-oxa-8-azaspiro[4.5]decane-3-carbaldehyde (60 mg, 0.14 mmol, 1.0 eq) and rac-(R)-3-((S)-1-oxo-1,3,5,5a,6,7,8,9-octahydro-2H-pyrazino[1′,2′:4,5][1,4]oxazino[2,3-e]isoindol-2-yl)piperidine-2,6-dione (48.3 mg, 0.14 mmol, 1.0 eq) in EtOH (5 mL) was added triethylamine (55 mg, 0.54 mmol, 4.0 eq), followed by the addition of AcOH (327 mg, 5.4 mmol, 40 eq) and stirred at 25° C. for 0.5 hour. The mixture was added NaBH(OAc)₃ (115 mg, 0.54 mmol, 4.0 eq) and stirred at 50° C. for 2 hours. LCMS showed the reaction was completed. The reaction was cooled to 20° C. and concentrated under vacuum. The residue was purified by prep-HPLC (ACN/0.05% FA) to afford (3S)-3-((5aR)-7-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (18 mg, 13%) as a white solid. LC-MS purity: 100% (UV at 254 nm), 800.1 [M+H]⁺. ¹H NMR (400 MHz, MeOD) δ 7.31 (d, J=8.3 Hz, 1H), 7.05 (dt, J=12.0, 6.0 Hz, 4H), 6.78-6.72 (m, 2H), 6.64 (d, J=8.3 Hz, 2H), 6.54-6.48 (m, 2H), 6.43 (dd, J=8.5, 2.1 Hz, 1H), 6.21 (d, J=2.1 Hz, 1H), 5.10 (dd, J=13.2, 5.1 Hz, 1H), 4.78 (d, J=5.3 Hz, 1H), 4.40-4.34 (m, 2H), 4.05 (dd, J=12.5, 6.7 Hz, 2H), 3.86 (d, J=11.8 Hz, 1H), 3.61 (t, J=8.0 Hz, 1H), 3.23 (s, 1H), 3.15 (s, 3H), 3.03 (s, 3H), 3.00 (d, J=5.5 Hz, 2H), 2.89 (dd, J=12.8, 4.9 Hz, 2H), 2.82-2.76 (m, 1H), 2.74-2.66 (m, 1H), 2.48 (d, J=7.2 Hz, 2H), 2.30 (s, 1H), 2.20-2.05 (m, 3H), 1.89 (d, J=18.7 Hz, 1H), 1.80 (s, 4H), 1.65 (s, 2H), 1.49 (dd, J=12.6, 8.3 Hz, 1H), 1.33 (d, J=18.0 Hz, 5H).

### Compound THP-A56. (3S)-3-((4aS)-3-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-8 -oxo-1,2,3,4,4a,5,8,10-octahydro-9H-pyrazino[1′,2′:4,5][1,4]oxazino[2,3-f]isoindol-9-yl)piperidine-2,6-dione

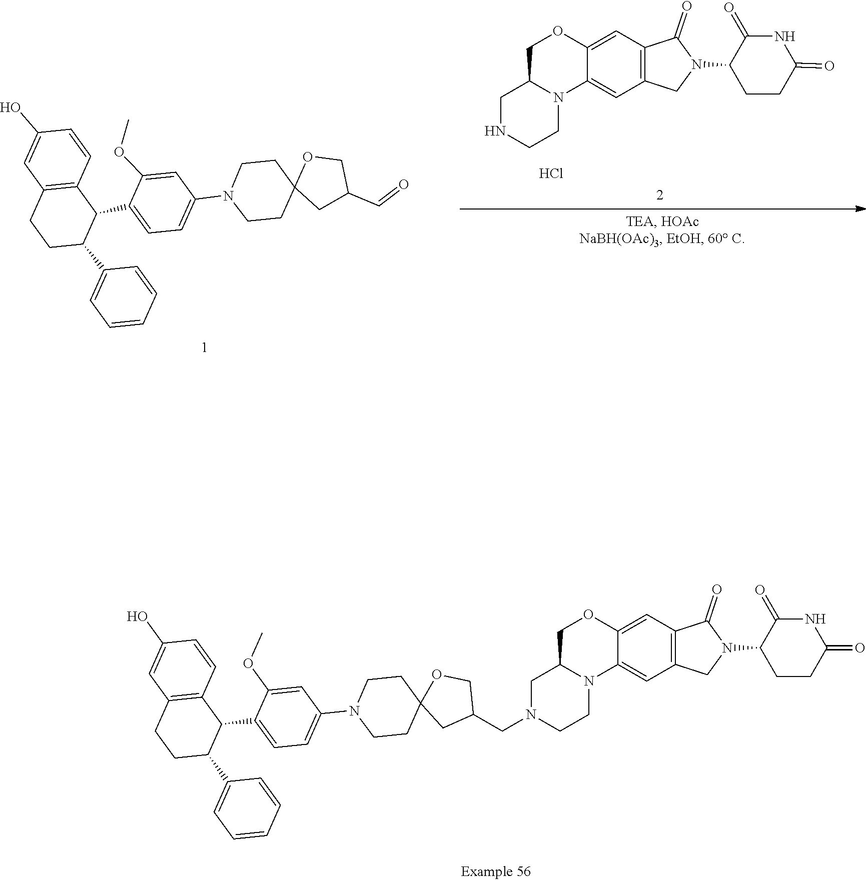 [SMILES: COc1cc(N2CCC3(CC2)CC(C=O)CO3)ccc1[C@H]1c2ccc(O)cc2CC[C@H]1c1ccccc1 ; O=C1CC[C@H](N2Cc3cc4c(cc3C2=O)OC[C@@H]2CNCCN42)C(=O)N1 ; Cl ; COc1cc(N2CCC3(CC2)CC(CN2CCN4c5cc6c(cc5OC[C@@H]4C2)C(=O)N([C@H]2CCC(=O)NC2=O)C6)CO3)ccc1[C@H]1c2ccc(O)cc2CC[C@H]1c1ccccc1]

To a mixture of 8-(4-((1R,2R)-6-hydroxy-2-phenyl-1,2,3,4-tetrahydronaphthalen-1-yl)-3-methoxyphenyl)-1-oxa-8-azaspiro[4.5]decane-3-carbaldehyde (60 mg, 0.12 mmol, 1.0 eq) and (R)-3-((R)-8-oxo-1,2,3,4,4a,5,8,10-octahydro-9H-pyrazino[1′,2′:4,5][1,4]oxazino[2,3-f]isoindol-9-yl)piperidine-2,6-dione hydrochloride (43 mg, 0.12 mmol, 1.0 eq) in EtOH (5 mL) was added triethylamine (48.6 mg, 0.48 mmol, 4.0 eq), followed by the addition of AcOH (288 mg, 4.11 mmol, 40 eq) and stirred at 25° C. for 0.5 hour. The mixture was added NaBH(OAc)₃ (102 mg, 0.48 mmol, 4.0 eq) and stirred at 50° C. for 2 hours. LCMS showed the reaction was completed. The reaction was cooled to 20° C. and concentrated under vacuum. The residue was purified by Prep-HPLC (ACN/0.05% FA) to afford (3S)-3-((4aS)-3-((8-(4-((1R,2R)-6-hydroxy-2-phenyl-1,2,3,4-tetrahydronaphthalen-1-yl)-3-methoxyphenyl)-1-oxa-8-azaspiro[4.5]decan-3-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (17.43 mg, 29%) as a white solid. LC-MS purity: 100% (UV at 254 nm), 838.6 [M+H]⁺. ¹H NMR (400 MHz, DMSO) δ 10.93 (s, 1H), 9.05 (s, 1H), 7.06 (t, J=5.8 Hz, 4H), 6.93 (s, 1H), 6.74-6.70 (m, 2H), 6.55 (d, J=8.2 Hz, 2H), 6.45 (dd, J=8.4, 2.5 Hz, 1H), 6.36 (d, J=8.4 Hz, 1H), 6.31 (d, J=7.9 Hz, 1H), 6.11 (s, 1H), 5.07-4.99 (m, 1H), 4.64 (d, J=4.9 Hz, 1H), 4.35-3.99 (m, 4H), 3.90 (s, 3H), 3.45 (s, 1H), 3.20 (d, J=14.6 Hz, 2H), 3.08 (s, 4H), 2.94 (s, 3H), 2.67 (d, J=1.8 Hz, 2H), 2.33 (d, J=1.8 Hz, 3H), 2.11 (d, J=28.9 Hz, 3H), 1.96 (d, J=5.5 Hz, 3H), 1.65 (s, 4H), 1.60 (s, 4H), 1.35 (s, 1H), 1.24 (s, 1H).

### Indole Series

### Compound IND-A48: (R)-3-((R)-3-((1-(4-((1R,3R)-2-(2,2-difluoroethyl)-3-methyl-2,3,4,9-tetrahydro-1H-pyrido[3,4-b]indol-1-yl)-3-methoxyphenyl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

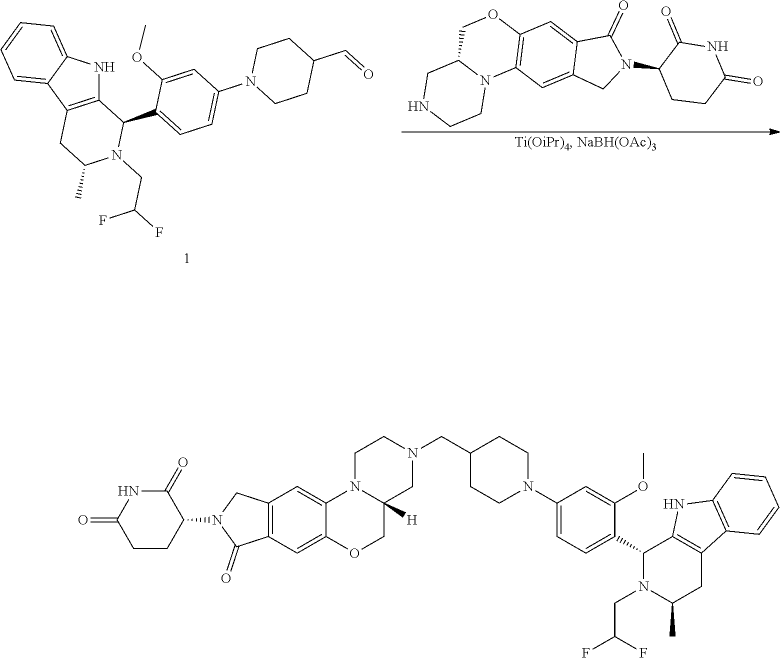 [SMILES: COc1cc(N2CCC(C=O)CC2)ccc1[C@@H]1c2[nH]c3ccccc3c2C[C@@H](C)N1CC(F)F ; O=C1CC[C@@H](N2Cc3cc4c(cc3C2=O)OC[C@H]2CNCCN42)C(=O)N1 ; COc1cc(N2CCC(CN3CCN4c5cc6c(cc5OC[C@H]4C3)C(=O)N([C@@H]3CCC(=O)NC3=O)C6)CC2)ccc1[C@@H]1c2[nH]c3ccccc3c2C[C@@H](C)N1CC(F)F]

To a mixture of 1-(4-((1R,3R)-2-(2,2-difluoroethyl)-3-methyl-2,3,4,9-tetrahydro-1H-pyrido[3,4-b]indol-1-yl)-3-methoxyphenyl)piperidine-4-carbaldehyde (60 mg, 0.13 mmol, 1 eq) and (R)-3-((R)-8-oxo-1,2,3,4,4a,5,8,10-octahydro-9H-pyrazino[1′,2′:4,5][1,4]oxazino[2,3-f]isoindol-9-yl)piperidine-2,6-dione (50 mg, 0.13 mmol, 1 eq) in THF (10 mL) was added Ti(OiPr)₄ (110 mg, 0.39 mmol, 3 eq), the mixture was stirred at 50° C. for 1 hour under N₂. The mixture was added NaBH(OAc)₃ (109 mg, 0.51 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (R)-3-((R)-3-((1-(4-((1R,3R)-2-(2,2-difluoroethyl)-3-methyl-2,3,4,9-tetrahydro-1H-pyrido[3,4-b]indol-1-yl)-3-methoxyphenyl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (27.88 mg) as a white solid.

LCMS purity (A50B50): 100% (UV at 254 nm), MS: 807.39 [M+1]; Retention time: 0.773 min

¹H NMR (400 MHz, DMSO) δ 10.95 (s, 1H), 10.52 (s, 1H), 9.67 (s, 1H), 7.43 (d, J=7.5 Hz, 1H), 7.21 (d, J=7.8 Hz, 2H), 7.08-6.93 (m, 3H), 6.63 (s, 1H), 6.40 (t, J=10.5 Hz, 2H), 6.09 (t, J=54.9 Hz, 1H), 5.21 (s, 1H), 5.05 (dd, J=13.0, 5.1 Hz, 1H), 4.33 (t, J=12.4 Hz, 2H), 4.16 (d, J=16.5 Hz, 2H), 4.11-4.01 (m, 1H), 3.90 (s, 4H), 3.71 (dd, J=23.2, 16.4 Hz, 5H), 3.29-3.19 (m, 2H), 3.08 (dd, J=18.9, 10.8 Hz, 3H), 2.90 (dd, J=21.6, 9.0 Hz, 2H), 2.71 (d, J=11.7 Hz, 4H), 2.44-2.29 (m, 1H), 1.97 (dd, J=14.0, 7.1 Hz, 2H), 1.83 (dd, J=27.7, 10.1 Hz, 2H), 1.40-1.21 (m, 4H), 1.07 (d, J=6.2 Hz, 3H).

### Compound IND-A49: (S)-3-((R)-7-((1-(4-((1R,3R)-2-(2-fluoro-2-methylpropyl)-3-methyl-2,3,4,9-tetrahydro-1H-pyrido[3,4-b]indol-1-yl)-3-methoxyphenyl)piperidin-4-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

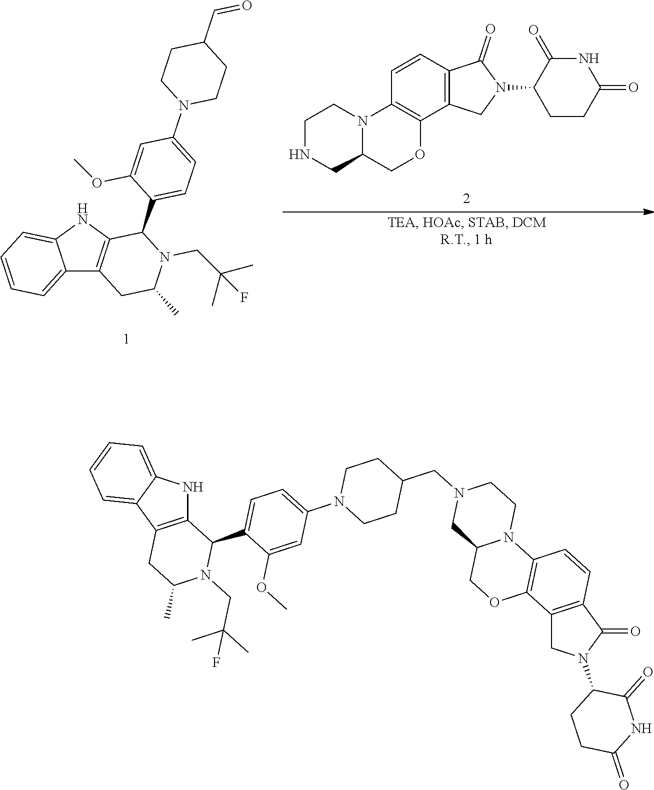 [SMILES: COc1cc(N2CCC(C=O)CC2)ccc1[C@@H]1c2[nH]c3ccccc3c2C[C@@H](C)N1CC(C)(C)F ; O=C1CC[C@H](N2Cc3c(ccc4c3OC[C@H]3CNCCN43)C2=O)C(=O)N1 ; COc1cc(N2CCC(CN3CCN4c5ccc6c(c5OC[C@H]4C3)CN([C@H]3CCC(=O)NC3=O)C6=O)CC2)ccc1[C@@H]1c2[nH]c3ccccc3c2C[C@@H](C)N1CC(C)(C)F]

To a mixture of 1-(4-((1R,3R)-2-(2-fluoro-2-methylpropyl)-3-methyl-2,3,4,9-tetrahydro-1H-pyrido[3,4-b]indol-1-yl)-3-methoxyphenyl)piperidine-4-carbaldehyde (30 mg, 0.063 mmol, 1 eq.), (S)-3-((R)-1-oxo-1,3,5,5a,6,7,8,9-octahydro-2H-pyrazino[1′,2′:4,5][1,4]oxazino[2,3-c]isoindol-2-yl)piperidine-2,6-dione formate (30 mg, 0.07 mmol, 1.2 eq.), TEA (9.6 mg, 0.095 mmol, 1.5 eq.) in DCM (2.0 mL) was added acetic acid (6.4 mg, 0.11 mmol, 1.7 eq.) followed by Sodium triacetoxyborohydride (27 mg, 0.13 mmol, 2 eq.). The mixture was stirred at room temperature for 30 minutes and concentrated. The residue was purified by reverse-phase chromatography (0-50% Acetonitrile/0.05% Formic acid)) to afford (S)-3-((R)-7-((1-(4-((1R,3R)-2-(2-fluoro-2-methylpropyl)-3-methyl-2,3,4,9-tetrahydro-1H-pyrido[3,4-b]indol-1-yl)-3-methoxyphenyl)piperidin-4-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (20.43 mg, 39.7% yield) as white solid. LC-MS purity: 100% (UV at 254 nm), 818.4 [M+H]⁺

¹H NMR (400 MHz, DMSO) δ 10.93 (s, 1H), 10.39 (s, 1H), 7.40 (d, J=7.4 Hz, 1H), 7.18 (d, J=8.2 Hz, 2H), 7.09-6.89 (m, 3H), 6.58-6.54 (m, 1H), 6.44 (d, J=8.0 Hz, 1H), 6.35-6.28 (m, 1H), 5.27 (s, 1H), 5.03 (dd, J=13.4, 5.2 Hz, 1H), 4.41-4.32 (m, 1H), 4.26 (d, J=16.8 Hz, 1H), 4.10 (d, J=16.8 Hz, 1H), 4.00-3.93 (m, 1H), 3.87-3.77 (m, 4H), 3.68 (d, J=12.0 Hz, 2H), 3.23-3.13 (m, 2H), 2.99-2.85 (m, 3H), 2.80-2.57 (m, 6H), 2.46-2.39 (m, 2H), 2.25-2.17 (m, 2H), 2.15-2.05 (m, 1H), 2.00-1.90 (m, 1H), 1.85-1.66 (m, 4H), 1.36 (d, J=22.0 Hz, 3H), 1.30-1.07 (m, 6H), 1.01 (d, J=6.6 Hz, 3H).

### Compound IND-A51. (4aR)-3-((1-(4-((1R,3R)-2-(2,2-difluoroethyl)-3-methyl-2,3,4,9-tetrahydro-1H-pyrido[3,4-b]indol-1-yl)-3-methoxyphenyl)piperidin-4-yl)methyl)-N-(2,6-dioxopiperidin-3-yl)-1,2,3,4,4a,5-hexahydropyrazino[1,2-d]pyrido[2,3-b][1,4]oxazine-8-carboxamide formate

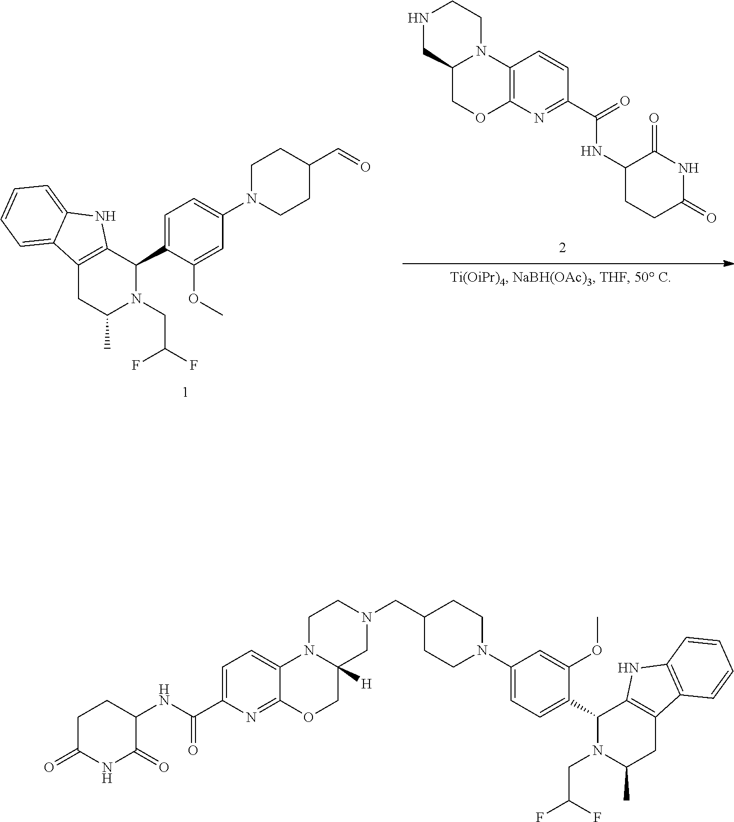 [SMILES: COc1cc(N2CCC(C=O)CC2)ccc1[C@@H]1c2[nH]c3ccccc3c2C[C@@H](C)N1CC(F)F ; O=C1CCC(NC(=O)c2ccc3c(n2)OC[C@H]2CNCCN32)C(=O)N1 ; COc1cc(N2CCC(CN3CCN4c5ccc(C(=O)NC6CCC(=O)NC6=O)nc5OC[C@H]4C3)CC2)ccc1[C@@H]1c2[nH]c3ccccc3c2C[C@@H](C)N1CC(F)F]

To a mixture of 1-(4-((1R,3R)-2-(2,2-difluoroethyl)-3-methyl-2,3,4,9-tetrahydro-1H-pyrido[3,4-b]indol-1-yl)-3-methoxyphenyl)piperidine-4-carbaldehyde (27.0 mg, 0.058 mmol, 1.0 eq) and (4aR)—N-(2,6-dioxopiperidin-3-yl)-1,2,3,4,4a,5-hexahydropyrazino[1,2-d]pyrido[2,3-b][1,4]oxazine-8-carboxamide (20 mg, 0.058 mmol, 1.0 eq) in THF (4 mL) was added Ti(OiPr)₄ (50.0 mg, 0.174 mmol, 3.0 eq), followed by the addition of NaBH(OAc)₃ (25.0 mg, 0.116 mmol, 2.0 eq) and stirred at 50° C. for 1 hour. And then NaBH(OAc)₃ (25.0 mg, 0.116 mmol, 2.0 eq) was added to the mixture and stirred at 50° C. for 1 hours. LCMS showed the reaction was completed. The reaction was cooled to 20° C. and concentrated under vacuum. The residue was purified by Prep-HPLC (ACN/0.05% FA) to afford (4aR)-3-((1-(4-((1R,3R)-2-(2,2-difluoroethyl)-3-methyl-2,3,4,9-tetrahydro-11H-pyrido[3,4-b]indol-1-yl)-3-methoxyphenyl)piperidin-4-yl)methyl)-N-(2,6-dioxopiperidin-3-yl)-1,2,3,4,4a,5-hexahydropyrazino[1,2-d]pyrido[2,3-b][1,4]oxazine-8-carboxamide (15.6 mg, 35%) as a Purple solid.

LCMS purity (L-A70B30): 100% (UV at 254 nm), MS: 842.4 [M+H]⁺; Retention time: 3.470 min

¹H NMR (400 MHz, DMSO) δ 10.84 (s, 1H), 10.51 (s, 1H), 9.79 (s, 1H), 8.55 (d, J=8.2 Hz, 1H), 7.64 (d, J=7.6 Hz, 1H), 7.44 (s, 2H), 7.20 (d, J=8.4 Hz, 1H), 7.08-6.93 (m, 2H), 6.63 (s, 1H), 6.40 (dt, J=21.5, 5.9 Hz, 2H), 6.09 (t, J=58.4 Hz, 1H), 5.20 (s, 1H), 4.77-4.67 (m, 1H), 4.51 (d, J=10.6 Hz, 1H), 4.18 (dd, J=21.2, 10.4 Hz, 2H), 3.89 (s, 3H), 3.79-3.62 (m, 6H), 3.11 (ddd, J=67.3, 39.1, 9.4 Hz, 7H), 2.84-2.59 (m, 5H), 2.23-2.08 (m, 1H), 2.04-1.93 (m, 2H), 1.83 (dd, J=23.2, 8.7 Hz, 2H), 1.30 (dd, J=12.8, 10.5 Hz, 3H), 1.06 (s, 3H).

### Compound IND-A54. (4aR)-3-((1-(4-((1S,3S)-2-(2,2-difluoroethyl)-3-methyl-2,3,4,9-tetrahydro-1H-pyrido[3,4-b]indol-1-yl)-3-methoxyphenyl)piperidin-4-yl)methyl)-N-(2,6-dioxopiperidin-3-yl)-1,2,3,4,4a,5-hexahydropyrazino[1,2-d]pyrido[2,3-b][1,4]oxazine-8-carboxamide

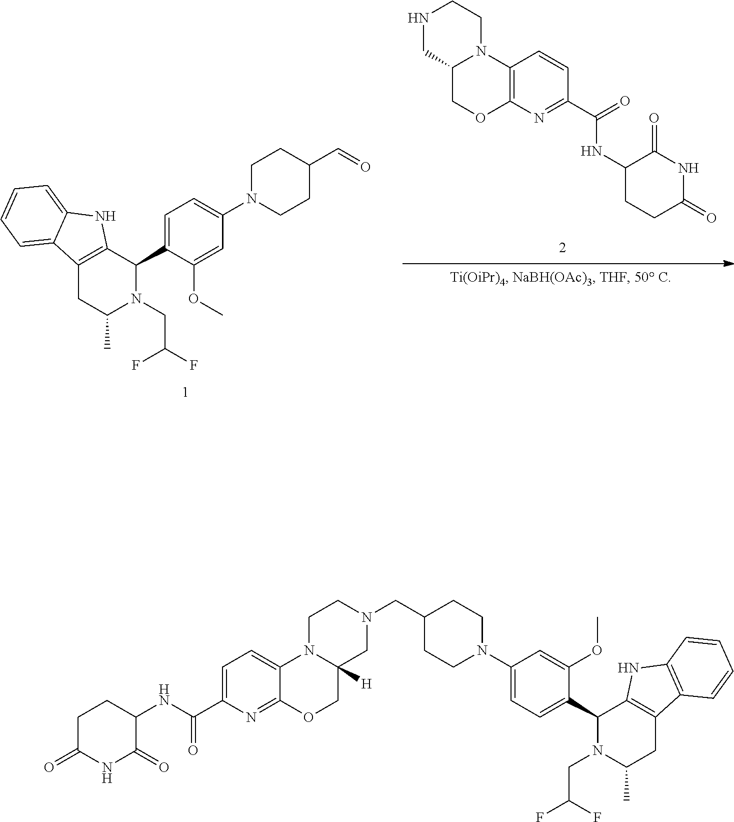 [SMILES: COc1cc(N2CCC(C=O)CC2)ccc1[C@@H]1c2[nH]c3ccccc3c2C[C@@H](C)N1CC(F)F ; O=C1CCC(NC(=O)c2ccc3c(n2)OC[C@@H]2CNCCN32)C(=O)N1 ; COc1cc(N2CCC(CN3CCN4c5ccc(C(=O)NC6CCC(=O)NC6=O)nc5OC[C@H]4C3)CC2)ccc1[C@H]1c2[nH]c3ccccc3c2C[C@H](C)N1CC(F)F]

To a mixture of 1-(4-((1R,3R)-2-(2,2-difluoroethyl)-3-methyl-2,3,4,9-tetrahydro-1H-pyrido[3,4-b]indol-1-yl)-3-methoxyphenyl)piperidine-4-carbaldehyde (27.0 mg, 0.058 mmol, 1.0 eq) and (4aS)—N-(2,6-dioxopiperidin-3-yl)-1,2,3,4,4a,5-hexahydropyrazino[1,2-d]pyrido[2,3-b][1,4]oxazine-8-carboxamide (20 mg, 0.058 mmol, 1.0 eq) in THF (4 mL) was added Ti(OiPr)₄ (50.0 mg, 0.174 mmol, 3.0 eq), followed by the addition of NaBH(OAc)₃ (25.0 mg, 0.116 mmol, 2.0 eq) and stirred at 50° C. for 1 hour. And then NaBH(OAc)₃ (25.0 mg, 0.116 mmol, 2.0 eq) was added to the mixture and stirred at 50° C. for 1 hours. LCMS showed the reaction was completed. The reaction was cooled to 20° C. and concentrated under vacuum. The residue was purified by Prep-HPLC (ACN/0.05% FA) to afford (4aR)-3-((1-(4-((1R,3R)-2-(2,2-difluoroethyl)-3-methyl-2,3,4,9-tetrahydro-1H-pyrido[3,4-b]indol-1-yl)-3-methoxyphenyl)piperidin-4-yl)methyl)-N-(2,6-dioxopiperidin-3-yl)-1,2,3,4,4a,5-hexahydropyrazino[1,2-d]pyrido [2,3-b][1,4]oxazine-8-carboxamide (11.57 mg, 27.5%) as a Purple solid.

LCMS purity (L-A70B30): 100% (UV at 254 nm), MS: 796.4 [M+H]⁺; Retention time: 3.451 min

¹H NMR (400 MHz, DMSO) δ 10.84 (s, 1H), 10.52 (s, 1H), 8.50 (d, J=8.2 Hz, 1H), 7.57 (d, J=8.1 Hz, 1H), 7.42 (d, J=7.5 Hz, 1H), 7.32 (d, J=8.0 Hz, 1H), 7.20 (d, J=8.1 Hz, 1H), 6.99 (dt, J=22.5, 6.9 Hz, 2H), 6.60 (s, 1H), 6.37 (dd, J=22.5, 8.2 Hz, 2H), 6.08 (t, J=56.0 Hz, 1H), 5.20 (s, 1H), 4.72 (t, J=6.3 Hz, 1H), 4.46 (d, J=8.4 Hz, 1H), 4.10 (t, J=10.0 Hz, 1H), 3.88 (s, 3H), 3.80 (d, J=11.9 Hz, 1H), 3.69 (d, J=9.1 Hz, 2H), 3.22 (d, J=17.1 Hz, 3H), 3.07-2.93 (m, 3H), 2.77 (dd, J=23.4, 9.2 Hz, 3H), 2.71-2.60 (m, 4H), 2.30-2.20 (m, 2H), 2.20-2.07 (m, 2H), 2.03-1.93 (m, 1H), 1.88-1.61 (m, 5H), 1.28-1.17 (m, 2H), 1.07 (d, J=6.6 Hz, 3H).

### Compound IND-A59. (R)-3-((S)-7-(((S)-8-(5-((1S,3S)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3 -yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione formate

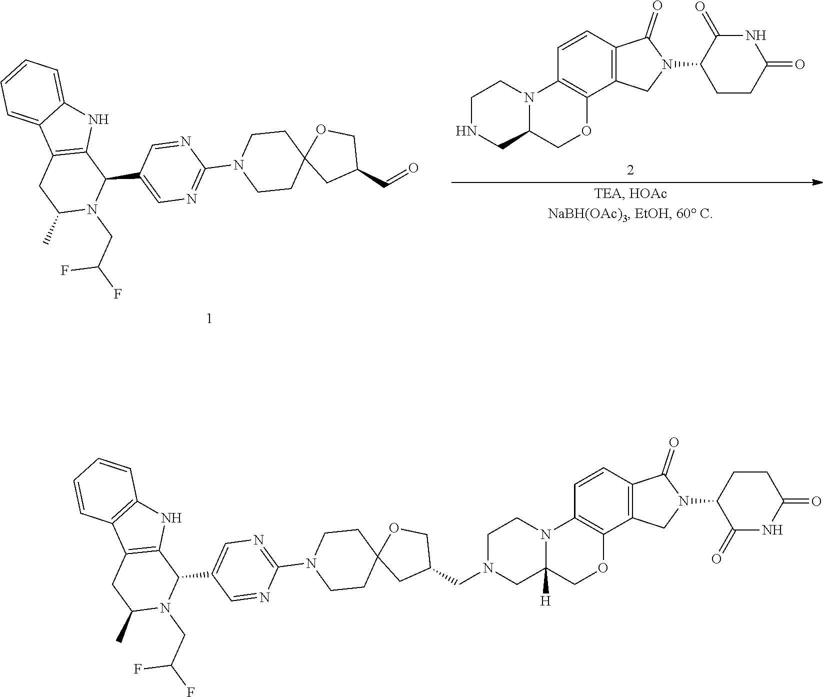 [SMILES: C[C@@H]1Cc2c([nH]c3ccccc23)[C@@H](c2cnc(N3CCC4(CC3)C[C@H](C=O)CO4)nc2)N1CC(F)F ; O=C1CC[C@H](N2Cc3c(ccc4c3OC[C@H]3CNCCN43)C2=O)C(=O)N1 ; C[C@H]1Cc2c([nH]c3ccccc23)[C@H](c2cnc(N3CCC4(CC3)C[C@@H](CN3CCN5c6ccc7c(c6OC[C@@H]5C3)CN([C@@H]3CCC(=O)NC3=O)C7=O)CO4)nc2)N1CC(F)F]

To a mixture of (S)-8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decane-3-carbaldehyde (28.0 mg, 0.06 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride (20.1 mg, 0.06 mmol, 1.0 eq) in EtOH (4 mL) was added triethylamine (0.05 mL), followed by the addition of AcOH (0.1 mL) and stirred at 25° C. for 0.5 hour. The mixture was added NaBH(OAc)₃ (47.9 mg, 0.23 mmol, 4.0 eq) and stirred at 60° C. for 2 hours. LCMS showed the reaction was completed. The reaction was cooled to 20° C. and concentrated under vacuum. The residue was purified by Prep-HPLC (ACN/0.05% FA) to afford (S)-3-((R)-7-(((R)-8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (5.4 mg, 11.4%) as a white solid.

LCMS purity (A70B30): 100% (UV at 254 nm), MS: 836.2 [M+H]⁺; Retention time: 5.778 min

¹H NMR (400 MHz, DMSO) δ 10.93 (s, 1H), 10.69 (s, 1H), 8.45 (s, 1H), 8.09 (s, 2H), 7.43 (d, J=7.8 Hz, 1H), 7.26 (d, J=8.0 Hz, 1H), 7.17 (d, J=8.3 Hz, 1H), 7.09-6.92 (m, 3H), 6.12 (t, J=58.1 Hz, 1H), 5.03 (dd, J=13.3, 5.0 Hz, 1H), 4.85 (s, 1H), 4.36 (d, J=9.3 Hz, 1H), 4.26 (d, J=16.9 Hz, 1H), 4.10 (d, J=16.8 Hz, 1H), 3.94 (dt, J=15.0, 8.8 Hz, 4H), 3.82 (d, J=12.3 Hz, 1H), 3.63-3.53 (m, 2H), 3.50-3.45 (m, 1H), 3.16 (dd, J=8.7, 7.0 Hz, 2H), 3.10-2.81 (m, 4H), 2.78-2.56 (m, 6H), 2.39-2.31 (m, 3H), 1.96 (td, J=11.5, 6.4 Hz, 2H), 1.80-1.67 (m, 1H), 1.62-1.54 (m, 3H), 1.53-1.44 (m, 1H), 1.37 (dd, J=11.6, 8.1 Hz, 1H), 1.30-1.22 (m, 1H), 1.09 (d, J=6.7 Hz, 3H).

### Compound IND-A61. (R)-3-((R)-3-(((S)-8-(5-((1S,3S)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

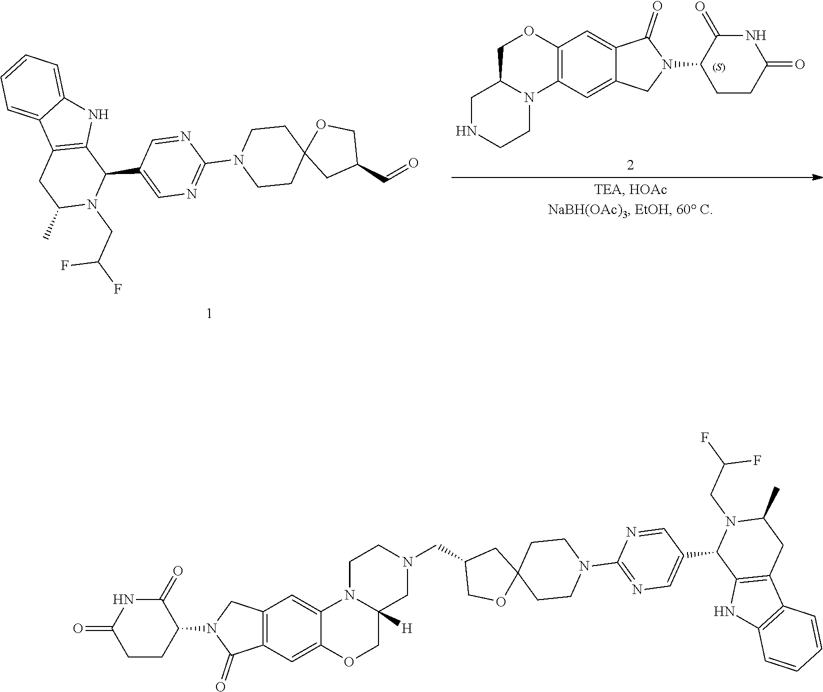

To a mixture of (S)-8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decane-3-carbaldehyde (31.0 mg, 0.06 mmol, 1.0 eq) and (S)-3-((S)-8-oxo-1,2,3,4,4a,5,8,10-octahydro-9H-pyrazino[1′,2′:4,5][1,4]oxazino[2,3-f]isoindol-9-yl)piperidine-2,6-dione hydrochloride (17.4 mg, 0.06 mmol, 1.0 eq) in EtOH (4 mL) was added triethylamine (0.05 mL), followed by the addition of AcOH (0.1 mL) and stirred at 25° C. for 0.5 hour. The mixture was added NaBH(OAc)₃ (53.1 mg, 0.25 mmol, 4.0 eq) and stirred at 60° C. for 2 hours. LCMS showed the reaction was completed. The reaction was cooled to 20° C. and concentrated under vacuum. The residue was purified by prep-HPLC (ACN/0.05% FA) to afford (S)-3-((S)-3-(((R)-8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (7.2 mg, 13.8%) as a yellow solid.

LCMS purity (A70B³⁰): 100% (UV at 254 nm), MS: 836.2 [M+H]⁺; Retention time: 5.730 min

¹H NMR (400 MHz, DMSO-d₆) δ 10.95 (s, 1H), 10.69 (s, 1H), 8.11 (s, 2H), 7.44 (d, J=7.8 Hz, 1H), 7.27 (d, J=7.9 Hz, 1H), 7.20 (d, J=8.2 Hz, 1H), 7.06 (dd, J=14.7, 6.6 Hz, 2H), 7.00-6.91 (m, 1H), 6.13 (t, J=56.3 Hz, 1H), 5.04 (dd, J=13.1, 5.0 Hz, 1H), 4.86 (s, 1H), 4.35 (d, J=8.8 Hz, 1H), 4.27-4.12 (m, 3H), 3.97 (ddd, J=19.3, 10.2, 4.9 Hz, 5H), 3.61 (dd, J=11.9, 4.9 Hz, 4H), 3.21-3.07 (m, 5H), 2.89 (dd, J=17.6, 9.8 Hz, 2H), 2.69 (d, J=19.5 Hz, 2H), 2.34 (dd, J=12.2, 5.9 Hz, 1H), 2.12 (t, J=11.0 Hz, 1H), 1.96 (dd, J=11.5, 5.4 Hz, 1H), 1.66-1.58 (m, 3H), 1.55-1.43 (m, 2H), 1.24 (s, 5H), 1.10 (d, J=6.6 Hz, 3H).

### Compound IND-A63. (4aR)-3-((8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3-yl)methyl)-N—((S)-2,6-dioxopiperidin-3-yl)-1,2,3,4,4a,5-hexahydropyrazino[1,2-d]pyrido[2,3-b][1,4]oxazine-8-carboxamide

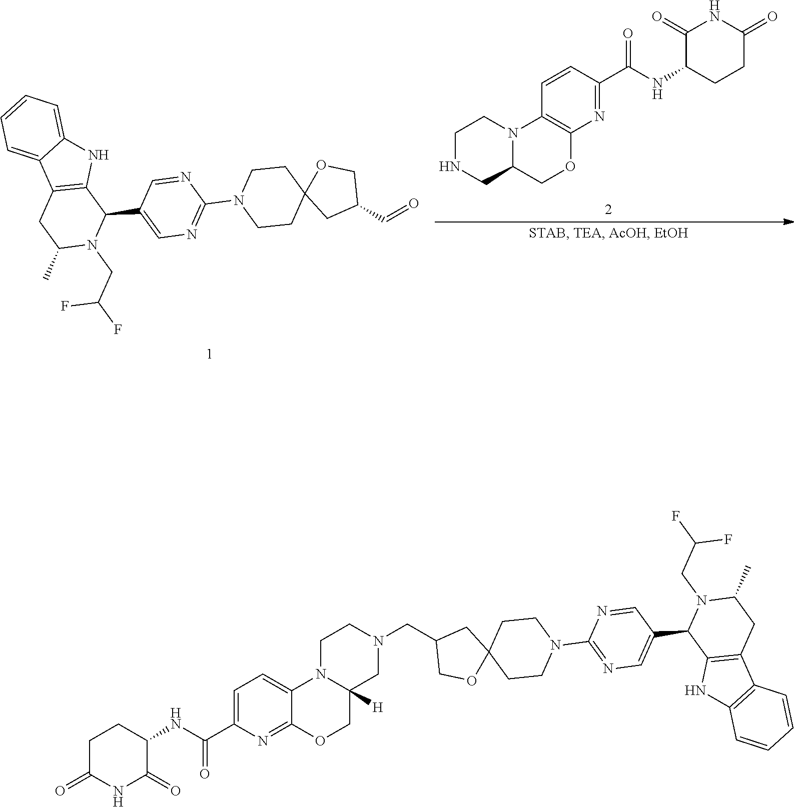 [SMILES: C[C@@H]1Cc2c([nH]c3ccccc23)[C@@H](c2cnc(N3CCC4(CC3)C[C@@H](C=O)CO4)nc2)N1CC(F)F ; O=C1CC[C@H](NC(=O)c2ccc3c(n2)OC[C@H]2CNCCN32)C(=O)N1 ; C[C@@H]1Cc2c([nH]c3ccccc23)[C@@H](c2cnc(N3CCC4(CC3)CC(CN3CCN5c6ccc(C(=O)N[C@H]7CCC(=O)NC7=O)nc6OC[C@H]5C3)CO4)nc2)N1CC(F)F]

To a mixture of 8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decane-3-carbaldehyde (30 mg, 0.06 mmol, 1 eq.) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride (21 mg, 0.06 mmol, 1 eq.) in DCM (2 mL) was added triethylamine (25 mg, 0.24 mmol, 4 eq.), NaBH(OAc)₃ (52 mg, 0.24 mmol, 4 eq.) followed by the addition of AcOH (146 mg, 2.4 mmol, 10 eq.). The reaction mixture was stirred at room temperature for 1 h and the mixture was concentrated and the residue was purified by reverse phase chromatography (0-50% Acetonitrile/0.05% Formic acid) to afford (4aR)-3-((8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3-yl)methyl)-N—((S)-2,6-dioxopiperidin-3-yl)-1,2,3,4,4a,5-hexahydropyrazino[1,2-d]pyrido[2,3-b][1,4]oxazine-8-carboxamide (8.4 mg, 16.8% yield) as a yellow solid.

LCMS purity: 100% (UV at 254 nm), 825.2 [M+H]⁺

¹H NMR (400 MHz, DMSO) δ 10.83 (s, 1H), 10.69 (s, 1H), 8.54 (s, 1H), 8.10 (s, 2H), 7.60 (s, 1H), 7.43 (d, J=7.8 Hz, 1H), 7.26 (d, J=8.0 Hz, 1H), 7.05 (t, J=7.0 Hz, 1H), 6.98 (t, J=7.7 Hz, 1H), 6.12 (t, J=56.0 Hz, 1H), 4.85 (s, 1H), 4.72 (t, J=13.2 Hz, 1H), 4.48 (s, 1H), 4.16 (dd, J=22.5, 12.6 Hz, 2H), 3.94 (dd, J=12.9, 5.1 Hz, 4H), 3.73-3.57 (m, 4H), 3.55-3.46 (m, 2H), 3.21-2.97 (m, 5H), 2.86-2.59 (m, 6H), 2.22-2.06 (m, 2H), 2.03-1.92 (m, 2H), 1.53 (dd, J=27.6, 19.1 Hz, 6H), 1.10 (d, J=6.6 Hz, 3H).

### Compound IND-A68. (R)-3-((S)-3-(((S)-8-(5-((1S,3S)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

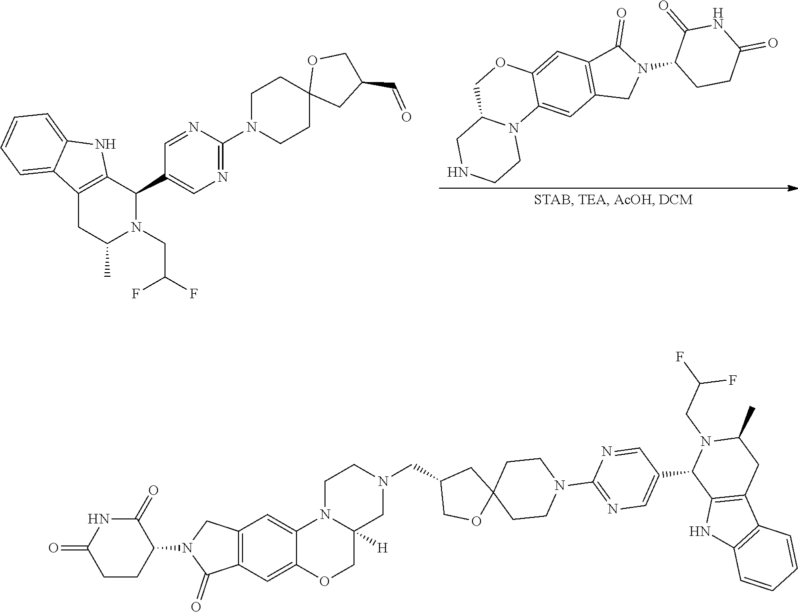 [SMILES: C[C@@H]1Cc2c([nH]c3ccccc23)[C@@H](c2cnc(N3CCC4(CC3)C[C@H](C=O)CO4)nc2)N1CC(F)F ; O=C1CC[C@H](N2Cc3cc4c(cc3C2=O)OC[C@H]2CNCCN42)C(=O)N1 ; C[C@H]1Cc2c([nH]c3ccccc23)[C@H](c2cnc(N3CCC4(CC3)C[C@@H](CN3CCN5c6cc7c(cc6OC[C@@H]5C3)C(=O)N([C@@H]3CCC(=O)NC3=O)C7)CO4)nc2)N1CC(F)F]

To a mixture of (S)-8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decane-3-carbaldehyde (35 mg, 0.07 mmol, 1 eq.) and (S)-3-((R)-8-oxo-1,2, 3,4,4a,5,8,10-octahydro-9-pyrazino[1′,2′:4,5][1,4]oxazino[2,3-f]isoindol-9-yl)piperidine-2,6-dione hydrochloride (33 mg, 0.08 mmol, 1.2 eq.) in DCM (3 mL) was added TEA (1 drop), AcOH (2 drops) and STAB (30 mg, 0.14 mmol, 2 eq.). The reaction mixture was stirred at room temperature for 1 h. The mixture was concentrated and the residue was purified by reverse phase chromatography (0-50% Acetonitrile/0.05% Formic acid)) to afford (S)-3-((R)-3-(((R)-8-(5-((1R,3R)-2-(2,2-difluoroethyl)-3-methyl-2,3,4,9-tetrahydro-1H-pyrido[3,4-b]indol-1-yl)pyrimidin-2-yl)-1-oxa-8-azaspiro[4.5]decan-3-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione as yellow solid (24.62 mg, 41% yield). LC-MS purity: 100% (UV at 254 nm), 836.3[M+H]⁺.

¹H NMR (400 MHz, DMSO) δ 10.93 (s, 1H), 10.70 (s, 1H), 8.09 (s, 2H), 7.43 (d, J=7.8 Hz, 1H), 7.26 (d, J=8.0 Hz, 1H), 7.09-6.91 (m, 4H), 6.28-5.95 (m, 1H), 5.07-4.98 (m, 1H), 4.85 (s, 1H), 4.33-4.22 (m, 2H), 4.13 (d, J=16.6 Hz, 1H), 4.00-3.77 (m, 5H), 3.65-3.55 (m, 2H), 3.48-3.46 (m, 1H), 3.21-2.98 (m, 5H), 2.95-2.83 (m, 2H), 2.79-2.67 (m, 2H), 2.66-2.53 (m, 3H), 2.42-2.27 (m, 3H), 2.13-1.89 (m, 3H), 1.75 (t, J=10.8 Hz, 1H), 1.65-1.43 (m, 4H), 1.41-1.32 (m, 1H), 1.09 (d, J=6.6 Hz, 3H).

### Chroman Series

### Compound CHR-A59: (S)-3-((S)-3-((7-(4-((3S,4R)-7-hydroxy-3-phenylchroman-4-yl)phenyl)-7-azaspiro[3.5]nonan-2-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

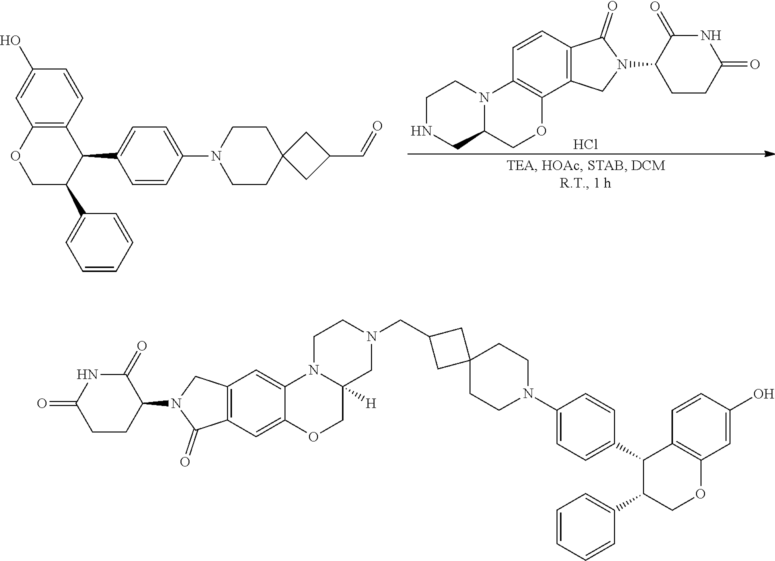 [SMILES: O=CC1CC2(CCN(c3ccc([C@@H]4c5ccc(O)cc5OC[C@@H]4c4ccccc4)cc3)CC2)C1 ; O=C1CC[C@H](N2Cc3c(ccc4c3OC[C@H]3CNCCN43)C2=O)C(=O)N1 ; Cl ; O=C1CC[C@H](N2Cc3cc4c(cc3C2=O)OC[C@@H]2CN(CC3CC5(CCN(c6ccc([C@@H]7c8ccc(O)cc8OC[C@@H]7c7ccccc7)cc6)CC5)C3)CCN42)C(=O)N1]

To a mixture of 7-(4-((3S,4R)-7-hydroxy-3-phenylchroman-4-yl)phenyl)-7-azaspiro[3.5]nonane-2-carbaldehyde (30 mg, 0.066 mmol, 1 eq.), (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride (31 mg, 0.079 mmol, 1.2 eq.), TEA (10 mg, 0.099 mmol, 1.5 eq.) in DCM (2.0 mL) was added acetic acid (6.7 mg, 0.11 mmol, 1.7 eq.) followed by sodium triacetoxyborohydride (28 mg, 0.132 mmol, 2 eq.). The mixture was stirred at room temperature for 30 minutes and concentrated. The residue was purified by reverse-phase chromatography (0-50% Acetonitrile/0.05% Formic acid)) to afford (S)-3-((S)-3-((7-(4-((3S,4R)-7-hydroxy-3-phenylchroman-4-yl)phenyl)-7-azaspiro[3.5]nonan-2-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (23 mg, 44% yield) as white solid. LC-MS purity: 100% (UV at 254 nm), 794.5 [M+H]⁺

¹H NMR (400 MHz, DMSO) δ 10.93 (s, 1H), 9.28 (s, 1H), 7.18-7.09 (m, 3H), 7.03 (s, 1H), 6.93 (s, 1H), 6.80-6.71 (m, 2H), 6.69-6.57 (m, 3H), 6.37 (d, J=8.6 Hz, 2H), 6.32-6.24 (m, 2H), 5.06-4.98 (m, 1H), 4.36-4.11 (m, 6H), 3.94-3.74 (m, 2H), 3.53-3.49 (m, 1H), 3.17-3.11 (m, 1H), 3.01-2.83 (m, 7H), 2.79-2.69 (m, 1H), 2.63-2.53 (m, 2H), 2.44-2.33 (m, 3H), 2.16-2.03 (m, 1H), 2.01-1.87 (m, 3H), 1.75-1.59 (m, 3H), 1.54-1.36 (m, 4H).

### Compound CHR-A89: (R)-3-((R)-7-((7-(4-((3S,4R)-7-hydroxy-3-phenylchroman-4-yl)phenyl)-7-azaspiro[3.5]nonan-2-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

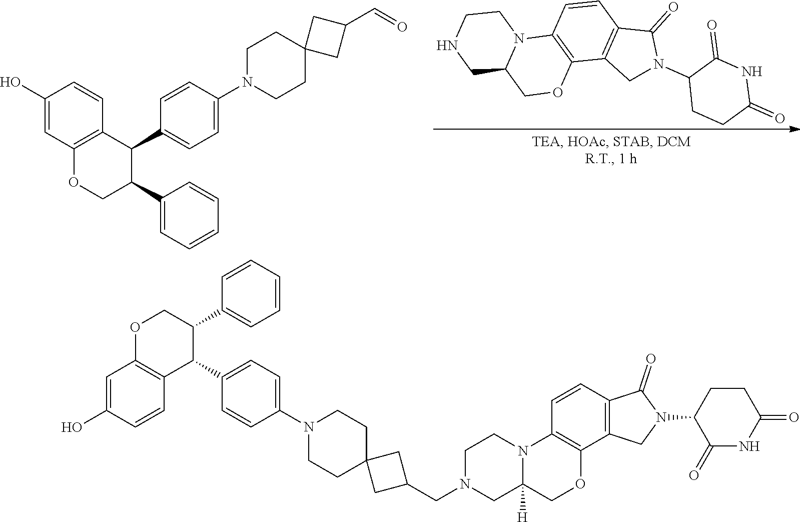

To a mixture of 7-(4-((3S,4R)-7-hydroxy-3-phenylchroman-4-yl)phenyl)-7-azaspiro[3.5]nonane-2-carbaldehyde (120 mg, 0.26 mmol, 1 eq.), (R)-3-((R)-1-oxo-1,3,5,5a,6,7,8,9-octahydro-2H-pyrazino[1′,2′:4,5][1,4]oxazino[2,3-e]isoindol-2-yl)piperidine-2,6 -dione hydrochloride (125 mg, 0.32 mmol, 1.2 eq.), TEA (40 mg, 0.396 mmol, 1.5 eq.) in DCM (5.0 mL) was added acetic acid (27 mg, 0.45 mmol, 1.7 eq.) followed by sodium triacetoxyborohydride (112 mg, 0.529 mmol, 2 eq.). The mixture was stirred at room temperature for 30 minutes and concentrated. The residue was purified by reverse-phase chromatography (0-50% acetonitrile/0.05% formic acid)) to afford (R)-3-((R)-7-((7-(4-((3S,4R)-7-hydroxy-3-phenylchroman-4-yl)phenyl)-7-azaspiro[3.5]nonan-2-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (70 mg, 34% yield) as white solid. LC-MS purity: 100% (UV at 254 nm), 794.3 [M+H]⁺

¹H NMR (400 MHz, DMSO) δ 10.92 (s, 1H), 8.23 (s, 1H), 7.24-7.08 (m, 4H), 7.00 (d, J=8.4 Hz, 1H), 6.80-6.72 (m, 2H), 6.65 (d, J=8.2 Hz, 1H), 6.60 (d, J=8.6 Hz, 2H), 6.37 (d, J=8.6 Hz, 2H), 6.33-6.23 (m, 2H), 5.09-4.90 (m, 1H), 4.36-4.08 (m, 6H), 4.01-3.90 (m, 1H), 3.80 (d, J=11.4 Hz, 1H), 3.52-3.47 (m, 1H), 3.18-3.12 (m, 1H), 3.01-2.94 (m, 2H), 2.92-2.82 (m, 5H), 2.77-2.67 (m, 1H), 2.61-2.53 (m, 2H), 2.43-2.37 (m, 3H), 2.10 (t, J=10.2 Hz, 1H), 2.00-1.88 (m, 3H), 1.73 (t, J=10.6 Hz, 1H), 1.67-1.58 (m, 2H), 1.56-1.36 (m, 4H).

### Compound CHR-A106: (R)-3-((S)-7-((1-(2-fluoro-4-((3S,4S)-7-hydroxy-3-phenylchroman-4-yl)-5-methoxyphenyl)piperidin-4-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

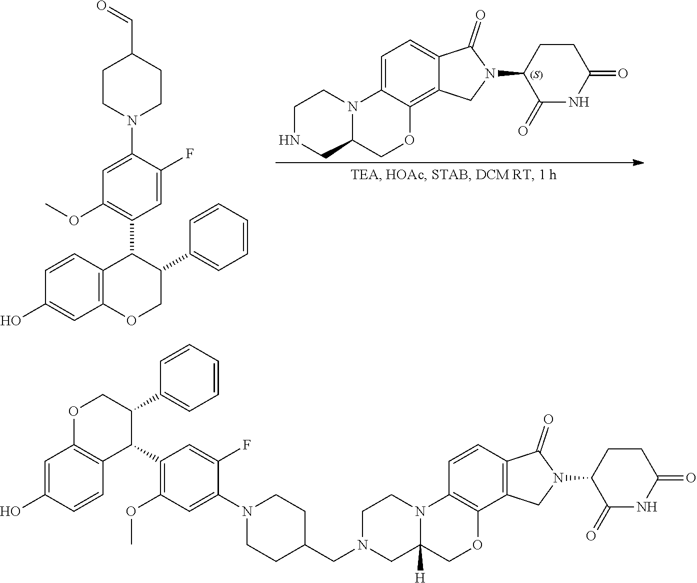

To a mixture of 1-(2-fluoro-4-((3R,4R)-7-hydroxy-3-phenylchroman-4-yl)-5-methoxyphenyl)piperidine-4-carbaldehyde (30 mg, 0.065 mmol, 1 eq.), (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione (28 mg, 0.078 mmol, 1.2 eq.), TEA (6.5 mg, 0.065 mmol, 1.0 eq.) in DCM (2.0 mL) was added acetic acid (7.8 mg, 0.130 mmol, 2.0 eq.) followed by sodium triacetoxyborohydride (28 mg, 0.130 mmol, 2 eq.). The mixture was stirred at room temperature for 30 minutes and concentrated. The residue was purified by reverse-phase chromatography (0-50% acetonitrile/0.05% formic acid)) to afford (R)-3-((S)-7-((1-(2-fluoro-4-((3S,4S)-7-hydroxy-3-phenylchroman- 4-yl)-5-methoxyphenyl)piperidin-4-yl)methyl)-1-oxo-1,3,5,5a,6,7,8,9-octahydro-2H-pyrazino[1′,2′:4,5][1,4]oxazino[2,3-c]isoindol-2-yl)piperidine-2,6-dione (28.18 mg, 54.1% yield) as a yellow solid. LC-MS purity: 98.4% (UV at 254 nm), 802.4 [M+H]⁺.

¹H NMR (400 MHz, DMSO) δ 10.93 (s, 1H), 9.32 (s, 1H), 7.22-6.96 (m, 5H), 6.77-6.55 (m, 3H), 6.36-6.20 (m, 4H), 5.03 (dd, J=13.2, 5.2 Hz, 1H), 4.66 (d, J=5.6 Hz, 1H), 4.38-4.20 (m, 3H), 4.14-4.04 (m, 2H), 4.03-3.91 (m, 1H), 3.83 (d, J=11.2 Hz, 1H), 3.52-3.45 (m, 1H), 3.29-3.22 (m, 2H), 3.21-3.14 (m, 1H), 3.06 (s, 3H), 2.97-2.85 (m, 3H), 2.80-2.68 (m, 1H), 2.66-2.52 (m, 3H), 2.43-2.32 (m, 1H), 2.27-2.18 (m, 2H), 2.14-2.04 (m, 1H), 2.01-1.90 (m, 1H), 1.85-1.59 (m, 4H), 1.36-1.17 (m, 2H).

### Compound CHR-A118: (R)-3-((R)-3-((2-(4-((3R,4S)-7-hydroxy-3-phenylchroman-4-yl)phenyl)-2-azaspiro[3.5]nonan-7-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

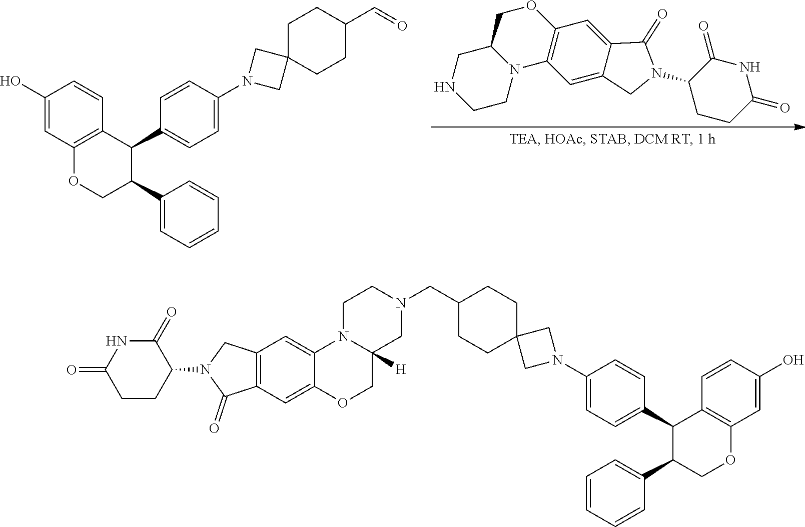 [SMILES: O=CC1CCC2(CC1)CN(c1ccc([C@@H]3c4ccc(O)cc4OC[C@@H]3c3ccccc3)cc1)C2 ; O=C1CC[C@H](N2Cc3cc4c(cc3C2=O)OC[C@@H]2CNCCN42)C(=O)N1 ; O=C1CC[C@@H](N2Cc3cc4c(cc3C2=O)OC[C@H]2CN(CC3CCC5(CC3)CN(c3ccc([C@H]6c7ccc(O)cc7OC[C@H]6c6ccccc6)cc3)C5)CCN42)C(=O)N1]

To a mixture of 2-(4-((3S,4R)-7-hydroxy-3-phenylchroman-4-yl)phenyl)-2-azaspiro[3.5]nonane-7-carbaldehyde (30 mg, 0.066 mmol, 1 eq.), (S)-3-((S)-8-oxo-1,2,3,4,4a,5,8,10-octahydro-9H-pyrazino[1′,2′:4,5][1,4]oxazino[2,3-f]isoindol-9-yl)piperidine-2,6-dione (26 mg, 0.072 mmol, 1.1 eq.), TEA (6.7 mg, 0.066 mmol, 1.0 eq.) in DCM (2.0 mL) was added acetic acid (7.9 mg, 0.132 mmol, 2.0 eq.) followed by sodium triacetoxyborohydride (28 mg, 0.132 mmol, 2 eq.). The mixture was stirred at room temperature for 30 minutes and concentrated. The residue was purified by reverse-phase chromatography (0-50% acetonitrile/0.05% formic acid)) to afford (R)-3-((R)-3-((2-(4-((3R,4S)-7-hydroxy-3-phenylchroman-4-yl)phenyl)-2-azaspiro[3.5]nonan-7-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (20.78 mg, 39.5% yield) as a yellow solid. LC-MS purity: 98.4% (UV at 254 nm), 794.4 [M+H]⁺.

¹H NMR (400 MHz, DMSO) δ 10.93 (s, 1H), 7.27-7.10 (m, 3H), 7.03 (s, 1H), 6.93 (s, 1H), 6.82-6.71 (m, 2H), 6.64 (d, J=8.4 Hz, 1H), 6.38-6.21 (m, 4H), 6.09 (d, J=8.4 Hz, 2H), 5.02 (dd, J=13.2, 5.2 Hz, 1H), 4.37-4.09 (m, 6H), 3.92-3.77 (m, 2H), 3.51-3.47 (m, 1H), 3.41-3.32 (m, 5H), 3.18-3.12 (m, 1H), 2.96-2.84 (m, 3H), 2.80-2.71 (m, 1H), 2.62-2.54 (m, 1H), 2.39-2.30 (m, 1H), 2.17-2.03 (m, 3H), 1.98-1.91 (m, 1H), 1.87-1.76 (m, 2H), 1.75-1.62 (m, 3H), 1.54-1.38 (m, 3H), 1.00-0.80 (m, 2H).

### Indazole Series

### Compound IDZ-A12. (R)-3-((S)-3-((7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonan-2-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

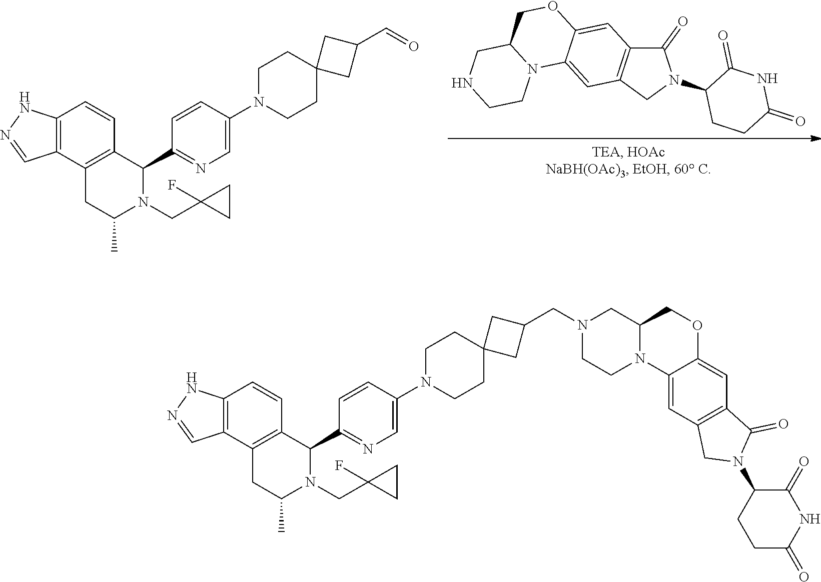 [SMILES: C[C@@H]1Cc2c(ccc3[nH]ncc23)[C@@H](c2ccc(N3CCC4(CC3)CC(C=O)C4)cn2)N1CC1(F)CC1 ; O=C1CC[C@@H](N2Cc3cc4c(cc3C2=O)OC[C@@H]2CNCCN42)C(=O)N1 ; C[C@@H]1Cc2c(ccc3[nH]ncc23)[C@@H](c2ccc(N3CCC4(CC3)CC(CN3CCN5c6cc7c(cc6OC[C@@H]5C3)C(=O)N([C@@H]3CCC(=O)NC3=O)C7)C4)cn2)N1CC1(F)CC1]

To a mixture of 7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonane-2-carbaldehyde (30 mg, 0.06 mmol, 1.0 eq) and (R)-3-((S)-8-oxo-1,2,3,4,4a,5,8,10-octahydro-9H-pyrazino[1′,2′:4,5][1,4]oxazino[2,3-f]isoindol-9-yl)piperidine-2,6-dione (24 mg, 0.06 mmol, 1.0 eq) in EtOH (5 mL) was added TEA (0.1 mL) and HOAc (0.2 mL), the mixture was stirred at 50° C. for 1 hour under N₂. The mixture was added NaBH(OAc)₃ (52 mg, 0.25 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (R)-3-((S)-3-((7-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-7-azaspiro[3.5]nonan-2-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (5.70 mg) as a yellow solid.

LC-MS purity: 99.7% (UV at 254 nm), 828.3 [M+H]⁺.

¹H NMR (400 MHz, DMSO) δ 13.17 (s, 1H), 10.95 (s, 1H), 10.12 (s, 1H), 8.27 (s, 1H), 8.16 (s, 1H), 7.38 (s, 2H), 7.19 (s, 2H), 7.04 (s, 1H), 6.91 (d, J=7.7 Hz, 1H), 5.04 (dd, J=13.3, 4.9 Hz, 1H), 4.41-4.15 (m, 5H), 4.10-3.93 (m, 2H), 3.57 (s, 4H), 3.14 (s, 7H), 2.99-2.79 (m, 3H), 2.71 (dd, J=18.1, 6.2 Hz, 1H), 2.59 (d, J=18.0 Hz, 1H), 2.38 (dd, J=17.9, 9.3 Hz, 1H), 2.10-1.95 (m, 3H), 1.75-1.50 (m, 7H), 1.27 (d, J=19.3 Hz, 3H), 1.14 (dd, J=21.0, 8.6 Hz, 2H), 0.83 (dd, J=32.7, 25.5 Hz, 2H).

### Compound IDZ-A16. (S)-3-((R)-7-((2-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonan-7-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

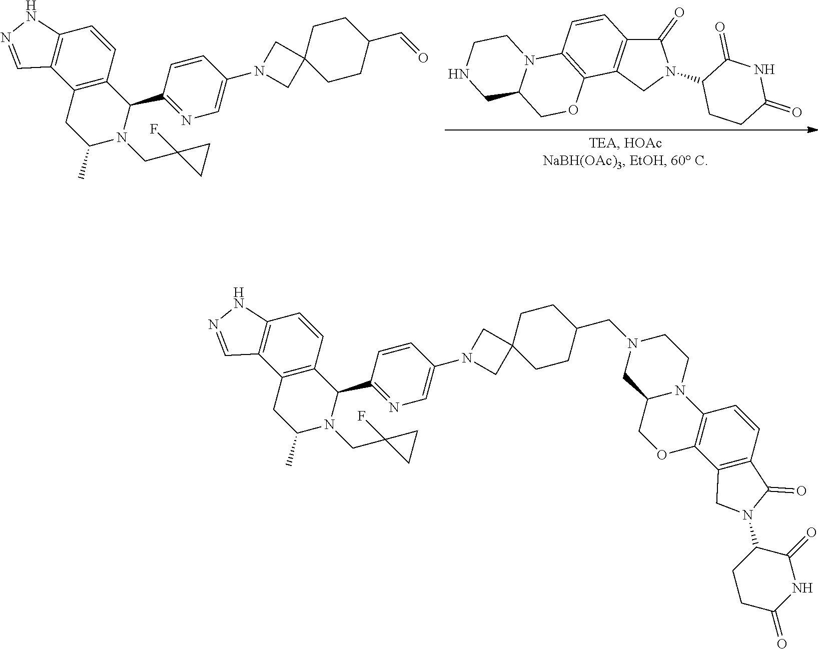

To a mixture of 2-(6-((8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonane-7-carbaldehyde (30 mg, 0.06 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione (24 mg, 0.06 mmol, 1.0 eq) in EtOH (5 mL) was added TEA (0.1 mL) and HOAc (0.2 mL), the mixture was stirred at 45° C. for 0.5 hour under N₂. The mixture was added NaBH(OAc)₃ (52 mg, 0.25 mmol, 4.0 eq) and stirred at 45° C. for 3 hours under N₂. LCMS showed the reaction was completed. The reaction was cooled to 20° C. and concentrated under vacuum. The residue was purified by Prep-HPLC (ACN/0.05% FA) to afford (R)-3-((R)-7-((2-(6-((6S,8R)-7-((1-fluorocyclopropyl)methyl)-8-methyl-6,7,8,9-tetrahydro-3H-pyrazolo[4,3-f]isoquinolin-6-yl)pyridin-3-yl)-2-azaspiro[3.5]nonan-7-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (14.87 mg) as a yellow solid.

LC-MS purity: 100% (UV at 254 nm), 828.3 [M+H]⁺.

¹H NMR (400 MHz, DMSO) δ 12.94 (s, 1H), 10.93 (s, 1H), 8.30 (s, 1H), 8.04 (s, 1H), 7.66 (d, J=2.7 Hz, 1H), 7.17 (d, J=8.4 Hz, 2H), 7.02 (dd, J=8.4, 4.9 Hz, 2H), 6.77 (d, J=8.7 Hz, 1H), 6.72 (dd, J=8.6, 2.7 Hz, 1H), 5.03 (dd, J=13.2, 5.0 Hz, 1H), 4.84 (s, 1H), 4.36 (d, J=8.4 Hz, 1H), 4.26 (d, J=16.8 Hz, 1H), 4.10 (d, J=16.9 Hz, 1H), 4.03-3.94 (m, 1H), 3.83 (d, J=11.7 Hz, 1H), 3.70 (d, J=5.3 Hz, 1H), 3.55 (s, 2H), 3.50 (s, 2H), 3.25-3.14 (m, 3H), 3.05-2.96 (m, 1H), 2.92 (d, J=13.6 Hz, 3H), 2.78-2.58 (m, 3H), 2.38 (dd, J=20.6, 11.9 Hz, 1H), 2.11 (dd, J=26.9, 8.3 Hz, 3H), 1.92 (dd, J=18.9, 9.1 Hz, 3H), 1.71 (t, J=10.4 Hz, 3H), 1.47 (t, J=11.6 Hz, 3H), 1.01 (d, J=6.5 Hz, 3H), 0.97-0.80 (m, 4H), 0.72-0.63 (m, 1H), 0.46 (dt, J=11.3, 8.0 Hz, 1H).

### Compound IDZ-A17. (S)-3-((R)-7-((2-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-2-azaspiro[3.5]nonan-7-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

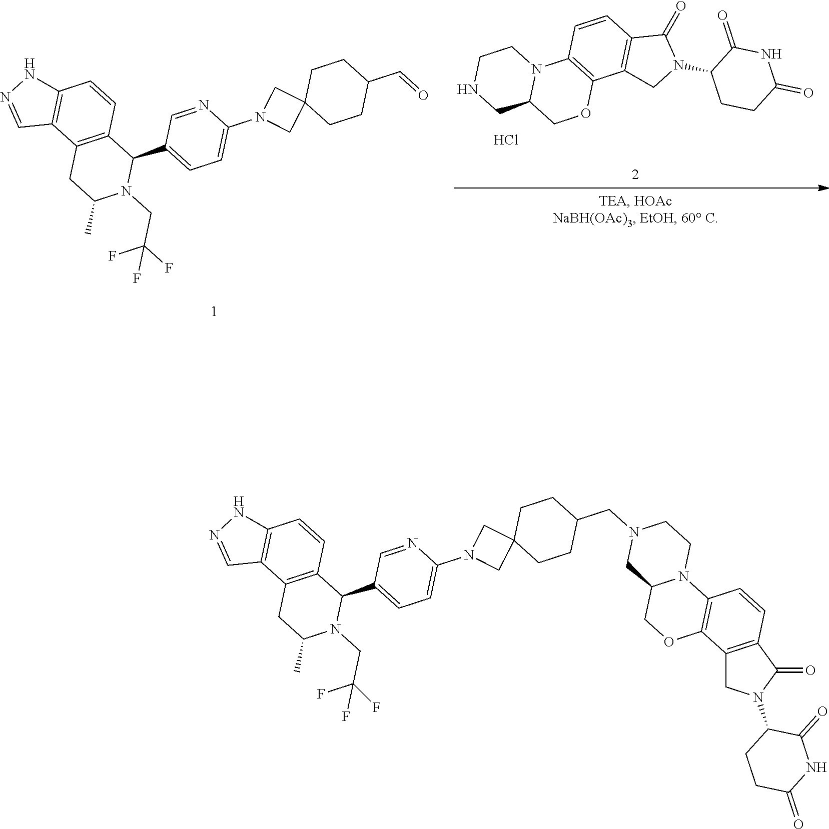 [SMILES: C[C@@H]1Cc2c(ccc3[nH]ncc23)[C@@H](c2ccc(N3CC4(CCC(C=O)CC4)C3)nc2)N1CC(F)(F)F ; O=C1CC[C@H](N2Cc3c(ccc4c3OC[C@H]3CNCCN43)C2=O)C(=O)N1 ; Cl ; C[C@@H]1Cc2c(ccc3[nH]ncc23)[C@@H](c2ccc(N3CC4(CCC(CN5CCN6c7ccc8c(c7OC[C@H]6C5)CN([C@H]5CCC(=O)NC5=O)C8=O)CC4)C3)nc2)N1CC(F)(F)F]

To a mixture of 2-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-2-azaspiro[3.5]nonane-7-carbaldehyde (190.0 mg, 0.38 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione hydrochloride (150.1 mg, 0.38 mmol, 1.0 eq) in EtOH (15 mL) was added triethylamine (0.21 mL, 1.53 mmol, 4.0 eq), followed by the addition of AcOH (0.87 mL, 15.27 mmol, 40 eq) and stirred at 25° C. for 0.5 hour. The mixture was added NaBH(OAc)₃ (323.7 mg, 1.53 mmol, 4.0 eq) and stirred at 60° C. for 2 hours. LCMS showed the reaction was completed. The reaction was cooled to 20° C. and concentrated under vacuum. The residue was purified by Prep-HPLC (ACN/0.05% FA) to afford (S)-3-((R)-7-((2-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-2-azaspiro[3.5]nonan-7-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (188.63 mg, 58.9%) as a white solid.

LCMS purity: 100% (UV at 254 nm), 838.2 [M+1]⁺.

¹H NMR (400 MHz, DMSO-d₆) δ 13.04 (s, 1H), 10.93 (s, 1H), 8.22 (dt, J=12.1, 10.0 Hz, 1H), 8.08 (s, 1H), 7.73 (d, J=2.0 Hz, 1H), 7.29 (d, J=8.5 Hz, 1H), 7.17 (d, J=8.4 Hz, 1H), 7.01 (d, J=8.5 Hz, 1H), 6.86 (d, J=8.5 Hz, 1H), 6.27 (d, J=8.7 Hz, 1H), 5.32 (t, J=4.9 Hz, 1H), 5.06 -4.93 (m, 2H), 4.38-4.33 (m, 1H), 4.28-4.23 (m, 1H), 4.10 (d, J=17.1 Hz, 1H), 3.99-3.93 (m, 1H), 3.82 (dd, J=11.0, 2.3 Hz, 1H), 3.54 (s, 3H), 3.01-2.84 (m, 7H), 2.67 (s, 1H), 2.33 (s, 1H), 2.13 (dd, J=9.7, 6.4 Hz, 2H), 1.99-1.95 (m, 2H), 1.86 (dd, J=10.6, 3.5 Hz, 2H), 1.70 (t, J=8.2 Hz, 2H), 1.45 (dd, J=18.7, 7.7 Hz, 3H), 1.24 (s, 4H), 1.07 (d, J=6.6 Hz, 3H), 0.96-0.92 (m, 1H), 0.85 (t, J=5.1 Hz, 1H).

### Compound IDZ-A19. (S)-3-((R)-7-((1-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

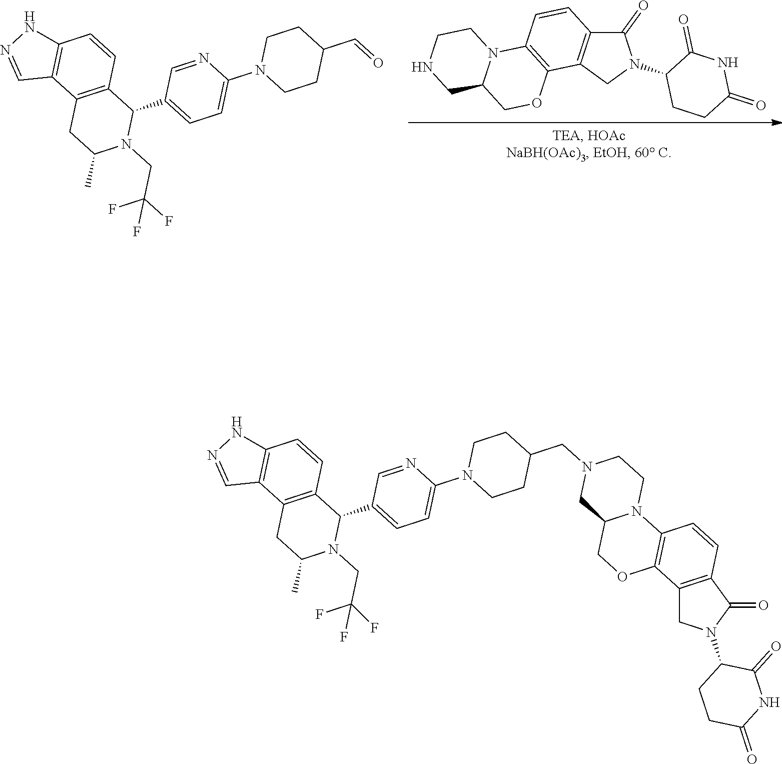 [SMILES: C[C@@H]1Cc2c(ccc3[nH]ncc23)[C@H](c2ccc(N3CCC(C=O)CC3)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@H](c2ccc(N3CCC(CN4CCN5c6ccc7c(c6OC[C@H]5C4)CN([C@H]4CCC(=O)NC4=O)C7=O)CC3)nc2)N1CC(F)(F)F]

To a mixture of 1-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)piperidine-4-carbaldehyde (30 mg, 0.07 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione (26 mg, 0.07 mmol, 1.0 eq) in EtOH (5 mL) was added TEA (0.1 mL) and HOAc (0.2 mL), the mixture was stirred at 50° C. for 1 hour under N₂. The mixture was added NaBH(OAc)₃ (56 mg, 0.26 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (S)-3-((R)-7-((1-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (3.85 mg) as a white solid.

LC-MS purity: 100% (UV at 254 nm), 798.1 [M+H]⁺.

¹H NMR (400 MHz, MeOD) δ 8.39 (s, 1H), 7.99 (s, 1H), 7.68 (s, 1H), 7.31 (d, J=7.3 Hz, 1H), 7.24 (d, J=8.3 Hz, 1H), 7.19 (d, J=8.4 Hz, 1H), 6.89 (dd, J=17.7, 8.5 Hz, 2H), 6.68 (d, J= 8.8 Hz, 1H), 4.98 (dd, J=13.5, 5.3 Hz, 1H), 4.91 (s, 1H), 4.24 (dd, J=17.3, 9.3 Hz, 3H), 4.12 (d, J=12.3 Hz, 2H), 3.93 (t, J=9.4 Hz, 1H), 3.73 (d, J=11.3 Hz, 1H), 3.33 (dd, J=19.5, 12.4 Hz, 2H), 2.98 (d, J=17.5 Hz, 2H), 2.87 (d, J=9.1 Hz, 2H), 2.83-2.73 (m, 5H), 2.67 (d, J=15.2 Hz, 1H), 2.37 (dd, J=23.7, 15.1 Hz, 1H), 2.19 (d, J=6.0 Hz, 2H), 2.15-2.02 (m, 2H), 1.83-1.73 (m, 4H), 1.17 (d, J=19.3 Hz, 3H), 1.06 (d, J=6.7 Hz, 3H).

### Compound IDZ-A21. (3S)-3-((5aR)-7-((8-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decan-3-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

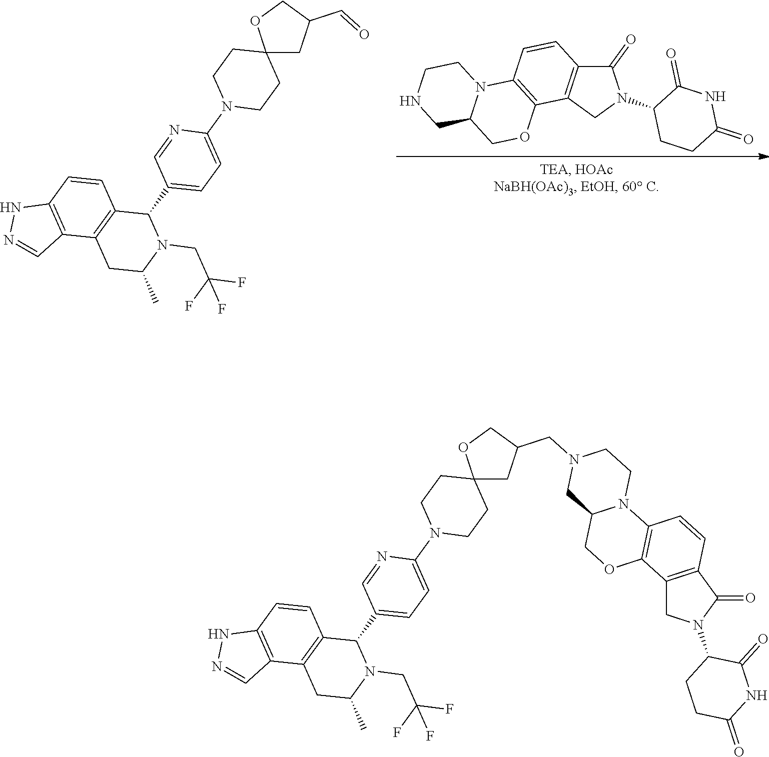 [SMILES: C[C@@H]1Cc2c(ccc3[nH]ncc23)[C@H](c2ccc(N3CCC4(CC3)CC(C=O)CO4)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@H](c2ccc(N3CCC4(CC3)CC(CN3CCN5c6ccc7c(c6OC[C@H]5C3)CN([C@H]3CCC(=O)NC3=O)C7=O)CO4)nc2)N1CC(F)(F)F]

To a mixture of 8-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decane-3-carbaldehyde (30 mg, 0.06 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione (23 mg, 0.06 mmol, 1.0 eq) in EtOH (5 mL) was added TEA (0.1 mL) and HOAc (0.2 mL), the mixture was stirred at 50° C. for 1 hour under N₂. The mixture was added NaBH(OAc)₃ (50 mg, 0.23 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (S)-3-((R)-7-(((R)-8-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-1-oxa-8-azaspiro[4.5]decan-3-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (3.11 mg) as a white solid.

LCMS purity: 100% (UV at 254 nm), 854.2 [M+1]⁺.

¹H NMR (400 MHz, DMSO) δ 13.05 (s, 1H), 10.93 (s, 1H), 8.32 (s, 1H), 8.08 (s, 1H), 7.77 (s, 1H), 7.30 (d, J=8.7 Hz, 1H), 7.22-7.17 (m, 1H), 7.01 (d, J=8.5 Hz, 1H), 6.88 (d, J=8.7 Hz, 1H), 6.76 (d, J=8.9 Hz, 1H), 5.05-5.00 (m, 1H), 4.95 (s, 1H), 4.36 (d, J=9.9 Hz, 1H), 4.26 (d, J=17.0 Hz, 1H), 4.10 (d, J=16.7 Hz, 1H), 3.98-3.90 (m, 2H), 3.83 (d, J=10.7 Hz, 1H), 3.61-3.57 (m, 2H), 3.46 (s, 2H), 3.18-3.10 (m, 2H), 3.00 (d, J=12.0 Hz, 2H), 2.91 (d, J=12.2 Hz, 2H), 2.84 (d, J=17.7 Hz, 2H), 2.74 (s, 1H), 2.67 (s, 1H), 2.35 (d, J=13.8 Hz, 3H), 2.15-2.06 (m, 1H), 1.97 (dd, J=14.7, 7.0 Hz, 2H), 1.77-1.70 (m, 1H), 1.62-1.53 (m, 4H), 1.33 (dd, J=26.5, 13.1 Hz, 2H), 1.08 (d, J=6.6 Hz, 3H).

### Compound IDZ-A24. (S)-3-((S)-3-((1-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione

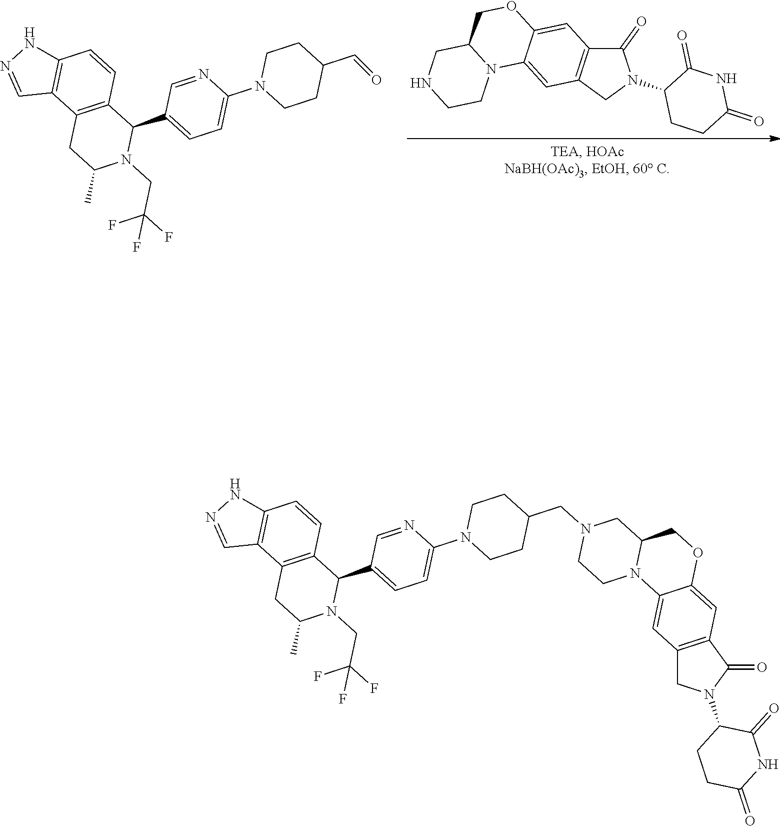 [SMILES: C[C@@H]1Cc2c(ccc3[nH]ncc23)[C@@H](c2ccc(N3CCC(C=O)CC3)nc2)N1CC(F)(F)F ; O=C1CC[C@H](N2Cc3cc4c(cc3C2=O)OC[C@@H]2CNCCN42)C(=O)N1 ; C[C@@H]1Cc2c(ccc3[nH]ncc23)[C@@H](c2ccc(N3CCC(CN4CCN5c6cc7c(cc6OC[C@@H]5C4)C(=O)N([C@H]4CCC(=O)NC4=O)C7)CC3)nc2)N1CC(F)(F)F]

To a mixture of 1-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)piperidine-4-carbaldehyde (17 mg, 0.04 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione (15 mg, 0.04 mmol, 1.0 eq) in EtOH (5 mL) was added TEA (0.1 mL) and HOAc (0.2 mL), the mixture was stirred at 50° C. for 1 hour under N₂. The mixture was added NaBH(OAc)₃ (32 mg, 0.15 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (S)-3-((S)-3-((1-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)piperidin-4-yl)methyl)-8-oxo-1,2,3,4,4a,5,8,10-octahydro-9H-pyrazino[1′,2′:4,5][1,4]oxazino[2,3-f]isoindol-9-yl)piperidine-2,6-dione (2.15 mg) as a white solid.

LCMS purity: 100% (UV at 254 nm), 798.2 [M+1]⁺.

¹H NMR (400 MHz, DMSO) δ 13.04 (s, 1H), 10.93 (s, 1H), 8.18 (s, 1H), 8.08 (s, 1H), 7.77 (s, 1H), 7.30 (d, J=8.6 Hz, 1H), 7.21 (d, J=6.8 Hz, 1H), 7.04 (s, 1H), 6.93 (s, 1H), 6.88 (d, J=8.6 Hz, 1H), 6.74 (d, J=8.9 Hz, 1H), 5.02 (dd, J=13.2, 5.0 Hz, 1H), 4.95 (s, 1H), 4.26 (dd, J=19.1, 11.3 Hz, 4H), 4.15 (d, J=16.7 Hz, 1H), 3.96-3.87 (m, 1H), 3.81 (d, J=10.7 Hz, 1H), 3.53 (dd, J=16.1, 9.7 Hz, 2H), 3.06-2.96 (m, 3H), 2.93 (d, J=6.4 Hz, 3H), 2.80-2.67 (m, 4H), 2.35 (d, J=12.9 Hz, 1H), 2.23-2.15 (m, 2H), 2.12-2.06 (m, 1H), 2.03-1.91 (m, 2H), 1.81-1.70 (m, 4H), 1.24 (s, 2H), 1.08 (d, J=6.6 Hz, 3H).

### Compound IDZ-A25. (S)-3-((R)-7-((7-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

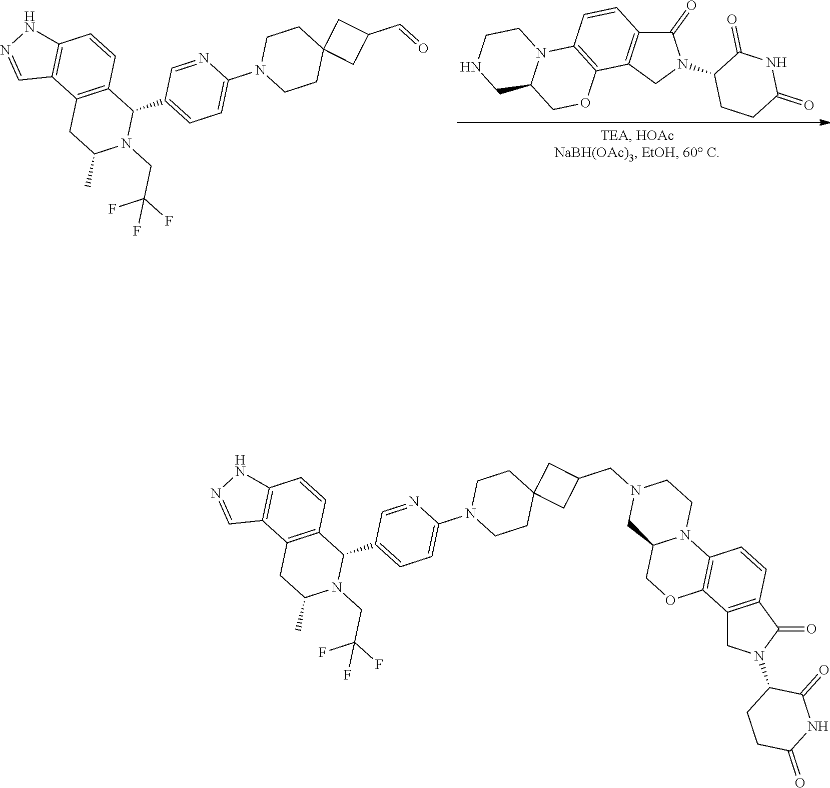 [SMILES: C[C@@H]1Cc2c(ccc3[nH]ncc23)[C@H](c2ccc(N3CCC4(CC3)CC(C=O)C4)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@H](c2ccc(N3CCC4(CC3)CC(CN3CCN5c6ccc7c(c6OC[C@H]5C3)CN([C@H]3CCC(=O)NC3=O)C7=O)C4)nc2)N1CC(F)(F)F]

To a mixture of 7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonane-2-carbaldehyde (25 mg, 0.05 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione (20 mg, 0.05 mmol, 1.0 eq) in EtOH (5 mL) was added TEA (0.1 mL) and HOAc (0.2 mL), the mixture was stirred at 50° C. for 1 hour under N₂. The mixture was added NaBH(OAc)₃ (43 mg, 0.20 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (S)-3-((R)-7-((7-(5-((6S,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (1.85 mg) as a white solid.

LCMS purity: 100% (UV at 254 nm), 838.2 [M+1]⁺.

¹H NMR (400 MHz, MeOD) δ 8.15 (s, 1H), 8.06 (d, J=9.5 Hz, 1H), 7.43 (d, J=8.6 Hz, 2H), 7.38 (d, J=8.3 Hz, 1H), 7.29 (s, 1H), 7.16 (d, J=8.5 Hz, 1H), 7.07 (d, J=8.7 Hz, 1H), 5.36 (t, J=4.6 Hz, 2H), 5.11 (d, J=11.1 Hz, 2H), 4.50-4.43 (m, 1H), 4.38 (d, J=7.8 Hz, 1H), 4.33-4.26 (m, 1H), 4.17 (dd, J=11.1, 7.3 Hz, 1H), 3.74-3.70 (m, 2H), 3.62 (t, J=6.9 Hz, 3H), 3.08 (d, J=5.4 Hz, 1H), 3.02-2.93 (m, 2H), 2.91-2.88 (m, 1H), 2.81 (t, J=4.8 Hz, 1H), 2.28 (t, J=10.5 Hz, 3H), 2.24-2.19 (m, 3H), 2.05 (dd, J=12.5, 6.5 Hz, 4H), 1.92 (dd, J=7.1, 4.7 Hz, 2H), 1.82-1.76 (m, 3H), 1.65-1.61 (m, 2H), 1.21 (d, J=6.6 Hz, 3H), 0.93 (d, J=6.6 Hz, 3H).

### Compound IDZ-A26. (S)-3-((R)-7-((7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione

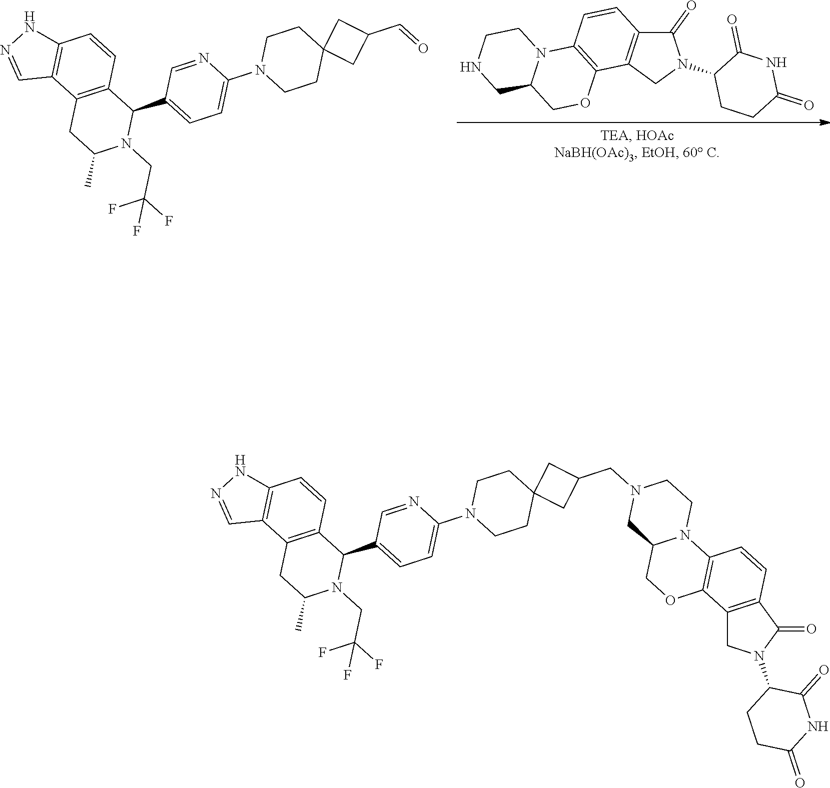 [SMILES: C[C@@H]1Cc2c(ccc3[nH]ncc23)[C@@H](c2ccc(N3CCC4(CC3)CC(C=O)C4)nc2)N1CC(F)(F)F ; O=C1CC[C@H](N2Cc3c(ccc4c3OC[C@H]3CNCCN43)C2=O)C(=O)N1 ; C[C@@H]1Cc2c(ccc3[nH]ncc23)[C@@H](c2ccc(N3CCC4(CC3)CC(CN3CCN5c6ccc7c(c6OC[C@H]5C3)CN([C@H]3CCC(=O)NC3=O)C7=O)C4)nc2)N1CC(F)(F)F]

To a mixture of 7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonane-2-carbaldehyde (25 mg, 0.05 mmol, 1.0 eq) and (S)-3-((R)-1-oxo-1,3,5,5a,6,7,8,9-octahydro-2H-pyrazino[1′,2′:4,5][1,4]oxazino[2,3-e]isoindol-2-yl)piperidine-2,6-dione (20 mg. 0.05 mmol, 1.0 eq) in EtOH (5 mL) was added TEA (0.1 mL) and HOAc (0.2 mL), the mixture was stirred at 50° C. for 1 hour under N₂. The mixture was added NaBH(OAc)₃ (43 mg, 0.20 mmol, 4.0 eq) and stirred at 50° C. for 2 hours under N₂. LCMS showed the reaction was completed. The reaction was cooled to 20° C. and concentrated under vacuum. The residue was purified by Prep-HPLC (ACN/0.05% FA) to afford (S)-3-((R)-7-((7-(5-((6R,8R)-8-methyl-7-(2,2,2-trifluoroethyl)-6,7,8,9-tetrahydro-3H-pyrazolo[4,3-f]isoquinolin-6-yl)pyridin-2-yl)-7-azaspiro[3.5]nonan-2-yl)methyl)-1-oxo-1,3,5,5a,6,7,8,9-octahydro-2H-pyrazino[1′,2′:4,5][1,4]oxazino[2,3-e]isoindol-2-yl)piperidine-2,6-dione (4.03 mg) as a white solid.

LCMS purity: 100% (UV at 254 nm), 838.2 [M+1]⁺.

¹H NMR (400 MHz, MeOD) δ 8.16 (s, 1H), 8.06 (dd, J=9.7, 1.9 Hz, 1H), 7.46-7.36 (m, 3H), 7.29 (s, 1H), 7.16 (d, J=8.3 Hz, 1H), 7.07 (d, J=8.6 Hz, 1H), 5.36 (t, J=4.8 Hz, 1H), 5.11 (d, J=11.6 Hz, 2H), 4.47 (dd, J=11.1, 2.4 Hz, 1H), 4.38 (d, J=7.9 Hz, 1H), 4.28 (dd, J=9.1, 3.7 Hz, 1H), 4.17 (dd, J=11.1, 7.0 Hz, 1H), 3.71 (d, J=4.4 Hz, 2H), 3.62 (s, 2H), 3.54 (dd, J=18.2, 11.7 Hz, 2H), 3.26-3.23 (m, 1H), 3.10-3.05 (m, 1H), 2.98 (d, J=8.8 Hz, 1H), 2.90 (d, J=13.4 Hz, 2H), 2.79 (d, J=15.6 Hz, 1H), 2.50 (dd, J=13.8, 5.5 Hz, 1H), 2.31-2.21 (m, 3H), 2.05 (d, J=5.8 Hz, 1H), 1.93 (s, 1H), 1.82-1.77 (m, 3H), 1.65-1.61 (m, 1H), 1.36-1.31 (m, 8H), 1.21 (d, J=6.7 Hz, 3H).

### Synthetic Procedures for Making Selective IKZF2 or IKZF1/3 Degraders

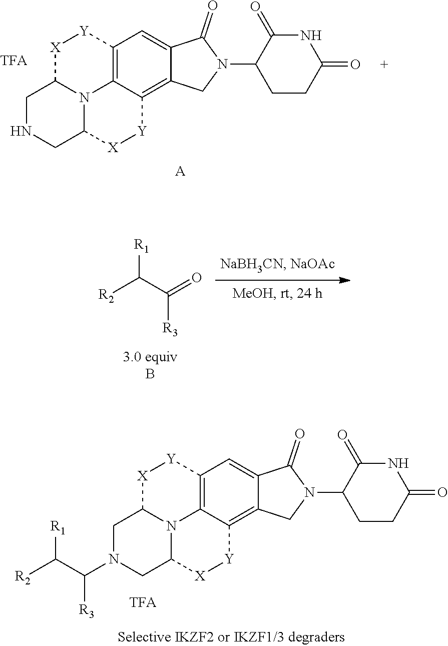

TABLE E4

| Com- | | | |
|---|---|---|---|
| pound | | LC-MS | |
| No. | Structures | [M + H]⁺ | NMR |
| C1 | 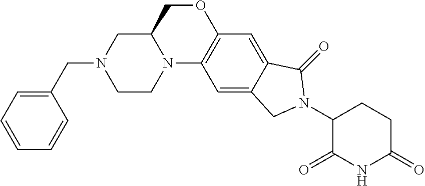 [SMILES: O=C1CCC(N2Cc3cc4c(cc3C2=O)OC[C@@H]2CN(Cc3ccccc3)CCN42)C(=O)N1] | 447.16 | ¹H NMR (400 MHz, Methanol-d₄) δ 7.58-7.49 (m, 5H), 7.17 (d, J = 1.0 Hz, 1H), 7.13 (s, 1H), 5.13-5.03 (m, 1H), 4.48-4.39 (m, 2H), 4.38-4.30 (m, 3H), 4.30-4.20 (m, 1H), 4.11-4.02 (m, 1H), 3.68-3.51 (m, 3H), 3.29-3.24 (m, 2H), 3.10-3.00 (m, 1H), 2.96-2.83 (m, 1H), 2.82-2.72 (m, 1H), 2.53-2.37 (m, 1H), 2.20-2.09 (m, 1H). |
| C2 | 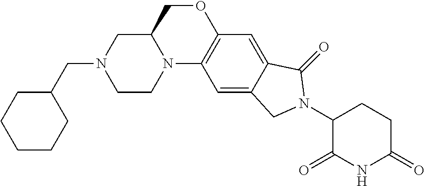 [SMILES: O=C1CCC(N2Cc3cc4c(cc3C2=O)OC[C@@H]2CN(CC3CCCCC3)CCN42)C(=O)N1] | 453.22 | |
| C3 | 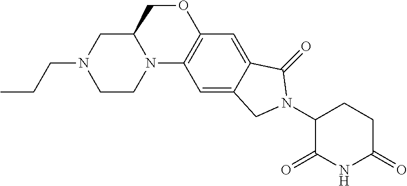 [SMILES: CCCN1CCN2c3cc4c(cc3OC[C@@H]2C1)C(=O)N(C1CCC(=O)NC1=O)C4] | 399.19 | |
| C4 | 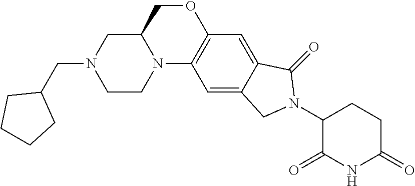 [SMILES: O=C1CCC(N2Cc3cc4c(cc3C2=O)OC[C@@H]2CN(CC3CCCC3)CCN42)C(=O)N1] | 439.20 | ¹H NMR (400 MHz, Methanol-d₄) δ 7.18 (s, 1H), 7.14 (d, J = 2.1 Hz, 1H), 5.15- 5.02 (m, 1H), 4.44-4.31 (m, 3H), 4.29- 4.19 (m, 1H), 4.14-4.03 (m, 1H), 3.79- 3.59 (m, 3H), 3.29-3.15 (m, 4H), 3.04- 2.83 (m, 2H), 2.82-2.72 (m, 1H), 2.53- 2.29 (m, 2H), 2.20-2.09 (m, 1H), 2.03- 1.91 (m, 2H), 1.79-1.61 (m, 4H), 1.37- 1.26 (m, 2H). |
| C5 | 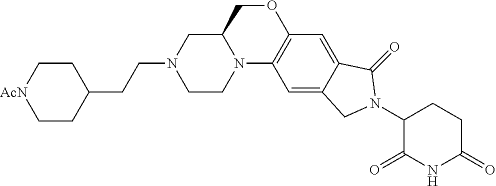 | 510.20 | |
| C6 | 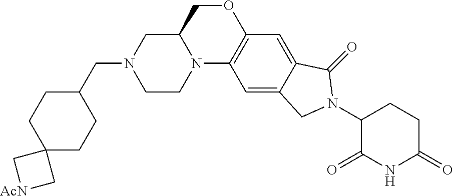 | 536.22 | ¹H NMR (400 MHz, Methanol-d₄) δ 7.18 (s, 1H), 7.15 (s, 1H), 5.12-5.04 (m, 1H), 4.42-4.30 (m, 3H), 4.28-4.18 (m, 1H), 4.13-4.04 (m, 1H), 3.88 (d, J = 23.7 Hz, 2H), 3.75-3.58 (m, 5H), 3.30-3.16 (m, 2H), 3.14-3.06 (m, 2H), 3.02-2.83 (m, 2H), 2.19-2.10 (m, 1H), 2.01- 1.78 (m, 8H), 1.66-1.54 (m, 2H), 1.21- 1.08 (m, 2H). |
| C7 | 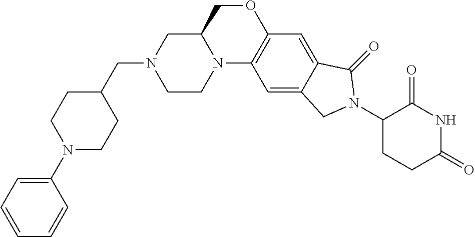 [SMILES: O=C1CCC(N2Cc3cc4c(cc3C2=O)OC[C@@H]2CN(CC3CCN(c5ccccc5)CC3)CCN42)C(=O)N1] | 529.11 | |
| C8 | 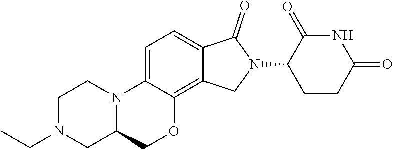 [SMILES: CCN1CCN2c3ccc4c(c3OC[C@H]2C1)CN([C@H]1CCC(=O)NC1=O)C4=O] | 385.12 | |
| C9 | 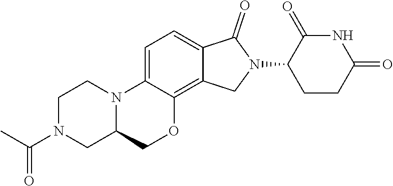 [SMILES: CC(=O)N1CCN2c3ccc4c(c3OC[C@H]2C1)CN([C@H]1CCC(=O)NC1=O)C4=O] | 399.18 | |
| C10 | 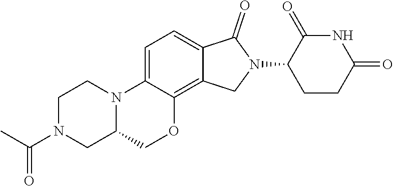 [SMILES: CC(=O)N1CCN2c3ccc4c(c3OC[C@@H]2C1)CN([C@H]1CCC(=O)NC1=O)C4=O] | 399.13 | |
| C11 | 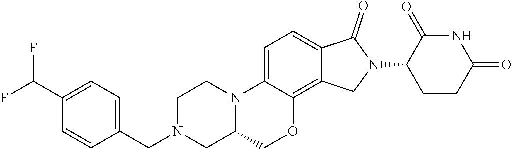 [SMILES: O=C1CC[C@H](N2Cc3c(ccc4c3OC[C@@H]3CN(Cc5ccc(C(F)F)cc5)CCN43)C2=O)C(=O)N1] | 497.19 | ¹H NMR (400 MHz, Methanol-d₄) δ 7.80-7.58 (m, 3H), 7.37 (dd, J = 8.4, 1.8 Hz, 1H), 7.14 (dd, J = 8.6, 1.5 Hz, 1H), 6.85 (t, J = 56.0 Hz, 1H), 5.11 (dt, J = 13.3, 5.5 Hz, 1H), 4.52-4.28 (m, 4H), 4.28-4.07 (m, 2H), 3.70-3.43 (m, 3H), 3.30-3.22 (m, 2H), 3.20 (d, J = 8.2 Hz, 2H), 3.05-2.71 (m, 3H), 2.49 (qt, J = 13.3, 5.0 Hz, 1H), 2.16 (dtd, J = 12.8, 2.4 Hz, 1H) |
| C12 | 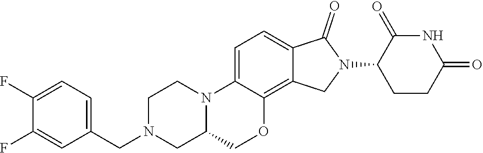 [SMILES: O=C1CC[C@H](N2Cc3c(ccc4c3OC[C@@H]3CN(Cc5ccc(F)c(F)c5)CCN43)C2=O)C(=O)N1] | 483.18 | ¹H NMR (400 MHz, Methanol-d₄) δ 7.52 (ddd, J = 11.0, 7.5, 2.1 Hz, 1H), 7.43 (dt, J = 9.9, 8.1 Hz, 1H), 7.37 (dq, J = 8.4, 2.4 Hz, 2H), 7.13 (dd, J = 8.4, 1.2 Hz, 1H), 5.11 (ddd, J = 13.0, 7.5, 5.2 Hz, 1H), 4.47-4.27 (m, 5H), 4.24 (brs, 1H), 4.15 (ddd, J = 11.0, 7.0, 5.8 Hz, 1H), 3.64-3.53 (m, 2H), 3.49 (d, J = 12.9 Hz, 1H), 3.26-3.11 (m, 2H), 3.01- 2.84 (m, 2H), 2.79 (ddd, J = 17.6, 4.7, |
| | | | 2.4 Hz, 1H), 2.49 (qt, J = 13.3, 5.2 Hz, |
| | | | 1H), 2.16 (dtd, J = 12.8, 5.2, 2.4 Hz, 1H). |
| C13 | 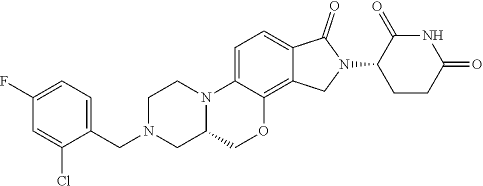 [SMILES: O=C1CC[C@H](N2Cc3c(ccc4c3OC[C@@H]3CN(Cc5ccc(F)cc5Cl)CCN43)C2=O)C(=O)N1] | 499.19 | ¹H NMR (400 MHz, Methanol-d₄) δ 7.75 (dd, J = 8.7, 5.9 Hz, 1H), 7.48 (ddd, J = 8.5, 2.6, 0.9 Hz, 1H), 7.37 (dd, J = 8.3, 1.5 Hz, 1H), 7.32-7.23 (m, 1H), 7.14 (d, J = 8.4 Hz, 1H), 5.11 (dt, J = 13.3, 5.5 Hz, 1H), 4.56 (s, 2H), 4.49-4.34 (m, 2H), 4.34-4.08 (m, 2H), 3.63 (d, J = 12.1 Hz, 3H), 3.30-3.22 (m, 2H), 3.29- 3.06 (m, 2H), 2.98-2.71 (m, 2H), 2.49 (dddd, J = 18.2, 13.1, 9.0, 4.9 Hz, 1H), 2.16 (dtd, J = 12.8, 5.3, 2.4 Hz, 1H). |
| C14 | 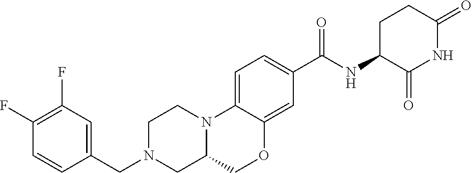 [SMILES: O=C1CC[C@H](NC(=O)c2ccc3c(c2)OC[C@@H]2CN(Cc4ccc(F)c(F)c4)CCN32)C(=O)N1] | 471.18 | ¹H NMR (400 MHz, Methanol-d₄) δ 7.51-7.32 (m, 5H), 7.01 (d, J = 8.7 Hz, 1H), 4.33 (dd, J = 11.0, 2.9 Hz, 1H), 4.23-4.11 (m, 3H), 4.06 (dd, J = 11.1, 7.4 Hz, 1H), 3.53-3.48 (m, 2H), 3.16- 3.04 (m, 3H), 2.86-2.66 (m, 4H), 2.25- 2.16 (m, 2H). |
| C15 | 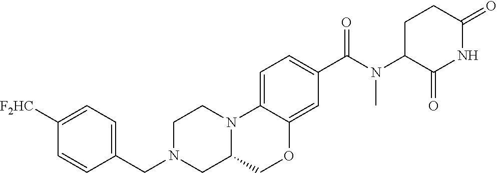 [SMILES: CN(C(=O)c1ccc2c(c1)OC[C@@H]1CN(Cc3ccc(C(F)F)cc3)CCN21)C1CCC(=O)NC1=O] | 499.21 | ¹H NMR (400 MHz, Methanol-d₄) δ 7.70 (q, J = 8.2 Hz, 4H), 7.14-6.94 (m, 3H), 6.79 (d, J = 55.9 Hz, 1H), 4.53- 4.38 (m, 2H), 4.38-4.27 (m, 1H), 4.18 (d, J = 13.6 Hz, 1H), 4.07 (dd, J = 11.1, 7.0 Hz, 1H), 3.54 (dd, J = 30.0, 11.8 Hz, 3H), 3.23 (d, J = 11.7 Hz, 1H), 31.9-3.08 (m, 1H), 2.99 (d, J = 37.5 Hz, 3H), 2.89- 2.58 (m, 2H), 2.50 (d, J = 13.0 Hz, 1H), 2.24-2.05 (m, 1H) |
| C16 | 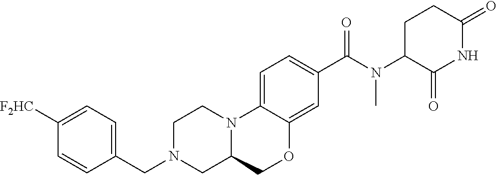 [SMILES: CN(C(=O)c1ccc2c(c1)OC[C@H]1CN(Cc3ccc(C(F)F)cc3)CCN21)C1CCC(=O)NC1=O] | 499.21 | ¹H NMR (400 MHz, Methanol-d₄) δ 7.71 (q, J = 8.2 Hz, 4H), 7.16-6.95 (m, 3H), 6.79 (d, J = 55.9 Hz, 1H), 4.48 (s, 2H), 4.34 (d, J = 11.0 Hz, 1H), 4.20 (d, J = 13.9 Hz, 1H), 4.07 (t, J = 9.2 Hz, 1H), 3.61 (s, 3H), 3.32 (d, J = 1.7 Hz, 3H), 3.13 (s, 1H), 2.99 (d, J = 37.0 Hz, 4H), 2.75 (q, J = 23.3 Hz, 2H), 2.59-2.38 (m, 1H), 2.15 (s, 1H). |
| C17 | 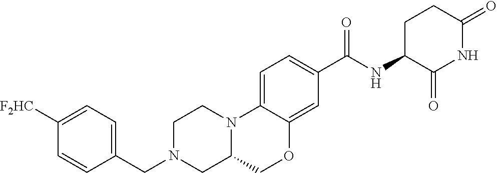 [SMILES: O=C1CC[C@H](NC(=O)c2ccc3c(c2)OC[C@@H]2CN(Cc4ccc(C(F)F)cc4)CCN32)C(=O)N1] | 485.18 | ¹H NMR (400 MHz, Methanol-d₄) δ 8.54 (d, J = 8.0 Hz, 1H), 7.69 (q, J = 8.2 Hz, 4H), 7.47 (dd, J = 8.6, 2.1 Hz, 1H), 7.36 (d, J = 2.1 Hz, 1H), 7.03 (d, J = 8.7 Hz, 1H), 6.85 (t, J = 56.0 Hz, 1H), 4.46- 4.29 (m, 3H), 4.19 (d, J = 11.0 Hz, 1H), 4.07 (dd, J = 11.1, 7.3 Hz, 1H), 3.60-3.41 (m, 3H), 3.40-3.35 (m, 2H), 3.22-3.03 (m, 2H), 2.99-2.64 (m, 3H), 2.27-2.12 (m, 2H). |
| C18 | 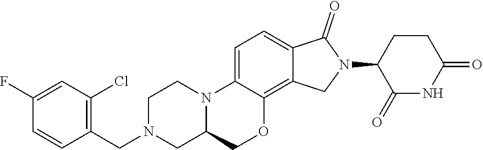 [SMILES: O=C1CC[C@H](N2Cc3c(ccc4c3OC[C@H]3CN(Cc5ccc(F)cc5Cl)CCN43)C2=O)C(=O)N1] | 499.15 | ¹H NMR (400 MHz, Methanol-d₄) δ 7.74 (dd, J = 8.6, 5.9 Hz, 1H), 7.47 (dd, J = 8.6, 2.6 Hz, 1H), 7.37 (dd, J = 8.3, 1.5 Hz, 1H), 7.34-7.21 (m, 1H), 7.14 (dd, J = 8.5, 1.4 Hz, 1H), 5.11 (dt, J = 13.3, 4.8 Hz, 1H), 4.52-4.29 (m, 4H), 4.29-4.09 (m, 2H), 3.67-3.47 (m, 3H), 3.41-3.34 (m, 1H), 3.30-3.12 (m, 2H), 3.05 (t, J = 11.2 Hz, 1H), 2.91 (ddd, J = 18.3, 13.5, 5.5 Hz, 1H), 2.79 (ddd, J = |
| | | | 17.7, 4.7, 2.4 Hz, 1H), 2.49 (qt, J = 13.2, |
| | | | 4.5 Hz, 1H), 2.16 (dtd, J = 12.8, 5.2, 2.4 |
| | | | Hz, 1H). |
| C19 | 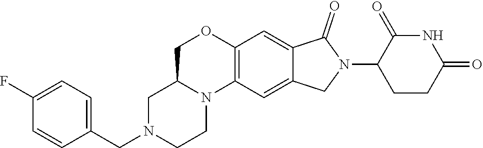 [SMILES: O=C1CCC(N2Cc3cc4c(cc3C2=O)OC[C@@H]2CN(Cc3ccc(F)cc3)CCN42)C(=O)N1] | 465.23 | |
| C20 | 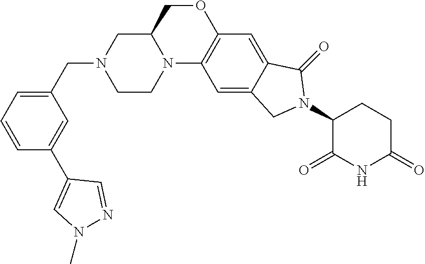 [SMILES: Cn1cc(-c2cccc(CN3CCN4c5cc6c(cc5OC[C@@H]4C3)C(=O)N([C@H]3CCC(=O)NC3=O)C6)c2)cn1] | 527.31 | |
| C21 | 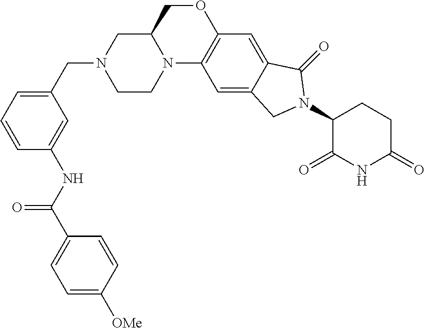 [SMILES: COc1ccc(C(=O)Nc2cccc(CN3CCN4c5cc6c(cc5OC[C@@H]4C3)C(=O)N([C@H]3CCC(=O)NC3=O)C6)c2)cc1] | 596.43 | |
| C22 | 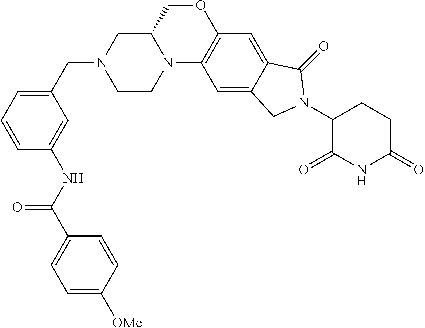 [SMILES: COc1ccc(C(=O)Nc2cccc(CN3CCN4c5cc6c(cc5OC[C@H]4C3)C(=O)N(C3CCC(=O)NC3=O)C6)c2)cc1] | 596.40 | |
| C23 | 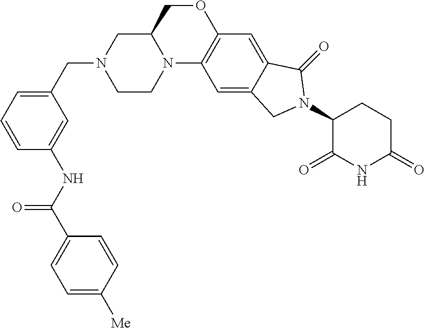 [SMILES: Cc1ccc(C(=O)Nc2cccc(CN3CCN4c5cc6c(cc5OC[C@@H]4C3)C(=O)N([C@H]3CCC(=O)NC3=O)C6)c2)cc1] | 580.43 | |
| C24 | 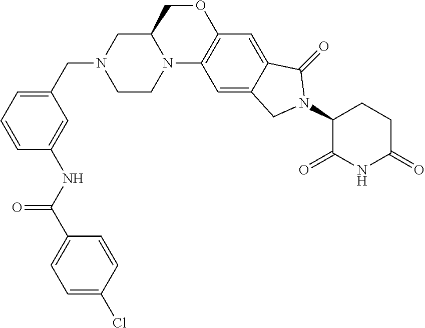 [SMILES: O=C1CC[C@H](N2Cc3cc4c(cc3C2=O)OC[C@@H]2CN(Cc3cccc(NC(=O)c5ccc(Cl)cc5)c3)CCN42)C(=O)N1] | 600.35 | |
| C25 | 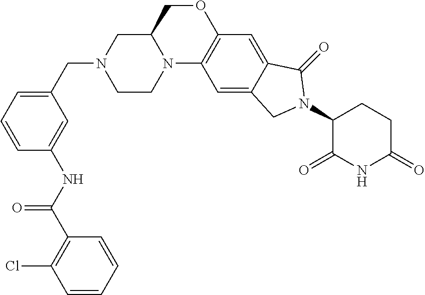 [SMILES: O=C1CC[C@H](N2Cc3cc4c(cc3C2=O)OC[C@@H]2CN(Cc3cccc(NC(=O)c5ccccc5Cl)c3)CCN42)C(=O)N1] | 600.38 | |
| C26 | 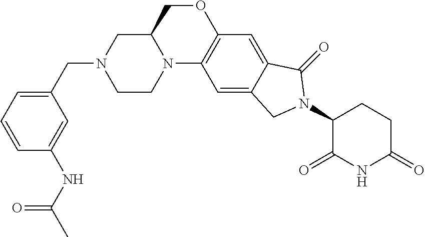 [SMILES: CC(=O)Nc1cccc(CN2CCN3c4cc5c(cc4OC[C@@H]3C2)C(=O)N([C@H]2CCC(=O)NC2=O)C5)c1] | 504.31 | |
| C27 | 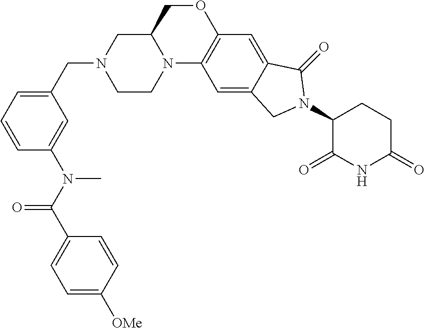 [SMILES: COc1ccc(C(=O)N(C)c2cccc(CN3CCN4c5cc6c(cc5OC[C@@H]4C3)C(=O)N([C@H]3CCC(=O)NC3=O)C6)c2)cc1] | 610.46 | |
| C28 | 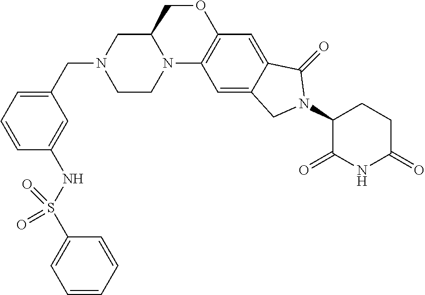 [SMILES: O=C1CC[C@H](N2Cc3cc4c(cc3C2=O)OC[C@@H]2CN(Cc3cccc(NS(=O)(=O)c5ccccc5)c3)CCN42)C(=O)N1] | 602.39 | |
| C29 | 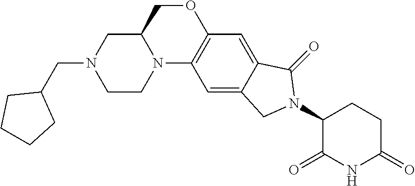 [SMILES: O=C1CC[C@H](N2Cc3cc4c(cc3C2=O)OC[C@@H]2CN(CC3CCCC3)CCN42)C(=O)N1] | 439.37 | |
| C30 | 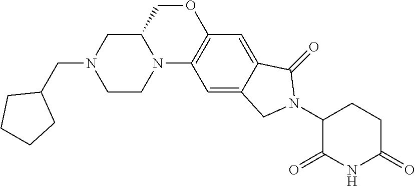 [SMILES: O=C1CCC(N2Cc3cc4c(cc3C2=O)OC[C@H]2CN(CC3CCCC3)CCN42)C(=O)N1] | 439.38 | |
| C31 | 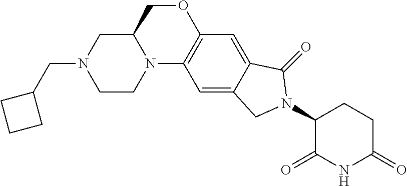 [SMILES: O=C1CC[C@H](N2Cc3cc4c(cc3C2=O)OC[C@@H]2CN(CC3CCC3)CCN42)C(=O)N1] | 425.25 | |
| C32 | 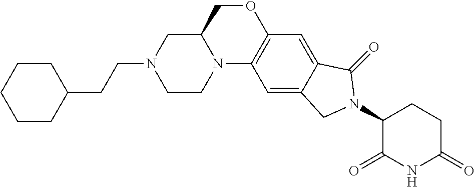 [SMILES: O=C1CC[C@H](N2Cc3cc4c(cc3C2=O)OC[C@@H]2CN(CCC3CCCCC3)CCN42)C(=O)N1] | 467.42 | |
| C33 | 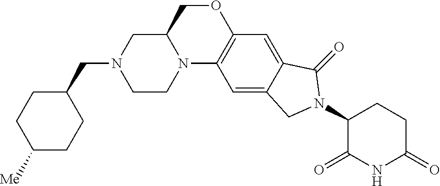 [SMILES: C[C@H]1CC[C@H](CN2CCN3c4cc5c(cc4OC[C@@H]3C2)C(=O)N([C@H]2CCC(=O)NC2=O)C5)CC1] | 467.41 | |
| C34 | 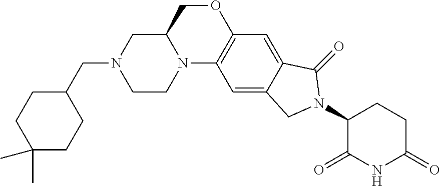 [SMILES: CC1(C)CCC(CN2CCN3c4cc5c(cc4OC[C@@H]3C2)C(=O)N([C@H]2CCC(=O)NC2=O)C5)CC1] | 481.36 | |
| C35 | 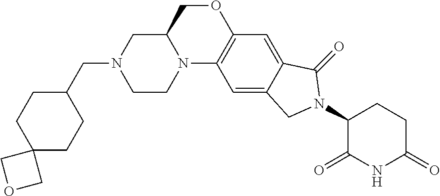 [SMILES: O=C1CC[C@H](N2Cc3cc4c(cc3C2=O)OC[C@@H]2CN(CC3CCC5(CC3)COC5)CCN42)C(=O)N1] | 495.31 | |
| C36 | 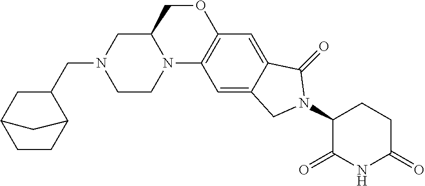 [SMILES: O=C1CC[C@H](N2Cc3cc4c(cc3C2=O)OC[C@@H]2CN(CC3CC5CCC3C5)CCN42)C(=O)N1] | 465.39 | |
| C37 | 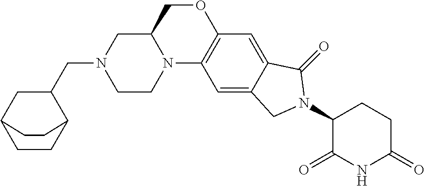 [SMILES: O=C1CC[C@H](N2Cc3cc4c(cc3C2=O)OC[C@@H]2CN(CC3CC5CCC3CC5)CCN42)C(=O)N1] | 479.36 | |
| C38 | 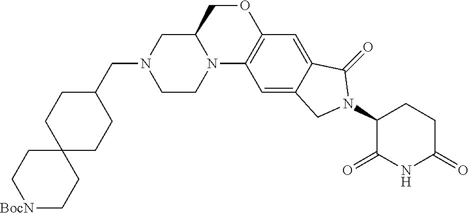 [SMILES: CC(C)(C)OC(=O)N1CCC2(CCC(CN3CCN4c5cc6c(cc5OC[C@@H]4C3)C(=O)N([C@H]3CCC(=O)NC3=O)C6)CC2)CC1] | 622.51 | |

### Biological Activity of Degraders

In-cell western blot analysis. a. seed cells in black-sided/clear bottom 96- or 384-well plates at 40,000 or 10,000 cells/well, overnight; b. add diluted compounds (final 0.5% DMSO), 16 hours. 16 h later, remove medium, add 100 μL or 25 μL of 3.7-4.0% formaldehyde (PBS:FA=9:1), RT 20 min, no shaking; c. wash with PBS, and permeabilized with 100 μL or 25 μL/well of 1×PBS+0.1% Triton X-100 10 minutes; d. block with 100 μL or 25 μL Licor blocking buffer (Li-Cor), RT 1 h, moderate shaking; d. Add 100 μL or 25 μL of anti-ER (cs-8644, 1:500-1,000)+GAPDH(Millipore MAB374, 1:1000) in Block+0.05% Tween 20. RT 2 h, gentle shaking. Negative control: cells plus secondary antibodies (no primary antibodies); e. wash×4 with PBS +0.05-0.1% Tween 20, gentel shaking; f. anti-rabbit-680 and anti-mouse-800 (both 1:1000 in LiCor block +0.05% Tween20, RT 1 h, gentle shaking, no light. LI-COR: 0.2% to reduce background; g. wash×4 with PBS+0.05% Tween 20, gental shaking; h. add 100 μL or 25 μL of PBS to each well and read on CLX plate reader. The relative ER percentage in treated cells were obtained by comparing the values of treated wells to those in untreated and DMSO-treated wells as 100%.

Western Blot Analysis. Western blot analysis was performed essentially as described previously. The cells treated with indicated compounds were lysed in Radioimmunoprecipitation Assay Protein Lysis and Extraction Buffer (25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail (Roche Diagnostics, Mannheim, Germany). Equal amounts of total protein were electrophoresed through 10% SDS-polyacrylamide gels after determination of protein concentration by BCA assay (Fisher Scientific, Pittsburgh, PA). The separated protein bands were transferred onto PVDF membranes (GE Healthcare Life Sciences, Marlborough, MA) and blotted against different antibodies, as indicated. The blots were scanned, and the band intensities were quantified using GelQuant.NET software provided by biochemlabsolutions.com. The relative mean intensity of target proteins was expressed after normalization to the intensity of glyceraldehyde-3-phosphate dehydrogenase bands.

### 1. ER Degraders

TABLE E5
ER degradation by in-cell western (ICW) assays

| Compound | MCF7 DC₅₀ | MCF7 Dₘₐₓ | T47D DC₅₀ | T47D Dₘₐₓ |
|---|---|---|---|---|
| No. | (nM) | (%) | (nM) | (%) |
| THP-A8 | A | A | A | A |
| THP-A21 | C | B | B | B |
| THP-A34 | A | A | A | A |
| THP-A35 | A | A | A | A |
| THP-A52 | A | B | A | A |
| THP-A56 | A | B | A | A |
| IND-A43 | A | B | A | B |
| IND-A48 | A | A | A | A |
| IND-A49 | A | A | A | B |
| IND-A50 | A | B | A | B |
| IND-A51 | A | A | A | A |
| IND-A54 | A | B | A | B |
| IND-A55 | A | B | A | B |
| IND-A59 | A | B | A | B |
| IND-A61 | A | B | A | B |
| IND-A63 | B | B | B | B |
| IND-A68 | A | B | A | A |
| IND-A70 | A | B | A | A |
| CHR-A59 | B | B | A | A |
| CHR-A73 | A | B | A | B |
| CHR-A89 | A | A | A | A |
| CHR-A100 | B | B | B | B |
| CHR-A106 | A | A | A | A |
| CHR-A117 | A | A | A | A |
| CHR-A118 | B | A | B | A |
| IDZ-A12 | A | A | A | A |
| IDZ-A15 | A | B | A | B |
| IDZ-A16 | A | A | A | A |
| IDZ-A17 | A | A | A | A |
| IDZ-A19 | A | B | A | B |
| IDZ-A21 | A | A | A | A |
| IDZ-A24 | A | B | A | A |
| IDZ-A25 | A | A | A | A |
| IDZ-A26 | A | A | A | A |

Note:
IC₅₀: “A”: <1 nM; “B”: 1-10 nM; “C”: >10 and <100 nM; “D”: >=100 nM.
Dₘₐₓ: “A”: >=75%; “B”: >50 and <75%; “C”: 25-50%; “D”: <25%.

### 2. IKZF 1/2/3 Degraders

TABLE E6
IKZF 1/2/3 degradation potency for selective
IKZF2 or IKZF1/3 degraders

IKZF2 degradation

| Compound No. | DC₅₀ (uM) | Dmax (%) |
|---|---|---|
| Control | 0.062 | 67 |
| (DKY709) | | |
| C1 | >2 | 27 |
| C2 | 1.4 | 56 |
| C3 | >2 | 58 |
| C4 | 0.43 | 61 |
| C5 | >2 | 38 |
| C6 | 0.33 | 62 |
| C7 | 0.32 | 50 |
| C19 | >2 | 0 |
| C20 | >10 | 27 |
| C21 | 0.009 | 85 |
| C22 | >1 | 44 |
| C23 | 0.018 | 77 |
| C24 | 0.025 | 77 |
| C25 | >1 | 45 |
| C26 | >1 | 33 |
| C27 | >10 | 21 |
| C28 | >1 | 11 |
| C29 | 0.402 | 61 |
| C30 | >1 | 0 |
| C31 | 1.261 | 58 |
| C32 | 0.324 | 71 |
| C33 | 0.515 | 67 |
| C34 | 0.414 | 68 |
| C35 | 0.933 | 66 |
| C36 | 0.376 | 68 |
| C37 | 0.318 | 69 |
| C38 | 0.424 | 57 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula II: 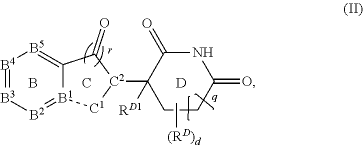
or a pharmaceutically acceptable salt, solvate, or stereoisomer thereof, wherein
B² is N or CR^{B2};
B³ is N or CR^{B3};
B⁴ is N or CR^{B4};
B⁵ is N or CR^{B5};
one of R^{B2} and R^{B3}, R^{B3} and R^{B4}, and R^{B4} and R^{B5}, together with the carbon atoms to which they are bonded, form Ring A, wherein Ring A is optionally substituted 7- to 16-membered fused carbocycle or optionally substituted 7- to 16-membered fused heterocycle;
the remaining two of R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; [symbol]
denotes an optional covalent bond between B¹ and C¹;
i) when the bond between B¹ and C¹ is present:
r is 1;
B¹ is C;
C¹ is —C(R^{C1})₂— or —C(═O)—;
each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{C1}, together with the carbon atom to which they are attached, form C₃₋₆ carbocycle or 3- to 6-membered heterocycle, wherein the carbocycle or heterocycle is optionally substituted with one or more R^{u}; and
C² is N:
ii) when the bond between B¹ and C₁ is absent:
r is 0 or 1;
B¹ is N or CR^{B1};
R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
C¹ is absent; or
C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR′R^{d}, —C(═O)R^{A}, —C(═O)OR^{b}, or —C(═O)NR^{c}R^{d}, wherein the alkyl, carbocyclyl, or heterocyclyl is optionally substituted with one or more R^{u};
C² is N or O;
wherein i) when C² is N, then C₁ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; and ii) when C² is O, then C¹ is absent;
R^{D1} is hydrogen, deuterium, or C₁₋₆ alkyl optionally substituted with one or more R^{U};
q is an integer from 0 to 2,
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
d is an integer selected from 0 to 5,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

2. A conjugate of Formula II: 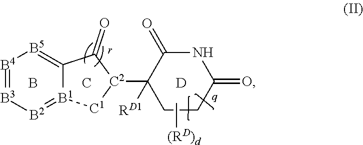
or a pharmaceutically acceptable salt, solvate, or stereoisomer thereof, wherein:
B² is N or CR^{B2};
B³ is N or CR^{B3};
B⁴ is N or CR^{B4};
B⁵ is N or CR^{B5};
one of R^{B2} and R^{B3}, R^{B3} and R^{B4}, and R^{B4} and R^{B5}, together with the carbon atoms to which they are bonded, form Ring A attached to -L-T, wherein Ring A is optionally substituted 7- to 16-membered fused carbocycle or optionally substituted 7- to 16-membered fused heterocycle;
the remaining two of R^{B2}, R^{B3}, R^{B4}, and R^{B5}, when applicable, are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NRCS(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; [symbol]
denotes an optional covalent bond between B¹ and C¹;
i) when the bond between B¹ and C¹ is present:
r is 1;
B¹ is C;
C¹ is —C(R^{C1})₂— or —C(═O)—;
each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{C1}, together with the carbon atom to which they are attached, form C₃₋₆ carbocycle or 3- to 6-membered heterocycle, wherein the carbocycle or heterocycle is optionally substituted with one or more R^{u}; and
C² is N:
ii) when the bond between B¹ and C¹ is absent:
r is 0 or 1;
B¹ is N or CR^{B1};
R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
C¹ is absent; or
C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
C² is N or O;
wherein i) when C² is N, then C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; and ii) when C² is O, then C¹ is absent;
R^{D1} is hydrogen, deuterium, or C₁₋₆ alkyl optionally substituted with one or more R^{u};
q is an integer from 0 to 2,
each R^{D} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
d is an integer selected from 0 to 5,
L is linker; and
T is a ligand for a protein,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

3. The compound or conjugate of claim 1 or 2, wherein
the compound is not 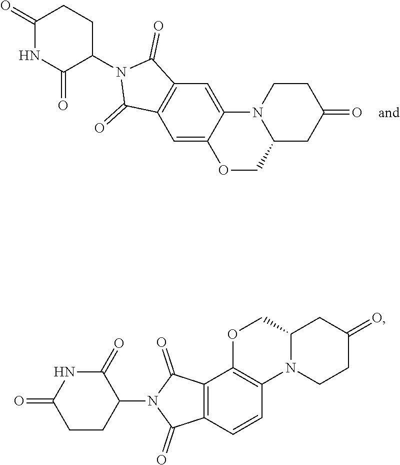 [SMILES: O=C1CCN2c3cc4c(cc3OC[C@H]2C1)C(=O)N(C1CCC(=O)NC1=O)C4=O ; O=C1CCN2c3ccc4c(c3OC[C@@H]2C1)C(=O)N(C1CCC(=O)NC1=O)C4=O]
or the conjugate is not

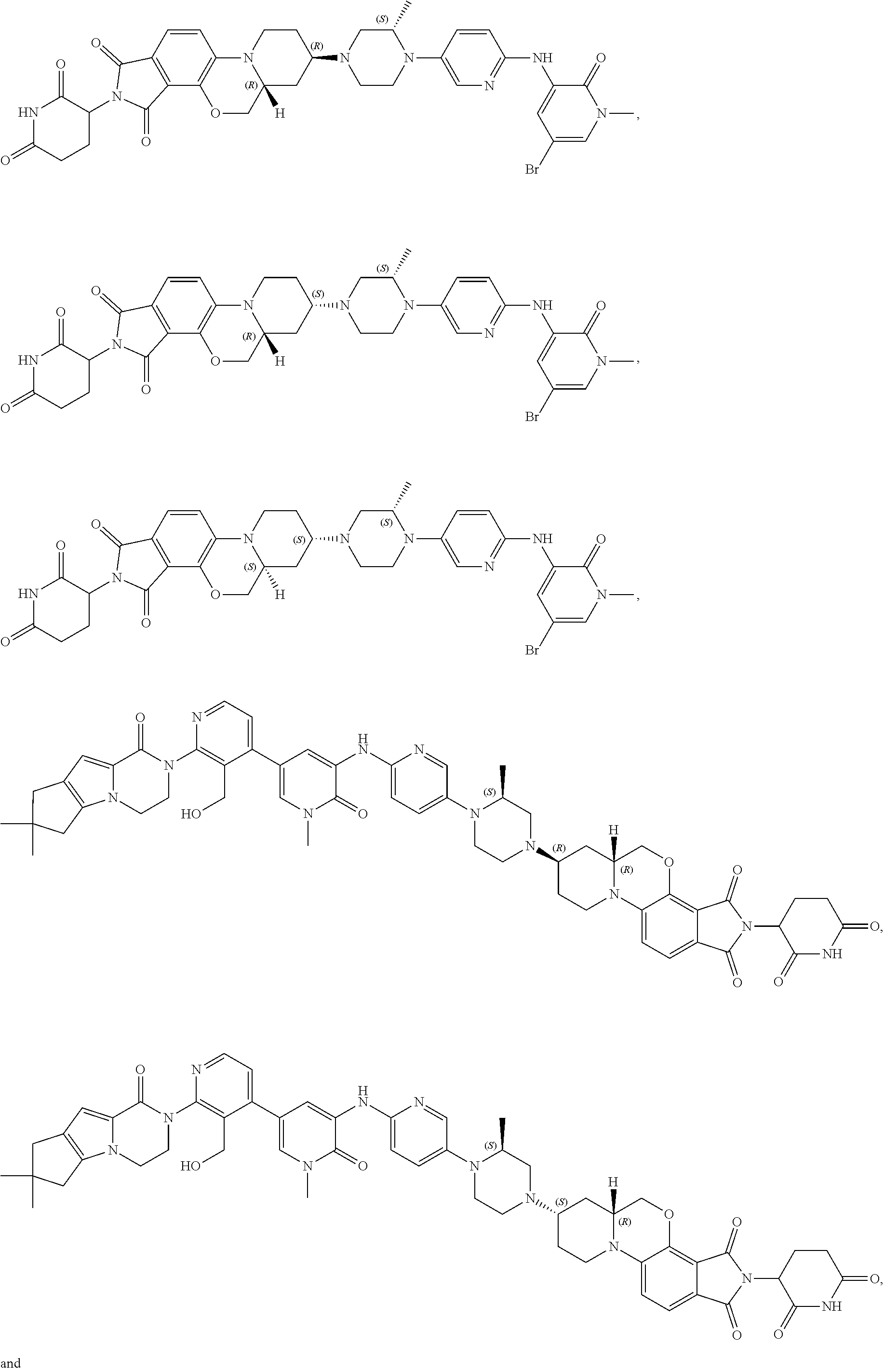

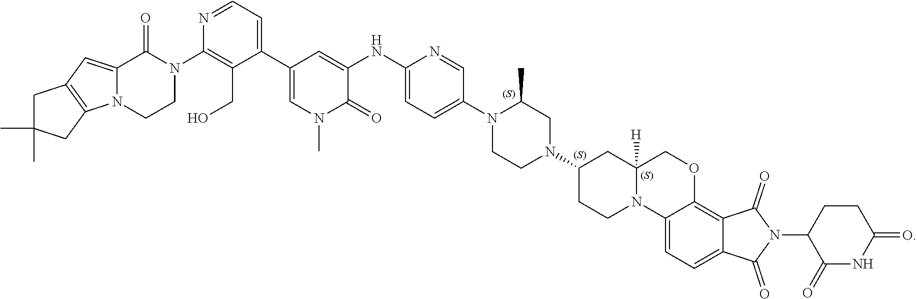

4. The compound or conjugate of any one of claims 1-3, wherein the compound or conjugate of Formula II is a compound or conjugate of Formula II-1 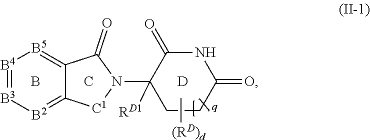
or a pharmaceutically acceptable salt, solvate, or stereoisomer thereof.

5. The compound or conjugate of claim 4, wherein C¹ is —CH₂—.

6. The compound or conjugate of any one of claims 1-3, wherein the compound or conjugate of Formula II is a compound or conjugate of Formula II-2 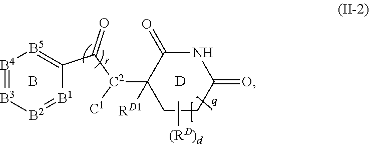
or a pharmaceutically acceptable salt, solvate, or stereoisomer thereof.

7. The compound or conjugate of claim 6, wherein C² is N and C₁ is hydrogen.

8. The compound or conjugate of claim 6, wherein B¹ is N.

9. The compound or conjugate of claim 6, wherein B¹ is CR^{B1}, wherein R^{B1} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

10. The compound or conjugate of claim 9, wherein R^{B1} is hydrogen or halogen.

11. The compound or conjugate of any one of claims 1-10, wherein
Ring A is 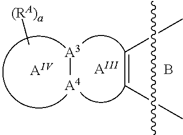
or
Ring A attached to -L-T is 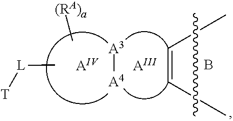
wherein:
Ring A^{III} and Ring A^{IV} are independently C₄₋₈ carbocycle or 4- to 8-membered heterocycle;
A³ and A⁴ are independently C, CR^{Ax}, or N;
R^{Ax} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits,
wherein R^{A} may be present on either Ring A^{III} or Ring A^{IV}.

12. The compound or conjugate of claim 11, wherein Ring A^{III} is 5- to 8-membered heterocycle comprising two nitrogen atoms.

13. The compound or conjugate of claim 11 or 12, wherein Ring A^{IV} is 5- to 8-membered heterocycle comprising two nitrogen atoms.

14. The compound or conjugate of claim 11, wherein
Ring A is 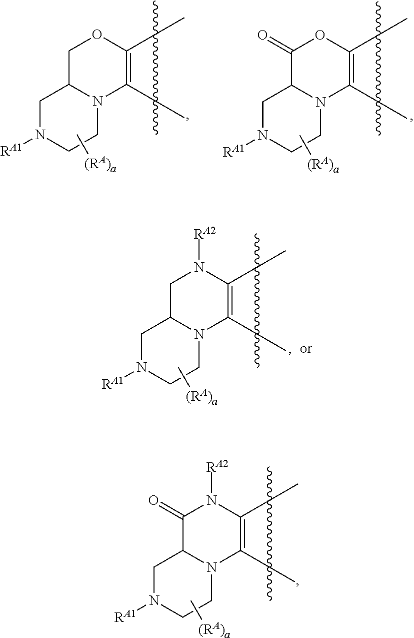
or
Ring A attached to L-T is 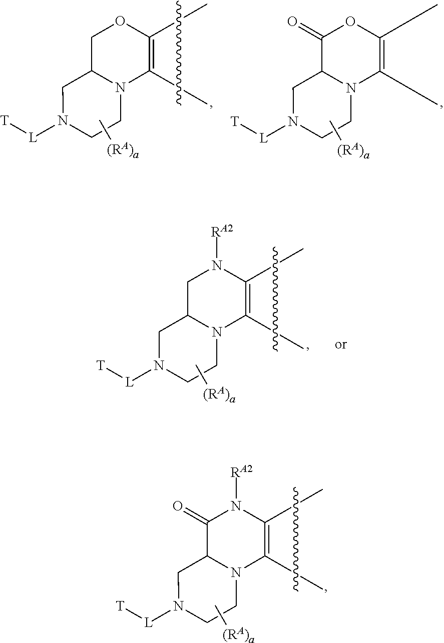
wherein:
R^{A1} is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), —(C₁₋₆ alkylene)-(5- to 10-membered heteroaryl), —S(═O)₂R^{A}, —S(═O)₂OR^{b}, —S(═O)₂NR′R^{d}, —C(═O)R^{A}, —C(═O)OR^{b}, or —C(═O)NR^{c}R^{d}, wherein the alkyl, alkylene, alkenyl, alkynyl, carbocyclyl, heterocyclyl, aryl, or heteroaryl is optionally substituted with one or more R^{u}; or
R^{A1} is an amino-protecting group; and
R^{A2} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; or
R^{A2} is an amino-protecting group.

15. The compound or conjugate of claim 14, wherein R^{A1} is hydrogen, C₁₋₆ alkyl, —(C₁₋₆ alkylene)-(C₆₋₁₀ aryl), —(C₁₋₆ alkylene)-(5- to 10-membered heteroaryl), —(C₁₋₆ alkylene)-(C₃₋₁₂ carbocyclyl), —(C₁₋₆ alkylene)-(3- to 12-membered heterocyclyl), —C(═O)R^{a}, or —C(═O)OR^{b}, wherein the alkyl, alkylene, carbocyclyl, heterocyclyl, or heteroaryl is optionally substituted with one or more R^{u}.

16. The compound or conjugate of claim 14 or 15, wherein R^{A2} is hydrogen or C₁₋₆ alkyl.

17. The compound or conjugate of any one of claims 11-16, wherein each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

18. The compound or conjugate of claim 17, wherein a is 0, 1, or 2.

19. The compound or conjugate of any one of claims 1-18, wherein R^{B2} and R^{B3}, together with the carbon atoms to which they are bonded, form Ring A or Ring A attached to -L-T.

20. The compound or conjugate of claim 19, wherein
the compound is a compound of Formula II-1-a-i, II-1-a-ii, II-1-a-iii, or II-1-a-iv 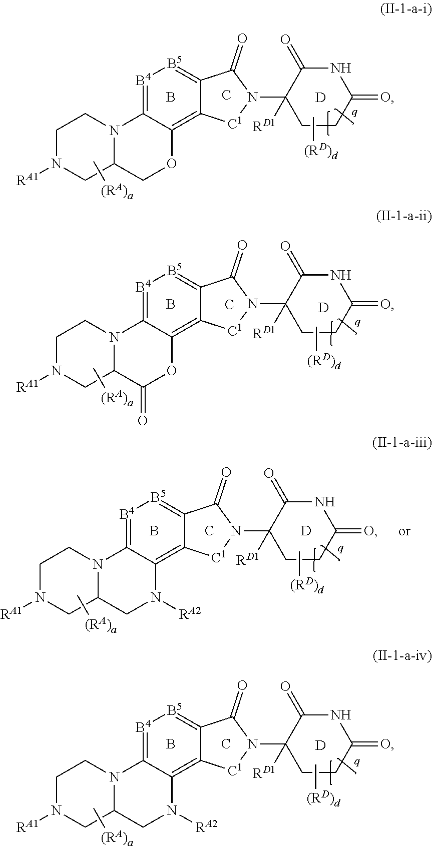
or a pharmaceutically acceptable salt, solvate, or stereoisomer thereof, or
the conjugate is a conjugate of Formula II-1-b-i, II-1-b-ii, II-1-b-iii, or II-1-b-iv: 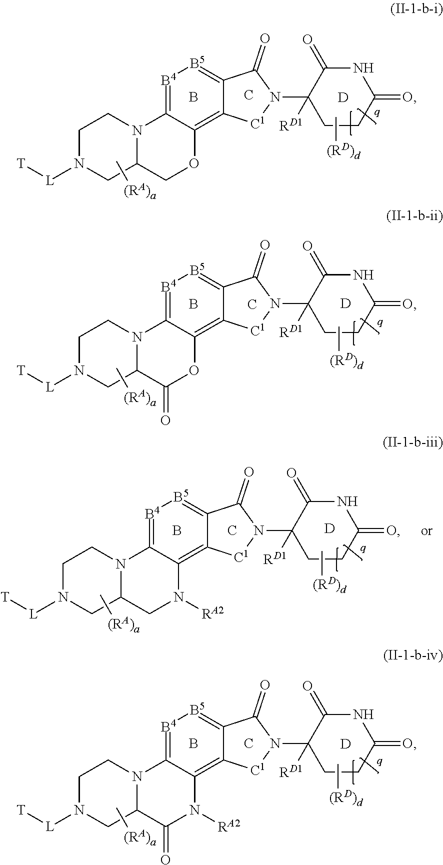
or a pharmaceutically acceptable salt, solvate, or stereoisomer thereof.

21. The compound or conjugate of claim 19, wherein
the compound is a compound of Formula II-2-a-i, II-2-a-ii, II-2-a-iii, or II-2-a-iv: 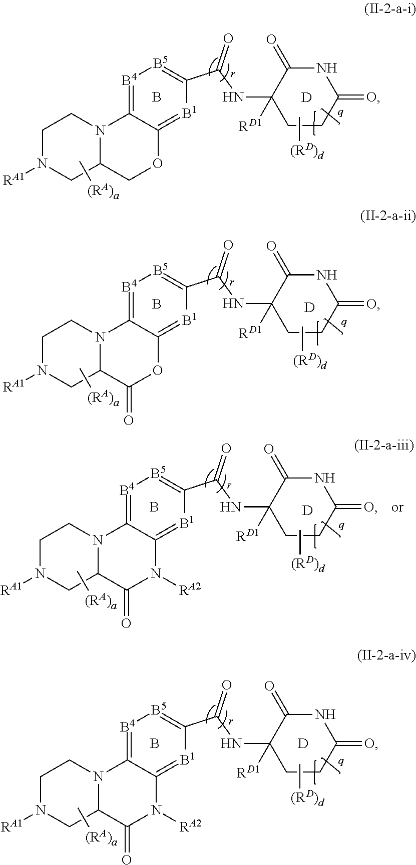
or a pharmaceutically acceptable salt, solvate, or stereoisomer thereof, or
the conjugate is a conjugate of Formula II-2-b-i, II-2-b-ii, II-2-b-iii, or II-2-b-iv 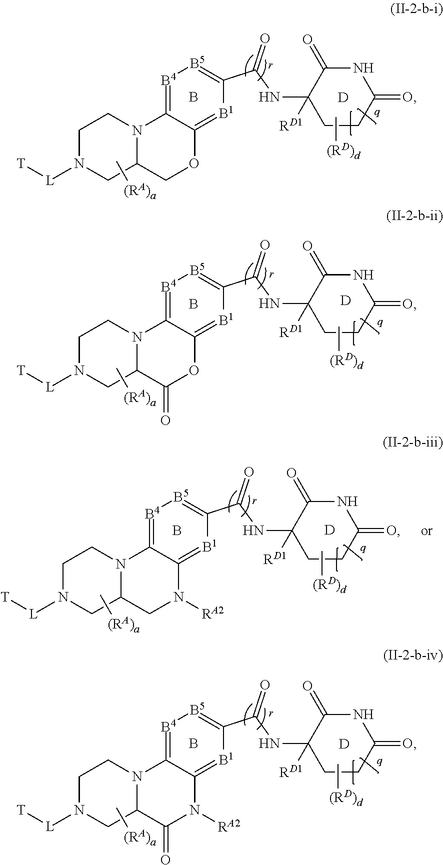
or a pharmaceutically acceptable salt, solvate, or stereoisomer thereof.

22. The compound or conjugate of any one of claims 19-21, wherein B⁴ is CR^{B4} and B⁵ is CR^{B5}, wherein R^{B4} and R^{B5} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

23. The compound or conjugate of claim 22, wherein each of R^{B4} and R^{B5} is hydrogen.

24. The compound or conjugate of any one of claims 1-16, wherein R^{B3} and R^{B4}, together with the carbon atoms to which they are bonded, form Ring A or Ring A attached to -L-T.

25. The compound or conjugate of claim 24, wherein
the compound is a compound of Formula II-1-a-v, II-1-a-vi, II-1-a-vii, or II-1-a-viii: 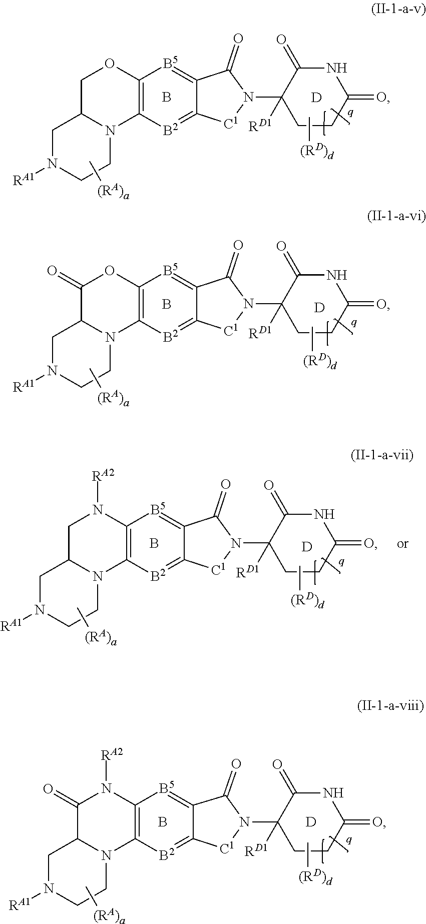
or a pharmaceutically acceptable salt, solvate, or stereoisomer thereof, or
the conjugate is a conjugate of Formula II-1-b-v, II-1-b-vi, II-1-b-vii, or II-1-b-viii 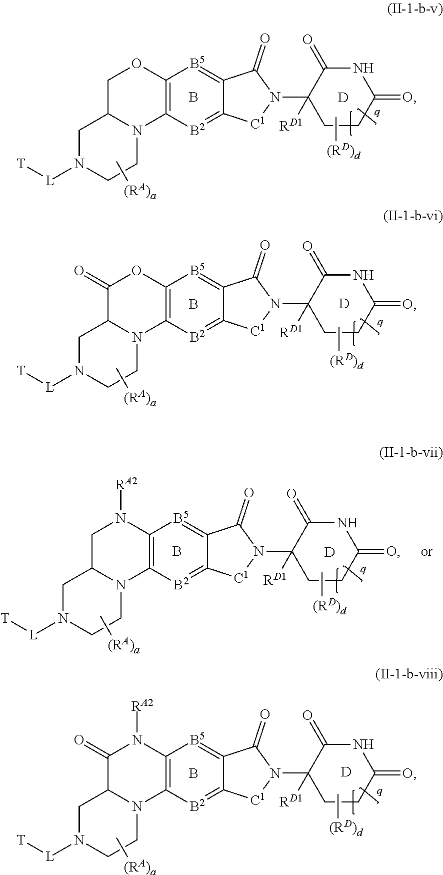
or a pharmaceutically acceptable salt, solvate, or stereoisomer thereof.

26. The compound or conjugate of claim 24, wherein
the compound is a compound of Formula II-2-a-v, II-2-a-vi, II-2-a-vii, or II-2-a-viii 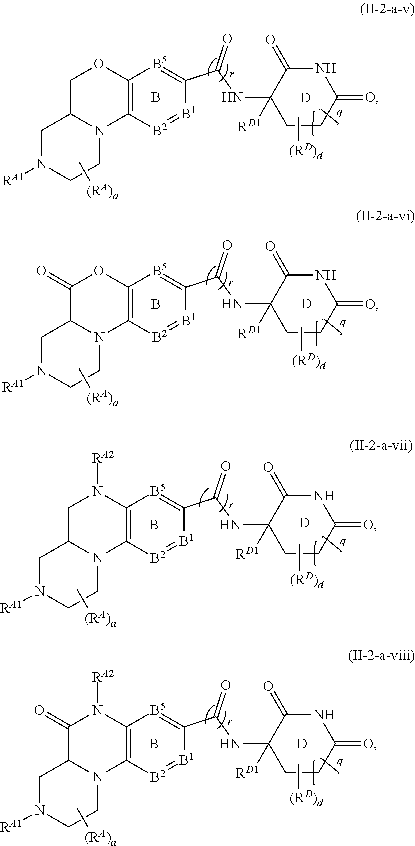
or a pharmaceutically acceptable salt, solvate, or stereoisomer thereof, or
the conjugate is a conjugate of Formula II-2-b-v, II-2-b-vi, II-2-b-vii, or II-2-b-viii 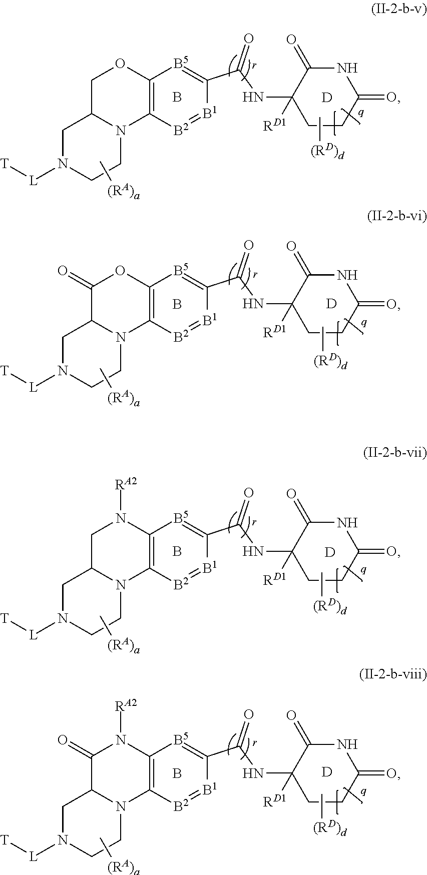
or a pharmaceutically acceptable salt, solvate, or stereoisomer thereof.

27. The compound or conjugate of any one of claims 24-26, wherein B² is CR^{B2} and B⁵ is CR^{B5}, wherein R^{B2} and R^{B5} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

28. The compound or conjugate of claim 27, wherein each of R^{B2} and R^{B5} is hydrogen.

29. The compound or conjugate of any one of claims 1-28, wherein R^{D1} is hydrogen.

30. The compound or conjugate of any one of claims 1-29, wherein d is 0.

31. The compound or conjugate of any one of claims 1-30, wherein q is 1.

32. A compound selected from the compounds in Tables 1-3 or a pharmaceutically acceptable salt thereof.

33. A pharmaceutical composition comprising the compound of any one of claims 1-32, and a pharmaceutically acceptable excipient.

34. A method of binding cereblon E3 ubiquitin ligase protein complex in a subject or biological sample comprising administering the compound of any one of claims 1-32 to the subject or contacting the biological sample with the compound of any one of claims 1-32.

35. Use of the compound of any one of claims 1-32 in the manufacture of a medicament for binding cereblon E3 ubiquitin ligase protein complex in a subject or biological sample.

36. A compound of any one of claims 1-32 for use in binding cereblon E3 ubiquitin ligase protein complex in a subject or biological sample.

37. A method of degrading a protein in a subject or biological sample comprising administering the conjugate of any one of claims 2-31 to the subject or contacting the biological sample with the conjugate of any one of claims 2-31.

38. Use of the conjugate of any one of claims 2-31 in the manufacture of a medicament for degrading a protein in a subject or biological sample.

39. A conjugate of any one of claims 2-31 for use in degrading a protein in a subject or biological sample.

40. The method, use, or compound for use of any one of claims 37-39, wherein the protein is an estrogen receptor, a STAT3 protein, an androgen receptor, a SMARCA2 protein, a SMARCA4 protein, a BRD4 protein, a BRD9 protein, or a CBP/p300 protein.
